SEQUENCE LISTING (70 sequences)

SEQ ID NO: 1 - DNA, 15108 bp, synthetic construct
  misc_feature 1..15108: note = CVB backbone nucleic acid sequence
  source 1..15108: mol_type = other DNA
accaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggt

SEQ ID NO: 2 - DNA, 16854 bp, synthetic construct
  misc_feature 1..16854: note = CVB-RSV-F nucleic acid sequence
  source 1..16854: mol_type = other DNA
accaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcgccgccaccatggagctgctgatcctgaaagccaacgctattaccacaatcctgaccgccgtgacattctgctttgcttccggacagaatatcaccgaggagttctaccagtcaacatgtagcgccgtgagcaaggggtacctgagtgcactgcgaaccggatggtatacatccgtgattactatcgagctgtctaacatcaagaaaaacaaatgcaatggcactgacgccaagatcaagctgatcaagcaggagctggataagtacaaaaatgctgtgaccgaactgcagctgctgatgcagagcactccagctaccaacaatcgcgcacggagagaactgccccggttcatgaactataccctgaacaatgcaaagaaaacaaatgtgactctgtccaagaaacgcaagaggcgcttcctgggatttctgctgggcgtggggtctgcaatcgccagtggagtggccgtctctaaagtcctgcacctggagggcgaagtgaacaagatcaaatcagcactgctgagcacaaacaaggccgtggtcagtctgtcaaatggcgtgtccgtcctgacttctaaggtgctggacctgaaaaattatattgataagcagctgctgcctatcgtcaacaaacagagctgttccatttctaatatcgagaccgtgatcgagttccagcagaagaacaatagacttctagagattacaagggaattttctgtgaacgctggcgtcactacccccgtgagtacctacatgctgacaaatagtgagctgctgtcactgattaacgacatgcctatcaccaatgatcagaagaaactgatgagtaacaatgtgcagatcgtcagacagcagagttactcaatcatgtcaatcattaaggaggaagtcctggcctacgtggtccagctgccactgtatggcgtgatcgacactccctgctggaaactgcatacctcacctctgtgcacaactaacaccaaggaggggagcaatatctgcctgacacgaactgaccggggatggtactgtgataacgccggcagcgtgtccttctttccccaggctgagacctgcaaggtccagtccaacagggtgttctgtgacactatgaatagcctgaccctgccttccgaagtcaacctgtgcaatgtggacatctttaatccaaagtacgattgtaagatcatgaccagcaagacagatgtcagctcgagtgtgattacatcactgggggctatcgtgagctgctacggaaagactaaatgtaccgcaagcaacaaaaatcgcggcatcattaagaccttcagtaacgggtgcgactatgtctcaaacaagggcgtggatacagtgagcgtcgggaacactctgtactatgtcaataagcaggagggaaaatccctgtacgtgaagggcgaaccaatcattaacttctatgaccccctggtgttcccttccgacgagtttgatgcctctattagtcaggtgaacgaaaaaatcaatcagagcctggcttttattcggaagtccgatgagctgctgcacaacgtgaatgcagtcaagtctaccacaaacattatgatcactaccatcattatcgtgattatcgtcattctgctgtccctgatcgcagtggggctgctgctgtactgtaaagccagatctacacccgtgactctgtctaaggatcagctgagtggaattaacaatatcgcctttagcaactgataggtcgaccacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggt

SEQ ID NO: 3 - DNA, 21375 bp, synthetic construct
  misc_feature 1..21375: note = nucleic acid sequence for CVL104
  source 1..21375: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaagtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 4 - DNA, 21231 bp, synthetic construct
  misc_feature 1..21231: note = nucleic acid sequence for CVL87
  source 1..21231: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaagtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 5 - DNA, 22653 bp, synthetic construct
  misc_feature 1..22653: note = nucleic acid sequence for CVL85
  source 1..22653: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcgcgcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgacgtacgacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaagtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 6 - DNA, 22647 bp, synthetic construct
  misc_feature 1..22647: note = nucleic acid sequence for CVL83
  source 1..22647: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcgcgcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgacgtacgacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgtgatcagcggcaccaatggcacaaagcggttcgacaatcccgtgctgccttttaacgatggcgtgtacttcgcctctatcgagaagagcaacatcatcagaggctggatctttggcaccacactggactccaagacacagtctctgctgatcgtgaacaatgccaccaacgtggtcatcaaggtgtgcgagttccagttttgtaatgatcccttcctggatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcatcgtgcgcgagcctgaggacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttgacgaggtgttcaacgcaacccgcttcgccagcgtgtacgcctggaataggaagcggatcagcaactgcgtggccgactatagcgtgctgtacaacctggcccccttcttcacctttaagtgctatggcgtgtcccccacaaagctgaatgacctgtgctttaccaacgtctacgccgattctttcgtgatcaggggcgacgaggtgcgccagatcgcccccggccagacaggcaatatcgcagactacaattataagctgccagacgatttcaccggctgcgtgatcgcctggaacagcaacaagctggattccaaagtgtccggcaactacaattatctgtaccggctgtttagaaagagcaatctgaagcccttcgagagggacatctctacagaaatctaccaggccggcaataagccttgcaatggcgtggccggctttaactgttatttcccactcaagtcctactccttccgccccacatatggcgtgggccaccagccttaccgcgtggtggtgctgagctttgagctgctgcacgccccagcaacagtgtgcggccccaagaagtccaccaatctggtgaagaacaagtgcgtgaacttcaacttcaacggcctgaagggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaagtctcacagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaagagagccctgacaggcatcgccgtggagcaggataagaacacacaggaggtgttcgcccaggtgaagcaaatctacaagaccccccctatcaagtactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaagggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcacaacgcccaggccctgaataccctggtgaagcagctgagcagcaagttcggcgccatctctagcgtgctgaatgacatctttagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaagtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 7 - DNA, 22641 bp, synthetic construct
  misc_feature 1..22641: note = nucleic acid sequence for CVL109
  source 1..22641: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcgcgcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgacgtacgacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccaccatgttcgtcttcctggtcctgctgcctctggtctcctcacagtgcgtcaatctgatcactcggactcagtcctatactaatagcttcaccagaggcgtgtactatcctgacaaggtgtttagaagctccgtgctgcactctacacaggatctgtttctgccattctttagcaacgtgacctggttccacgccatcagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctggacgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctgggccgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttgacgaggtgttcaacgcaacccgcttcgccagcgtgtacgcctggaataggaagcggatcagcaactgcgtggccgactatagcgtgctgtacaacctggcccccttcttcgcctttaagtgctatggcgtgtcccccacaaagctgaatgacctgtgctttaccaacgtctacgccgattctttcgtgatcaggggcaacgaggtgtcccagatcgcccccggccagacaggcaatatcgcagactacaattataagctgccagacgatttcaccggctgcgtgatcgcctggaacagcaacaagctggattccaaagtgggcggcaactacaattatagataccggctgtttagaaagagcaatctgaagcccttcgagagggacatctctacagaaatctaccaggccggcaataagccttgcaatggcgtggccggcgtgaactgttatttcccactccagtcctacggcttccgccccacatatggcgtgggccaccagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaagtctcacagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaagagagccctgacaggcatcgccgtggagcaggataagaacacacaggaggtgttcgcccaggtgaagcaaatctacaagaccccccctatcaagtactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcacaacgcccaggccctgaataccctggtgaagcagctgagcagcaagttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaagtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 8 - DNA, 21279 bp, synthetic construct
  misc_feature 1..21279: note = nucleic acid sequence for CVL110
  source 1..21279: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgcacgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgtgaaatcaaattggggtctgttcaagaaaactaaatcactgcctaacctgacggagctggaatgactgacctctaatcgagactacaccgccgcaaactataggtgggtggtacctcagtgattaatcttgtaagcactgaccgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccaaaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcagctagccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcgcgcggccgccaccatgttcgtcttcctggtcctgctgcctctggtctcctcacagtgcgtcaatctgatcactcggactcagtcctatactaatagcttcaccagaggcgtgtactatcctgacaaggtgtttagaagctccgtgctgcactctacacaggatctgtttctgccattctttagcaacgtgacctggttccacgccatcagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctggacgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctgggccgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttgacgaggtgttcaacgcaacccgcttcgccagcgtgtacgcctggaataggaagcggatcagcaactgcgtggccgactatagcgtgctgtacaacctggcccccttcttcgcctttaagtgctatggcgtgtcccccacaaagctgaatgacctgtgctttaccaacgtctacgccgattctttcgtgatcaggggcaacgaggtgtcccagatcgcccccggccagacaggcaatatcgcagactacaattataagctgccagacgatttcaccggctgcgtgatcgcctggaacagcaacaagctggattccaaagtgggcggcaactacaattatagataccggctgtttagaaagagcaatctgaagcccttcgagagggacatctctacagaaatctaccaggccggcaataagccttgcaatggcgtggccggcgtgaactgttatttcccactccagtcctacggcttccgccccacatatggcgtgggccaccagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaagtctcacagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaagagagccctgacaggcatcgccgtggagcaggataagaacacacaggaggtgttcgcccaggtgaagcaaatctacaagaccccccctatcaagtactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcacaacgcccaggccctgaataccctggtgaagcagctgagcagcaagttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaacgcgtgtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 9 - DNA, 17511 bp, synthetic construct
  misc_feature 1..17511: note = nucleic acid sequence for CVL112
  source 1..17511: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgagctagcggcagaagccttcaggtctgacccagccaatcaaagcattataccagaccatggcctaccatcggctttaaagaaaaaaataggcccggacgggttagcaacaagcggcggccgccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaagtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 10 - DNA, 22659 bp, synthetic construct
  misc_feature 1..22659: note = nucleic acid sequence for CVL119
  source 1..22659: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcgcgcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgacgtacgacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaataaggtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 11 - DNA, 22071 bp, synthetic construct
  misc_feature 1..22071: note = nucleic acid sequence for CVL120
  source 1..22071: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcgcgcgccaccatggccgatagcaacggcaccatcaccgtggaagagcttaaaaagctgctcgagcagtggaacctggttatcggctttctgttcctgacctggatctgcctgctccagttcgcttatgccaaccggaacaggttcctgtacatcatcaagctgatcttcctgtggctgctgtggcctgtgacactggcctgcttcgtgctggccgctgtgtaccggattaactggatcaccggcggaatcgccatcgccatggcctgtctggtcggcctgatgtggctgtcttacttcatcgccagcttcagactgtttgccagaaccagaagcatgtggtccttcaaccccgagacaaacatcctgctgaatgtgccactgcacggcaccatcctgacaagacctctgctggaaagcgagctggtgattggagccgtgatcctgagaggccatctgcggatcgctggccaccacctgggaagatgcgacatcaaggacctgcctaaggaaatcacagtggctacatctcggaccctgagctactacaaactgggcgcctctcaaagagtggccggcgacagcggcttcgccgcctacagcagatacagaatcggcaactacaagctgaataccgatcacagcagctccagcgacaacatcgccctgctggtgcagtgatgacgtacgacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaataaggtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 12 - DNA, 22467 bp, synthetic construct
  misc_feature 1..22467: note = nucleic acid sequence for CVL121
  source 1..22467: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcgcgcgccaccatggccgatagcaacggcaccatcaccgtggaagagcttaaaaagctgctcgagcagtggaacctggttatcggctttctgttcctgacctggatctgcctgctccagttcgcttatgccaaccggaacaggttcctgtacatcatcaagctgatcttcctgtggctgctgtggcctgtgacactggcctgcttcgtgctggccgctgtgtaccggattaactggatcaccggcggaatcgccatcgccatggcctgtctggtcggcctgatgtggctgtcttacttcatcgccagcttcagactgtttgccagaaccagaagcatgtggtccttcaaccccgagacaaacatcctgctgaatgtgccactgcacggcaccatcctgacaagacctctgctggaaagcgagctggtgattggagccgtgatcctgagaggccatctgcggatcgctggccaccacctgggaagatgcgacatcaaggacctgcctaaggaaatcacagtggctacatctcggaccctgagctactacaaactgggcgcctctcaaagagtggccggcgacagcggcttcgccgcctacagcagatacagaatcggcaactacaagctgaataccgatcacagcagctccagcgacaacatcgccctgctggtgcagtgatgacgtacggatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacgcgtgccaccatgtattctttcgtgtctgaagagacaggcaccctgatcgttaatagcgtgctgctgtttctggccttcgtggtgttcctgctggtgaccctggctatcctgaccgccctgagactgtgtgcctactgctgcaacatcgtgaacgtgtccctggtcaagcctagcttctacgtgtacagccgggtgaagaacctgaacagcagcagagtgcccgacctgctcgtgtaagctagcacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaataaggtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 13 - DNA, 23847 bp, synthetic construct
  misc_feature 1..23847: note = nucleic acid sequence for CVL128
  source 1..23847: mol_type = other DNA
gccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggctaatacgactcactatagggaccaaggggaaaatgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacgctcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattactcacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacacctttcgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgacgaataatccagaatgtaatccggaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaactagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaacaagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctgtcactgggacaactcaggtgcccgctgcagcaacaggtgacacactcgagagttacatggcagcggatcgactgaggcagagatatgctgatgcaggcacccatgatgatgagatgccaccattggaagaggaggaagaggacgacacatctgcaggtccacgcactggaccaactcttgaacaagtggccttggacatccagaacgcagcagttggagctcccatccatacagatgacctgaatgccgcactgggtgatcttgacatctagacaattcagatcccaatctaaaattgacatacctaattgattagttagatggaactacagtggattccataaggttcctgcctaccatcggctttaaagaaaaaaataggcccggacgggttagcaacaagcgactgccggtgccaacagcgcaatccacaatctacaatggatcccactgatctgagcttctccccagatgagatcaataagctcatagagacaggcctgaatactgtagagtattttacttcccaacaagtcacaggaacatcctctcttggaaagaatacaataccaccaggggtcacaggactactaaccaatgctgcagaggcaaagatccaagagtcaactaaccatcagaagggctcagttggtgggggtgcaaaaccaaagaaaccgcgaccaaaaattgccattgtgccagcagatgacaaaacagtgcccggaaagccgatcccaaaccctctattaggtctggactccaccccgagcacccaaactgtgcttgatctaagtgggaaaacattaccatcaggatcctataagggggttaagcttgcgaaatttggaaaagaaaatctgatgacacggttcatcgaggaacccagagagaatcctatcgcaaccagtttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagacactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagattacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacactacctgaggcagccatgccatccatcagcattcccgcagaccccaccaatccacgtcaatcaataaaagcgttcccaattgtgatcaacagtgatgggggtgagaaaggccgcttggttaaacaactacgcacaacctacttgaatgacctagatactcatgagccactggtgacattcataaatacctatggattcatctacgaacaggatcgggggaataccattgtcggagaggatcaacttgggaagaaaagagaggctgtgaccgctgcaatggttacccttggatgtgggcctaatctaccatcattagggaatgtcctgggacaactgagggaattccaggtcactgttaggaagacatccagcaaagcggaagagatggtctttgaaattgttaagtatccgagaatatttcggggtcatacattaatccagaaaggactagtctgtgtctccgcagaaaaatttgttaagtcaccagggaaaatacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatacccctgacaaggagggaagaggatgtcttgtatcagtatggctgcatgtatgcaacatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaactaaatcactgcctaacctgacggagctggaatgactgacctctaatcgagactacaccgccgcaaactataggtgggtggtacctcagtgattaatcttgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagccttgatccagcagccctcatgcaaatcggtgtcattccaacaaatgtccggcaacttatgtattatactgaagcttcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtggcactagtaaaggcaaatgaaaatgctgcggctatactcaatctcaaaaatgcaatccaaaaaacaaatgcagcagttgcagatgtggtccaggccacacaatcactaggaacggcagttcaagcagttcaagatcacataaacagtgtggtaagtccagcaattacagcagccaattgtaaggcccaagatgctatcattggctcaatcctcaatctctatttgaccgagttgacaaccatcttccacaatcaaattacaaaccctgcattgagtcccattacaattcaagctttaaggatcctactggggagtaccttgccgactgtggtcgaaaaatctttcaatacccagataagtgcagctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttatggtgactggcagcttgatccaagcctatcccgcatcgcaatgcaccattacacccaacactgtgtactgtaggtataatgatgcccaagtactctcagatgatactatggcttgcctccaaggtaacttgacaagatgcaccttctctccagtggttgggagctttctcactcgattcgtgctgttcgatggaatagtttatgcaaattgcaggtcgatgttgtgcaagtgcatgcaacctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaatcatcccagatcttgtctattgatccgttggatatatcccagaatctagctgcggtgaataagagtctaagtgatgcactacaacacttagcacaaagtgacacatatctttctgcaatcacatcagctacgactacaagtgtattatccataatagcaatctgtcttggatcgttaggtttaatattaataatcttgctcagtgtagttgtgtggaagttattgaccattgtcgttgctaatcgaaatagaatggagaattttgtttatcataaataagcattccaccactcacgatctgatctcagtgagaaaaatcaacctgcaactcttggaacaagataagacagtcatccattagtaatttttaagaaaaaaacgataggaccgaacctagtattgaaagaaccgtctcggtcaatctaggtaatcgagctgataccgtctcggaaagctcaaatcgcgcgccaccatggccgatagcaacggcaccatcaccgtggaagagcttaaaaagctgctcgagcagtggaacctggttatcggctttctgttcctgacctggatctgcctgctccagttcgcttatgccaaccggaacaggttcctgtacatcatcaagctgatcttcctgtggctgctgtggcctgtgacactggcctgcttcgtgctggccgctgtgtaccggattaactggatcaccggcggaatcgccatcgccatggcctgtctggtcggcctgatgtggctgtcttacttcatcgccagcttcagactgtttgccagaaccagaagcatgtggtccttcaaccccgagacaaacatcctgctgaatgtgccactgcacggcaccatcctgacaagacctctgctggaaagcgagctggtgattggagccgtgatcctgagaggccatctgcggatcgctggccaccacctgggaagatgcgacatcaaggacctgcctaaggaaatcacagtggctacatctcggaccctgagctactacaaactgggcgcctctcaaagagtggccggcgacagcggcttcgccgcctacagcagatacagaatcggcaactacaagctgaataccgatcacagcagctccagcgacaacatcgccctgctggtgcagtgatgacgtacgagaaacacccaattacactacactggtatgacactgtactaaccctgagggttttagaaaaaacgattaacgataaataagcccgaacactacacactacctgaggcagccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgacgtacggatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacgcgtgccaccatgtattctttcgtgtctgaagagacaggcaccctgatcgttaatagcgtgctgctgtttctggccttcgtggtgttcctgctggtgaccctggctatcctgaccgccctgagactgtgtgcctactgctgcaacatcgtgaacgtgtccctggtcaagcctagcttctacgtgtacagccgggtgaagaacctgaacagcagcagagtgcccgacctgctcgtgtaagctagcacctgctataggctatccactgcatcatctctcctgccatacttcctactcacatcatatctattttaaagaaaaaataggcccgaacactaatcgtgccggcagtgccactgcacacacaacactacacatacaatacactacaatggttgcagaagatgcccctgttagggccacttgccgagtattatttcgaacaacaactttaatctttctatgcacactactagcattaagcatctctatcctttatgagagtttaataacccaaaagcaaatcatgagccaagcaggctcaactggatctaattctggattaggaagtatcactgatcttcttaataatattctctctgtcgcaaatcagattatatataactctgcagtcgctctacctctacaattggacactcttgaatcaacactccttacagccattaagtctcttcaaaccagtgacaagctagaacagaactgctcgtggagtgctgcactgattaatgataatagatacattaatggcatcaatcagttctatttttcaattgctgagggtcgcaatctgacacttggcccacttcttaatatgcctagtttcattccaactgccacgacaccagagggctgcaccaggatcccatcattctcgctcactaagacacactggtgttatacacacaatgttatcctgaatggatgccaggatcatgtatcctcaaatcaatttgtttctatgggaatcattgaacccacttctgccgggtttccattctttcgaaccctaaagactctatatctcagcgatggggtcaatcgtaagagctgctctatcagtacagttccggggggttgtatgatgtactgttttgtttctactcaaccagagagggatgactacttttctgccgctcctccagaacaacgaattattataatgtactataatgatacaatcgtggagcgcataattaatccacccggggtactagatgtatgggcaacattgaacccaggaacaggaagcggggtatattatttaggttgggtgctctttccaatatatggcggcgtgattaaaggtacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcgacaggatctaaccaatgagtgtttagttctgcccttttctaatgatcaggtgcttatgggtgctgaagggagattatacatgtatggtgactcggtgtattactatcaaagaagcaatagttggtggcctatgaccatgctgtataaggtaaccataacattcactaatggtcagccatctgctatatcagctcagaatgtgcccacacagcaggtccctagacctgggacaggagactgctctgcaaccaatagatgtcccggtttttgcttgacaggagtgtatgccgatgcctggttactgaccaacccttcgtctaccagtacatttggatcagaagcaaccttcactggttcttatctcaacacagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaataaggtcgactcatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgaccttgacgatttaggtccattacataatcaaaattggaatcagatagcacatgaagagtctaacttagctcaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctgctcaaaaattgggacaagttagttaaagaatcatgttcagtaatcaatgcaggtacttcccagtgcattcagaatctcagctatggactgacaggtcgtgggaacctctttacacgatcacgtgaactctctggtgaccgcagggatattgatcttaagacagttgtggcagcatggcatgactcagactggaaaagaataagtgatttttggattatgatcaaattccagatgagacaattaattgttaggcaaacagatcataatgattctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaagaggaactaatagtttcacccaggatgaattaagaactgtgacaactaatttgatatccccattccaagatctgaccccagatcttacggctgaattgctctgtataatgaggctttggggacaccccatgctcactgccagtcaagctgcaggaaaggtacgcgagtctatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgaaataagctatgaattcacacttaagcattggaaggaagtctctcttataaaattcaagaaatgttttgatgcagacgcaggtgaggaactcagtatatttatgaaagataaggcaattagtgccccaaaacaagactggatgagtgtgtttagaagaagcctaatcaaacagcgccatcagcatcatcaggtccccctaccaaatccattcaatcgacggctgttgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaagtcaactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctgtgtcaaaaggcttggacaatgatatctatcgctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgcacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttttgccatcccagttaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccctccaactacagctcttaagaggcacacccaacaagctctgatggaactcagtacgaatccaatgttacgtggcatattctctgacaatgcacaggcagaagaaaataaccttgctaggtttctcctggatagggaggtgatctttccgcgtgtagctcacatcatcattgagcaaaccagtgtcgggaggagaaaacagattcaaggatatttggattcaactagatcgataatgaggaaatcactagaaattaagcccttatccaataggaagcttaatgaaatactggattacaacatcaattacctagcttacaatttggcattactcaagaatgctattgaacctccgacttatttgaaggcaatgacacttgaaacatgtagcatcgacattgcaaggaacctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcttaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaatcttagaaacatggaataatcccccaatcaatagaacattcgaagaatctaccctacatttgcacactggtgcatcatgttgtgtccgacctgtggactcctgcattctctcagaagcattaacagtcaagccacatattacagtaccgtacagcaataaatttgtatttgatgaggacccgctatctgaatatgaaactgcaaaactggaatcgttatcattccaagcccaattaggcaacattgatgctgtagatatgacaggtaaattaacattattgtcccaattcactgcaaggcagattatcaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggcacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgctgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaacgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctgatccagctaatcatgaggtttatttaatttgtatccttgccaacaactttgcattccagactgtctcgcaggcaacaggaatggcgatgactttaactgatcaagggtttactttgatatcacctgaaagaataaatcagtattgggatggtcacttgaagcaagaacgtatcgtagcagaagcaattgataaggtggttctaggagaaaatgctctatttaattcgagtgataatgaattaattctcaaatgtggagggacaccaaatgcacggaatctcatcgatatcgagccagtcgcaactttcatagaatttgaacaattgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattgcacaattaactgcaggctacattaggaaattgattgagggggattgcaatatcgatctaaccagacctatccaaaagcaaatctggaaagcattaggttgtgtagtctattgtcacgatccaatggatcaaagggagtcaacagagtttattgatataaatattaatgaagaaatagaccgcgggatcgatggcgaggaaatctaaacatatcaagaatcagaattagtttaagaaaaaagaagaggattaatcttggttttccccttggtgggtcggcatggcatctccacctcctcgcggtccgacctgggcatccgaaggaggacgcacgtccactcggatggctaagggagcggccggggatccggctgctaacaaagcccgaaaggaagctgagttggctgctgccaccgctgagcaataactagcataaccccttggggcctctaaacgggtcttgaggggttttttgctgaaaggaggaactatatccggatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagt

SEQ ID NO: 14 - DNA, 25 bp, synthetic construct
  misc_feature 1..25: note = Synthetic: CVL1
  source 1..25: mol_type = other DNA
accaaggggaaaatgaagtggtgac

SEQ ID NO: 15 - DNA, 24 bp, synthetic construct
  misc_feature 1..24: note = Synthetic: CVL6
  source 1..24: mol_type = other DNA
ggagaagctcagatcagtgggatc

SEQ ID NO: 16 - DNA, 25 bp, synthetic construct
  misc_feature 1..25: note = Synthetic: CVL5
  source 1..25: mol_type = other DNA
acagtggattccataaggttcctgc

SEQ ID NO: 17 - DNA, 27 bp, synthetic construct
  misc_feature 1..27: note = Synthetic: CVL11
  source 1..27: mol_type = other DNA
cactgacagaaatgtcggaatgaagag

SEQ ID NO: 18 - DNA, 25 bp, synthetic construct
  misc_feature 1..25: note = Synthetic: CVL10
  source 1..25: mol_type = other DNA
tccaggtcactgttaggaagacatc

SEQ ID NO: 19 - DNA, 26 bp, synthetic construct
  misc_feature 1..26: note = Synthetic: CVL20
  source 1..26: mol_type = other DNA
gctcgattacctagattgaccgagac

SEQ ID NO: 20 - DNA, 23 bp, synthetic construct
  misc_feature 1..23: note = Synthetic: CVL17
  source 1..23: mol_type = other DNA
caagatgcaccttctctccagtg

SEQ ID NO: 21 - DNA, 24 bp, synthetic construct
  misc_feature 1..24: note = Synthetic: CVL154
  source 1..24: mol_type = other DNA
atcacactcgagctgacatctgtc

SEQ ID NO: 22 - DNA, 22 bp, synthetic construct
  misc_feature 1..22: note = Synthetic: CVL152
  source 1..22: mol_type = other DNA
cgtgagtacctacatgctgaca

SEQ ID NO: 23 - DNA, 23 bp, synthetic construct
  misc_feature 1..23: note = Synthetic: CVL25
  source 1..23: mol_type = other DNA
cggattgatacgctgagttgctg

SEQ ID NO: 24 - DNA, 23 bp, synthetic construct
  misc_feature 1..23: note = Synthetic: CVL19
  source 1..23: mol_type = other DNA
taactctgcagtcgctctacctc

SEQ ID NO: 25 - DNA, 22 bp, synthetic construct
  misc_feature 1..22: note = Synthetic: CVL27
  source 1..22: mol_type = other DNA
cagtctgagtcatgccatgctg

SEQ ID NO: 26 - DNA, 26 bp, synthetic construct
  misc_feature 1..26: note = Synthetic: CVL26
  source 1..26: mol_type = other DNA
gcattcagaatctcagctatggactg

SEQ ID NO: 27 - DNA, 24 bp, synthetic construct
  misc_feature 1..24: note = Synthetic: CVL33
  source 1..24: mol_type = other DNA
tcaatgatgatgtgagctacacgc

SEQ ID NO: 28 - DNA, 25 bp, synthetic construct
  misc_feature 1..25: note = Synthetic: CVL32
  source 1..25: mol_type = other DNA
gctctgatggaactcagtacgaatc

SEQ ID NO: 29 - DNA, 26 bp, synthetic construct
  misc_feature 1..26: note = Synthetic: CVL37
  source 1..26: mol_type = other DNA
ggttctctattgaactcaaccctgag

SEQ ID NO: 30 - DNA, 25 bp, synthetic construct
  misc_feature 1..25: note = Synthetic: CVL36
  source 1..25: mol_type = other DNA
gtgaccgatccatgaatctcattgc

SEQ ID NO: 31 - DNA, 26 bp, synthetic construct
  misc_feature 1..26: note = Synthetic: CVL42
  source 1..26: mol_type = other DNA
accaaggggaaaaccaagattaatcc

SEQ ID NO: 32 - DNA, 25 bp, synthetic construct
  misc_feature 1..25: note = Synthetic: oCVL433
  source 1..25: mol_type = other DNA
gcaaattgcaggtcgatgttgtgca

SEQ ID NO: 33 - DNA, 25 bp, synthetic construct
  misc_feature 1..25: note = Synthetic: oCVL294
  source 1..25: mol_type = other DNA
atttgagctttccgagacggtatca

SEQ ID NO: 34 - DNA, 59 bp, synthetic construct
  misc_feature 1..59: note = Synthetic: oCVL449
  source 1..59: mol_type = other DNA
gctgataccgtctcggaaagctcaaatcctgctataggctatccactgcatcatctctc

SEQ ID NO: 35 - DNA, 29 bp, synthetic construct
  misc_feature 1..29: note = Synthetic: oCVL451
  source 1..29: mol_type = other DNA
ccgccgcttgttgctaacccgtccgggcc

SEQ ID NO: 36 - DNA, 56 bp, synthetic construct
  misc_feature 1..56: note = Synthetic: oCVL448
  source 1..56: mol_type = other DNA
ggcccggacgggttagcaacaagcggcggccgccatgttcgtcttcctggtcctgc

SEQ ID NO: 37 - DNA, 55 bp, synthetic construct
  misc_feature 1..55: note = Synthetic: oCVL293
  source 1..55: mol_type = other DNA
taatgtttggtatgcattccatgagtcgacttcatttatgataaacaaaattctc

SEQ ID NO: 38 - DNA, 25 bp, synthetic construct
  misc_feature 1..25: note = Synthetic: oCVL433
  source 1..25: mol_type = other DNA
gcaaattgcaggtcgatgttgtgca

SEQ ID NO: 39 - DNA, 28 bp, synthetic construct
  misc_feature 1..28: note = Synthetic: oCVL445
  source 1..28: mol_type = other DNA
gcggccgccatgttcgtcttcctggtcc

SEQ ID NO: 40 - DNA, 43 bp, synthetic construct
  misc_feature 1..43: note = Synthetic: oCVL446
  source 1..43: mol_type = other DNA
ggaccaggaagacgaacatggcggccgccgcttgttgctaacc

SEQ ID NO: 41 - DNA, 56 bp, synthetic construct
  misc_feature 1..56: note = Synthetic: oCVL447
  source 1..56: mol_type = other DNA
ataatgtttggtatgcattccatgagtcgacttcatttatgataaacaaaattctc

SEQ ID NO: 42 - DNA, 25 bp, synthetic construct
  misc_feature 1..25: note = Synthetic: oCVL433
  source 1..25: mol_type = other DNA
gcaaattgcaggtcgatgttgtgca

SEQ ID NO: 43 - DNA, 34 bp, synthetic construct
  misc_feature 1..34: note = Synthetic: oCVL133
  source 1..34: mol_type = other DNA
gcgcgccaccatgtcagataacggaccacagaac

SEQ ID NO: 44 - DNA, 25 bp, synthetic construct
  misc_feature 1..25: note = Synthetic: oCVL134
  source 1..25: mol_type = other DNA
tcaagcctgggtggagtcggcactg

SEQ ID NO: 45 - DNA, 51 bp, synthetic construct
  misc_feature 1..51: note = Synthetic: oCVL363
  source 1..51: mol_type = other DNA
gtccgttatctgacatggtggcgcgcgatttgagctttccgagacggtatc

SEQ ID NO: 46 - DNA, 54 bp, synthetic construct
  misc_feature 1..54: note = Synthetic: oCVL364
  source 1..54: mol_type = other DNA
cagtgccgactccacccaggcttgacgtacgacctgctataggctatccactgc

SEQ ID NO: 47 - DNA, 20 bp, synthetic construct
  misc_feature 1..20: note = Synthetic: oCVL450
  source 1..20: mol_type = other DNA
agaagtgggccaagtgtcag

SEQ ID NO: 48 - DNA, 50 bp, synthetic construct
  misc_feature 1..50: note = Synthetic: oCVL365
  source 1..50: mol_type = other DNA
ggcccggacgggttagcaacaagcggcggccgccaccatgttcgtcttcc

SEQ ID NO: 49 - DNA, 55 bp, synthetic construct; identical to SEQ ID NO: 37
  misc_feature 1..55: note = Synthetic: oCVL293
  source 1..55: mol_type = other DNA

SEQ ID NO: 50 - DNA, 60 bp, synthetic construct
  misc_feature 1..60: note = Synthetic: oCVL616
  source 1..60: mol_type = other DNA
ggcccggacgggttagcaacaagcgcgcggccgccaccatgttcgtcttcctggtcctgc

SEQ ID NO: 51 - DNA, 60 bp, synthetic construct
  misc_feature 1..60: note = Synthetic: oCVL444
  source 1..60: mol_type = other DNA
aataatgtttggtatgcattccatgagtcgacacgcgttcatttatgataaacaaaattc

SEQ ID NO: 52 - DNA, 59 bp, synthetic construct
  misc_feature 1..59: note = Synthetic: oCVL617
  source 1..59: mol_type = other DNA
ggcccggacgggttagcaacaagcggcggccgccaccatgttcgtcttcctggtcctgc

SEQ ID NO: 53 - DNA, 55 bp, synthetic construct; identical to SEQ ID NO: 37
  misc_feature 1..55: note = Synthetic: oCVL293
  source 1..55: mol_type = other DNA

SEQ ID NO: 54 - DNA, 25 bp, synthetic construct
  misc_feature 1..25: note = Synthetic: oCVL5
  source 1..25: mol_type = other DNA
acagtggattccataaggttcctgc

SEQ ID NO: 55 - DNA, 24 bp, synthetic construct
  misc_feature 1..24: note = Synthetic: oCVL622
  source 1..24: mol_type = other DNA
agctcattccagctccgtcaggtt

SEQ ID NO: 56 - DNA, 51 bp, synthetic construct
  misc_feature 1..51: note = Synthetic: oCVL623
  source 1..51: mol_type = other DNA
aacctgacggagctggaatgagctagcggcagaagccttcaggtctgaccc

SEQ ID NO: 57 - DNA, 19 bp, synthetic construct
  misc_feature 1..19: note = Synthetic: oCVL67
  source 1..19: mol_type = other DNA
cgataggcagatccaccag

SEQ ID NO: 58 - DNA, 52 bp, synthetic construct
  misc_feature 1..52: note = Synthetic: oCVL670
  source 1..52: mol_type = other DNA
ggcccggacgggttagcaacaagcggcggccgccgccaccatgttcgtcttc

SEQ ID NO: 59 - DNA, 59 bp, synthetic construct
  misc_feature 1..59: note = Synthetic: oCVL671
  source 1..59: mol_type = other DNA
caataatgtttggtatgcattccatgagtcgaccttattaggtgtaatgcagcttcacg

SEQ ID NO: 60 - DNA, 57 bp, synthetic construct
  misc_feature 1..57: note = Synthetic: oCVL672
  source 1..57: mol_type = other DNA
tgataccgtctcggaaagctcaaatcgcgcgccaccatggccgatagcaacggcacc

SEQ ID NO: 61 - DNA, 56 bp, synthetic construct
  misc_feature 1..56: note = Synthetic: oCVL673
  source 1..56: mol_type = other DNA
atgcagtggatagcctatagcaggtcgtacgtcatcactgcaccagcagggcgatg

SEQ ID NO: 62 - DNA, 54 bp, synthetic construct
  misc_feature 1..54: note = Synthetic: oCVL674
  source 1..54: mol_type = other DNA
acatcgccctgctggtgcagtgatgacgtacggatcccaatcttaaatcgacac

SEQ ID NO: 63 - DNA, 26 bp, synthetic construct
  misc_feature 1..26: note = Synthetic: oCVL675
  source 1..26: mol_type = other DNA
acgcgtggattgtgttattggcatcg

SEQ ID NO: 64 - DNA, 53 bp, synthetic construct
  misc_feature 1..53: note = Synthetic: oCVL676
  source 1..53: mol_type = other DNA
cgatgccaataacacaatccacgcgtgccaccatgtattctttcgtgtctgaa

SEQ ID NO: 65 - DNA, 25 bp, synthetic construct
  misc_feature 1..25: note = Synthetic: oCVL677
  source 1..25: mol_type = other DNA
gctagcttacacgagcaggtcgggc

SEQ ID NO: 66 - DNA, 50 bp, synthetic construct
  misc_feature 1..50: note = Synthetic: oCVL678
  source 1..50: mol_type = other DNA
gcccgacctgctcgtgtaagctagcacctgctataggctatccactgcat

SEQ ID NO: 67 - DNA, 20 bp, synthetic construct
  misc_feature 1..20: note = Synthetic: oCVL450
  source 1..20: mol_type = other DNA
agaagtgggccaagtgtcag

SEQ ID NO: 68 - DNA, 90 bp, synthetic construct
  misc_feature 1..90: note = Synthetic: oCVL723
  source 1..90: mol_type = other DNA
atcgccctgctggtgcagtgatgacgtacgagaaacacccaattacactacactggtatgacactgtactaaccctgagggttttagaaa

SEQ ID NO: 69 - DNA, 90 bp, synthetic construct
  misc_feature 1..90: note = Synthetic: oCVL724
  source 1..90: mol_type = other DNA
cctgagggttttagaaaaaacgattaacgataaataagcccgaacactacacactacctgaggcagccaccatgtcagataacggaccac

SEQ ID NO: 70 - DNA, 46 bp, synthetic construct
  misc_feature 1..46: note = Synthetic: oCVL725
  source 1..46: mol_type = other DNA
gtgtgtcgatttaagattgggatccgtacgtcaagcctgggtggag

(19) United States
(12) Patent Application Publication
(10) Pub. No.: US 2024/0287544 A1
(43) Pub. Date: Aug. 29, 2024

(54) MODIFIED PIV5 VACCINE VECTORS: METHODS OF MAKING AND USES

(71) Applicant: CYANVAC LLC, Athens, GA (US)

(72) Inventors: Biao HE, Watkinsville, GA (US); Zhuo LI, Bogart, GA (US); Hong JIN, Cupertino, CA (US); Maria Cristina GINGERICH, Athens, GA (US)

(73) Assignee: CYANVAC LLC, Athens, GA (US)

(21) Appl. No.: 18/433,149

(22) Filed: Feb. 5, 2024

Related U.S. Application Data
(60) Provisional application No. 63/483,377, filed on Feb. 6, 2023.

(51) Int. Cl. C12N 15/86; A61K 39/00; A61K 39/215; A61P 31/14

(52) U.S. Cl. CPC C12N 15/86; A61K 39/215; A61P 31/14; A61K 2039/525; A61K 2039/543; A61K 2039/575; C12N 2760/18522; C12N 2760/18534; C12N 2760/18722; C12N 2760/18743; C12N 2770/20022; C12N 2770/20034; C12N 2770/32343

(57) ABSTRACT

A CVB viral expression vector comprising a PIV5 W3A viral genome that contains mutations at amino acid residue S157 or S156 in the P/V gene and a deletion of the small hydrophobic (SH) gene of the PIV5 W3A viral genome, wherein the amino acid substitution at amino acid residue S157 or S156 comprises a substitution of serine (S) to phenylalanine (F) or asparagine (N) and wherein the SH gene has a deletion of the SH open reading frame or a deletion of an entire SH gene transcript unit. The CVB viral expression vector wherein the vector expresses a heterologous polypeptide comprising a SARS-CoV-2 spike (S), and/or nucleocapsid (N) and/or membrane (M) proteins, RSV fusion protein (F) or other antigens.

### REFERENCE TO RELATED APPLICATIONS

This application is a US utility patent application of U.S. Provisional Application Ser. No. 63/483,377, filed Feb. 6, 2023, which is hereby incorporated by this reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 22, 2024, is named “065095.005US1.xml” and is 363,048 bytes in size. The sequence listing contained in this .XML file is part of the specification and is hereby incorporated by reference herein in its entirety.

### FIELD

The invention is generally related to the field of vaccination, and more particularly to compositions and methods of using modified PIV5 vaccine vectors such as PIV5W3AΔSH or PIV5W3AΔSH with P/V gene mutation (CVB) for modulating immune responses in a subject having or susceptible to infectious agents such as RSV and SARS-CoV-2.

### BACKGROUND

Parainfluenza virus type 5 (Parainfluenza virus 5, PIV5) belongs to the family Paramyxoviridae and the genus Rubulavirus which also includes mumps virus, and its genome is negative single strand RNA with a length of 15246 nt. The genome full-length structure of PIV5 is 3′-Leader-NP-V/P-M-F-SH-HN-L-Trailer-5′, namely, from 3′end to 5′ end, Nucleocapsid Protein (NP), V protein/phosphorylated protein (P), Matrix protein (M), Fusion protein (F), Small hydrophobic protein (SH), Hemagglutinin-neuraminidase protein (HN) and polymerase protein (Large protein, L) are encoded in sequence. Wherein, the V protein and the SH protein are non-structural proteins.

PIV5 is an excellent viral vector for vaccine development, and research on PIV5 recombinant vaccines is underway. In recent years, researchers have continuously explored the feasibility of using PIV5 as a vaccine vector. A common approach is to insert a protective antigen gene from a virus or bacteria into PIV5, and to express the inserted foreign gene by replication and translation of PIV5 vector. Given that PIV5 can infect respiratory tract without causing any illness, researchers often take advantage of this and focus on controlling certain respiratory viral infections. Therefore, the deep research on the molecular biological characteristics, the replication mechanism and the like of the virus is beneficial to the comprehensive and thorough understanding of the PIV5, so that a foundation is laid for the research on the PIV5 as a genetic engineering vaccine vector and the gene function research of the virus. However, there remains a need for more potent and high yield PIV5 vaccine vectors.

Thus, the disclosure provided herein provides a more potent and high yield PIV5 CVB backbone which contains the P/V gene S156N or S157F mutation, and the generation of new SARS-CoV-2 CVB-vectored vaccines for intranasal immunization. The phosphoprotein (P) protein can be phosphorylated at serine residues at positions 36, 126, and 157 and a threonine residue at position 286. In addition, host cell Polo-like kinase 1 (PLK1) can phosphorylate a serine residue at position 308. Mutation of the serine residues to alanine residues at either position 157 or 308 prevents phosphorylation at these residues significantly enhancing the activity of the vRdRP in mini-genome assays and the replication of recombinant viruses that bear these mutations. The mutation at residue 156 and 157 are hypothesized to upregulate viral transcription and replication, improving vaccine virus yield. The change in the amino acids 155-159 TSSPI motif of the PIV5 P protein will change virus phenotype and growth property in vitro and in vivo.

### SUMMARY

In accordance with the purpose(s) of this invention, as embodied and broadly described herein, this invention, in one aspect, relates to a modified PIV5 (termed CVB) viral expression vector comprising a PIV5 W3A viral genome having a mutations at amino acid residue S157 or S156 of the P/V gene and a deletion of the small hydrophobic (SH) gene of the PIV5 W3A viral genome. This modified CVB backbone has been shown to grow better in cell culture such as in serum-free Vero cells and be more immunogenic, such as in the cotton rat animal model. The modified CVB backbone can be used as an effective vaccine platform.

In some embodiments, the mutation at amino acid residue S157 or S156 comprises the substitution of serine (S) with an amino acid residue selected from a group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), selenocysteine (U), valine (V), tryptophan (W), and tyrosine (Y). In one embodiment, the amino acid substitution at amino acid residue S157 comprises a substitution of serine (S) to phenylalanine (F) or S156 comprises a substitution of serine (S) to asparagine (N).

In further embodiments, the SH gene has a deletion of the SH open reading frame or a deletion of an entire SH gene transcript unit.

In one embodiment, the CVB viral expression vector expresses a heterologous polypeptide comprising a viral antigen selected from a group consisting of SARS-CoV-2, RSV or other viral or bacterial antigens. In another embodiment, the PIV5 genome has a heterologous nucleic acid sequence with at least 98% sequence identity to SEQ ID NOs: 2, 3, 4, 5, 6, 7, 8, 9, 10, 11, 12, or 13 and wherein the viral expression vector expresses a heterologous polypeptide comprising a coronavirus spike (S) and/or nucleocapsid (N) proteins, RSV-F proteins, or viral or bacterial antigens.

In one embodiment, the coronavirus S protein is a coronavirus S protein of the severe acute respiratory syndrome coronavirus 2 (SARS-CoV-2), a variant of interest or a variant of concern of SARS-CoV-2 and the coronavirus N protein is the coronavirus N protein of SARS-CoV-2, a variant of interest or a variant of concern of SARS-CoV-2. In some embodiment, the coronavirus S protein is the coronavirus S protein of a SARS-CoV-2 Wuhan strain, a SARS-CoV-2 beta variant, a SARS-CoV-2 gamma variant, a SARS-CoV-2 delta variant, or a SARS-CoV-2 omicron variant and the coronavirus N protein is the coronavirus N protein of a SARS-CoV-2 Wuhan strain, a SARS-CoV-2 beta variant, a SARS-CoV-2 gamma variant, a SARS-CoV-2 delta variant, or a SARS-CoV-2 omicron variant. In some embodiments, the SARS-CoV-2 omicron variant is SARS-CoV-2 Omicron BA.1 or SARS-CoV-2 Omicron BA.5, BQ1 or XBB1 or any future emerging variants.

In one embodiment, the coronavirus S protein comprises the coronavirus S protein of SARS-CoV-2 and wherein the cytoplasmic tail of the coronavirus S protein has been replaced with the cytoplasmic tail of the fusion (F) protein of CVB.

In one embodiment, the PIV5 W3A viral genome comprises open reading frame deletion mutations of the SH gene and the S gene of SARS-CoV-2 Wuhan strain is inserted between the PIV5 hemagglutinin (HN) and polymerase (L) genes of CVB.

In some embodiments, the entire SH gene transcript unit of PIV5 W3A viral genome is deleted and the S gene of the SARS-CoV-2 Wuhan strain is placed between the HN and L genes of CVB.

In some embodiments, the N gene of SARS-CoV-2 Wuhan strain is inserted to replace the SH gene of PIV5, and the S gene of SARS-CoV-2 Wuhan strain is inserted between the HN and L genes of CVB.

In some embodiments, the N gene of SARS-CoV-2 Wuhan strain is inserted to replace the SH gene of PIV5, and the S gene of SARS-CoV-2 Omicron BA.1 variant is inserted between the HN and L genes of CVB

In some embodiments, the S gene of SARS-CoV-2 Omicron BA.5 variant is inserted between the HN and L genes of CVB.

In some embodiments, the S gene of SARS-CoV-2 Omicron BA.5 variant is inserted between the HN and L genes and the N gene of SARS-CoV-2 Wuhan strain is inserted to replace the SH gene of CVB.

In some embodiments, the PIV5 F and HN genes are deleted and wherein the S gene of SARS-CoV-2 Wuhan strain is between M and L genes of CVB.

In some embodiments, the N gene of the SARS-CoV-2 Wuhan strain is inserted between F and HN, and the S gene of the SARS-CoV-2 Wuhan strain is inserted between the HN and L genes of CVB.

In some embodiments, the M gene from the SARS-CoV-2 Wuhan strain is inserted between F and HN, and the S gene of the SARS-CoV-2 Wuhan strain is inserted between HN and L of CVB.

In some embodiments, the M gene from the SARS-CoV-2 Wuhan strain is inserted after F of PIV5, the E gene from the SARS-CoV-2 Wuhan strain inserted between the M gene of SARS-CoV-2 and HN, and the S gene of SARS-CoV-2 Wuhan strain is inserted between HN and L of CVB.

In some embodiments, the M gene from the SARS-CoV-2 Wuhan strain is inserted after F of PIV5, the N gene from the SARS-CoV-2 Wuhan strain is inserted between the M gene and the E gene of SARS-CoV-2, the E gene from the SARS-CoV-2 Wuhan strain inserted between the N gene of SARS-CoV-2 and HN, and the S gene of SARS-CoV-2 Wuhan strain is inserted between HN and L of CVB.

In some embodiments, the F gene from respiratory syncytial virus (RSV) is inserted between the SH and HN genes of CVB backbone.

In one embodiment, a viral particle comprises the viral expression vector.

In another aspect, the invention relates to a composition comprising a CVB viral expression vector having a nucleic acid sequence with at least 98% sequence identity to SEQ ID NOs: 2, 3, 4, 5, 6, 7, 8, 9, 10, 11, 12, or 13 and wherein the viral expression vector expresses a heterologous polypeptide comprising a coronavirus spike (S) and/or nucleocapsid (N) proteins. In one embodiment, the heterologous coronavirus spike (S) and nucleocapsid (N) proteins are expressed in a cell by contacting the cell with the composition.

In yet another aspect, the invention relates to a method of inducing an immune response in a subject having or at risk of having SARS-COV-2, RSV or other viral or bacterial infections, the method comprising administering the composition of to the subject, wherein the immune response comprises a humoral immune response and/or a cellular immune response.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed. In one embodiment, the subject is vaccinated against COVID-19, RSV or other viral or bacterial infections the method comprising administering the composition to the subject, wherein the composition is administered intranasally, intramuscularly, topically, or orally. The method further comprises administering a PIV5 booster vaccine composition comprising a viral expression vector or a viral particle having a PIV5 genome comprising a heterologous nucleic acid sequence with at least 98% sequence identity to SEQ ID NOs: 27, 28, 29, 30, 31, 32, or 33, wherein said subject has previously received a primary vaccination against SARS-COV-2, RSV or other viral or bacterial infections.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIG. 1 is a diagram of W3AΔSH, CPI and CVB genomic structure.

FIGS. 2A-2B show RSV F-specific cellular immune responses in W3AΔSH-RSV-F vs CPI-RSV-F-immunized AGMs. RSV F-specific cellular immune response in immunized African green monkeys (AGMs). PBMCs were isolated from immunized AGMs one day prior to vaccination, and at 14 and 28 days post-immunization. The PBMCs were stimulated with an RSV F peptide pool, and different cytokine levels in CD4⁺ (FIG. 2A) and CD8⁺ (FIG. 2B) cells were quantified by ICS and expressed at % sum of all cytokines.

FIG. 3 shows a lung RSV viral titer following RSV challenge following immunization with indicated controls or antigens.

FIG. 4 shows a RSV titer following RSV challenge in nasal wash.

FIG. 5 shows RSV neutralizing antibody responses.

FIG. 6 shows anti-RSV F protein IgG antibody responses by ELISA.

FIG. 7 shows an illustrative overview of virus rescue.

FIG. 8 shows schematic diagrams of the PIV5 CVB based SARS-CoV-2 vaccine constructs indicating the location of SARS-CoV-2 genes and their corresponding variant of origin.

FIGS. 9A-9D show immune responses in animals induced by CVXGA16, CVXGA17 and CVXGA18. IgG antibodies against SARS-CoV-2 S protein (FIG. 9A) or S-RBD (FIG. 9B) in mice. The mice were immunized intranasally with 50 μL of PBS or 10⁵ PFU CVXGA1, CVXGA16, CVXGA17 and CVXGA18. At D32, blood was collected. Anti-S WAl or S WAl RBD IgG antibodies were determined using ELISA. FIG. 9C shows T-cell responses against S or N peptides in mice. The mice were immunized as above. Spleen was collected at D32. S or N protein-specific IFNγ-secreting cells were determined using ELISPOT. FIG. 9D shows IgG antibodies against S protein in hamsters. The hamsters were immunized intramuscularly with 50 μL of PBS or with 2 μg of COVID-19 mRNA vaccine. After boost at D21, hamsters from Group 1 were boosted intranasally with 50 μL of PBS, and hamsters from Group 2 were boosted a second time intramuscularly with 2 μg of COVID-19 mRNA (Group 2A), or intranasally with 2×10⁶ PFU of CVXGA1 (Group 2B), or CVXGA18 (Group 2C) at D42. At 63 days post-immunization, blood was collected. Anti-S IgG antibodies were determined using ELISA.

FIG. 10 shows growth curves of CVXGA29, CVXGA30, CVXGA31, and CVXGA32 expressing wt SARS-CoV-2 S compared to CVXGA17 expressing SARS-CoV-2 S with PIV5 F tail.

FIGS. 11A-11B show anti-SARS-CoV-2 S immunogenicity (FIG. 11A) and anti-SARS-CoV-2 N immunogenicity (FIG. 11B) for CVXGA1, CVXGA18, CVXGA29, CVXGA30, CVXGA31, or CVXGA32

FIG. 12 show the determination of percent RSV F protein expression in CVB-F-infected cells. Vero-SF cells were infected with CVB-F pre-MVS virus at dilutions −3 to −5. After 1-hour incubation at 37° C., media was changed, and cells were incubated for 18 hours at 37° C. Immunostaining was performed using mouse anti-CVB-HN and human anti-F (Palivizumab) antibodies followed by anti-mouse Alexa 488 and anti-human Cy3 secondary antibodies, respectively. Three representative images of wells infected with CVB-F pre-MVS showing both green and red cells. 100% CVB-infected cells expressed both PIV5 and RSV F proteins. Images were taken at 10×.

FIGS. 13A-13E show the replication of CVB-F at 35° C. vs 37° C. and day 2 vs day 4 serum free Vero cells.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

As described herein, the term “vaccinating” designates typically the sequential administration of one or more antigens to a subject, to produce and/or enhance an immune response against the antigen(s). The sequential administration includes a priming immunization followed by one or several boosting immunizations.

Within the context of the present invention, the term “pathogen” refers to any agent that can cause a pathological condition. Examples of “pathogens” include, without limitation, cells (e.g., bacteria cells, diseased mammal cells, cancer mammal cells), fungus, parasites, viruses, prions or toxins. Preferred pathogens are infectious pathogens. In a particular embodiment, the infectious pathogen is a virus, such as the coronaviruses.

An antigen, as used therein, designates any molecule which can cause a T-cell or B-cell immune response in a subject. An antigen specific for a pathogen is, typically, an element obtained or derived from said pathogen, which contains an epitope, and which can cause an immune response against the pathogen. Depending on the pathogenic agent, the antigen may be of various nature, such as a (poly)peptide, protein, nucleic acid, lipid, cell, etc. Live weakened forms of pathogens (e.g., bacteria, viruses), or killed or inactivated forms thereof may be used as well, or purified material therefrom such as proteins, peptides, lipids, etc. The antigen may be naturally-occurring or artificially created. It may be exogenous to the treated mammal, or endogenous (e.g., tumor antigens). The antigen may be produced by techniques known per se in the art, such as for instance synthetic or recombinant technologies, or enzymatic approaches.

In a particular embodiment, the antigen is a protein, polypeptide and/or peptide. The terms “polypeptide”, “peptide” and “protein” are used interchangeably herein to refer to a polymer of amino acid residues. The terms also apply to amino acid polymers in which one or more amino acid residues may be modified or non-naturally occurring residues, such as an artificial chemical mimetic of a corresponding naturally occurring amino acid. It should be understood that the term “protein” also includes fragments or variants of different antigens, such as epitope-containing fragments, or proteins obtained from a pathogen and subsequently enzymatically, chemically, mechanically or thermally modified.

A “therapeutically effective amount” means the amount of a compound (e.g., a CVB-based composition as described herein) that, when administered to a subject for treating a state, disorder or condition, is sufficient to effect such treatment. The “therapeutically effective amount” will vary depending on the compound or bacteria administered as well as the disease and its severity and the age, weight, physical condition and responsiveness of the mammal to be treated.

The phrase “pharmaceutically acceptable”, as used in connection with compositions of the disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “pharmaceutically acceptable composition” as used herein refers to a composition comprising at least one compound as disclosed herein formulated together with one or more pharmaceutically acceptable carriers.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

The term “dose” means a single amount of a compound or an agent that is being administered thereto; and/or “regimen: which means a plurality of pre-determined doses that can be different in amounts or similar, given at various time intervals, which can be different or similar in terms of duration. In some embodiments, a regimen also encompasses a time of a delivery period (e.g., agent administration period, or treatment period). Alternatively, a regimen is a plurality of predetermined plurality pre-determined vaporized amounts given at pre-determined time intervals.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

As used herein, the term “parainfluenza virus 5” (PIV5) includes, for example and not limitation, strains KNU-11, CC-14, D277, 1168-1, and 08-1990. Non-limiting examples of PIV5 genomes are listed in GenBank Accession Nos. NC_006430.1, AF052755.1, KC852177.1, KP893891.1, KC237065.1, KC237064.1 and KC237063.1, which are hereby incorporated by reference.

As used herein, the term “expression” refers to the process by which polynucleic acids are transcribed into mRNA and translated into peptides, polypeptides, or proteins. If the polynucleic acid is derived from genomic DNA, expression may, if an appropriate eukaryotic host cell or organism is selected, include splicing of the mRNA. In the context of the present invention, the term also encompasses the yield of the F gene mRNA and F proteins achieved following expression thereof.

As used herein, the term “F protein” or “Fusion protein” or “F protein polypeptide” or “Fusion protein polypeptide” refers to a polypeptide or protein having all or part of an amino acid sequence of an RSV Fusion protein polypeptide. Numerous RSV Fusion and Attachment proteins have been described and are known to those of skill in the art. WO/2008/114149, which is herein incorporated by reference in its entirety, sets out exemplary F and G protein variants (for example, naturally occurring variants).

As used herein, the term “combination” of a CVB-based composition as described herein and at least a second pharmaceutically active ingredient means at least two, but any desired combination of compounds can be delivered simultaneously or sequentially (e.g., within a 24-hour period). It is contemplated that when used to treat various diseases, the compositions and methods of the present disclosure can be utilized with other therapeutic methods/agents suitable for the same or similar diseases. Such other therapeutic methods/agents can be co-administered (simultaneously or sequentially, in any order) to generate additive or synergistic effects. Suitable therapeutically effective dosages for each agent may be lowered due to the additive action or synergy. Also, two or more embodiments of the disclosure may be also co-administered to generate additive or synergistic effects.

The term “coronavirus” refers to a group of related RNA viruses that cause diseases in mammals and birds. In humans, these viruses cause respiratory tract infections that can range from mild to lethal. Mild illnesses include some cases of the common cold (which is caused also by certain other viruses, predominantly rhinoviruses), while more lethal varieties can cause SARS, MERS, and COVID-19. There are presently no vaccines or antiviral drugs to prevent or treat human coronavirus infections.

The term “SARS” or “severe acute respiratory syndrome” refers to a viral respiratory disease of zoonotic origin that surfaced in the early 2000s caused by severe acute respiratory syndrome coronavirus (SARS-CoV or SARS-CoV-1), the first-identified strain of the SARS coronavirus species severe acute respiratory syndrome-related coronavirus (SARS-CoV). The syndrome caused the 2002-2004 SARS outbreak. In 2019, its successor, the related virus strain Severe Acute Respiratory Syndrome Coronavirus 2 (SARS-CoV-2), was discovered. The term “Covid-19” or “Coronavirus disease 2019” refers to a severe acute respiratory syndrome (SARS) caused by a virus known as SARS-Coronavirus 2 (SARS-CoV-2).

The mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the disclosure are intended to be illustrative and not restrictive. Many suitable materials that would perform the same or a similar function as the materials described herein are intended to be embraced within the scope of the disclosure. Such other materials not described herein can include, but are not limited to, for example, materials that are developed after the time of the development of the disclosure.

### II. CPI-RSV-F, W3AΔSH and CVB-RSV-F Compositions

Respiratory syncytial virus (RSV) is a member of the genus Pneumoviridae. Human RSV (HRSV) is the leading cause of severe lower respiratory tract disease in young children and is responsible for considerable morbidity and mortality in humans. RSV is also recognized as an important agent of disease in immunocompromised adults and in the elderly. Due to incomplete resistance to RSV in the infected host after a natural infection, RSV may infect multiple times during childhood and adult life.

This virus has a genome comprised of a single strand negative-sense RNA, which is tightly associated with viral protein to form the nucleocapsid. The viral envelope is composed of a plasma membrane derived lipid bilayer that contains virally encoded structural proteins. A viral polymerase is packaged with the virion and transcribes genomic RNA into mRNA. The RSV genome encodes three transmembrane structural proteins, F, G, and SH, two matrix proteins, M and M2, three nucleocapsid proteins N, P, and L, and two nonstructural proteins, NS1 and NS2.

Fusion of HRSV and cell membranes is thought to occur at the cell surface and is a necessary step for the transfer of viral ribonucleoprotein into the cell cytoplasm during the early stages of infection. This process is mediated by the fusion (F) protein, which also promotes fusion of the membrane of infected cells with that of adjacent cells to form a characteristic syncytia, which is both a prominent cytopathic effect and an additional mechanism of viral spread. Accordingly, neutralization of fusion activity is important in host immunity. Indeed, monoclonal antibodies developed against the F protein have been shown to neutralize virus infectivity and inhibit membrane fusion (Calder et al., 2000, Virology 271: 122-131).

The F protein of RSV shares structural features and limited, but significant amino acid sequence identity with F glycoproteins of other paramyxoviruses. It is synthesized as an inactive precursor of 574 amino acids (F0) that is cotranslationally glycosylated on asparagines in the endoplasmic reticulum, where it assembles into homo-oligomers. Before reaching the cell surface, the F0 precursor is cleaved by a protease into F2 from the N terminus and F1 from the C terminus. The F2 and F1 chains remain covalently linked by one or more disulfide bonds.

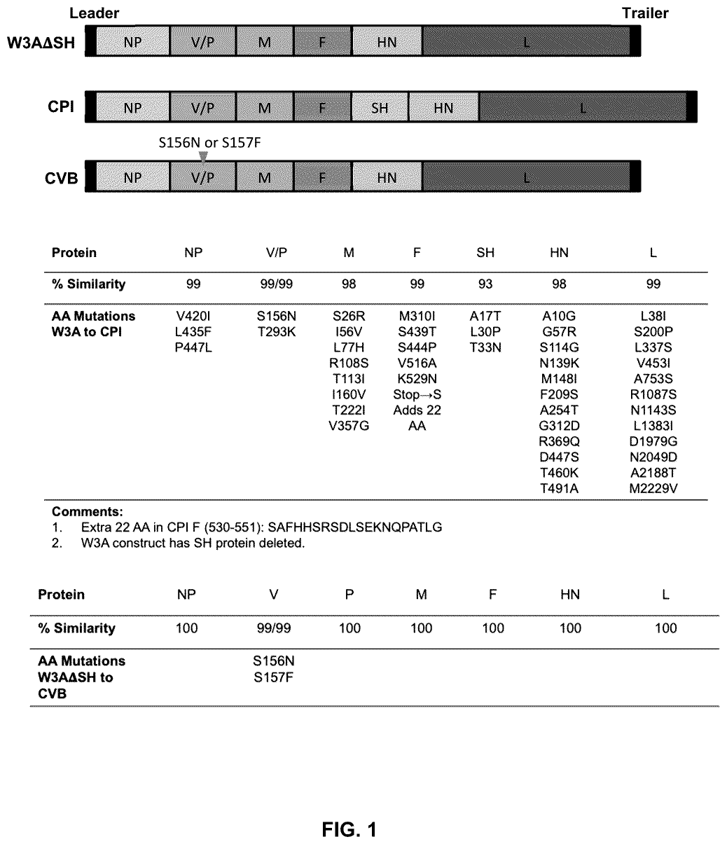

CPI-RSV-F is a parainfluenza virus (PIV5) based RSV vaccine expressing the RSV F protein, is provided herein as prophylactic intranasal vaccines to prevent RSV infection and serious complications associated with RSV infection. CPI-RSV-F was designed to induce immune responses to the F protein of RSV, which is the main antigenic protein that is highly conserved between the RSV subgroups A and B. Anti-F antibodies inhibit virus entry into host cells and RSV F is a proven vaccine target based on the efficacy data from the commercial product palivizumab (RSV F monoclonal antibody). W3AΔSH-RSV-F and CVB-RSV are modified RSV vaccine, their backbone difference from the CPI-RSV-F is summarized in Figure. 1.

The disclosure provides CPI-RSV-F, W3AΔSH-RSV-F and CVB-RSV compositions, systems and methods for their use in multiple applications including functional genomics, drug discovery, target validation, protein production (e.g., therapeutic proteins, vaccines, monoclonal antibodies), gene therapy, and therapeutic treatments such as cancer therapy.

### A. Pharmacology Summary of RSV Studies

The disclosure provides CPI-RSV-F, W3AΔSH-RSV-F and CVB-RSV compositions, systems and methods for their use in multiple applications including functional genomics, drug discovery, target validation, protein production (e.g., therapeutic proteins, vaccines, monoclonal antibodies), gene therapy, and therapeutic treatments such as cancer therapy.

Previously published studies based on W3A strain of PIV5 construct engineered to express RSV-F protein included immunogenicity and challenge studies in mice, cotton rats and African green monkeys (1,2,3). As part of these studies, permissiveness for PIV5 replication was confirmed in cotton rats and African monkeys (1, 2). In addition, the following studies were performed: 1) compared vector constructs based on CPI vs. W3A PIV5 strain; 2) compared vaccine constructs expressing RSV pre-fusion versus wt F-protein in place of PIV5 SH gene (ΔSH) or inserted between PIV5 SH and NH (SH-NH) or between HN and L (HN-L). These studies led to the selection of CPI-RSV-F (CPI-RSV-F), containing the full length of RSV F-protein inserted between SH and HN gene for use in the initial human studies.

In addition, the protective efficacy of CPI-RSV-F vaccine has been evaluated in RSV challenge studies conducted in mice and cotton rats. Immunization with a single intranasal dose protected animals from RSV infection based on significantly reduced RSV viral titers observed in lung and nasal washes of immunized animals compared to controls.

More recent preclinical proof of concept studies conducted with the vaccine vector construct CPI-RSV-F included immunogenicity and challenge studies conducted by Blue Lake Biotechnology Inc. in mice and African green monkeys. In addition, preclinical data from a NIH sponsored study of the CPI-RSV-F construct in a cotton rat challenge study are summarized herein. The CPI-RSV-F vaccine used in these more recent non-clinical studies used a prior vaccine vector construct (rescued from BHK cells) that is the same as the vector construct used for clinical lot material (rescued from 293/Vero cells), and similarly produced using serum-free Vero cells as the substrate and formulated with sucrose phosphate glutamate (SPG) buffer. These non-clinical studies included the control (PIV5 W3A strain with SH gene deleted, W3AΔSH-RSV-F, engineered to express RSV F protein) as an active comparator.

In summary, the non-clinical studies in various animal models have demonstrated the ability of CPI-RSV-F, W3AΔSH-RSV-F and CVB-RSV to induce RSV F specific immune responses, as observed by F-specific antibody and cell mediated immune responses following a single intranasal dose. All of these vaccine candidates were well tolerated in these animal models and no indication of sensitization following vaccination.

TABLE 1
Non-clinical study overview of prior vaccine constructs
studies and recent studies with CPI-RSV-F construct.

| | | | Vaccine | Reference |
|---|---|---|---|---|
| Study type | Species | Vaccine vector | route | report |
| Immunogenicity/ | Mice | W3A strain with wt | i.n. | Phan et al. 2014 |
| challenge study | | RSV F protein inserted | | |
| | | into PIV5 HN-L region | | |
| Immunogenicity/ | Mice and Cotton | W3A strain with wt | i.n. and s.c. | Phan et al 2017/ |
| challenge/safety | rats | RSV or prefusion RSV | | Sigmovir Protocol |
| study | | F protein inserted at | | #XV-131 Study |
| | | deleted SH region or | | report |
| | | between SH and HN | | |
| Immunogenicity/ | Cotton rats and | W3A strain with RSV F | i.n | Wang et al 2017 |
| challenge/safety | African Green | protein inserted into the | | |
| study | monkeys | intergenic regions of | | |
| | | PIV5 HN and L | | |

Recent studies with W3AΔSH-RSV-F (construct to be used in clinical study and used in
toxicology study)

| | | | | |
|---|---|---|---|---|
| Immunogenicity/ | Mice | CPI strain with RSV F | i.n. | Report 1 |
| challenge study | | protein between SH and | | |
| | | NH (CPI-RSV-F, CPI- | | |
| | | RSV-F) or W3A strain | | |
| | | with RSV F protein | | |
| | | replacing SH gene | | |
| | | (W3AΔSH-RSV-F) | | |
| Immunogenicity | African green | CPI-RSV-F or | i.n. | No report. |
| study | Monkeys | W3AΔSH-RSV-F | | |
| Immunogenicity/ | Cotton rats | CPI-RSV-F or | i.n. | Sigmovir |
| challenge/safety | | W3AΔSH-RSV-F | | Study XV- |
| study | | containing minor | | 238 report |
| | | population of BLB-205 | | (NIH |
| | | | | sponsored) |

i. Studies in Mice and Cotton Rats, and Monkeys with Related W3A PIV5 Vector Expressing RSV F Protein

PIV5 (W3A)-RSV-F and RSV-G protein study in Balb c mice: In this study Balb/c mice received a single intranasal dose of W3A-RSV-F (10⁶ PFU dose in 50 μl) followed by RSV challenge. The PIV5 W3A vaccine vector construct in this study consisted of wild type RSV F protein inserted into the PIV5 HN and L intergenic regions. A single intranasal dose resulted in IgG2a/IgG1 RSV responses similar to that observed after wild-type RSV A2 infection at Day 21 after immunization.

PIV5 (W3A) expressing wild-type or Prefusion RSV F protein challenge study in mice and cotton rats: This study evaluated PIV5 vectored vaccines that were improved by changing location of F-protein insertion (inserted at SH-HN junction of PIV5 or replacing the SH with the RSV F protein gene). In addition, this study evaluated both the wild type (wt) F-protein or a prefusion conformation F-protein (pF).

Mice were immunized intranasally with single dose of W3AΔSH-RSV-F (RSV F protein gene inserted at deleted SH region of PIV5) expression wild type F protein or prefusion stabilized RSV F mutant (W3AΔSH-RSV-pF) or improved vector W3AΔSH-RSV-F or W3A-RSV-pF SH-HN (the F-protein gene inserted at the SH-HN junction) at 1×10⁶ PFU. The study groups were as follows (Table 2).

TABLE 2
Study groups in mice challenge study

| | Number of | |
|---|---|---|
| Vaccine/route | animals | Challenge virus |
| PBS | 10 | 1 × 10⁶ PFU RSV A/A2 |
| RSV A2 | 5 | 1 × 10⁶ PFU RSV A/A2 |
| W3A(HN-L)-RSV- | 10 | 1 × 10⁶ PFU RSV A/A2 |
| F, 10⁶ PFU, i.n. | | |
| W3A(SH-HN)- | 10 | 1 × 10⁶ PFU RSV A/A2 |
| RSV-F, 10⁶ PFU, | | |
| i.n. | | |
| W3A(SH-HN)- | 10 | 1 × 10⁶ PFU RSV A/A2 |
| RSV-pF, 10⁶ PFU, | | |
| i.n. | | |
| W3AΔSH-RSV-F, | 10 | 1 × 10⁶ PFU RSV A/A2 |
| 10⁶ PFU, i.n. | | |
| W3AΔSH-RSV-pF, | 10 | 1 × 10⁶ PFU RSV A/A2 |
| 10⁶ PFU, i.n. | | |

Following immunization, both humoral and cell mediated immune responses were observed. The highest neutralizing antibody responses were detected with vaccine construct using the wt F protein. Also, vaccine constructs containing the wt F inserted prior to the HN junction with ΔSH seemed to be most immunogenic. The level of cell-mediated immune responses (based on IFN-gamma using ELISPOT) was similar between the various vaccine constructs.

Mice were challenged 28 days after immunization with RSV A2 to determine protective efficacy. Challenge virus was only obtained from one of five mice at day 4 post challenge in the W3AΔSH-RSV-F group with none of the mice in the other vaccination groups. In the PBS control group challenge virus was recovered from all mice.

A similar study was conducted in cotton rats which are more permissive to RSV infection. Rats were immunized with low dose of 10³ PFU of the modified vectors (W3A-RSV-F (SH-HN), W3A-RSV-pF, W3AΔSH-RSV-F) containing wild type or perfusion stabilized F-protein (pF) or 10² PFU of W3AΔSH-RSV-pF.

Immune responses were observed in all groups including neutralizing antibody responses against RSV A Tracy strain (97% identical to RSV A/A2 strain). Similar to the mouse study, the groups immunized with vaccine constructs containing the wt F protein had higher antibody levels compared to the pF groups, with F insertion prior to the HN gene junction with ΔSH having the highest values (approx. titer of 128). The neutralization antibody titers to RSV/B/18537 were significantly lower than to RSV/A Tracy strain, only the W3A(SH-HN)-RSV-F and W3AΔSH-RSV-F groups had significant antibody levels (approx. titer of 8) detected. After RSV challenge on Day 28 with 1.21×10⁵ PFU of RSV/A/Tracy, reduction in RSV viral load in nasal washes (1.4-1.66 Log₁₀ reduction) and lung lavage fluid (2-3 Log₁₀ reduction) was observed for all vaccine dose groups. The RSV challenge virus was recovered from all mice in the PBS control group (approx. 10⁵ PFU per nasal wash, or 10⁵ PFU/g lung wash).

Overall, these initial studies in the mouse and cotton rat models did not show evidence of the prefusion F-protein being more immunogenic and protective then the wild type F protein. Also, no difference was observed in the vector performance between ΔSH and the SH-HN insertion vector construct for the intranasal route.

Sigmovir Protocol #XV-131 Study report (Phan et al. 2017) challenge studies in cotton rat using intranasal and subcutaneous route: In a follow-on study (1, Phan et al. 2017) the efficacy, immunogenicity and safety of higher doses 10⁵ and 10⁶ PFU of W3A(SH-NH)-RSV-F and W3AΔSH-RSV-F were tested in a cotton rat challenge study evaluating two different routes intranasal (i.n.) and subcutaneous (s.c). This study also included a positive control for enhancement of disease (animals immunized with FI-RSV followed by RSV challenge and a positive control group consisting of animals that were pre-infected with RSV followed by RSV challenge. Animals in this study were challenged with RSV A2 on Day 49 followed by necropsy and histology 5 days later (Day 54). The study groups in this study were as follows (Table 3).

TABLE 3
Study groups in Protocol #XV-131.

| | N = number | |
|---|---|---|
| Vaccine/route | of animals | RSV/A2 Challenge |
| PBS i.m. | 5 | PBS |
| PBS i.m | 5 | 5Log₁₀ PFU |
| FI-RSV i.m | 5 | 5Log₁₀ PFU |
| RSV/A2 Live, i.n. | 5 | 5Log₁₀ PFU |
| (positive control) | | |
| W3A(SH-NH)- | 5 | 5Log₁₀ PFU |
| RSV-F, 10⁵ PFU | | |
| i.n. | | |
| W3A(SH-NH)- | 5 | 5Log₁₀ PFU |
| RSV-F, 10⁵ PFU | | |
| s.c | | |
| W3A(SH-NH)- | 5 | 5Log₁₀ PFU |
| RSV-F, 10⁶ PFU | | |
| s.c | | |
| W3AΔSH-RSV-F, | 5 | 5Log₁₀ PFU |
| 10⁵ PFU i.n. | | |
| W3AΔSH-RSV-F, | 5 | 5Log₁₀ PFU |
| 10⁵ PFU s.c. | | |
| W3AΔSH-RSV-F, | 5 | 5Log₁₀ PFU |
| 10⁶ s.c. | | |

Immunogenicity: W3A(SH-HN)-RSV-F resulted in similar neutralizing antibody titer in 10⁵ and 10⁶ dose between i.n and s.c group. W3AΔSH-RSV-F vaccination induced slightly higher neutralizing antibody titers in i.n group compared to s.c group, with 10⁶ dose resulting in a slightly higher titer, although it was not statistically significant.

Efficacy: W3A(SH-HN)-RSV-F resulted in complete protection in the lower respiratory tract by either i.n or s.c administration. Animals also had significantly lower viral loads in the upper respiratory tract.

W3A(SH-HN)-RSV-F vaccination resulted in complete protection in the lower respiratory tract by s.c administration and almost complete protection by i.n administration based on reduced titers (average titer of 10³ PFU) observed in lung and nasal washes compared to control animals sham immunized with PBS (average titer of 10⁵ PFU).

Safety: Lung sections from the different groups were examined for hallmarks of pulmonary inflammation: peribronchiolitis, perivasculitis, interstitial pneumonia, and alveolitis and scored for severity. The largest lesions were observed in the FI-RSV-immunized, RSV-challenged group (positive control group). Pulmonary changes were moderate in the groups immunized with W3A-RSV-F (SH-HN) or W3AΔSH-RSV-F (intranasally or subcutaneously), with levels below those observed in the RSV-immunized, RSV-challenged positive control group and similar to those in the PBS sham-immunized, RSV-challenged group.

Cytokine levels measured in lung tissues at 5 days post-challenge by quantitative real-time PCR (qPCR) were not indicative of potential of enhanced infection. The IL-4 mRNA level was significantly elevated only in the FI-RSV-vaccinated group, consistent with the histopathology results and the enhanced disease phenotype. IFNγ-mRNA levels were the highest in the sham-immunized and FI-RSV-immunized groups. IFN-mRNA levels were similarly low between groups immunized with the PIV5-based candidates and the RSV-immunized group. IL-2 mRNA levels were similar in all groups, but the average IL-2 level in the FI-RSV immunized group was significantly higher than that in the other groups.

After intranasal challenge of immunized mice with RSV A2 (10⁶ PFU in 50 μl) at Day 28 after immunization, lung sections obtained at 4 days after challenge showed no exacerbation of lung lesions relative to RSV A2-immunized mice. Protective immunity was observed as assessed by viral load in lung tissues (n=5 mice per group).

PIV5 (W3A) expressing RSV F or G protein challenge study in cotton rats and African green monkeys: This study evaluated PIV5 vectored RSV F protein in cotton rats and African green monkeys for replication, immunogenicity and efficacy of protection against RSV challenge. In this study, the F protein was inserted into the intergenic regions of PIV5 HN and L gene.

PIV5 replication permissiveness in cotton rats and African green monkeys: Cotton rats (n=4 per group) were inoculated intranasally with 1×10⁵ PFU of PIV5 at volumes of 10 μl or 100 μl. Viral titers were observed in nose homogenates (up 1×10⁴ PFU) at day 4 which cleared mostly by day 6. The larger inoculum volume (100 μl) resulted in observation of vaccine virus in the lungs of all the animals at day 6, which was only observed for one animal inoculated with the smaller volume of 10 μl.

To evaluate PIV5 permissiveness in African green monkeys, 60 animals were screened for anti-PIV5 antibodies and all were found to be negative. Animals (n=3 per group) were infected intranasally with 1×10² up to 1×10⁸ PFU of PIV5 in 0.25 mL dose volume. Nasal wash and bronchoalveolar washes were assessed for virus shedding on days 3, 5, 7, 10 and 14. Virus was shed from both nose and lungs for up to 10-day period with peak replication at Day 5 for the dose as low as 1×10² PFU. This data demonstrates permissiveness of African green monkeys for PIV5.

Immunogenicity in cotton rats and African green monkeys after single dose W3A-RSV-F: Cotton rats were immunized intranasally with 1×10³, 1×10⁴, 1×10⁵ and 1×10⁶ PFU of W3A-RSV-F. At all dose levels, IgG antibody responses were noted at Day 28 with comparable responses per dose group. Neutralizing antibodies were observed in all dose group ranging from titer of 64 to 256. In addition, IgA responses in lung homogenates were observed in all dose groups at Day 21 post inoculation.

African green monkeys (PIV5 and RSV serum negative) received a single intranasal immunization with 1×10⁴ or 1×10⁶ PFU W3A-RSV-F. Sera obtained at Day 21 post inoculation showed high titers of F-specific antibody responses. In addition, neutralizing antibody responses were observed at Day 21 at low levels (52 in 1×10⁶ PFU dose group). Nasal swabs obtained at 21 days post immunization showed significant levels of F-protein IgA responses. Cell mediated responses as assessed by gamma interferon showed low level responses in the 1×10⁶ PFU dose group.

Protection in cotton rats (Study #131) and African green monkeys from RSV challenge: Cotton rats immunized intranasally with single dose ranging from 1×10³ to 1×10⁶ PFU W3A-RSV-F were challenged with RSV A2 strain at Day 28 after immunization. Protection was assessed by measuring viral loads in nose and lung tissues. Reduced viral loads were observed in a dose-dependent manner except for the 1×10⁶ PFU group which showed higher levels relative to 1×10³ PFU group but still reduced compared to control group. In the 1×10⁵ dose group, no virus was detected in the lung and reduced titers (order of 3 log₁₀) were observed in the nose.

African green monkeys immunized with 1×10⁴ or 1×10⁶ PFU W3A-RSV-F were challenged 28 days after immunization with RSV A2. Nasal and BAL samples were assessed for RSV viral load Days 3-14 after challenge. Immunization with W3A-RSV-F did not shorten virus shedding, however, peak viral RSV loads were reduced 10 to 100-fold in both dose groups, with the highest reduction observed in the 1×10⁶ PFU dose group.

Responses to W3A-RSV-F in RSV exposed African green monkeys: Prior exposure to RSV did not interfere with ability to boost RSV neutralization antibody titers (50-fold increase) in African green monkeys (seroconverted by intranasal infection with RSV A2).

Lung pathology in W3A-RSV-F immunized cotton rats after RSV challenge: In animals immunized at Day 0 with dose 1×10⁶ PFU PIV5-RSV-F and challenged at Day 49 with RSV A2, lungs obtained five days after challenge were blindly examined by histopathology for alveolitis, interstitial pneumonitis, perivasculitis, and peribroncholitis and compared to lungs obtained from control animals inoculated with Formalin-inactivated RSV. The scores of positive control animals inoculated with formalin-inactivated RSV were significantly higher than PBS control animals but not the scores of W3A-RSV-F animals (ANOVA paired t-test).

### B. Parainfluenza Virus 5 (PIV5)

Parainfluenza virus 5 (PIV5), a negative-stranded RNA virus, is a member of the Rubulavirus genus of the family Paramyxoviridae which includes many important human and animal pathogens such as mumps virus, human parainfluenza virus type 2 and type 4, Newcastle disease virus, Sendai virus, HPIV3, measles virus, canine distemper virus, rinderpest virus and respiratory syncytial virus. PIV5 was previously known as Simian Virus-5 (SV5). Although PIV5 is a virus that infects many animals and humans, no known symptoms or diseases in humans have been associated with PIV5. Unlike most paramyxoviruses, PIV5 infect normal cells with little cytopathic effect. As a negative stranded RNA virus, the genome of PIV5 is very stable. As PIV5 does not have a DNA phase in its life cycle and it replicates solely in cytoplasm, PIV5 is unable to integrate into the host genome. Therefore, using PIV5 as a vector avoids possible unintended consequences from genetic modifications of host cell DNAs. PIV5 can grow to high titers in cells, including Vero cells which have been approved for vaccine production by WHO and FDA. Thus, PIV5 presents many advantages as a vaccine vector.

A PIV5-based vaccine vector of the present invention may be based on any of a variety of wild type, mutant, or recombinant (rPIV5) strains. Wild type strains include, but are not limited to, the PIV5 strains W3A, WR (ATCC® Number VR-288™), canine parainfluenza virus strain 78-238 (ATCC number VR-1573) (Evermann et al., 1980, J Am Vet Med Assoc; 177:1132-1134; and Evermann et al., 1981, Arch Virol; 68:165-172), canine parainfluenza virus strain D008 (ATCC number VR-399) (Binn et al., 1967, Proc Soc Exp Biol Med; 126:140-145), MIL, DEN, LN, MEL, cryptovirus, CPI+, CPI−, H221, 78524, T1 and SER. See, for example, Chatziandreou et al., 2004, J Gen Virol; 85(Pt 10):3007-16; Choppin, 1964, Virology: 23:224-233; and Baumgartner et al., 1987, Intervirology; 27:218-223. Additionally, PIV5 strains used in commercial kennel cough vaccines, such as, for example, BI, FD, Merck, and Merial vaccines, may be used.

### C. PIV5 CPI Strain Vector Backbone

The PIV5 CPI strain vector backbone differs from that of PIV5 W3A strain vector as follows (FIG. 1). The most notable difference is in the PIV5 F protein of the CPI strain that consists of an additional 22 amino acid extension as part of its cytoplasmic tail. The extension of the F protein is thought to result in inhibition of the fusogenic properties of the virus (5,6). CPI based viruses are more lytic and produce more progeny virus in infected cells compared to the W3A based viruses that does not have the extended PIV5 F protein tail and possess additional amino acid difference compared with CPI (4). CVB backbone with S156N or S157F in PIV5 W3A improves virus yield in cell culture and is more immunogenic in vivo than CPI backbone.

Previously, recombinant PIV5 viruses expressing foreign genes from numerous pathogens, including Influenza, Rabies, Respiratory Syncytial Virus, Tuberculosis, Burkholderia, and MERS-CoV, SARS-CoV-2 have been generated and tested as vaccine candidates (Li, Z., et al., J Virol, 87(1):354 (2013); Chen, Z., et al., J Virol, 87(6): 2986 (2013); Wang, D., et al., J Virol, 91(11) (2017); Chen, Z., et al., Vaccine, 33(51):7217 (2015); Lafontaine, E. R., et al., Vaccine X., 1:100002 (2018); Li, K., et al., mBio, 11(2) (2020); An et al., Sci Adv, July 2; 7, 2021). Because it actively replicates in the respiratory tract following intranasal immunization, PIV5-vectored vaccines can generate mucosal immunity that includes antigen-specific IgA antibodies and long-lived IgA plasma cells (Wang, D., et al., J Virol, 91(11) (2017). Xiao, P., et al., Front Immunol,. 12:623996 (2021)).

### D. PIV5 CVB Backbone

Provided herein is a modified PIV5 vector, CVB. The CVB backbone vector is immunogenic and can be used as an effective vaccine platform. The CVB backbone comprises mutations at amino acid residue S157 or S156 of the P/V gene, wherein a phosphorylation site is removed resulting in higher transcription activities thereby improving virus titer in cell culture.

In some embodiments, the mutation at amino acid residue S157 or S156 comprises the substitution of serine (S) with an amino acid residue selected from a group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), selenocysteine (U), valine (V), tryptophan (W), and tyrosine (Y). In one embodiment, the amino acid substitution at amino acid residue S157 comprises a substitution of serine (S) to phenylalanine (F) or S156 comprises a substitution of serine (S) to asparagine (N).

i. CVB Backbone

The CVB backbone nucleic acid sequence is provided herein:

(SEQ ID NO: 1)
ACCAAGGGGAAAATGAAGTGGTGACTCAAATCATCGAAGACCCTC
GAGATTACATAGGTCCGGAACCTATGGCCTTCGTGACCGACCTCG
AGTCAGAGTAGTTCAATAAGGACCTATCAAGTTTGGGCAATTTTT
CGTCCCCGACACAAAAATGTCATCCGTGCTTAAAGCATATGAGCG
ATTCACGCTCACTCAAGAACTGCAAGATCAGAGTGAGGAAGGTAC
AATCCCACCTACAACACTAAAACCGGTAATCAGGGTATTTATACT
AACCTCTAATAACCCAGAGCTAAGATCCCGGCTTCTTCTATTCTG
CCTACGGATTGTTCTCAGTAATGGTGCAAGGGATTCCCATCGCTT
TGGAGCATTACTCACAATGTTTTCGCTACCATCAGCCACAATGCT
CAATCATGTCAAATTAGCTGACCAGTCACCAGAAGCTGATATCGA
AAGGGTAGAGATCGATGGCTTTGAGGAGGGATCATTCCGCTTAAT
CCCCAATGCTCGTTCAGGTATGAGCCGTGGAGAGATCAATGCCTA
TGCTGCACTTGCAGAAGATCTACCTGACACACTAAACCATGCAAC
ACCTTTCGTTGATTCCGAAGTCGAGGGAACTGCATGGGATGAGAT
TGAGACTTTCTTAGATATGTGTTACAGTGTCCTAATGCAGGCATG
GATAGTGACTTGCAAGTGCATGACTGCGCCAGACCAACCTGCTGC
TTCTATTGAGAAACGCCTGCAAAAATATCGTCAGCAAGGCAGGAT
CAACCCGAGATATCTCCTGCAACCGGAGGCTCGACGAATAATCCA
GAATGTAATCCGGAAGGGAATGGTGGTCAGACATTTCCTCACCTT
TGAACTGCAGCTTGCCCGAGCACAAAGCCTTGTATCAAATAGGTA
TTATGCTATGGTAGGGGATGTTGGAAAGTATATAGAGAATTGTGG
AATGGGAGGCTTCTTTTTGACACTAAAATATGCATTAGGAACTAG
ATGGCCCACACTTGCTTTAGCTGCATTTTCAGGAGAGCTAACAAA
GCTAAAGTCCCTCATGGCATTATACCAGACCCTTGGTGAGCAGGC
CCGATATTTGGCCCTATTGGAGTCACCACATTTGATGGATTTTGC
TGCAGCAAACTACCCACTGCTATATAGCTATGCTATGGGAATAGG
CTATGTGTTAGATGTCAACATGAGGAACTACGCTTTCTCCAGATC
ATACATGAACAAGACATATTTCCAATTGGGAATGGAAACTGCAAG
AAAACAACAGGGTGCAGTTGACATGAGGATGGCAGAAGATCTCGG
TCTAACTCAAGCCGAACGCACCGAGATGGCAAATACACTTGCCAA
ATTGACCACAGCAAATCGAGGGGCAGACACCAGGGGAGGAGTCAA
CCCGTTCTCATCTGTCACTGGGACAACTCAGGTGCCCGCTGCAGC
AACAGGTGACACACTCGAGAGTTACATGGCAGCGGATCGACTGAG
GCAGAGATATGCTGATGCAGGCACCCATGATGATGAGATGCCACC
ATTGGAAGAGGAGGAAGAGGACGACACATCTGCAGGTCCACGCAC
TGGACCAACTCTTGAACAAGTGGCCTTGGACATCCAGAACGCAGC
AGTTGGAGCTCCCATCCATACAGATGACCTGAATGCCGCACTGGG
TGATCTTGACATCTAGACAATTCAGATCCCAATCTAAAATTGACA
TACCTAATTGATTAGTTAGATGGAACTACAGTGGATTCCATAAGG
TTCCTGCCTACCATCGGCTTTAAAGAAAAAAATAGGCCCGGACGG
GTTAGCAACAAGCGACTGCCGGTGCCAACAGCGCAATCCACAATC
TACAATGGATCCCACTGATCTGAGCTTCTCCCCAGATGAGATCAA
TAAGCTCATAGAGACAGGCCTGAATACTGTAGAGTATTTTACTTC
CCAACAAGTCACAGGAACATCCTCTCTTGGAAAGAATACAATACC
ACCAGGGGTCACAGGACTACTAACCAATGCTGCAGAGGCAAAGAT
CCAAGAGTCAACTAACCATCAGAAGGGCTCAGTTGGTGGGGGTGC
AAAACCAAAGAAACCGCGACCAAAAATTGCCATTGTGCCAGCAGA
TGACAAAACAGTGCCCGGAAAGCCGATCCCAAACCCTCTATTAGG
TCTGGACTCCACCCCGAGCACCCAAACTGTGCTTGATCTAAGTGG
GAAAACATTACCATCAGGATCCTATAAGGGGGTTAAGCTTGCGAA
ATTTGGAAAAGAAAATCTGATGACACGGTTCATCGAGGAACCCAG
AGAGAATCCTATCGCAACCAGTTtCCCCATCGATTTTAAGAGGGG
CAGGGATACCGGCGGGTTCCATAGAAGGGAGTACTCAATCGGATG
GGTGGGAGATGAAGTCAAGGTCACTGAGTGGTGCAATCCATCCTG
TTCTCCAATCACCGCTGCAGCAAGGCGATTTGAATGCACTTGTCA
CCAGTGTCCAGTCACTTGCTCTGAATGTGAACGAGATACTTAATA
CAGTGAGAAATTTGGACTCTCGGATGAATCAACTGGAGACAAAAG
TAGATCGCATTCTCTCATCTCAGTCTCTAATCCAGACCATCAAGA
ATGACATAGTTGGACTTAAAGCAGGGATGGCTACTTTAGAAGGAA
TGATTACAACTGTGAAAATCATGGACCCGGGAGTTCCCAGTAATG
TTACTGTGGAAGATGTACGCAAGACACTAAGTAACCATGCTGTTG
TTGTGCCAGAATCATTCAATGATAGTTTCTTGACTCAATCTGAAG
ATGTAATTTCACTTGATGAGTTGGCTCGACCAACTGCAACAAGTG
TTAAGAAGATTGTCAGGAAGGTTCCTCCTCAGAAGGATCTGACTG
GATTGAAGATTACACTAGAGCAATTGGCAAAGGATTGCATCAGCA
AACCGAAGATGAGGGAAGAGTATCTCCTCAAAATCAACCAGGCTT
CCAGTGAGGCTCAGCTAATTGACCTCAAGAAAGCAATCATCCGCA
GTGCAATTTGATCAAGAAACACCCAATTACACTACACTGGTATGA
CACTGTACTAACCCTGAGGGTTTTAGAAAAAACGATTAACGATAA
ATAAGCCCGAACACTACACACTACCTGAGGCAGCCATGCCATCCA
TCAGCATTCCCGCAGACCCCACCAATCCACGTCAATCAATAAAAG
CGTTCCCAATTGTGATCAACAGTGATGGGGGTGAGAAAGGCCGCT
TGGTTAAACAACTACGCACAACCTACTTGAATGACCTAGATACTC
ATGAGCCACTGGTGACATTCATAAATACCTATGGATTCATCTACG
AACAGGATCGGGGGAATACCATTGTCGGAGAGGATCAACTTGGGA
AGAAAAGAGAGGCTGTGACCGCTGCAATGGTTACCCTTGGATGTG
GGCCTAATCTACCATCATTAGGGAATGTCCTGGGACAACTGAGGG
AATTCCAGGTCACTGTTAGGAAGACATCCAGCAAAGCGGAAGAGA
TGGTCTTTGAAATTGTTAAGTATCCGAGAATATTTCGGGGTCATA
CATTAATCCAGAAAGGACTAGTCTGTGTCTCCGCAGAAAAATTTG
TTAAGTCACCAGGGAAAATACAATCTGGAATGGACTATCTCTTCA
TTCCGACATTTCTGTCAGTGACTTACTGTCCAGCTGCAATCAAAT
TTCAGGTACCTGGCCCCATGTTGAAAATGAGATCAAGATACACTC
AGAGCTTACAACTTGAACTAATGATAAGAATCCTGTGTAAGCCCG
ATTCGCCACTTATGAAGGTCCATACCCCTGACAAGGAGGGAAGAG
GATGTCTTGTATCAGTATGGCTGCATGTATGCAACATCTTCAAAT
CAGGAAACAAGAATGGCAGTGAGTGGCAGGAATACTGGATGAGAA
AGTGTGCTAACATGCAACTTGAAGTGTCGATTGCAGATATGTGGG
GACCAACTATCATAATTCATGCCAGAGGTCACATTCCCAAAAGTG
CTAAGTTGTTTTTTGGAAAGGGTGGATGGAGCTGCCATCCACTTC
ACGAAGTTGTTCCAAGTGTCACTAAAACACTATGGTCCGTGGGCT
GTGAGATTACAAAGGCGAAGGCAATAATACAAGAGAGTAGCATCT
CTCTTCTCGTGGAGACTACTGACATCATAAGTCCAAAAGTCAAAA
TTTCATCTAAGCATCGCCGCTTTGGGAAATCAAATTGGGGTCTGT
TCAAGAAAACTAAATCACTGCCTAACCTGACGGAGCTGGAATGAC
TGACCTCTAATCGAGACTACACCGCCGCAAACTATAGGTGGGTGG
TACCTCAGTGATTAATCTTGTAAGCACTGATCGTAGGCTACAACA
CACTAATATTATCCAGATTAGAGAGCTTAATTAGCTCTGTATTAA
TAATAACACTACTATTCCAATAACTGGAATCACCAGCTTGATTTA
TCTCCAAAATGATTCAAAGAAAACAAATCATATTAAGACTATCCT
AAGCACGAACCCATATCGTCCTTCAAATCATGGGTACTATAATTC
AATTTCTGGTGGTCTCCTGTCTATTGGCAGGAGCAGGCAGCCTTG
ATCCAGCAGCCCTCATGCAAATCGGTGTCATTCCAACAAATGTCC
GGCAACTTATGTATTATACTGAAGCTTCATCAGCATTCATTGTTG
TGAAGTTAATGCCTACAATTGACTCGCCGATTAGTGGATGTAATA
TAACATCAATTTCAAGCTATAATGCAACAGTGACAAAACTCCTAC
AGCCGATCGGTGAGAATTTGGAGACGATTAGGAACCAGTTGATTC
CAACTCGGAGGAGACGCCGGTTTGCAGGGGTGGTGATTGGATTAG
CTGCATTAGGAGTAGCTACTGCCGCACAGGTCACTGCCGCAGTGG
CACTAGTAAAGGCAAATGAAAATGCTGCGGCTATACTCAATCTCA
AAAATGCAATCCAAAAAACAAATGCAGCAGTTGCAGATGTGGTCC
AGGCCACACAATCACTAGGAACGGCAGTTCAAGCAGTTCAAGATC
ACATAAACAGTGTGGTAAGTCCAGCAATTACAGCAGCCAATTGTA
AGGCCCAAGATGCTATCATTGGCTCAATCCTCAATCTCTATTTGA
CCGAGTTGACAACCATCTTCCACAATCAAATTACAAACCCTGCAT
TGAGTCCCATTACAATTCAAGCTTTAAGGATCCTACTGGGGAGTA
CCTTGCCGACTGTGGTCGAAAAATCTTTCAATACCCAGATAAGTG
CAGCTGAGCTTCTCTCATCAGGGTTATTGACAGGCCAGATTGTGG
GATTAGATTTGACCTATATGCAGATGGTCATAAAAATTGAGCTGC
CAACTTTAACTGTACAACCTGCAACCCAGATCATAGATCTGGCCA
CCATTTCTGCATTCATTAACAATCAAGAAGTCATGGCCCAATTAC
CAACACGTGTTATGGTGACTGGCAGCTTGATCCAAGCCTATCCCG
CATCGCAATGCACCATTACACCCAACACTGTGTACTGTAGGTATA
ATGATGCCCAAGTACTCTCAGATGATACTATGGCTTGCCTCCAAG
GTAACTTGACAAGATGCACCTTCTCTCCAGTGGTTGGGAGCTTTC
TCACTCGATTCGTGCTGTTCGATGGAATAGTTTATGCAAATTGCA
GGTCGATGTTGTGCAAGTGCATGCAACCTGCTGCTGTGATCCTAC
AGCCGAGTTCATCCCCTGTAACTGTCATTGACATGTACAAATGTG
TGAGTCTGCAGCTTGACAATCTCAGATTCACCATCACTCAATTGG
CCAATGTAACCTACAATAGCACCATCAAGCTTGAATCATCCCAGA
TCTTGTCTATTGATCCGTTGGATATATCCCAGAATCTAGCTGCGG
TGAATAAGAGTCTAAGTGATGCACTACAACACTTAGCACAAAGTG
ACACATATCTTTCTGCAATCACATCAGCTACGACTACAAGTGTAT
TATCCATAATAGCAATCTGTCTTGGATCGTTAGGTTTAATATTAA
TAATCTTGCTCAGTGTAGTTGTGTGGAAGTTATTGACCATTGTCG
TTGCTAATCGAAATAGAATGGAGAATTTTGTTTATCATAAATAAG
CATTCCACCACTCACGATCTGATCTCAGTGAGAAAAATCAACCTG
CAACTCTTGGAACAAGATAAGACAGTCATCCATTAGTAATTTTTA
AGAAAAAAACGATAGGACCGAACCTAGTATTGAAAGAACCGTCTC
GGTCAATCTAGGTAATCGAGCTGATACCGTCTCGGAAAGCTCAAA
TCCTGCTATAGGCTATCCACTGCATCATCTCTCCTGCCATACTTC
CTACTCACATCATATCTATTTTAAAGAAAAAATAGGCCCGAACAC
TAATCGTGCCGGCAGTGCCACTGCACACACAACACTACACATACA
ATACACTACAATGGTTGCAGAAGATGCCCCTGTTAGGGCCACTTG
CCGAGTATTATTTCGAACAACAACTTTAATCTTTCTATGCACACT
ACTAGCATTAAGCATCTCTATCCTTTATGAGAGTTTAATAACCCA
AAAGCAAATCATGAGCCAAGCAGGCTCAACTGGATCTAATTCTGG
ATTAGGAAGTATCACTGATCTTCTTAATAATATTCTCTCTGTCGC
AAATCAGATTATATATAACTCTGCAGTCGCTCTACCTCTACAATT
GGACACTCTTGAATCAACACTCCTTACAGCCATTAAGTCTCTTCA
AACCAGTGACAAGCTAGAACAGAACTGCTCGTGGAGTGCTGCACT
GATTAATGATAATAGATACATTAATGGCATCAATCAGTTCTATTT
TTCAATTGCTGAGGGTCGCAATCTGACACTTGGCCCACTTCTTAA
TATGCCTAGTTTCATTCCAACTGCCACGACACCAGAGGGCTGCAC
CAGGATCCCATCATTCTCGCTCACTAAGACACACTGGTGTTATAC
ACACAATGTTATCCTGAATGGATGCCAGGATCATGTATCCTCAAA
TCAATTTGTTTCTATGGGAATCATTGAACCCACTTCTGCCGGGTT
TCCATTCTTTCGAACCCTAAAGACTCTATATCTCAGCGATGGGGT
CAATCGTAAGAGCTGCTCTATCAGTACAGTTCCGGGGGGTTGTAT
GATGTACTGTTTTGTTTCTACTCAACCAGAGAGGGATGACTACTT
TTCTGCCGCTCCTCCAGAACAACGAATTATTATAATGTACTATAA
TGATACAATCGTGGAGCGCATAATTAATCCACCCGGGGTACTAGA
TGTATGGGCAACATTGAACCCAGGAACAGGAAGCGGGGTATATTA
TTTAGGTTGGGTGCTCTTTCCAATATATGGCGGCGTGATTAAAGG
TACGAGTTTATGGAATAATCAAGCAAATAAATACTTTATCCCCCA
GATGGTTGCTGCTCTCTGCTCACAAAACCAGGCAACTCAAGTCCA
AAATGCTAAGTCATCATACTATAGCAGCTGGTTTGGCAATCGAAT
GATTCAGTCTGGGATCCTGGCATGTCCTCTTCGACAGGATCTAAC
CAATGAGTGTTTAGTTCTGCCCTTTTCTAATGATCAGGTGCTTAT
GGGTGCTGAAGGGAGATTATACATGTATGGTGACTCGGTGTATTA
CTATCAAAGAAGCAATAGTTGGTGGCCTATGACCATGCTGTATAA
GGTAACCATAACATTCACTAATGGTCAGCCATCTGCTATATCAGC
TCAGAATGTGCCCACACAGCAGGTCCCTAGACCTGGGACAGGAGA
CTGCTCTGCAACCAATAGATGTCCCGGTTTTTGCTTGACAGGAGT
GTATGCCGATGCCTGGTTACTGACCAACCCTTCGTCTACCAGTAC
ATTTGGATCAGAAGCAACCTTCACTGGTTCTTATCTCAACACAGC
AACTCAGCGTATCAATCCGACGATGTATATCGCGAACAACACACA
GATCATAAGCTCACAGCAATTTGGATCAAGCGGTCAAGAAGCAGC
ATATGGCCACACAACTTGTTTTAGGGACACAGGCTCTGTTATGGT
ATACTGTATCTATATTATTGAATTGTCCTCATCTCTCTTAGGACA
ATTTCAGATTGTCCCATTTATCCGTCAGGTGACACTATCCTAAAG
GCAGAAGCCTTCAGGTCTGACCCAGCCAATCAAAGCATTATACCA
GACCATGGAATGCATACCAAACATTATTGACACTAATGACACACA
AAATTGGTTTTAAGAAAAACCAAGAGAACAATAGGCCAGAATGGC
TGGGTCTCGGGAGATATTACTCCCTGAAGTCCATCTCAATTCACC
AATTGTAAAGCATAAGCTATACTATTACATTCTACTTGGAAACCT
CCCAAATGAGATCGACCTTGACGATTTAGGTCCATTACATAATCA
AAATTGGAATCAGATAGCACATGAAGAGTCTAACTTAGCTCAACG
CTTGGTAAATGTAAGAAATTTTCTAATTACCCACATCCCTGATCT
TAGAAAGGGCCATTGGCAAGAGTATGTCAATGTAATACTGTGGCC
GCGAATTCTTCCCTTGATCCCGGATTTTAAAATCAATGACCAATT
GCCTCTGCTCAAAAATTGGGACAAGTTAGTTAAAGAATCATGTTC
AGTAATCAATGCAGGTACTTCCCAGTGCATTCAGAATCTCAGCTA
TGGACTGACAGGTCGTGGGAACCTCTTTACACGATCACGTGAACT
CTCTGGTGACCGCAGGGATATTGATCTTAAGACAGTTGTGGCAGC
ATGGCATGACTCAGACTGGAAAAGAATAAGTGATTTTTGGATTAT
GATCAAATTCCAGATGAGACAATTAATTGTTAGGCAAACAGATCA
TAATGATTCTGATTTAATCACGTATATCGAAAATAGAGAAGGCAT
AATCATCATAACCCCTGAACTGGTAGCATTATTTAACACTGAGAA
TCATACACTAACATACATGACCTTTGAAATTGTACTGATGGTTTC
AGATATGTACGAAGGTCGTCACAACATTTTATCACTATGCACAGT
TAGCACTTACCTGAATCCTCTGAAGAAAAGAATAACATATTTATT
GAGCCTTGTAGATAACTTAGCTTTTCAGATAGGTGATGCTGTATA
TAACATAATTGCTTTGCTAGAATCCTTTGTATATGCACAGTTGCA
AATGTCAGATCCCATCCCAGAACTCAGAGGACAATTCCATGCATT
CGTATGTTCTGAGATTCTTGATGCACTAAGAGGAACTAATAGTTT
CACCCAGGATGAATTAAGAACTGTGACAACTAATTTGATATCCCC
ATTCCAAGATCTGACCCCAGATCTTACGGCTGAATTGCTCTGTAT
AATGAGGCTTTGGGGACACCCCATGCTCACTGCCAGTCAAGCTGC
AGGAAAGGTACGCGAGTCTATGTGTGCTGGAAAAGTATTAGACTT
TCCCACCATTATGAAAACACTAGCCTTTTTCCATACTATTCTGAT
CAATGGATACAGGAGGAAGCATCATGGAGTATGGCCACCCTTAAA
CTTACCGGGTAATGCTTCAAAGGGTCTCACGGAACTTATGAATGA
CAATACTGAAATAAGCTATGAATTCACACTTAAGCATTGGAAGGA
AGTCTCTCTTATAAAATTCAAGAAATGTTTTGATGCAGACGCAGG
TGAGGAACTCAGTATATTTATGAAAGATAAGGCAATTAGTGCCCC
AAAACAAGACTGGATGAGTGTGTTTAGAAGAAGCCTAATCAAACA
GCGCCATCAGCATCATCAGGTCCCCCTACCAAATCCATTCAATCG
ACGGCTGTTGCTAAACTTTCTCGGAGATGACAAATTCGACCCGAA
TGTGGAGCTACAGTATGTAACATCAGGTGAGTATCTACATGATGA
CACGTTTTGTGCATCATATTCACTAAAAGAGAAGGAAATTAAACC
TGATGGTCGAATTTTTGCAAAGTTGACTAAGAGAATGAGATCATG
TCAAGTTATAGCAGAATCTCTTTTAGCGAACCATGCTGGGAAGTT
AATGAAAGAGAATGGTGTTGTGATGAATCAGCTATCATTAACAAA
ATCACTATTAACAATGAGTCAGATTGGAATAATATCCGAGAAAGC
TAGAAAGTCAACTCGAGATAACATAAATCAACCTGGTTTCCAGAA
TATCCAGAGAAATAAATCACATCACTCCAAGCAAGTCAATCAGCG
AGATCCAAGTGATGACTTTGAATTGGCAGCATCTTTTTTAACTAC
TGATCTCAAAAAATATTGTTTACAATGGAGGTACCAGACAATTAT
CCCATTTGCTCAATCATTAAACAGAATGTATGGTTATCCTCATCT
CTTTGAGTGGATTCACTTACGGCTAATGCGTAGTACACTTTACGT
GGGGGATCCCTTCAACCCACCAGCAGATACCAGTCAATTTGATCT
AGATAAAGTAATTAATGGAGATATCTTCATTGTATCACCCAGAGG
TGGAATTGAAGGGCTGTGTCAAAAGGCTTGGACAATGATATCTAT
CGCTGTGATAATTCTATCTGCCACAGAGTCTGGCACACGAGTAAT
GAGTATGGTGCAGGGAGATAATCAAGCAATTGCTGTCACCACACG
AGTACCAAGGAGCCTGCCGACTCTTGAGAAAAAGACTATTGCTTT
TAGATCTTGTAATCTATTCTTTGAGAGGTTAAAATGTAATAATTT
TGGATTAGGTCACCATTTGAAAGAACAAGAGACTATCATTAGTTC
TCACTTCTTTGTTTATAGCAAGAGAATATTCTATCAGGGGAGGAT
TCTAACGCAAGCCTTAAAAAATGCTAGTAAGCTCTGCTTGACAGC
TGATGTCCTAGGAGAATGCACCCAATCATCATGTTCTAATCTTGC
AACTACTGTCATGAGGTTAACTGAGAATGGTGTTGAAAAAGATAT
CTGTTTCTACTTGAATATCTATATGACCATCAAACAGCTCTCCTA
TGATATCATCTTCCCTCAAGTGTCAATTCCTGGAGATCAGATCAC
ATTAGAATACATAAATAATCCACACCTGGTATCACGATTGGCTCT
TTTGCCATCCCAGTTAGGAGGTCTAAACTACCTGTCATGCAGTAG
GCTGTTCAATCGAAACATAGGCGACCCGGTGGTTTCCGCAGTTGC
AGATCTTAAGAGATTAATTAAATCAGGATGTATGGATTACTGGAT
CCTTTATAACTTATTAGGGAGAAAACCGGGAAACGGCTCATGGGC
TACTTTAGCAGCTGACCCGTACTCAATCAATATAGAGTATCAATA
CCCTCCAACTACAGCTCTTAAGAGGCACACCCAACAAGCTCTGAT
GGAACTCAGTACGAATCCAATGTTACGTGGCATATTCTCTGACAA
TGCACAGGCAGAAGAAAATAACCTTGCTAGGTTTCTCCTGGATAG
GGAGGTGATCTTTCCGCGTGTAGCTCACATCATCATTGAGCAAAC
CAGTGTCGGGAGGAGAAAACAGATTCAAGGATATTTGGATTCAAC
TAGATCGATAATGAGGAAATCACTAGAAATTAAGCCCTTATCCAA
TAGGAAGCTTAATGAAATACTGGATTACAACATCAATTACCTAGC
TTACAATTTGGCATTACTCAAGAATGCTATTGAACCTCCGACTTA
TTTGAAGGCAATGACACTTGAAACATGTAGCATCGACATTGCAAG
GAACCTCCGGAAGCTCTCCTGGGCCCCACTCTTGGGTGGGAGAAA
TCTTGAAGGATTAGAGACGCCAGATCCCATTGAAATTACTGCAGG
AGCATTAATTGTTGGATCGGGCTACTGTGAACAGTGTGCTGCAGG
AGACAATCGATTCACATGGTTTTTCTTGCCATCTGGTATCGAGAT
AGGAGGGGATCCCCGTGATAATCCTCCTATCCGTGTACCGTACAT
TGGCTCCAGGACTGATGAGAGGAGGGTAGCCTCAATGGCATACAT
CAGGGGTGCCTCGAGTAGCTTAAAAGCAGTTCTTAGACTGGCGGG
AGTGTACATCTGGGCATTCGGAGATACTCTGGAGAATTGGATAGA
TGCACTGGATTTGTCTCACACTAGAGTTAACATCACACTTGAACA
GCTGCAATCCCTCACCCCACTTCCAACCTCTGCCAATCTAACCCA
TCGGTTGGATGATGGCACAACTACCCTAAAGTTTACTCCTGCGAG
CTCTTACACCTTTTCAAGTTTCACTCATATATCAAATGATGAGCA
ATACCTGACAATTAATGACAAAACTGCAGATTCAAATATAATCTA
CCAACAGTTAATGATCACTGGACTCGGAATCTTAGAAACATGGAA
TAATCCCCCAATCAATAGAACATTCGAAGAATCTACCCTACATTT
GCACACTGGTGCATCATGTTGTGTCCGACCTGTGGACTCCTGCAT
TCTCTCAGAAGCATTAACAGTCAAGCCACATATTACAGTACCGTA
CAGCAATAAATTTGTATTTGATGAGGACCCGCTATCTGAATATGA
AACTGCAAAACTGGAATCGTTATCATTCCAAGCCCAATTAGGCAA
CATTGATGCTGTAGATATGACAGGTAAATTAACATTATTGTCCCA
ATTCACTGCAAGGCAGATTATCAATGCAATCACTGGACTCGATGA
GTCTGTCTCTCTTACTAATGATGCCATTGTTGCATCAGACTATGT
CTCCAATTGGATTAGTGAATGCATGTATACCAAATTAGATGAATT
ATTTATGTATTGTGGGTGGGAACTACTATTGGAACTATCCTATCA
AATGTATTATCTGAGGGTAGTTGGGTGGAGTAATATAGTGGATTA
TTCTTACATGATCTTGAGAAGAATCCCGGGTGCAGCATTAAACAA
TCTGGCATCTACATTAAGTCATCCAAAACTTTTCCGACGAGCTAT
CAACCTAGATATAGTTGCCCCCTTAAATGCTCCTCATTTTGCATC
TCTGGACTACATCAAGATGAGTGTGGATGCAATACTCTGGGGCTG
TAAAAGAGTCATCAATGTGCTCTCCAATGGAGGGGACTTAGAATT
AGTTGTGACATCTGAAGATAGCCTTATTCTCAGTGACCGATCCAT
GAATCTCATTGCAAGGAAATTAACTTTATTATCACTGATTCACCA
TAATGGTTTGGAACTACCAAAGATTAAGGGGTTCTCTCCTGATGA
GAAGTGTTTCGCTTTGACAGAATTTTTGAGGAAAGTGGTGAACTC
AGGGTTGAGTTCAATAGAGAACCTATCAAATTTTATGTACAATGT
GGAGAACCCACGGCTTGCAGCATTCGCCAGCAACAATTACTACCT
GACCAGAAAATTATTGAATTCAATACGAGATACTGAGTCGGGTCA
AGTAGCAGTCACCTCATATTATGAATCATTAGAATATATTGATAG
TCTTAAGCTAACCCCACATGTGCCTGGCACCTCATGCATTGAGGA
TGATAGTCTATGTACAAATGATTACATAATCTGGATCATAGAGTC
TAATGCAAACTTGGAGAAGTATCCAATTCCAAATAGCCCTGAGGA
TGATTCCAATTTCCATAACTTTAAGTTGAATGCTCCATCGCACCA
TACCTTACGCCCATTAGGGTTGTCATCAACTGCTTGGTATAAGGG
TATAAGCTGCTGCAGGTACCTTGAGCGATTAAAGCTACCACAAGG
TGATCATTTATATATTGCAGAAGGTAGTGGTGCCAGTATGACAAT
CATAGAATACCTATTCCCAGGAAGAAAGATATATTACAATTCTTT
ATTTAGTAGTGGTGACAATCCCCCACAAAGAAATTATGCACCAAT
GCCTACTCAGTTCATTGAGAGTGTCCCATACAAGCTCTGGCAAGC
ACACACAGATCAATATCCCGAGATTTTTGAGGACTTCATCCCTCT
ATGGAACGGAAACGCCGCCATGACTGACATAGGAATGACAGCTTG
TGTAGAATTCATCATCAATCGAGTCGGCCCAAGGACTTGCAGTTT
AGTACATGTAGATTTGGAATCAAGTGCAAGCTTAAATCAACAATG
CCTGTCAAAGCCGATAATTAATGCTATCATCACTGCTACAACTGT
TTTGTGCCCTCATGGGGTGCTTATTCTGAAATATAGTTGGTTGCC
ATTTACTAGATTTAGTACTTTGATCACTTTCTTATGGTGCTACTT
TGAGAGAATCACTGTTCTTAGGAGCACATATTCTGATCCAGCTAA
TCATGAGGTTTATTTAATTTGTATCCTTGCCAACAACTTTGCATT
CCAGACTGTCTCGCAGGCAACAGGAATGGCGATGACTTTAACTGA
TCAAGGGTTTACTTTGATATCACCTGAAAGAATAAATCAGTATTG
GGATGGTCACTTGAAGCAAGAACGTATCGTAGCAGAAGCAATTGA
TAAGGTGGTTCTAGGAGAAAATGCTCTATTTAATTCGAGTGATAA
TGAATTAATTCTCAAATGTGGAGGGACACCAAATGCACGGAATCT
CATCGATATCGAGCCAGTCGCAACTTTCATAGAATTTGAACAATT
GATCTGCACAATGTTGACAACCCACTTGAAGGAAATAATTGATAT
AACAAGGTCTGGAACCCAGGATTATGAAAGTTTATTACTCACTCC
TTACAATTTAGGTCTTCTTGGTAAAATCAGTACGATAGTGAGATT
ATTAACAGAAAGGATTCTAAATCATACTATCAGGAATTGGTTGAT
CCTCCCACCTTCGCTCCGGATGATCGTGAAGCAGGACTTGGAATT
CGGCATATTCAGGATTACTTCCATCCTCAATTCTGATCGGTTCCT
GAAGCTTTCTCCAAATAGGAAATACTTGATTGCACAATTAACTGC
AGGCTACATTAGGAAATTGATTGAGGGGGATTGCAATATCGATCT
AACCAGACCTATCCAAAAGCAAATCTGGAAAGCATTAGGTTGTGT
AGTCTATTGTCACGATCCAATGGATCAAAGGGAGTCAACAGAGTT
TATTGATATAAATATTAATGAAGAAATAGACCGCGGGATCGATGG
CGAGGAAATCTAAACATATCAAGAATCAGAATTAGTTTAAGAAAA
AAGAAGAGGATTAATCTTGGTTTTCCCCTTGGT.

### III. CVB-Based RSV Compositions

Provided herein are the CVB-RSV-F genomic sequences.

### A. CVB-RSV-F Sequence

The CVB-RSV-F nucleic acid sequence is provided herein:

(SEQ ID NO: 2)
ACCAAGGGGAAAATGAAGTGGTGACTCAAATCATCGAAGACCCTC
GAGATTACATAGGTCCGGAACCTATGGCCTTCGTGACCGACCTCG
AGTCAGAGTAGTTCAATAAGGACCTATCAAGTTTGGGCAATTTTT
CGTCCCCGACACAAAAATGTCATCCGTGCTTAAAGCATATGAGCG
ATTCACGCTCACTCAAGAACTGCAAGATCAGAGTGAGGAAGGTAC
AATCCCACCTACAACACTAAAACCGGTAATCAGGGTATTTATACT
AACCTCTAATAACCCAGAGCTAAGATCCCGGCTTCTTCTATTCTG
CCTACGGATTGTTCTCAGTAATGGTGCAAGGGATTCCCATCGCTT
TGGAGCATTACTCACAATGTTTTCGCTACCATCAGCCACAATGCT
CAATCATGTCAAATTAGCTGACCAGTCACCAGAAGCTGATATCGA
AAGGGTAGAGATCGATGGCTTTGAGGAGGGATCATTCCGCTTAAT
CCCCAATGCTCGTTCAGGTATGAGCCGTGGAGAGATCAATGCCTA
TGCTGCACTTGCAGAAGATCTACCTGACACACTAAACCATGCAAC
ACCTTTCGTTGATTCCGAAGTCGAGGGAACTGCATGGGATGAGAT
TGAGACTTTCTTAGATATGTGTTACAGTGTCCTAATGCAGGCATG
GATAGTGACTTGCAAGTGCATGACTGCGCCAGACCAACCTGCTGC
TTCTATTGAGAAACGCCTGCAAAAATATCGTCAGCAAGGCAGGAT
CAACCCGAGATATCTCCTGCAACCGGAGGCTCGACGAATAATCCA
GAATGTAATCCGGAAGGGAATGGTGGTCAGACATTTCCTCACCTT
TGAACTGCAGCTTGCCCGAGCACAAAGCCTTGTATCAAATAGGTA
TTATGCTATGGTAGGGGATGTTGGAAAGTATATAGAGAATTGTGG
AATGGGAGGCTTCTTTTTGACACTAAAATATGCATTAGGAACTAG
ATGGCCCACACTTGCTTTAGCTGCATTTTCAGGAGAGCTAACAAA
GCTAAAGTCCCTCATGGCATTATACCAGACCCTTGGTGAGCAGGC
CCGATATTTGGCCCTATTGGAGTCACCACATTTGATGGATTTTGC
TGCAGCAAACTACCCACTGCTATATAGCTATGCTATGGGAATAGG
CTATGTGTTAGATGTCAACATGAGGAACTACGCTTTCTCCAGATC
ATACATGAACAAGACATATTTCCAATTGGGAATGGAAACTGCAAG
AAAACAACAGGGTGCAGTTGACATGAGGATGGCAGAAGATCTCGG
TCTAACTCAAGCCGAACGCACCGAGATGGCAAATACACTTGCCAA
ATTGACCACAGCAAATCGAGGGGCAGACACCAGGGGAGGAGTCAA
CCCGTTCTCATCTGTCACTGGGACAACTCAGGTGCCCGCTGCAGC
AACAGGTGACACACTCGAGAGTTACATGGCAGCGGATCGACTGAG
GCAGAGATATGCTGATGCAGGCACCCATGATGATGAGATGCCACC
ATTGGAAGAGGAGGAAGAGGACGACACATCTGCAGGTCCACGCAC
TGGACCAACTCTTGAACAAGTGGCCTTGGACATCCAGAACGCAGC
AGTTGGAGCTCCCATCCATACAGATGACCTGAATGCCGCACTGGG
TGATCTTGACATCTAGACAATTCAGATCCCAATCTAAAATTGACA
TACCTAATTGATTAGTTAGATGGAACTACAGTGGATTCCATAAGG
TTCCTGCCTACCATCGGCTTTAAAGAAAAAAATAGGCCCGGACGG
GTTAGCAACAAGCGACTGCCGGTGCCAACAGCGCAATCCACAATC
TACAATGGATCCCACTGATCTGAGCTTCTCCCCAGATGAGATCAA
TAAGCTCATAGAGACAGGCCTGAATACTGTAGAGTATTTTACTTC
CCAACAAGTCACAGGAACATCCTCTCTTGGAAAGAATACAATACC
ACCAGGGGTCACAGGACTACTAACCAATGCTGCAGAGGCAAAGAT
CCAAGAGTCAACTAACCATCAGAAGGGCTCAGTTGGTGGGGGTGC
AAAACCAAAGAAACCGCGACCAAAAATTGCCATTGTGCCAGCAGA
TGACAAAACAGTGCCCGGAAAGCCGATCCCAAACCCTCTATTAGG
TCTGGACTCCACCCCGAGCACCCAAACTGTGCTTGATCTAAGTGG
GAAAACATTACCATCAGGATCCTATAAGGGGGTTAAGCTTGCGAA
ATTTGGAAAAGAAAATCTGATGACACGGTTCATCGAGGAACCCAG
AGAGAATCCTATCGCAACCAGTTTCCCCATCGATTTTAAGAGGGG
CAGGGATACCGGCGGGTTCCATAGAAGGGAGTACTCAATCGGATG
GGTGGGAGATGAAGTCAAGGTCACTGAGTGGTGCAATCCATCCTG
TTCTCCAATCACCGCTGCAGCAAGGCGATTTGAATGCACTTGTCA
CCAGTGTCCAGTCACTTGCTCTGAATGTGAACGAGATACTTAATA
CAGTGAGAAATTTGGACTCTCGGATGAATCAACTGGAGACAAAAG
TAGATCGCATTCTCTCATCTCAGTCTCTAATCCAGACCATCAAGA
ATGACATAGTTGGACTTAAAGCAGGGATGGCTACTTTAGAAGGAA
TGATTACAACTGTGAAAATCATGGACCCGGGAGTTCCCAGTAATG
TTACTGTGGAAGATGTACGCAAGACACTAAGTAACCATGCTGTTG
TTGTGCCAGAATCATTCAATGATagtTTCTTGACTCAATCTGAAG
ATGTAATTTCACTTGATGAGTTGGCTCGACCAACTGCAACAAGTG
TTAAGAAGATTGTCAGGAAGGTTCCTCCTCAGAAGGATCTGACTG
GATTGAAGATTACACTAGAGCAATTGGCAAAGGATTGCATCAGCA
AACCGAAGATGAGGGAAGAGTATCTCCTCAAAATCAACCAGGCTT
CCAGTGAGGCTCAGCTAATTGACCTCAAGAAAGCAATCATCCGCA
GTGCAATTTGATCAAGAAACACCCAATTACACTACACTGGTATGA
CACTGTACTAACCCTGAGGGTTTTAGAAAAAACGATTAACGATAA
ATAAGCCCGAACACTACACACTACCTGAGGCAGCCATGCCATCCA
TCAGCATTCCCGCAGACCCCACCAATCCACGTCAATCAATAAAAG
CGTTCCCAATTGTGATCAACAGTGATGGGGGTGAGAAAGGCCGCT
TGGTTAAACAACTACGCACAACCTACTTGAATGACCTAGATACTC
ATGAGCCACTGGTGACATTCATAAATACCTATGGATTCATCTACG
AACAGGATCGGGGGAATACCATTGTCGGAGAGGATCAACTTGGGA
AGAAAAGAGAGGCTGTGACCGCTGCAATGGTTACCCTTGGATGTG
GGCCTAATCTACCATCATTAGGGAATGTCCTGGGACAACTGAGGG
AATTCCAGGTCACTGTTAGGAAGACATCCAGCAAAGCGGAAGAGA
TGGTCTTTGAAATTGTTAAGTATCCGAGAATATTTCGGGGTCATA
CATTAATCCAGAAAGGACTAGTCTGTGTCTCCGCAGAAAAATTTG
TTAAGTCACCAGGGAAAATACAATCTGGAATGGACTATCTCTTCA
TTCCGACATTTCTGTCAGTGACTTACTGTCCAGCTGCAATCAAAT
TTCAGGTACCTGGCCCCATGTTGAAAATGAGATCAAGATACACTC
AGAGCTTACAACTTGAACTAATGATAAGAATCCTGTGTAAGCCCG
ATTCGCCACTTATGAAGGTCCATACCCCTGACAAGGAGGGAAGAG
GATGTCTTGTATCAGTATGGCTGCATGTATGCAACATCTTCAAAT
CAGGAAACAAGAATGGCAGTGAGTGGCAGGAATACTGGATGAGAA
AGTGTGCTAACATGCAACTTGAAGTGTCGATTGCAGATATGTGGG
GACCAACTATCATAATTCATGCCAGAGGTCACATTCCCAAAAGTG
CTAAGTTGTTTTTTGGAAAGGGTGGATGGAGCTGCCATCCACTTC
ACGAAGTTGTTCCAAGTGTCACTAAAACACTATGGTCCGTGGGCT
GTGAGATTACAAAGGCGAAGGCAATAATACAAGAGAGTAGCATCT
CTCTTCTCGTGGAGACTACTGACATCATAAGTCCAAAAGTCAAAA
TTTCATCTAAGCATCGCCGCTTTGGGAAATCAAATTGGGGTCTGT
TCAAGAAAACTAAATCACTGCCTAACCTGACGGAGCTGGAATGAC
TGACCTCTAATCGAGACTACACCGCCGCAAACTATAGGTGGGTGG
TACCTCAGTGATTAATCTTGTAAGCACTGATCGTAGGCTACAACA
CACTAATATTATCCAGATTAGAGAGCTTAATTAGCTCTGTATTAA
TAATAACACTACTATTCCAATAACTGGAATCACCAGCTTGATTTA
TCTCCAAAATGATTCAAAGAAAACAAATCATATTAAGACTATCCT
AAGCACGAACCCATATCGTCCTTCAAATCATGGGTACTATAATTC
AATTTCTGGTGGTCTCCTGTCTATTGGCAGGAGCAGGCAGCCTTG
ATCCAGCAGCCCTCATGCAAATCGGTGTCATTCCAACAAATGTCC
GGCAACTTATGTATTATACTGAAGCTTCATCAGCATTCATTGTTG
TGAAGTTAATGCCTACAATTGACTCGCCGATTAGTGGATGTAATA
TAACATCAATTTCAAGCTATAATGCAACAGTGACAAAACTCCTAC
AGCCGATCGGTGAGAATTTGGAGACGATTAGGAACCAGTTGATTC
CAACTCGGAGGAGACGCCGGTTTGCAGGGGTGGTGATTGGATTAG
CTGCATTAGGAGTAGCTACTGCCGCACAGGTCACTGCCGCAGTGG
CACTAGTAAAGGCAAATGAAAATGCTGCGGCTATACTCAATCTCA
AAAATGCAATCCAAAAAACAAATGCAGCAGTTGCAGATGTGGTCC
AGGCCACACAATCACTAGGAACGGCAGTTCAAGCAGTTCAAGATC
ACATAAACAGTGTGGTAAGTCCAGCAATTACAGCAGCCAATTGTA
AGGCCCAAGATGCTATCATTGGCTCAATCCTCAATCTCTATTTGA
CCGAGTTGACAACCATCTTCCACAATCAAATTACAAACCCTGCAT
TGAGTCCCATTACAATTCAAGCTTTAAGGATCCTACTGGGGAGTA
CCTTGCCGACTGTGGTCGAAAAATCTTTCAATACCCAGATAAGTG
CAGCTGAGCTTCTCTCATCAGGGTTATTGACAGGCCAGATTGTGG
GATTAGATTTGACCTATATGCAGATGGTCATAAAAATTGAGCTGC
CAACTTTAACTGTACAACCTGCAACCCAGATCATAGATCTGGCCA
CCATTTCTGCATTCATTAACAATCAAGAAGTCATGGCCCAATTAC
CAACACGTGTTATGGTGACTGGCAGCTTGATCCAAGCCTATCCCG
CATCGCAATGCACCATTACACCCAACACTGTGTACTGTAGGTATA
ATGATGCCCAAGTACTCTCAGATGATACTATGGCTTGCCTCCAAG
GTAACTTGACAAGATGCACCTTCTCTCCAGTGGTTGGGAGCTTTC
TCACTCGATTCGTGCTGTTCGATGGAATAGTTTATGCAAATTGCA
GGTCGATGTTGTGCAAGTGCATGCAACCTGCTGCTGTGATCCTAC
AGCCGAGTTCATCCCCTGTAACTGTCATTGACATGTACAAATGTG
TGAGTCTGCAGCTTGACAATCTCAGATTCACCATCACTCAATTGG
CCAATGTAACCTACAATAGCACCATCAAGCTTGAATCATCCCAGA
TCTTGTCTATTGATCCGTTGGATATATCCCAGAATCTAGCTGCGG
TGAATAAGAGTCTAAGTGATGCACTACAACACTTAGCACAAAGTG
ACACATATCTTTCTGCAATCACATCAGCTACGACTACAAGTGTAT
TATCCATAATAGCAATCTGTCTTGGATCGTTAGGTTTAATATTAA
TAATCTTGCTCAGTGTAGTTGTGTGGAAGTTATTGACCATTGTCG
TTGCTAATCGAAATAGAATGGAGAATTTTGTTTATCATAAATAAG
CATTCCACCACTCACGATCTGATCTCAGTGAGAAAAATCAACCTG
CAACTCTTGGAACAAGATAAGACAGTCATCCATTAGTAATTTTTA
AGAAAAAAACGATAGGACCGAACCTAGTATTGAAAGAACCGTCTC
GGTCAATCTAGGTAATCGAGCTGATACCGTCTCGGAAAGCTCAAA
TCGCCGCCACCatggagctgctgatcctgaaagccaacgctatta
ccacaatcctgaccgccgtgacattctgctttgcttccggacaga
atatcaccgaggagttctaccagtcaacatgtagcgccgtgagca
aggggtacctgagtgcactgcgaaccggatggtatacatccgtga
ttactatcgagctgtctaacatcaagaaaaacaaatgcaatggca
ctgacgccaagatcaagctgatcaagcaggagctggataagtaca
aaaatgctgtgaccgaactgcagctgctgatgcagagcactccag
ctaccaacaatcgcgcacggagagaactgccccggttcatgaact
ataccctgaacaatgcaaagaaaacaaatgtgactctgtccaaga
aacgcaagaggcgcttcctgggatttctgctgggcgtggggtctg
caatcgccagtggagtggccgtctctaaagtcctgcacctggagg
gcgaagtgaacaagatcaaatcagcactgctgagcacaaacaagg
ccgtggtcagtctgtcaaatggcgtgtccgtcctgacttctaagg
tgctggacctgaaaaattatattgataagcagctgctgcctatcg
tcaacaaacagagctgttccatttctaatatcgagaccgtgatcg
agttccagcagaagaacaatagacttctagagattacaagggaat
tttctgtgaacgctggcgtcactacccccgtgagtacctacatgc
tgacaaatagtgagctgctgtcactgattaacgacatgcctatca
ccaatgatcagaagaaactgatgagtaacaatgtgcagatcgtca
gacagcagagttactcaatcatgtcaatcattaaggaggaagtcc
tggcctacgtggtccagctgccactgtatggcgtgatcgacactc
cctgctggaaactgcatacctcacctctgtgcacaactaacacca
aggaggggagcaatatctgcctgacacgaactgaccggggatggt
actgtgataacgccggcagcgtgtccttctttccccaggctgaga
cctgcaaggtccagtccaacagggtgttctgtgacactatgaata
gcctgaccctgccttccgaagtcaacctgtgcaatgtggacatct
ttaatccaaagtacgattgtaagatcatgaccagcaagacagatg
tcagctcgagtgtgattacatcactgggggctatcgtgagctgct
acggaaagactaaatgtaccgcaagcaacaaaaatcgcggcatca
ttaagaccttcagtaacgggtgcgactatgtctcaaacaagggcg
tggatacagtgagcgtcgggaacactctgtactatgtcaataagc
aggagggaaaatccctgtacgtgaagggcgaaccaatcattaact
tctatgaccccctggtgttcccttccgacgagtttgatgcctcta
ttagtcaggtgaacgaaaaaatcaatcagagcctggcttttattc
ggaagtccgatgagctgctgcacaacgtgaatgcagtcaagtcta
ccacaaacattatgatcactaccatcattatcgtgattatcgtca
ttctgctgtccctgatcgcagtggggctgctgctgtactgtaaag
ccagatctacacccgtgactctgtctaaggatcagctgagtggaa
ttaacaatatcgcctttagcaactgaTAGGTCGACCACCTGCTAT
AGGCTATCCACTGCATCATCTCTCCTGCCATACTTCCTACTCACA
TCATATCTATTTTAAAGAAAAAATAGGCCCGAACACTAATCGTGC
CGGCAGTGCCACTGCACACACAACACTACACATACAATACACTAC
AATGGTTGCAGAAGATGCCCCTGTTAGGGCCACTTGCCGAGTATT
ATTTCGAACAACAACTTTAATCTTTCTATGCACACTACTAGCATT
AAGCATCTCTATCCTTTATGAGAGTTTAATAACCCAAAAGCAAAT
CATGAGCCAAGCAGGCTCAACTGGATCTAATTCTGGATTAGGAAG
TATCACTGATCTTCTTAATAATATTCTCTCTGTCGCAAATCAGAT
TATATATAACTCTGCAGTCGCTCTACCTCTACAATTGGACACTCT
TGAATCAACACTCCTTACAGCCATTAAGTCTCTTCAAACCAGTGA
CAAGCTAGAACAGAACTGCTCGTGGAGTGCTGCACTGATTAATGA
TAATAGATACATTAATGGCATCAATCAGTTCTATTTTTCAATTGC
TGAGGGTCGCAATCTGACACTTGGCCCACTTCTTAATATGCCTAG
TTTCATTCCAACTGCCACGACACCAGAGGGCTGCACCAGGATCCC
ATCATTCTCGCTCACTAAGACACACTGGTGTTATACACACAATGT
TATCCTGAATGGATGCCAGGATCATGTATCCTCAAATCAATTTGT
TTCTATGGGAATCATTGAACCCACTTCTGCCGGGTTTCCATTCTT
TCGAACCCTAAAGACTCTATATCTCAGCGATGGGGTCAATCGTAA
GAGCTGCTCTATCAGTACAGTTCCGGGGGGTTGTATGATGTACTG
TTTTGTTTCTACTCAACCAGAGAGGGATGACTACTTTTCTGCCGC
TCCTCCAGAACAACGAATTATTATAATGTACTATAATGATACAAT
CGTGGAGCGCATAATTAATCCACCCGGGGTACTAGATGTATGGGC
AACATTGAACCCAGGAACAGGAAGCGGGGTATATTATTTAGGTTG
GGTGCTCTTTCCAATATATGGCGGCGTGATTAAAGGTACGAGTTT
ATGGAATAATCAAGCAAATAAATACTTTATCCCCCAGATGGTTGC
TGCTCTCTGCTCACAAAACCAGGCAACTCAAGTCCAAAATGCTAA
GTCATCATACTATAGCAGCTGGTTTGGCAATCGAATGATTCAGTC
TGGGATCCTGGCATGTCCTCTTCGACAGGATCTAACCAATGAGTG
TTTAGTTCTGCCCTTTTCTAATGATCAGGTGCTTATGGGTGCTGA
AGGGAGATTATACATGTATGGTGACTCGGTGTATTACTATCAAAG
AAGCAATAGTTGGTGGCCTATGACCATGCTGTATAAGGTAACCAT
AACATTCACTAATGGTCAGCCATCTGCTATATCAGCTCAGAATGT
GCCCACACAGCAGGTCCCTAGACCTGGGACAGGAGACTGCTCTGC
AACCAATAGATGTCCCGGTTTTTGCTTGACAGGAGTGTATGCCGA
TGCCTGGTTACTGACCAACCCTTCGTCTACCAGTACATTTGGATC
AGAAGCAACCTTCACTGGTTCTTATCTCAACACAGCAACTCAGCG
TATCAATCCGACGATGTATATCGCGAACAACACACAGATCATAAG
CTCACAGCAATTTGGATCAAGCGGTCAAGAAGCAGCATATGGCCA
CACAACTTGTTTTAGGGACACAGGCTCTGTTATGGTATACTGTAT
CTATATTATTGAATTGTCCTCATCTCTCTTAGGACAATTTCAGAT
TGTCCCATTTATCCGTCAGGTGACACTATCCTAAAGGCAGAAGCC
TTCAGGTCTGACCCAGCCAATCAAAGCATTATACCAGACCATGGA
ATGCATACCAAACATTATTGACACTAATGACACACAAAATTGGTT
TTAAGAAAAACCAAGAGAACAATAGGCCAGAATGGCTGGGTCTCG
GGAGATATTACTCCCTGAAGTCCATCTCAATTCACCAATTGTAAA
GCATAAGCTATACTATTACATTCTACTTGGAAACCTCCCAAATGA
GATCGACCTTGACGATTTAGGTCCATTACATAATCAAAATTGGAA
TCAGATAGCACATGAAGAGTCTAACTTAGCTCAACGCTTGGTAAA
TGTAAGAAATTTTCTAATTACCCACATCCCTGATCTTAGAAAGGG
CCATTGGCAAGAGTATGTCAATGTAATACTGTGGCCGCGAATTCT
TCCCTTGATCCCGGATTTTAAAATCAATGACCAATTGCCTCTGCT
CAAAAATTGGGACAAGTTAGTTAAAGAATCATGTTCAGTAATCAA
TGCAGGTACTTCCCAGTGCATTCAGAATCTCAGCTATGGACTGAC
AGGTCGTGGGAACCTCTTTACACGATCACGTGAACTCTCTGGTGA
CCGCAGGGATATTGATCTTAAGACAGTTGTGGCAGCATGGCATGA
CTCAGACTGGAAAAGAATAAGTGATTTTTGGATTATGATCAAATT
CCAGATGAGACAATTAATTGTTAGGCAAACAGATCATAATGATTC
TGATTTAATCACGTATATCGAAAATAGAGAAGGCATAATCATCAT
AACCCCTGAACTGGTAGCATTATTTAACACTGAGAATCATACACT
AACATACATGACCTTTGAAATTGTACTGATGGTTTCAGATATGTA
CGAAGGTCGTCACAACATTTTATCACTATGCACAGTTAGCACTTA
CCTGAATCCTCTGAAGAAAAGAATAACATATTTATTGAGCCTTGT
AGATAACTTAGCTTTTCAGATAGGTGATGCTGTATATAACATAAT
TGCTTTGCTAGAATCCTTTGTATATGCACAGTTGCAAATGTCAGA
TCCCATCCCAGAACTCAGAGGACAATTCCATGCATTCGTATGTTC
TGAGATTCTTGATGCACTAAGAGGAACTAATAGTTTCACCCAGGA
TGAATTAAGAACTGTGACAACTAATTTGATATCCCCATTCCAAGA
TCTGACCCCAGATCTTACGGCTGAATTGCTCTGTATAATGAGGCT
TTGGGGACACCCCATGCTCACTGCCAGTCAAGCTGCAGGAAAGGT
ACGCGAGTCTATGTGTGCTGGAAAAGTATTAGACTTTCCCACCAT
TATGAAAACACTAGCCTTTTTCCATACTATTCTGATCAATGGATA
CAGGAGGAAGCATCATGGAGTATGGCCACCCTTAAACTTACCGGG
TAATGCTTCAAAGGGTCTCACGGAACTTATGAATGACAATACTGA
AATAAGCTATGAATTCACACTTAAGCATTGGAAGGAAGTCTCTCT
TATAAAATTCAAGAAATGTTTTGATGCAGACGCAGGTGAGGAACT
CAGTATATTTATGAAAGATAAGGCAATTAGTGCCCCAAAACAAGA
CTGGATGAGTGTGTTTAGAAGAAGCCTAATCAAACAGCGCCATCA
GCATCATCAGGTCCCCCTACCAAATCCATTCAATCGACGGCTGTT
GCTAAACTTTCTCGGAGATGACAAATTCGACCCGAATGTGGAGCT
ACAGTATGTAACATCAGGTGAGTATCTACATGATGACACGTTTTG
TGCATCATATTCACTAAAAGAGAAGGAAATTAAACCTGATGGTCG
AATTTTTGCAAAGTTGACTAAGAGAATGAGATCATGTCAAGTTAT
AGCAGAATCTCTTTTAGCGAACCATGCTGGGAAGTTAATGAAAGA
GAATGGTGTTGTGATGAATCAGCTATCATTAACAAAATCACTATT
AACAATGAGTCAGATTGGAATAATATCCGAGAAAGCTAGAAAGTC
AACTCGAGATAACATAAATCAACCTGGTTTCCAGAATATCCAGAG
AAATAAATCACATCACTCCAAGCAAGTCAATCAGCGAGATCCAAG
TGATGACTTTGAATTGGCAGCATCTTTTTTAACTACTGATCTCAA
AAAATATTGTTTACAATGGAGGTACCAGACAATTATCCCATTTGC
TCAATCATTAAACAGAATGTATGGTTATCCTCATCTCTTTGAGTG
GATTCACTTACGGCTAATGCGTAGTACACTTTACGTGGGGGATCC
CTTCAACCCACCAGCAGATACCAGTCAATTTGATCTAGATAAAGT
AATTAATGGAGATATCTTCATTGTATCACCCAGAGGTGGAATTGA
AGGGCTGTGTCAAAAGGCTTGGACAATGATATCTATCGCTGTGAT
AATTCTATCTGCCACAGAGTCTGGCACACGAGTAATGAGTATGGT
GCAGGGAGATAATCAAGCAATTGCTGTCACCACACGAGTACCAAG
GAGCCTGCCGACTCTTGAGAAAAAGACTATTGCTTTTAGATCTTG
TAATCTATTCTTTGAGAGGTTAAAATGTAATAATTTTGGATTAGG
TCACCATTTGAAAGAACAAGAGACTATCATTAGTTCTCACTTCTT
TGTTTATAGCAAGAGAATATTCTATCAGGGGAGGATTCTAACGCA
AGCCTTAAAAAATGCTAGTAAGCTCTGCTTGACAGCTGATGTCCT
AGGAGAATGCACCCAATCATCATGTTCTAATCTTGCAACTACTGT
CATGAGGTTAACTGAGAATGGTGTTGAAAAAGATATCTGTTTCTA
CTTGAATATCTATATGACCATCAAACAGCTCTCCTATGATATCAT
CTTCCCTCAAGTGTCAATTCCTGGAGATCAGATCACATTAGAATA
CATAAATAATCCACACCTGGTATCACGATTGGCTCTTTTGCCATC
CCAGTTAGGAGGTCTAAACTACCTGTCATGCAGTAGGCTGTTCAA
TCGAAACATAGGCGACCCGGTGGTTTCCGCAGTTGCAGATCTTAA
GAGATTAATTAAATCAGGATGTATGGATTACTGGATCCTTTATAA
CTTATTAGGGAGAAAACCGGGAAACGGCTCATGGGCTACTTTAGC
AGCTGACCCGTACTCAATCAATATAGAGTATCAATACCCTCCAAC
TACAGCTCTTAAGAGGCACACCCAACAAGCTCTGATGGAACTCAG
TACGAATCCAATGTTACGTGGCATATTCTCTGACAATGCACAGGC
AGAAGAAAATAACCTTGCTAGGTTTCTCCTGGATAGGGAGGTGAT
CTTTCCGCGTGTAGCTCACATCATCATTGAGCAAACCAGTGTCGG
GAGGAGAAAACAGATTCAAGGATATTTGGATTCAACTAGATCGAT
AATGAGGAAATCACTAGAAATTAAGCCCTTATCCAATAGGAAGCT
TAATGAAATACTGGATTACAACATCAATTACCTAGCTTACAATTT
GGCATTACTCAAGAATGCTATTGAACCTCCGACTTATTTGAAGGC
AATGACACTTGAAACATGTAGCATCGACATTGCAAGGAACCTCCG
GAAGCTCTCCTGGGCCCCACTCTTGGGTGGGAGAAATCTTGAAGG
ATTAGAGACGCCAGATCCCATTGAAATTACTGCAGGAGCATTAAT
TGTTGGATCGGGCTACTGTGAACAGTGTGCTGCAGGAGACAATCG
ATTCACATGGTTTTTCTTGCCATCTGGTATCGAGATAGGAGGGGA
TCCCCGTGATAATCCTCCTATCCGTGTACCGTACATTGGCTCCAG
GACTGATGAGAGGAGGGTAGCCTCAATGGCATACATCAGGGGTGC
CTCGAGTAGCTTAAAAGCAGTTCTTAGACTGGCGGGAGTGTACAT
CTGGGCATTCGGAGATACTCTGGAGAATTGGATAGATGCACTGGA
TTTGTCTCACACTAGAGTTAACATCACACTTGAACAGCTGCAATC
CCTCACCCCACTTCCAACCTCTGCCAATCTAACCCATCGGTTGGA
TGATGGCACAACTACCCTAAAGTTTACTCCTGCGAGCTCTTACAC
CTTTTCAAGTTTCACTCATATATCAAATGATGAGCAATACCTGAC
AATTAATGACAAAACTGCAGATTCAAATATAATCTACCAACAGTT
AATGATCACTGGACTCGGAATCTTAGAAACATGGAATAATCCCCC
AATCAATAGAACATTCGAAGAATCTACCCTACATTTGCACACTGG
TGCATCATGTTGTGTCCGACCTGTGGACTCCTGCATTCTCTCAGA
AGCATTAACAGTCAAGCCACATATTACAGTACCGTACAGCAATAA
ATTTGTATTTGATGAGGACCCGCTATCTGAATATGAAACTGCAAA
ACTGGAATCGTTATCATTCCAAGCCCAATTAGGCAACATTGATGC
TGTAGATATGACAGGTAAATTAACATTATTGTCCCAATTCACTGC
AAGGCAGATTATCAATGCAATCACTGGACTCGATGAGTCTGTCTC
TCTTACTAATGATGCCATTGTTGCATCAGACTATGTCTCCAATTG
GATTAGTGAATGCATGTATACCAAATTAGATGAATTATTTATGTA
TTGTGGGTGGGAACTACTATTGGAACTATCCTATCAAATGTATTA
TCTGAGGGTAGTTGGGTGGAGTAATATAGTGGATTATTCTTACAT
GATCTTGAGAAGAATCCCGGGTGCAGCATTAAACAATCTGGCATC
TACATTAAGTCATCCAAAACTTTTCCGACGAGCTATCAACCTAGA
TATAGTTGCCCCCTTAAATGCTCCTCATTTTGCATCTCTGGACTA
CATCAAGATGAGTGTGGATGCAATACTCTGGGGCTGTAAAAGAGT
CATCAATGTGCTCTCCAATGGAGGGGACTTAGAATTAGTTGTGAC
ATCTGAAGATAGCCTTATTCTCAGTGACCGATCCATGAATCTCAT
TGCAAGGAAATTAACTTTATTATCACTGATTCACCATAATGGTTT
GGAACTACCAAAGATTAAGGGGTTCTCTCCTGATGAGAAGTGTTT
CGCTTTGACAGAATTTTTGAGGAAAGTGGTGAACTCAGGGTTGAG
TTCAATAGAGAACCTATCAAATTTTATGTACAATGTGGAGAACCC
ACGGCTTGCAGCATTCGCCAGCAACAATTACTACCTGACCAGAAA
ATTATTGAATTCAATACGAGATACTGAGTCGGGTCAAGTAGCAGT
CACCTCATATTATGAATCATTAGAATATATTGATAGTCTTAAGCT
AACCCCACATGTGCCTGGCACCTCATGCATTGAGGATGATAGTCT
ATGTACAAATGATTACATAATCTGGATCATAGAGTCTAATGCAAA
CTTGGAGAAGTATCCAATTCCAAATAGCCCTGAGGATGATTCCAA
TTTCCATAACTTTAAGTTGAATGCTCCATCGCACCATACCTTACG
CCCATTAGGGTTGTCATCAACTGCTTGGTATAAGGGTATAAGCTG
CTGCAGGTACCTTGAGCGATTAAAGCTACCACAAGGTGATCATTT
ATATATTGCAGAAGGTAGTGGTGCCAGTATGACAATCATAGAATA
CCTATTCCCAGGAAGAAAGATATATTACAATTCTTTATTTAGTAG
TGGTGACAATCCCCCACAAAGAAATTATGCACCAATGCCTACTCA
GTTCATTGAGAGTGTCCCATACAAGCTCTGGCAAGCACACACAGA
TCAATATCCCGAGATTTTTGAGGACTTCATCCCTCTATGGAACGG
AAACGCCGCCATGACTGACATAGGAATGACAGCTTGTGTAGAATT
CATCATCAATCGAGTCGGCCCAAGGACTTGCAGTTTAGTACATGT
AGATTTGGAATCAAGTGCAAGCTTAAATCAACAATGCCTGTCAAA
GCCGATAATTAATGCTATCATCACTGCTACAACTGTTTTGTGCCC
TCATGGGGTGCTTATTCTGAAATATAGTTGGTTGCCATTTACTAG
ATTTAGTACTTTGATCACTTTCTTATGGTGCTACTTTGAGAGAAT
CACTGTTCTTAGGAGCACATATTCTGATCCAGCTAATCATGAGGT
TTATTTAATTTGTATCCTTGCCAACAACTTTGCATTCCAGACTGT
CTCGCAGGCAACAGGAATGGCGATGACTTTAACTGATCAAGGGTT
TACTTTGATATCACCTGAAAGAATAAATCAGTATTGGGATGGTCA
CTTGAAGCAAGAACGTATCGTAGCAGAAGCAATTGATAAGGTGGT
TCTAGGAGAAAATGCTCTATTTAATTCGAGTGATAATGAATTAAT
TCTCAAATGTGGAGGGACACCAAATGCACGGAATCTCATCGATAT
CGAGCCAGTCGCAACTTTCATAGAATTTGAACAATTGATCTGCAC
AATGTTGACAACCCACTTGAAGGAAATAATTGATATAACAAGGTC
TGGAACCCAGGATTATGAAAGTTTATTACTCACTCCTTACAATTT
AGGTCTTCTTGGTAAAATCAGTACGATAGTGAGATTATTAACAGA
AAGGATTCTAAATCATACTATCAGGAATTGGTTGATCCTCCCACC
TTCGCTCCGGATGATCGTGAAGCAGGACTTGGAATTCGGCATATT
CAGGATTACTTCCATCCTCAATTCTGATCGGTTCCTGAAGCTTTC
TCCAAATAGGAAATACTTGATTGCACAATTAACTGCAGGCTACAT
TAGGAAATTGATTGAGGGGGATTGCAATATCGATCTAACCAGACC
TATCCAAAAGCAAATCTGGAAAGCATTAGGTTGTGTAGTCTATTG
TCACGATCCAATGGATCAAAGGGAGTCAACAGAGTTTATTGATAT
AAATATTAATGAAGAAATAGACCGCGGGATCGATGGCGAGGAAAT
CTAAACATATCAAGAATCAGAATTAGTTTAAGAAAAAAGAAGAGG
ATTAATCTTGGTTTTCCCCTTGGT.

The inserted RSV-F sequence is in the lowercase and the CVB sequence is in the uppercase.

### IV. CVB-based SARS-CoV-2 Compositions

The disclosure provides CVB-based SARS-CoV-2 compositions, systems and methods for their use in multiple applications including functional genomics, drug discovery, target validation, protein production (e.g., therapeutic proteins, vaccines, monoclonal antibodies), gene therapy, and therapeutic treatments such as cancer therapy.

### A. SARS-CoV-2

With the present invention, constructs of the modified parainfluenza virus type-5 (PIV5) virus, CVB, expressing the SARS-CoV-2 envelope spike (S) and nucleocapsid (N) protein have been generated for use as vaccines against COVID. These constructs demonstrate effectiveness as vaccines, with single dose intranasal immunization inducing protective immunity in ferrets and cats.

Coronavirus disease 2019 (COVID-19) is a newly emerging infectious disease currently spreading across the world. It is caused by a novel coronavirus, severe acute respiratory syndrome coronavirus 2 (SARS-CoV-2) (Zhu et al., 2020, N Engl J Med; 382:727-733). SARS-CoV-2 was first identified in Wuhan, China in December 2019, and has subsequently spread globally to cause the COVID-19 pandemic. The virus has infected more than 221 million persons world-wide, caused more than 4,574,000 deaths as of Sep. 8, 2021, and is poised to continue to spread in the absence of herd immunity. While vaccines and antibody therapies have been introduced worldwide, the emergence of multiple viral variants which are rapidly replacing the original virus identified in Wuhan has allowed for immune escape in vaccinated populations, presenting a need for improved vaccine efficacy.

SARS-CoV-2 is a single-stranded RNA-enveloped virus belonging to the ß coronavirus family (Lu et al., 2020, Lancet; 395:565-74). An RNA-based metagenomic next-generation sequencing approach has been applied to characterize its entire genome, which is 29,881 nucleotides (nt) in length (GenBank Sequence Accession MN908947) encoding 9860 amino acids (Chen et al., 2020, Emerg Microbes Infect; 9:313-9). Full-genome sequenced genomes available at GenBank include isolate 2019-nCoV WHU01 (GenBank accession number MN988668) and NC_045512 for SARS-CoV-2, both isolates from Wuhan, China, and at least seven additional sequences (MN938384.1, MN975262.1, MN985325.1, MN988713.1, MN994467.1, MN994468.1, and MN997409.1) which are >99.9% identical and are hereby incorporate by reference.

i. SARS-CoV-2 Variants

Since SARS-CoV-2 was first identified in 2019, multiple genetic variants of SARS-CoV-2 have been emerging and circulating around the world. Viral mutations and variants in the United States are routinely monitored through sequence-based surveillance, laboratory studies, and epidemiological investigations. The US government SARS-CoV-2 Interagency Group (SIG) developed a Variant Classification scheme that defines three classes of SARS-CoV-2 variants: variant of interest, variant of concern and variant of high consequence.

a. Variant of Interest

A SARS-CoV-2 variant of interest is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

A variant of interest might require one or more appropriate public health actions, including enhanced sequence surveillance, enhanced laboratory characterization, or epidemiological investigations to assess how easily the virus spreads to others, the severity of disease, the efficacy of therapeutics and whether currently approved or authorized vaccines offer protection. The growing list variants of interest that are being monitored and characterized include, but are not limited to, Eta, Iota, Kappa, Lambda and Mu.

b. Variant of Concern

A SARS-CoV-2 variant of concern is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures. Possible attributes of a variant of concern include evidence of impact on diagnostics, treatments, or vaccines, widespread interference with diagnostic test targets, evidence of substantially decreased susceptibility to one or more class of therapies, evidence of significant decreased neutralization by antibodies generated during previous infection or vaccination, evidence of reduced vaccine-induced protection from severe disease, evidence of increased transmissibility and evidence of increased disease severity.

Variants of concern might require one or more appropriate public health actions, such as notification to WHO under the International Health Regulations, reporting to CDC, local or regional efforts to control spread, increased testing, or research to determine the effectiveness of vaccines and treatments against the variant. Based on the characteristics of the variant, additional considerations may include the development of new diagnostics or the modification of vaccines or treatments. The growing list of variants of concern that are being closely monitored and characterized, includes, but is not limited to, Alpha, Beta, Delta, Gamma and Omicron.

c. Variant of High Consequence

A SARS-CoV-2 variant of high consequence has clear evidence that prevention measures or medical countermeasures (MCMs) have significantly reduced effectiveness relative to previously circulating variants. Possible attributes of a variant of high consequence include a demonstrated failure of diagnostic test targets, evidence to suggest a significant reduction in vaccine effectiveness, a disproportionately high number of vaccine breakthrough cases, or very low vaccine-induced protection against severe disease, significantly reduced susceptibility to multiple Emergency Use Authorization (EUA) or approved therapeutics and more severe clinical disease and increased hospitalizations.

A variant of high consequence would require notification to WHO under the International Health Regulations, reporting to CDC, an announcement of strategies to prevent or contain transmission, and recommendations to update treatments and vaccines. Currently, there are no SARS-CoV-2 variants that rise to the level of high consequence.

### B. PIV5 CVB Vectored SARS-CoV-2 Constructs

A PIV5 vaccine vector of the present invention may be constructed using any of a variety of methods, including, but not limited to, the reverse genetics system described in more detail in He et al. (Virology; 237(2):249-60, 1997). PIV5 encodes eight viral proteins. Nucleocapsid protein (NP), phosphoprotein (P) and large RNA polymerase (L) protein are important for transcription and replication of the viral RNA genome. The V protein plays important roles in viral pathogenesis as well as viral RNA synthesis. The fusion (F) protein, a glycoprotein, mediates both cell-to-cell and virus-to-cell fusion in a pH-independent manner that is essential for virus entry into cells. The structures of the F protein have been determined and critical amino acid residues for efficient fusion have been identified. The hemagglutinin-neuraminidase (HN) glycoprotein is also involved in virus entry and release from the host cells. The matrix (M) protein plays an important role in virus assembly and budding. The hydrophobic (SH) protein is a 44-residue hydrophobic integral membrane protein and is oriented in membranes with its N terminus in the cytoplasm. For reviews of the molecular biology of paramyxoviruses see, for example, Whelan et al., 2004, Curr Top Microbiol Immunol; 283:61-119; and Lamb & Parks, (2006). Paramyxoviridae: the viruses and their replication. In Fields Virology, 5th edn, pp. 1449-1496. Edited by D. M. Knipe & P. M. Howley. Philadelphia, PA: Lippincott Williams & Wilkins.

Because PIV5 actively replicates in the respiratory tract following intranasal immunization, PIV5-vectored vaccines can generate mucosal immunity that includes antigen-specific IgA antibodies and long-lived IgA plasma cells (Wang, D., et al., J Virol, 91(11) (2017). Xiao, P., et al., Front Immunol, 12:623996 (2021)). Recently a PIV5-vectored vaccine expressing the spike protein from SARS-CoV-2 Wuhan (WA1; CVXGA1) has been shown to be efficacious in mice and ferrets. A single, intranasal dose of CVXGA1 induced WA1-neutralizing antibodies and protected K18-hACE2 mice against lethal infection with SARS-CoV-2 WA1. Furthermore, a single, intranasal dose of CVXGA1 protected ferrets from infection with SARS-CoV-2 WA1 and blocked transmission to cohoused naïve ferrets (An, D., et al., Sci Adv, 7(27) (2021)). While these studies determined its efficacy against SARS-CoV-2 WA1, further studies were necessary to establish its efficacy against SARS-CoV-2 variants.

A PIV5 viral vaccine of the present invention may also have a mutation, alteration, or deletion in one or more of these eight proteins of the PIV5 genome. For example, a PIV5 viral expression vector may include one or more mutations, including, but not limited to any of those described herein. In some aspects, a combination of two or more (two, three, four, five, six, seven, or more) mutations may be advantageous and may demonstrated enhanced activity.

In one embodiment, the PIV5 vector was further modified by introducing the mutations in the PIV5 V/P gene and by deletion of the PIV5 SH gene, further enhancing vaccine efficiency. S157F and S308A in the PIV5 V/P genes have been shown previously to increase viral polymerase activities and improve viral titer or yield (Timani K A, Sun D, Sun M, et al. J Virol., 82(18):9123-9133 (2008); Sun D, Luthra P, Li Z, He B., PLoS Pathog., 5(7):e1000525 (2009)). PIV5 W3A strain-based RSV vaccine with a single S157F mutation was shown to induce higher levels of neutralizing antibodies than PIV5 CPI-vectored RSV vaccine in cotton rats (See table 19). PIV5 W3A strain lacking the SH gene and expressing influenza virus H5 HA protein induced higher levels of antibodies and provided better protection against influenza virus challenge (Li Z, Gabbard J D, Mooney A, et al., J Virol., 87(17): 9604-9609 (2013)). In one embodiment, a newly generated modified PIV5 viral vector backbone is presented herein and named as CVB through introducing S157F into the V/P gene, and deleting the SH gene from the PIV5 W3A viral genome. In another embodiment, CVB-vectored SARS-CoV-2 vaccines for intranasal immunization were generated.

A mutation includes, but is not limited to, a mutation of the V/P gene, a mutation of the shared N-terminus of the V and P proteins, a mutation of residues 26, 32, 33, 50, 102, 156, and/or 157 of the shared N-terminus of the V and P proteins, a mutation lacking the C-terminus of the V protein, a mutation lacking the small hydrophobic (SH) protein, a mutation of the fusion (F) protein, a mutation of the phosphoprotein (P), a mutation of the large RNA polymerase (L) protein, a mutation incorporating residues from canine parainfluenza virus, and/or a mutation that enhances syncytial formation.

A mutation may include, but is not limited to, rPIV5-V/P-CPI−, rPIV5-CPI−, rPIV5-CPI+, rPIV5V ΔC, rPIV-Rev, rPIV5-RL, rPIV5-P-S156N, rPIV5-P-S157A, rPIV5-P-S308A, rPIV5-L-A1981D and rPIV5-F-S443P, rPIV5-MDA7, rPIV5 ΔSH-CPI−, rPIV5 ΔSH-Rev, and combinations thereof.

PIV5 can infect cells productively with little cytopathic effect (CPE) in many cell types. In some cell types, PIV5 infection causes formation of syncytia, i.e., fusion of many cells together, leading to cell death. A mutation may include one or more mutations that promote syncytia formation (see, for example Paterson et al., 2000, Virology; 270:17-30).

The V protein of PIV5 plays a critical role in blocking apoptosis induced by virus. Recombinant PIV5 lacking the conserved cysteine-rich C-terminus (rPIV5V AC) of the V protein induces apoptosis in a variety of cells through an intrinsic apoptotic pathway, likely initiated through endoplasmic reticulum (ER)-stress (Sun et al., 2004, J Virol; 78:5068-5078). Mutant recombinant PIV5 with mutations in the N-terminus of the V/P gene products, such as rPIV5-CPI−, also induce apoptosis (Wansley and Parks, 2002, J Virol; 76:10109-10121). A mutation includes, but is not limited to, rPIV5 ΔSH, rPIV5-CPI−, rPIV5 VΔC, and combinations thereof.

i. CVB-Based Vaccine Vectors Encoding the SARS-CoV-2 Spike (S) Protein

With the CVB-based vaccine vectors of the present invention, a heterologous nucleotide sequence encoding the spike (S) protein of a coronavirus, including, but not limited to, the S protein of SARS-CoV-2, is inserted in the CVB genome. Coronavirus entry into host cells is mediated by the transmembrane S glycoprotein (Tortorici and Veesler, 2019, Adv Virus Res; 105:93-116). As the coronavirus S glycoprotein is surface-exposed and mediates entry into host cells, it is the main target of neutralizing antibodies upon infection and the focus of therapeutic and vaccine design. The spike S protein of SARS-CoV-2 is composed of two subunits, S1 and S2. The S1 subunit contains a receptor-binding domain that recognizes and binds to the host receptor angiotensin-converting enzyme 2, while the S2 subunit mediates viral cell membrane fusion by forming a six-helical bundle via the two-heptad repeat domain. (Huang et al., 2020, Acta Pharmacol Sinica; 0:1-9 (available on the worldwide web at doi.org/10.1038/s41401-020-0485-4).

The total length of SARS-CoV-2 S is 1273 amino acids (aa) and consists of a signal peptide (amino acids 1-13) located at the N-terminus, the Sl subunit (14-685 residues), and the S2 subunit (686-1273 residues); the last two regions are responsible for receptor binding and membrane fusion, respectively. In the S1 subunit, there is an N-terminal domain (14-305 residues) and a receptor-binding domain (RBD, 319-541 residues); the fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) comprise the S2 subunit (Xia et al., 2020, Cell Mol Immunol; 17:765-7).

In some CVB-based vaccine vectors of the present invention, the heterologous nucleotide sequence encoding the spike (S) protein of a coronavirus, including, but not limited to, the S protein of SARS-CoV-2, has been modified so that the cytoplasmic tail of the coronavirus S protein has been replaced with the cytoplasmic tail of the fusion (F) protein of PIV5.

In some CVB-based vaccine vectors of the present invention, the heterologous nucleotide sequence encoding the coronavirus S protein, including but not limited to, the S protein of SARS-CoV-2, has been modified so that the S protein includes an amino acid substitution at amino acid residue W886 and/or F888. In some aspects, the amino acid substitution at amino acid residue W886 includes a substitution of tryptophan (W) to arginine (R) and/or the amino acid substitution at amino acid residue W888 includes a substitution of phenylalanine (F) to arginine (R).

In some CVB-based vaccine vectors of the present invention, the heterologous nucleotide sequence encoding the spike (S) protein of a coronavirus, including, but not limited to, the S protein of SARS-CoV-2, includes both a modification so that the cytoplasmic tail of the coronavirus S protein has been replaced with the cytoplasmic tail of the fusion (F) protein of PIV5 and includes an amino acid substitution at amino acid residue W886 and/or F888. In some aspects, the amino acid substitution at amino acid residue W886 includes a substitution of tryptophan (W) to arginine (R) and/or the amino acid substitution at amino acid residue W888 includes a substitution of phenylalanine (F) to arginine (R).

The heterologous nucleotide sequence encoding the coronavirus S protein, including but not limited to the S protein of SARS-CoV-2, may be inserted in any of a variety of locations in the CVB genome.

CVB vaccine vectors encoding SARS-COV-2 variants of concern or variants of interest are disclosed herein. The CVB-based vaccine vectors may comprise inserting the SARS-CoV-2 spike (S) or the nucleocapsid (N) gene from different variants into CVB vector.

The CVB-based vaccine vectors of the present invention, have a CVB viral expression vector comprising mutations at amino acid residue S156 or S157 and a deletion of the small hydrophobic (SH) gene of the PIV5 W3A viral genome.

In some CVB-based vaccine vectors of the present invention, the mutation at amino acid residue S157 comprises the substitution of serine (S) with an amino acid residue selected from a group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), selenocysteine (U), valine (V), tryptophan (W), tyrosine (Y).

In some CVB-based vaccine vectors of the present invention, the amino acid substitution at amino acid residue S157 comprises a substitution of serine (S) to phenylalanine (F) or S156N comprises a substitution of serine (S) to asparagine (N).

In some CVB-based vaccine vectors of the present invention, the SH gene has a deletion of the SH open reading frame or a deletion of an entire SH gene transcript unit.

In some CVB-based vaccine vectors of the present invention, the PIV5 genome has a heterologous nucleic acid sequence with at least 98% sequence identity to SEQ ID NOs: 27, 28, 29, 30, 31, 32, or 33 and wherein the viral expression vector expresses a heterologous polypeptide comprising a coronavirus spike (S) and/or nucleocapsid (N) proteins.

In some CVB-based vaccine vectors of the present invention, the coronavirus S protein is a coronavirus S protein of the severe acute respiratory syndrome coronavirus 2 (SARS-CoV-2), a variant of interest or a variant of concern of SARS-CoV-2 and the coronavirus N protein is the coronavirus N protein of SARS-CoV-2, a variant of interest or a variant of concern of SARS-CoV-2.

In another embodiment, the coronavirus S protein is the coronavirus S protein of a SARS-CoV-2 Wuhan strain, a SARS-CoV-2 beta variant, a SARS-CoV-2 gamma variant, a SARS-CoV-2 delta variant, or a SARS-CoV-2 omicron variant and the coronavirus N protein is the coronavirus N protein of a SARS-CoV-2 Wuhan strain, a SARS-CoV-2 beta variant, a SARS-CoV-2 gamma variant, a SARS-CoV-2 delta variant, or a SARS-CoV-2 omicron variant.

In some CVB-based vaccine vectors of the present invention, the coronavirus S protein comprises the coronavirus S protein of SARS-CoV-2 and wherein the cytoplasmic tail of the coronavirus S protein has been replaced with the cytoplasmic tail of the fusion (F) protein of CVB.

In some CVB-based vaccine vectors of the present invention, the PIV5 W3A viral genome comprises open reading frame deletion mutations of the SH gene and the S gene of SARS-CoV-2 Wuhan strain is inserted between the PIV5 hemagglutinin (HN) and polymerase (L) genes of PIV5.

In some CVB-based vaccine vectors of the present invention, the entire SH gene transcript unit of PIV5 W3A viral genome is deleted and the S gene of the SARS-CoV-2 Wuhan strain is placed between the HN and L genes of PIV5.

In some CVB-based vaccine vectors of the present invention, the N gene of SARS-CoV-2 Wuhan strain is inserted to replace the SH gene of PIV5, and the S gene of SARS-CoV-2 Wuhan strain is inserted between the HN and L genes of PIV5.

In some CVB-based vaccine vectors of the present invention, the S gene of SARS-CoV-2 Omicron BA.1 variant is inserted to replace the S gene of SARS-CoV-2 Wuhan strain and the N gene of SARS-CoV-2 Wuhan strain is inserted in the place of SH gene of PIV5.

In some CVB-based vaccine vectors of the present invention, the S gene of SARS-CoV-2 Omicron BA.5 variant is inserted between the HN and L genes of PIV5 to replace the S gene of Wuhan strain.

In some CVB-based vaccine vectors of the present invention, the S gene of SARS-CoV-2 Omicron BA.5 variant is inserted to replace the S gene of Wuhan strain and the N gene of SARS-CoV-2 Wuhan strain is inserted to replace the SH gene of PIV5.

In some CVB-based vaccine vectors of the present invention, the PIV5 F and HN genes are deleted and wherein the S gene of SARS-CoV-2 Wuhan strain is between HN and L genes of PIV5.

Also included in the present invention are virions and infectious viral particles that include a PIV5 genome including a heterologous nucleotide sequence encoding a coronavirus S protein, including but not limited to the S protein of SARS-CoV-2.

Also included in the present invention are compositions including one or more of the PIV5 viral constructs or virions, as described herein. Such a composition may include a pharmaceutically acceptable carrier. As used, a pharmaceutically acceptable carrier refers to one or more compatible solid or liquid fillers, diluents or encapsulating substances which are suitable for administration to a human or other vertebrate animal. Such a carrier may be pyrogen free. The present invention also includes methods of making and using the viral vectors and compositions described herein.

The compositions of the present disclosure may be formulated in pharmaceutical preparations in a variety of forms adapted to the chosen route of administration. One of skill will understand that the composition will vary depending on mode of administration and dosage unit.

The agents of this invention can be administered in a variety of ways, including, but not limited to, intravenous, topical, oral, intranasal, subcutaneous, intraperitoneal, intramuscular, and intratumor deliver. In some aspects, the agents of the present invention may be formulated for controlled or sustained release. One advantage of intranasal immunization is the potential to induce a mucosal immune response.

### C. CVB-S Backbone Vaccine Virus Sequences

The CVB-vectored SARS-CoV-2 vaccine virus sequences from the constructs in Table 1 are listed below. The inserted N of SARS-CoV-2 sequences are in the underlined lowercase, M sequences are italicized lowercase, E sequences are bolded lowercase and S is in the lowercase:

i. CVL104

The nucleic acid sequence for CVL104 is:

(SEQ ID NO: 3)
GCCACCTGACGTCTAAGAAACCATTATTATCATGACATTAACCTA
TAAAAATAGGCGTATCACGAGGCCCTTTCGTCTCGCGCGTTTCGG
TGATGACGGTGAAAACCTCTGACACATGCAGCTCCCGGAGACGGT
CACAGCTTGTCTGTAAGCGGATGCCGGGAGCAGACAAGCCCGTCA
GGGCGCGTCAGCGGGTGTTGGCGGGTGTCGGGGCTGGCTTAACTA
TGCGGCATCAGAGCAGATTGTACTGAGAGTGCACCATATATGCGG
TGTGAAATACCGCACAGATGCGTAAGGAGAAAATACCGCATCAGG
CTAATACGACTCACTATAGGGACCAAGGGGAAAATGAAGTGGTGA
CTCAAATCATCGAAGACCCTCGAGATTACATAGGTCCGGAACCTA
TGGCCTTCGTGACCGACCTCGAGTCAGAGTAGTTCAATAAGGACC
TATCAAGTTTGGGCAATTTTTCGTCCCCGACACAAAAATGTCATC
CGTGCTTAAAGCATATGAGCGATTCACGCTCACTCAAGAACTGCA
AGATCAGAGTGAGGAAGGTACAATCCCACCTACAACACTAAAACC
GGTAATCAGGGTATTTATACTAACCTCTAATAACCCAGAGCTAAG
ATCCCGGCTTCTTCTATTCTGCCTACGGATTGTTCTCAGTAATGG
TGCAAGGGATTCCCATCGCTTTGGAGCATTACTCACAATGTTTTC
GCTACCATCAGCCACAATGCTCAATCATGTCAAATTAGCTGACCA
GTCACCAGAAGCTGATATCGAAAGGGTAGAGATCGATGGCTTTGA
GGAGGGATCATTCCGCTTAATCCCCAATGCTCGTTCAGGTATGAG
CCGTGGAGAGATCAATGCCTATGCTGCACTTGCAGAAGATCTACC
TGACACACTAAACCATGCAACACCTTTCGTTGATTCCGAAGTCGA
GGGAACTGCATGGGATGAGATTGAGACTTTCTTAGATATGTGTTA
CAGTGTCCTAATGCAGGCATGGATAGTGACTTGCAAGTGCATGAC
TGCGCCAGACCAACCTGCTGCTTCTATTGAGAAACGCCTGCAAAA
ATATCGTCAGCAAGGCAGGATCAACCCGAGATATCTCCTGCAACC
GGAGGCTCGACGAATAATCCAGAATGTAATCCGGAAGGGAATGGT
GGTCAGACATTTCCTCACCTTTGAACTGCAGCTTGCCCGAGCACA
AAGCCTTGTATCAAATAGGTATTATGCTATGGTAGGGGATGTTGG
AAAGTATATAGAGAATTGTGGAATGGGAGGCTTCTTTTTGACACT
AAAATATGCATTAGGAACTAGATGGCCCACACTTGCTTTAGCTGC
ATTTTCAGGAGAGCTAACAAAGCTAAAGTCCCTCATGGCATTATA
CCAGACCCTTGGTGAGCAGGCCCGATATTTGGCCCTATTGGAGTC
ACCACATTTGATGGATTTTGCTGCAGCAAACTACCCACTGCTATA
TAGCTATGCTATGGGAATAGGCTATGTGTTAGATGTCAACATGAG
GAACTACGCTTTCTCCAGATCATACATGAACAAGACATATTTCCA
ATTGGGAATGGAAACTGCAAGAAAACAACAGGGTGCAGTTGACAT
GAGGATGGCAGAAGATCTCGGTCTAACTCAAGCCGAACGCACCGA
GATGGCAAATACACTTGCCAAATTGACCACAGCAAATCGAGGGGC
AGACACCAGGGGAGGAGTCAACCCGTTCTCATCTGTCACTGGGAC
AACTCAGGTGCCCGCTGCAGCAACAGGTGACACACTCGAGAGTTA
CATGGCAGCGGATCGACTGAGGCAGAGATATGCTGATGCAGGCAC
CCATGATGATGAGATGCCACCATTGGAAGAGGAGGAAGAGGACGA
CACATCTGCAGGTCCACGCACTGGACCAACTCTTGAACAAGTGGC
CTTGGACATCCAGAACGCAGCAGTTGGAGCTCCCATCCATACAGA
TGACCTGAATGCCGCACTGGGTGATCTTGACATCTAGACAATTCA
GATCCCAATCTAAAATTGACATACCTAATTGATTAGTTAGATGGA
ACTACAGTGGATTCCATAAGGTTCCTGCCTACCATCGGCTTTAAA
GAAAAAAATAGGCCCGGACGGGTTAGCAACAAGCGACTGCCGGTG
CCAACAGCGCAATCCACAATCTACAATGGATCCCACTGATCTGAG
CTTCTCCCCAGATGAGATCAATAAGCTCATAGAGACAGGCCTGAA
TACTGTAGAGTATTTTACTTCCCAACAAGTCACAGGAACATCCTC
TCTTGGAAAGAATACAATACCACCAGGGGTCACAGGACTACTAAC
CAATGCTGCAGAGGCAAAGATCCAAGAGTCAACTAACCATCAGAA
GGGCTCAGTTGGTGGGGGTGCAAAACCAAAGAAACCGCGACCAAA
AATTGCCATTGTGCCAGCAGATGACAAAACAGTGCCCGGAAAGCC
GATCCCAAACCCTCTATTAGGTCTGGACTCCACCCCGAGCACCCA
AACTGTGCTTGATCTAAGTGGGAAAACATTACCATCAGGATCCTA
TAAGGGGGTTAAGCTTGCGAAATTTGGAAAAGAAAATCTGATGAC
ACGGTTCATCGAGGAACCCAGAGAGAATCCTATCGCAACCAGTTt
CCCCATCGATTTTAAGAGGGGCAGGGATACCGGCGGGTTCCATAG
AAGGGAGTACTCAATCGGATGGGTGGGAGATGAAGTCAAGGTCAC
TGAGTGGTGCAATCCATCCTGTTCTCCAATCACCGCTGCAGCAAG
GCGATTTGAATGCACTTGTCACCAGTGTCCAGTCACTTGCTCTGA
ATGTGAACGAGATACTTAATACAGTGAGAAATTTGGACTCTCGGA
TGAATCAACTGGAGACAAAAGTAGATCGCATTCTCTCATCTCAGT
CTCTAATCCAGACCATCAAGAATGACATAGTTGGACTTAAAGCAG
GGATGGCTACTTTAGAAGGAATGATTACAACTGTGAAAATCATGG
ACCCGGGAGTTCCCAGTAATGTTACTGTGGAAGATGTACGCAAGA
CACTAAGTAACCATGCTGTTGTTGTGCCAGAATCATTCAATGATA
GTTTCTTGACTCAATCTGAAGATGTAATTTCACTTGATGAGTTGG
CTCGACCAACTGCAACAAGTGTTAAGAAGATTGTCAGGAAGGTTC
CTCCTCAGAAGGATCTGACTGGATTGAAGATTACACTAGAGCAAT
TGGCAAAGGATTGCATCAGCAAACCGAAGATGAGGGAAGAGTATC
TCCTCAAAATCAACCAGGCTTCCAGTGAGGCTCAGCTAATTGACC
TCAAGAAAGCAATCATCCGCAGTGCAATTTGATCAAGAAACACCC
AATTACACTACACTGGTATGACACTGTACTAACCCTGAGGGTTTT
AGAAAAAACGATTAACGATAAATAAGCCCGAACACTACACACTAC
CTGAGGCAGCCATGCCATCCATCAGCATTCCCGCAGACCCCACCA
ATCCACGTCAATCAATAAAAGCGTTCCCAATTGTGATCAACAGTG
ATGGGGGTGAGAAAGGCCGCTTGGTTAAACAACTACGCACAACCT
ACTTGAATGACCTAGATACTCATGAGCCACTGGTGACATTCATAA
ATACCTATGGATTCATCTACGAACAGGATCGGGGGAATACCATTG
TCGGAGAGGATCAACTTGGGAAGAAAAGAGAGGCTGTGACCGCTG
CAATGGTTACCCTTGGATGTGGGCCTAATCTACCATCATTAGGGA
ATGTCCTGGGACAACTGAGGGAATTCCAGGTCACTGTTAGGAAGA
CATCCAGCAAAGCGGAAGAGATGGTCTTTGAAATTGTTAAGTATC
CGAGAATATTTCGGGGTCATACATTAATCCAGAAAGGACTAGTCT
GTGTCTCCGCAGAAAAATTTGTTAAGTCACCAGGGAAAATACAAT
CTGGAATGGACTATCTCTTCATTCCGACATTTCTGTCAGTGACTT
ACTGTCCAGCTGCAATCAAATTTCAGGTACCTGGCCCCATGTTGA
AAATGAGATCAAGATACACTCAGAGCTTACAACTTGAACTAATGA
TAAGAATCCTGTGTAAGCCCGATTCGCCACTTATGAAGGTCCATA
CCCCTGACAAGGAGGGAAGAGGATGTCTTGTATCAGTATGGCTGC
ATGTATGCAACATCTTCAAATCAGGAAACAAGAATGGCAGTGAGT
GGCAGGAATACTGGATGAGAAAGTGTGCTAACATGCAACTTGAAG
TGTCGATTGCAGATATGTGGGGACCAACTATCATAATTCATGCCA
GAGGTCACATTCCCAAAAGTGCTAAGTTGTTTTTTGGAAAGGGTG
GATGGAGCTGCCATCCACTTCACGAAGTTGTTCCAAGTGTCACTA
AAACACTATGGTCCGTGGGCTGTGAGATTACAAAGGCGAAGGCAA
TAATACAAGAGAGTAGCATCTCTCTTCTCGTGGAGACTACTGACA
TCATAAGTCCAAAAGTCAAAATTTCATCTAAGCATCGCCGCTTTG
GGAAATCAAATTGGGGTCTGTTCAAGAAAACTAAATCACTGCCTA
ACCTGACGGAGCTGGAATGACTGACCTCTAATCGAGACTACACCG
CCGCAAACTATAGGTGGGTGGTACCTCAGTGATTAATCTTGTAAG
CACTGATCGTAGGCTACAACACACTAATATTATCCAGATTAGAGA
GCTTAATTAGCTCTGTATTAATAATAACACTACTATTCCAATAAC
TGGAATCACCAGCTTGATTTATCTCCAAAATGATTCAAAGAAAAC
AAATCATATTAAGACTATCCTAAGCACGAACCCATATCGTCCTTC
AAATCATGGGTACTATAATTCAATTTCTGGTGGTCTCCTGTCTAT
TGGCAGGAGCAGGCAGCCTTGATCCAGCAGCCCTCATGCAAATCG
GTGTCATTCCAACAAATGTCCGGCAACTTATGTATTATACTGAAG
CTTCATCAGCATTCATTGTTGTGAAGTTAATGCCTACAATTGACT
CGCCGATTAGTGGATGTAATATAACATCAATTTCAAGCTATAATG
CAACAGTGACAAAACTCCTACAGCCGATCGGTGAGAATTTGGAGA
CGATTAGGAACCAGTTGATTCCAACTCGGAGGAGACGCCGGTTTG
CAGGGGTGGTGATTGGATTAGCTGCATTAGGAGTAGCTACTGCCG
CACAGGTCACTGCCGCAGTGGCACTAGTAAAGGCAAATGAAAATG
CTGCGGCTATACTCAATCTCAAAAATGCAATCCAAAAAACAAATG
CAGCAGTTGCAGATGTGGTCCAGGCCACACAATCACTAGGAACGG
CAGTTCAAGCAGTTCAAGATCACATAAACAGTGTGGTAAGTCCAG
CAATTACAGCAGCCAATTGTAAGGCCCAAGATGCTATCATTGGCT
CAATCCTCAATCTCTATTTGACCGAGTTGACAACCATCTTCCACA
ATCAAATTACAAACCCTGCATTGAGTCCCATTACAATTCAAGCTT
TAAGGATCCTACTGGGGAGTACCTTGCCGACTGTGGTCGAAAAAT
CTTTCAATACCCAGATAAGTGCAGCTGAGCTTCTCTCATCAGGGT
TATTGACAGGCCAGATTGTGGGATTAGATTTGACCTATATGCAGA
TGGTCATAAAAATTGAGCTGCCAACTTTAACTGTACAACCTGCAA
CCCAGATCATAGATCTGGCCACCATTTCTGCATTCATTAACAATC
AAGAAGTCATGGCCCAATTACCAACACGTGTTATGGTGACTGGCA
GCTTGATCCAAGCCTATCCCGCATCGCAATGCACCATTACACCCA
ACACTGTGTACTGTAGGTATAATGATGCCCAAGTACTCTCAGATG
ATACTATGGCTTGCCTCCAAGGTAACTTGACAAGATGCACCTTCT
CTCCAGTGGTTGGGAGCTTTCTCACTCGATTCGTGCTGTTCGATG
GAATAGTTTATGCAAATTGCAGGTCGATGTTGTGCAAGTGCATGC
AACCTGCTGCTGTGATCCTACAGCCGAGTTCATCCCCTGTAACTG
TCATTGACATGTACAAATGTGTGAGTCTGCAGCTTGACAATCTCA
GATTCACCATCACTCAATTGGCCAATGTAACCTACAATAGCACCA
TCAAGCTTGAATCATCCCAGATCTTGTCTATTGATCCGTTGGATA
TATCCCAGAATCTAGCTGCGGTGAATAAGAGTCTAAGTGATGCAC
TACAACACTTAGCACAAAGTGACACATATCTTTCTGCAATCACAT
CAGCTACGACTACAAGTGTATTATCCATAATAGCAATCTGTCTTG
GATCGTTAGGTTTAATATTAATAATCTTGCTCAGTGTAGTTGTGT
GGAAGTTATTGACCATTGTCGTTGCTAATCGAAATAGAATGGAGA
ATTTTGTTTATCATAAATAAGCATTCCACCACTCACGATCTGATC
TCAGTGAGAAAAATCAACCTGCAACTCTTGGAACAAGATAAGACA
GTCATCCATTAGTAATTTTTAAGAAAAAAACGATAGGACCGAACC
TAGTATTGAAAGAACCGTCTCGGTCAATCTAGGTAATCGAGCTGA
TACCGTCTCGGAAAGCTCAAATCCTGCTATAGGCTATCCACTGCA
TCATCTCTCCTGCCATACTTCCTACTCACATCATATCTATTTTAA
AGAAAAAATAGGCCCGAACACTAATCGTGCCGGCAGTGCCACTGC
ACACACAACACTACACATACAATACACTACAATGGTTGCAGAAGA
TGCCCCTGTTAGGGCCACTTGCCGAGTATTATTTCGAACAACAAC
TTTAATCTTTCTATGCACACTACTAGCATTAAGCATCTCTATCCT
TTATGAGAGTTTAATAACCCAAAAGCAAATCATGAGCCAAGCAGG
CTCAACTGGATCTAATTCTGGATTAGGAAGTATCACTGATCTTCT
TAATAATATTCTCTCTGTCGCAAATCAGATTATATATAACTCTGC
AGTCGCTCTACCTCTACAATTGGACACTCTTGAATCAACACTCCT
TACAGCCATTAAGTCTCTTCAAACCAGTGACAAGCTAGAACAGAA
CTGCTCGTGGAGTGCTGCACTGATTAATGATAATAGATACATTAA
TGGCATCAATCAGTTCTATTTTTCAATTGCTGAGGGTCGCAATCT
GACACTTGGCCCACTTCTTAATATGCCTAGTTTCATTCCAACTGC
CACGACACCAGAGGGCTGCACCAGGATCCCATCATTCTCGCTCAC
TAAGACACACTGGTGTTATACACACAATGTTATCCTGAATGGATG
CCAGGATCATGTATCCTCAAATCAATTTGTTTCTATGGGAATCAT
TGAACCCACTTCTGCCGGGTTTCCATTCTTTCGAACCCTAAAGAC
TCTATATCTCAGCGATGGGGTCAATCGTAAGAGCTGCTCTATCAG
TACAGTTCCGGGGGGTTGTATGATGTACTGTTTTGTTTCTACTCA
ACCAGAGAGGGATGACTACTTTTCTGCCGCTCCTCCAGAACAACG
AATTATTATAATGTACTATAATGATACAATCGTGGAGCGCATAAT
TAATCCACCCGGGGTACTAGATGTATGGGCAACATTGAACCCAGG
AACAGGAAGCGGGGTATATTATTTAGGTTGGGTGCTCTTTCCAAT
ATATGGCGGCGTGATTAAAGGTACGAGTTTATGGAATAATCAAGC
AAATAAATACTTTATCCCCCAGATGGTTGCTGCTCTCTGCTCACA
AAACCAGGCAACTCAAGTCCAAAATGCTAAGTCATCATACTATAG
CAGCTGGTTTGGCAATCGAATGATTCAGTCTGGGATCCTGGCATG
TCCTCTTCGACAGGATCTAACCAATGAGTGTTTAGTTCTGCCCTT
TTCTAATGATCAGGTGCTTATGGGTGCTGAAGGGAGATTATACAT
GTATGGTGACTCGGTGTATTACTATCAAAGAAGCAATAGTTGGTG
GCCTATGACCATGCTGTATAAGGTAACCATAACATTCACTAATGG
TCAGCCATCTGCTATATCAGCTCAGAATGTGCCCACACAGCAGGT
CCCTAGACCTGGGACAGGAGACTGCTCTGCAACCAATAGATGTCC
CGGTTTTTGCTTGACAGGAGTGTATGCCGATGCCTGGTTACTGAC
CAACCCTTCGTCTACCAGTACATTTGGATCAGAAGCAACCTTCAC
TGGTTCTTATCTCAACACAGCAACTCAGCGTATCAATCCGACGAT
GTATATCGCGAACAACACACAGATCATAAGCTCACAGCAATTTGG
ATCAAGCGGTCAAGAAGCAGCATATGGCCACACAACTTGTTTTAG
GGACACAGGCTCTGTTATGGTATACTGTATCTATATTATTGAATT
GTCCTCATCTCTCTTAGGACAATTTCAGATTGTCCCATTTATCCG
TCAGGTGACACTATCCTAAAGGCAGAAGCCTTCAGGTCTGACCCA
GCCAATCAAAGCATTATACCAGACCATGGCCTACCATCGGCTTTA
AAGAAAAAAATAGGCCCGGACGGGTTAGCAACAAGCGGCGGCCGC
Catgttcgtcttcctggtcctgctgcctctggtctcctcacagtg
cgtcaatctgacaactcggactcagctgccacctgcttatactaa
tagcttcaccagaggcgtgtactatcctgacaaggtgtttagaag
ctccgtgctgcactctacacaggatctgtttctgccattctttag
caacgtgacctggttccacgccatccacgtgagcggcaccaatgg
cacaaagcggttcgacaatcccgtgctgccttttaacgatggcgt
gtacttcgcctctaccgagaagagcaacatcatcagaggctggat
ctttggcaccacactggactccaagacacagtctctgctgatcgt
gaacaatgccaccaacgtggtcatcaaggtgtgcgagttccagtt
ttgtaatgatcccttcctgggcgtgtactatcacaagaacaataa
gagctggatggagtccgagtttagagtgtattctagcgccaacaa
ctgcacatttgagtacgtgagccagcctttcctgatggacctgga
gggcaagcagggcaatttcaagaacctgagggagttcgtgtttaa
gaatatcgacggctacttcaaaatctactctaagcacacccccat
caacctggtgcgcgacctgcctcagggcttcagcgccctggagcc
cctggtggatctgcctatcggcatcaacatcacccggtttcagac
actgctggccctgcacagaagctacctgacacccggcgactcctc
tagcggatggaccgccggcgctgccgcctactatgtgggctacct
ccagccccggaccttcctgctgaagtacaacgagaatggcaccat
cacagacgcagtggattgcgccctggaccccctgagcgagacaaa
gtgtacactgaagtcctttaccgtggagaagggcatctatcagac
atccaatttcagggtgcagccaaccgagtctatcgtgcgctttcc
taatatcacaaacctgtgcccatttggcgaggtgttcaacgcaac
ccgcttcgccagcgtgtacgcctggaataggaagcggatcagcaa
ctgcgtggccgactatagcgtgctgtacaactccgcctctttcag
cacctttaagtgctatggcgtgtcccccacaaagctgaatgacct
gtgctttaccaacgtctacgccgattctttcgtgatcaggggcga
cgaggtgcgccagatcgcccccggccagacaggcaagatcgcaga
ctacaattataagctgccagacgatttcaccggctgcgtgatcgc
ctggaacagcaacaatctggattccaaagtgggcggcaactacaa
ttatctgtaccggctgtttagaaagagcaatctgaagcccttcga
gagggacatctctacagaaatctaccaggccggcagcaccccttg
caatggcgtggagggctttaactgttatttcccactccagtccta
cggcttccagcccacaaacggcgtgggctatcagccttaccgcgt
ggtggtgctgagctttgagctgctgcacgccccagcaacagtgtg
cggccccaagaagtccaccaatctggtgaagaacaagtgcgtgaa
cttcaacttcaacggcctgaccggcacaggcgtgctgaccgagtc
caacaagaagttcctgccatttcagcagttcggcagggacatcgc
agataccacagacgccgtgcgcgacccacagaccctggagatcct
ggacatcacaccctgctctttcggcggcgtgagcgtgatcacacc
cggcaccaatacaagcaaccaggtggccgtgctgtatcaggacgt
gaattgtaccgaggtgcccgtggctatccacgccgatcagctgac
cccaacatggcgggtgtacagcaccggctccaacgtcttccagac
aagagccggatgcctgatcggagcagagcacgtgaacaattccta
tgagtgcgacatcccaatcggcgccggcatctgtgcctcttacca
gacccagacaaactctcccagaagagcccggagcgtggcctccca
gtctatcatcgcctataccatgtccctgggcgccgagaacagcgt
ggcctactctaacaatagcatcgccatcccaaccaacttcacaat
ctctgtgaccacagagatcctgcccgtgtccatgaccaagacatc
tgtggactgcacaatgtatatctgtggcgattctaccgagtgcag
caacctgctgctccagtacggcagcttttgtacccagctgaatag
agccctgacaggcatcgccgtggagcaggataagaacacacagga
ggtgttcgcccaggtgaagcaaatctacaagaccccccctatcaa
ggactttggcggcttcaatttttcccagatcctgcctgatccatc
caagccttctaagcggagctttatcgaggacctgctgttcaacaa
ggtgaccctggccgatgccggcttcatcaagcagtatggcgattg
cctgggcgacatcgcagccagggacctgatctgcgcccagaagtt
taatggcctgaccgtgctgccacccctgctgacagatgagatgat
cgcacagtacacaagcgccctgctggccggcaccatcacatccgg
atggaccttcggcgcaggagccgccctccagatcccctttgccat
gcagatggcctataggttcaacggcatcggcgtgacccagaatgt
gctgtacgagaaccagaagctgatcgccaatcagtttaactccgc
catcggcaagatccaggacagcctgtcctctacagccagcgccct
gggcaagctccaggatgtggtgaatcagaacgcccaggccctgaa
taccctggtgaagcagctgagcagcaacttcggcgccatctctag
cgtgctgaatgacatcctgagccggctggacaaggtggaggcaga
ggtgcagatcgaccggctgatcaccggccggctccagagcctcca
gacctatgtgacacagcagctgatcagggccgccgagatcagggc
cagcgccaatctggcagcaaccaagatgtccgagtgcgtgctggg
ccagtctaagagagtggacttttgtggcaagggctatcacctgat
gtccttccctcagtctgccccacacggcgtggtgtttctgcacgt
gacctacgtgcccgcccaggagaagaacttcaccacagcccctgc
catctgccacgatggcaaggcccactttccaagggagggcgtgtt
cgtgtccaacggcacccactggtttgtgacacagcgcaatttcta
cgagccccagatcatcaccacagacaacaccttcgtgagcggcaa
ctgtgacgtggtcatcggcatcgtgaacaataccgtgtatgatcc
actccagcccgagctggacagctttaaggaggagctggataagta
tttcaagaatcacacctcccctgacgtggatctgggcgacatcag
cggcatcaatgcctccgtggtgaacatccagaaggagatcgaccg
cctgaacgaggtggctaagaatctgaacgagagcctgatcgacct
ccaggagctgggcaagtatgagcagtacatcaagtggccctggta
catctggctgggcttcatcgccggcctgatcgccatcgtgatggt
gaccatcatgctgtgctgtatgacatcctgctgttcttgcctgaa
gggctgctgtagctgtggctcctgctgtaagttattgaccattgt
cgttgctaatcgaaatagaatggagaattttgtttatcataaatg
aAGTCGACTCATGGAATGCATACCAAACATTATTGACACTAATGA
CACACAAAATTGGTTTTAAGAAAAACCAAGAGAACAATAGGCCAG
AATGGCTGGGTCTCGGGAGATATTACTCCCTGAAGTCCATCTCAA
TTCACCAATTGTAAAGCATAAGCTATACTATTACATTCTACTTGG
AAACCTCCCAAATGAGATCGACCTTGACGATTTAGGTCCATTACA
TAATCAAAATTGGAATCAGATAGCACATGAAGAGTCTAACTTAGC
TCAACGCTTGGTAAATGTAAGAAATTTTCTAATTACCCACATCCC
TGATCTTAGAAAGGGCCATTGGCAAGAGTATGTCAATGTAATACT
GTGGCCGCGAATTCTTCCCTTGATCCCGGATTTTAAAATCAATGA
CCAATTGCCTCTGCTCAAAAATTGGGACAAGTTAGTTAAAGAATC
ATGTTCAGTAATCAATGCAGGTACTTCCCAGTGCATTCAGAATCT
CAGCTATGGACTGACAGGTCGTGGGAACCTCTTTACACGATCACG
TGAACTCTCTGGTGACCGCAGGGATATTGATCTTAAGACAGTTGT
GGCAGCATGGCATGACTCAGACTGGAAAAGAATAAGTGATTTTTG
GATTATGATCAAATTCCAGATGAGACAATTAATTGTTAGGCAAAC
AGATCATAATGATTCTGATTTAATCACGTATATCGAAAATAGAGA
AGGCATAATCATCATAACCCCTGAACTGGTAGCATTATTTAACAC
TGAGAATCATACACTAACATACATGACCTTTGAAATTGTACTGAT
GGTTTCAGATATGTACGAAGGTCGTCACAACATTTTATCACTATG
CACAGTTAGCACTTACCTGAATCCTCTGAAGAAAAGAATAACATA
TTTATTGAGCCTTGTAGATAACTTAGCTTTTCAGATAGGTGATGC
TGTATATAACATAATTGCTTTGCTAGAATCCTTTGTATATGCACA
GTTGCAAATGTCAGATCCCATCCCAGAACTCAGAGGACAATTCCA
TGCATTCGTATGTTCTGAGATTCTTGATGCACTAAGAGGAACTAA
TAGTTTCACCCAGGATGAATTAAGAACTGTGACAACTAATTTGAT
ATCCCCATTCCAAGATCTGACCCCAGATCTTACGGCTGAATTGCT
CTGTATAATGAGGCTTTGGGGACACCCCATGCTCACTGCCAGTCA
AGCTGCAGGAAAGGTACGCGAGTCTATGTGTGCTGGAAAAGTATT
AGACTTTCCCACCATTATGAAAACACTAGCCTTTTTCCATACTAT
TCTGATCAATGGATACAGGAGGAAGCATCATGGAGTATGGCCACC
CTTAAACTTACCGGGTAATGCTTCAAAGGGTCTCACGGAACTTAT
GAATGACAATACTGAAATAAGCTATGAATTCACACTTAAGCATTG
GAAGGAAGTCTCTCTTATAAAATTCAAGAAATGTTTTGATGCAGA
CGCAGGTGAGGAACTCAGTATATTTATGAAAGATAAGGCAATTAG
TGCCCCAAAACAAGACTGGATGAGTGTGTTTAGAAGAAGCCTAAT
CAAACAGCGCCATCAGCATCATCAGGTCCCCCTACCAAATCCATT
CAATCGACGGCTGTTGCTAAACTTTCTCGGAGATGACAAATTCGA
CCCGAATGTGGAGCTACAGTATGTAACATCAGGTGAGTATCTACA
TGATGACACGTTTTGTGCATCATATTCACTAAAAGAGAAGGAAAT
TAAACCTGATGGTCGAATTTTTGCAAAGTTGACTAAGAGAATGAG
ATCATGTCAAGTTATAGCAGAATCTCTTTTAGCGAACCATGCTGG
GAAGTTAATGAAAGAGAATGGTGTTGTGATGAATCAGCTATCATT
AACAAAATCACTATTAACAATGAGTCAGATTGGAATAATATCCGA
GAAAGCTAGAAAGTCAACTCGAGATAACATAAATCAACCTGGTTT
CCAGAATATCCAGAGAAATAAATCACATCACTCCAAGCAAGTCAA
TCAGCGAGATCCAAGTGATGACTTTGAATTGGCAGCATCTTTTTT
AACTACTGATCTCAAAAAATATTGTTTACAATGGAGGTACCAGAC
AATTATCCCATTTGCTCAATCATTAAACAGAATGTATGGTTATCC
TCATCTCTTTGAGTGGATTCACTTACGGCTAATGCGTAGTACACT
TTACGTGGGGGATCCCTTCAACCCACCAGCAGATACCAGTCAATT
TGATCTAGATAAAGTAATTAATGGAGATATCTTCATTGTATCACC
CAGAGGTGGAATTGAAGGGCTGTGTCAAAAGGCTTGGACAATGAT
ATCTATCGCTGTGATAATTCTATCTGCCACAGAGTCTGGCACACG
AGTAATGAGTATGGTGCAGGGAGATAATCAAGCAATTGCTGTCAC
CACACGAGTACCAAGGAGCCTGCCGACTCTTGAGAAAAAGACTAT
TGCTTTTAGATCTTGTAATCTATTCTTTGAGAGGTTAAAATGTAA
TAATTTTGGATTAGGTCACCATTTGAAAGAACAAGAGACTATCAT
TAGTTCTCACTTCTTTGTTTATAGCAAGAGAATATTCTATCAGGG
GAGGATTCTAACGCAAGCCTTAAAAAATGCTAGTAAGCTCTGCTT
GACAGCTGATGTCCTAGGAGAATGCACCCAATCATCATGTTCTAA
TCTTGCAACTACTGTCATGAGGTTAACTGAGAATGGTGTTGAAAA
AGATATCTGTTTCTACTTGAATATCTATATGACCATCAAACAGCT
CTCCTATGATATCATCTTCCCTCAAGTGTCAATTCCTGGAGATCA
GATCACATTAGAATACATAAATAATCCACACCTGGTATCACGATT
GGCTCTTTTGCCATCCCAGTTAGGAGGTCTAAACTACCTGTCATG
CAGTAGGCTGTTCAATCGAAACATAGGCGACCCGGTGGTTTCCGC
AGTTGCAGATCTTAAGAGATTAATTAAATCAGGATGTATGGATTA
CTGGATCCTTTATAACTTATTAGGGAGAAAACCGGGAAACGGCTC
ATGGGCTACTTTAGCAGCTGACCCGTACTCAATCAATATAGAGTA
TCAATACCCTCCAACTACAGCTCTTAAGAGGCACACCCAACAAGC
TCTGATGGAACTCAGTACGAATCCAATGTTACGTGGCATATTCTC
TGACAATGCACAGGCAGAAGAAAATAACCTTGCTAGGTTTCTCCT
GGATAGGGAGGTGATCTTTCCGCGTGTAGCTCACATCATCATTGA
GCAAACCAGTGTCGGGAGGAGAAAACAGATTCAAGGATATTTGGA
TTCAACTAGATCGATAATGAGGAAATCACTAGAAATTAAGCCCTT
ATCCAATAGGAAGCTTAATGAAATACTGGATTACAACATCAATTA
CCTAGCTTACAATTTGGCATTACTCAAGAATGCTATTGAACCTCC
GACTTATTTGAAGGCAATGACACTTGAAACATGTAGCATCGACAT
TGCAAGGAACCTCCGGAAGCTCTCCTGGGCCCCACTCTTGGGTGG
GAGAAATCTTGAAGGATTAGAGACGCCAGATCCCATTGAAATTAC
TGCAGGAGCATTAATTGTTGGATCGGGCTACTGTGAACAGTGTGC
TGCAGGAGACAATCGATTCACATGGTTTTTCTTGCCATCTGGTAT
CGAGATAGGAGGGGATCCCCGTGATAATCCTCCTATCCGTGTACC
GTACATTGGCTCCAGGACTGATGAGAGGAGGGTAGCCTCAATGGC
ATACATCAGGGGTGCCTCGAGTAGCTTAAAAGCAGTTCTTAGACT
GGCGGGAGTGTACATCTGGGCATTCGGAGATACTCTGGAGAATTG
GATAGATGCACTGGATTTGTCTCACACTAGAGTTAACATCACACT
TGAACAGCTGCAATCCCTCACCCCACTTCCAACCTCTGCCAATCT
AACCCATCGGTTGGATGATGGCACAACTACCCTAAAGTTTACTCC
TGCGAGCTCTTACACCTTTTCAAGTTTCACTCATATATCAAATGA
TGAGCAATACCTGACAATTAATGACAAAACTGCAGATTCAAATAT
AATCTACCAACAGTTAATGATCACTGGACTCGGAATCTTAGAAAC
ATGGAATAATCCCCCAATCAATAGAACATTCGAAGAATCTACCCT
ACATTTGCACACTGGTGCATCATGTTGTGTCCGACCTGTGGACTC
CTGCATTCTCTCAGAAGCATTAACAGTCAAGCCACATATTACAGT
ACCGTACAGCAATAAATTTGTATTTGATGAGGACCCGCTATCTGA
ATATGAAACTGCAAAACTGGAATCGTTATCATTCCAAGCCCAATT
AGGCAACATTGATGCTGTAGATATGACAGGTAAATTAACATTATT
GTCCCAATTCACTGCAAGGCAGATTATCAATGCAATCACTGGACT
CGATGAGTCTGTCTCTCTTACTAATGATGCCATTGTTGCATCAGA
CTATGTCTCCAATTGGATTAGTGAATGCATGTATACCAAATTAGA
TGAATTATTTATGTATTGTGGGTGGGAACTACTATTGGAACTATC
CTATCAAATGTATTATCTGAGGGTAGTTGGGTGGAGTAATATAGT
GGATTATTCTTACATGATCTTGAGAAGAATCCCGGGTGCAGCATT
AAACAATCTGGCATCTACATTAAGTCATCCAAAACTTTTCCGACG
AGCTATCAACCTAGATATAGTTGCCCCCTTAAATGCTCCTCATTT
TGCATCTCTGGACTACATCAAGATGAGTGTGGATGCAATACTCTG
GGGCTGTAAAAGAGTCATCAATGTGCTCTCCAATGGAGGGGACTT
AGAATTAGTTGTGACATCTGAAGATAGCCTTATTCTCAGTGACCG
ATCCATGAATCTCATTGCAAGGAAATTAACTTTATTATCACTGAT
TCACCATAATGGTTTGGAACTACCAAAGATTAAGGGGTTCTCTCC
TGATGAGAAGTGTTTCGCTTTGACAGAATTTTTGAGGAAAGTGGT
GAACTCAGGGTTGAGTTCAATAGAGAACCTATCAAATTTTATGTA
CAATGTGGAGAACCCACGGCTTGCAGCATTCGCCAGCAACAATTA
CTACCTGACCAGAAAATTATTGAATTCAATACGAGATACTGAGTC
GGGTCAAGTAGCAGTCACCTCATATTATGAATCATTAGAATATAT
TGATAGTCTTAAGCTAACCCCACATGTGCCTGGCACCTCATGCAT
TGAGGATGATAGTCTATGTACAAATGATTACATAATCTGGATCAT
AGAGTCTAATGCAAACTTGGAGAAGTATCCAATTCCAAATAGCCC
TGAGGATGATTCCAATTTCCATAACTTTAAGTTGAATGCTCCATC
GCACCATACCTTACGCCCATTAGGGTTGTCATCAACTGCTTGGTA
TAAGGGTATAAGCTGCTGCAGGTACCTTGAGCGATTAAAGCTACC
ACAAGGTGATCATTTATATATTGCAGAAGGTAGTGGTGCCAGTAT
GACAATCATAGAATACCTATTCCCAGGAAGAAAGATATATTACAA
TTCTTTATTTAGTAGTGGTGACAATCCCCCACAAAGAAATTATGC
ACCAATGCCTACTCAGTTCATTGAGAGTGTCCCATACAAGCTCTG
GCAAGCACACACAGATCAATATCCCGAGATTTTTGAGGACTTCAT
CCCTCTATGGAACGGAAACGCCGCCATGACTGACATAGGAATGAC
AGCTTGTGTAGAATTCATCATCAATCGAGTCGGCCCAAGGACTTG
CAGTTTAGTACATGTAGATTTGGAATCAAGTGCAAGCTTAAATCA
ACAATGCCTGTCAAAGCCGATAATTAATGCTATCATCACTGCTAC
AACTGTTTTGTGCCCTCATGGGGTGCTTATTCTGAAATATAGTTG
GTTGCCATTTACTAGATTTAGTACTTTGATCACTTTCTTATGGTG
CTACTTTGAGAGAATCACTGTTCTTAGGAGCACATATTCTGATCC
AGCTAATCATGAGGTTTATTTAATTTGTATCCTTGCCAACAACTT
TGCATTCCAGACTGTCTCGCAGGCAACAGGAATGGCGATGACTTT
AACTGATCAAGGGTTTACTTTGATATCACCTGAAAGAATAAATCA
GTATTGGGATGGTCACTTGAAGCAAGAACGTATCGTAGCAGAAGC
AATTGATAAGGTGGTTCTAGGAGAAAATGCTCTATTTAATTCGAG
TGATAATGAATTAATTCTCAAATGTGGAGGGACACCAAATGCACG
GAATCTCATCGATATCGAGCCAGTCGCAACTTTCATAGAATTTGA
ACAATTGATCTGCACAATGTTGACAACCCACTTGAAGGAAATAAT
TGATATAACAAGGTCTGGAACCCAGGATTATGAAAGTTTATTACT
CACTCCTTACAATTTAGGTCTTCTTGGTAAAATCAGTACGATAGT
GAGATTATTAACAGAAAGGATTCTAAATCATACTATCAGGAATTG
GTTGATCCTCCCACCTTCGCTCCGGATGATCGTGAAGCAGGACTT
GGAATTCGGCATATTCAGGATTACTTCCATCCTCAATTCTGATCG
GTTCCTGAAGCTTTCTCCAAATAGGAAATACTTGATTGCACAATT
AACTGCAGGCTACATTAGGAAATTGATTGAGGGGGATTGCAATAT
CGATCTAACCAGACCTATCCAAAAGCAAATCTGGAAAGCATTAGG
TTGTGTAGTCTATTGTCACGATCCAATGGATCAAAGGGAGTCAAC
AGAGTTTATTGATATAAATATTAATGAAGAAATAGACCGCGGGAT
CGATGGCGAGGAAATCTAAACATATCAAGAATCAGAATTAGTTTA
AGAAAAAAGAAGAGGATTAATCTTGGTTTTCCCCTTGGTGGGTCG
GCATGGCATCTCCACCTCCTCGCGGTCCGACCTGGGCATCCGAAG
GAGGACGCACGTCCACTCGGATGGCTAAGGGAGCGGCCGGGGATC
CGGCTGCTAACAAAGCCCGAAAGGAAGCTGAGTTGGCTGCTGCCA
CCGCTGAGCAATAACTAGCATAACCCCTTGGGGCCTCTAAACGGG
TCTTGAGGGGTTTTTTGCTGAAAGGAGGAACTATATCCGGATCAT
GTGAGCAAAAGGCCAGCAAAAGGCCAGGAACCGTAAAAAGGCCGC
GTTGCTGGCGTTTTTCCATAGGCTCCGCCCCCCTGACGAGCATCA
CAAAAATCGACGCTCAAGTCAGAGGTGGCGAAACCCGACAGGACT
ATAAAGATACCAGGCGTTTCCCCCTGGAAGCTCCCTCGTGCGCTC
TCCTGTTCCGACCCTGCCGCTTACCGGATACCTGTCCGCCTTTCT
CCCTTCGGGAAGCGTGGCGCTTTCTCATAGCTCACGCTGTAGGTA
TCTCAGTTCGGTGTAGGTCGTTCGCTCCAAGCTGGGCTGTGTGCA
CGAACCCCCCGTTCAGCCCGACCGCTGCGCCTTATCCGGTAACTA
TCGTCTTGAGTCCAACCCGGTAAGACACGACTTATCGCCACTGGC
AGCAGCCACTGGTAACAGGATTAGCAGAGCGAGGTATGTAGGCGG
TGCTACAGAGTTCTTGAAGTGGTGGCCTAACTACGGCTACACTAG
AAGAACAGTATTTGGTATCTGCGCTCTGCTGAAGCCAGTTACCTT
CGGAAAAAGAGTTGGTAGCTCTTGATCCGGCAAACAAACCACCGC
TGGTAGCGGTGGTTTTTTTGTTTGCAAGCAGCAGATTACGCGCAG
AAAAAAAGGATCTCAAGAAGATCCTTTGATCTTTTCTACGGGGTC
TGACGCTCAGTGGAACGAAAACTCACGTTAAGGGATTTTGGTCAT
GAGATTATCAAAAAGGATCTTCACCTAGATCCTTTTAAATTAAAA
ATGAAGTTTTAAATCAATCTAAAGTATATATGAGTAAACTTGGTC
TGACAGTTACCAATGCTTAATCAGTGAGGCACCTATCTCAGCGAT
CTGTCTATTTCGTTCATCCATAGTTGCCTGACTCCCCGTCGTGTA
GATAACTACGATACGGGAGGGCTTACCATCTGGCCCCAGTGCTGC
AATGATACCGCGAGACCCACGCTCACCGGCTCCAGATTTATCAGC
AATAAACCAGCCAGCCGGAAGGGCCGAGCGCAGAAGTGGTCCTGC
AACTTTATCCGCCTCCATCCAGTCTATTAATTGTTGCCGGGAAGC
TAGAGTAAGTAGTTCGCCAGTTAATAGTTTGCGCAACGTTGTTGC
CATTGCTACAGGCATCGTGGTGTCACGCTCGTCGTTTGGTATGGC
TTCATTCAGCTCCGGTTCCCAACGATCAAGGCGAGTTACATGATC
CCCCATGTTGTGCAAAAAAGCGGTTAGCTCCTTCGGTCCTCCGAT
CGTTGTCAGAAGTAAGTTGGCCGCAGTGTTATCACTCATGGTTAT
GGCAGCACTGCATAATTCTCTTACTGTCATGCCATCCGTAAGATG
CTTTTCTGTGACTGGTGAGTACTCAACCAAGTCATTCTGAGAATA
GTGTATGCGGCGACCGAGTTGCTCTTGCCCGGCGTCAATACGGGA
TAATACCGCGCCACATAGCAGAACTTTAAAAGTGCTCATCATTGG
AAAACGTTCTTCGGGGCGAAAACTCTCAAGGATCTTACCGCTGTT
GAGATCCAGTTCGATGTAACCCACTCGTGCACCCAACTGATCTTC
AGCATCTTTTACTTTCACCAGCGTTTCTGGGTGAGCAAAAACAGG
AAGGCAAAATGCCGCAAAAAAGGGAATAAGGGCGACACGGAAATG
TTGAATACTCATACTCTTCCTTTTTCAATATTATTGAAGCATTTA
TCAGGGTTATTGTCTCATGAGCGGATACATATTTGAATGTATTTA
GAAAAATAAACAAATAGGGGTTCCGCGCACATTTCCCCGAAAAGT.

ii. CVL87

The nucleic acid sequence for CVL87 is:

(SEQ ID NO: 4)
GCCACCTGACGTCTAAGAAACCATTATTATCATGACATTAACCTAT
AAAAATAGGCGTATCACGAGGCCCTTTCGTCTCGCGCGTTTCGGT
GATGACGGTGAAAACCTCTGACACATGCAGCTCCCGGAGACGGTC
ACAGCTTGTCTGTAAGCGGATGCCGGGAGCAGACAAGCCCGTCAG
GGCGCGTCAGCGGGTGTTGGCGGGTGTCGGGGCTGGCTTAACTAT
GCGGCATCAGAGCAGATTGTACTGAGAGTGCACCATATATGCGGT
GTGAAATACCGCACAGATGCGTAAGGAGAAAATACCGCATCAGGC
TAATACGACTCACTATAGGGACCAAGGGGAAAATGAAGTGGTGAC
TCAAATCATCGAAGACCCTCGAGATTACATAGGTCCGGAACCTAT
GGCCTTCGTGACCGACCTCGAGTCAGAGTAGTTCAATAAGGACCT
ATCAAGTTTGGGCAATTTTTCGTCCCCGACACAAAAATGTCATCC
GTGCTTAAAGCATATGAGCGATTCACGCTCACTCAAGAACTGCAA
GATCAGAGTGAGGAAGGTACAATCCCACCTACAACACTAAAACCG
GTAATCAGGGTATTTATACTAACCTCTAATAACCCAGAGCTAAGA
TCCCGGCTTCTTCTATTCTGCCTACGGATTGTTCTCAGTAATGGT
GCAAGGGATTCCCATCGCTTTGGAGCATTACTCACAATGTTTTCG
CTACCATCAGCCACAATGCTCAATCATGTCAAATTAGCTGACCAG
TCACCAGAAGCTGATATCGAAAGGGTAGAGATCGATGGCTTTGAG
GAGGGATCATTCCGCTTAATCCCCAATGCTCGTTCAGGTATGAGC
CGTGGAGAGATCAATGCCTATGCTGCACTTGCAGAAGATCTACCT
GACACACTAAACCATGCAACACCTTTCGTTGATTCCGAAGTCGAG
GGAACTGCATGGGATGAGATTGAGACTTTCTTAGATATGTGTTAC
AGTGTCCTAATGCAGGCATGGATAGTGACTTGCAAGTGCATGACT
GCGCCAGACCAACCTGCTGCTTCTATTGAGAAACGCCTGCAAAAA
TATCGTCAGCAAGGCAGGATCAACCCGAGATATCTCCTGCAACCG
GAGGCTCGACGAATAATCCAGAATGTAATCCGGAAGGGAATGGTG
GTCAGACATTTCCTCACCTTTGAACTGCAGCTTGCCCGAGCACAA
AGCCTTGTATCAAATAGGTATTATGCTATGGTAGGGGATGTTGGA
AAGTATATAGAGAATTGTGGAATGGGAGGCTTCTTTTTGACACTA
AAATATGCATTAGGAACTAGATGGCCCACACTTGCTTTAGCTGCA
TTTTCAGGAGAGCTAACAAAGCTAAAGTCCCTCATGGCATTATAC
CAGACCCTTGGTGAGCAGGCCCGATATTTGGCCCTATTGGAGTCA
CCACATTTGATGGATTTTGCTGCAGCAAACTACCCACTGCTATAT
AGCTATGCTATGGGAATAGGCTATGTGTTAGATGTCAACATGAGG
AACTACGCTTTCTCCAGATCATACATGAACAAGACATATTTCCAA
TTGGGAATGGAAACTGCAAGAAAACAACAGGGTGCAGTTGACATG
AGGATGGCAGAAGATCTCGGTCTAACTCAAGCCGAACGCACCGAG
ATGGCAAATACACTTGCCAAATTGACCACAGCAAATCGAGGGGCA
GACACCAGGGGAGGAGTCAACCCGTTCTCATCTGTCACTGGGACA
ACTCAGGTGCCCGCTGCAGCAACAGGTGACACACTCGAGAGTTAC
ATGGCAGCGGATCGACTGAGGCAGAGATATGCTGATGCAGGCACC
CATGATGATGAGATGCCACCATTGGAAGAGGAGGAAGAGGACGAC
ACATCTGCAGGTCCACGCACTGGACCAACTCTTGAACAAGTGGCC
TTGGACATCCAGAACGCAGCAGTTGGAGCTCCCATCCATACAGAT
GACCTGAATGCCGCACTGGGTGATCTTGACATCTAGACAATTCAG
ATCCCAATCTAAAATTGACATACCTAATTGATTAGTTAGATGGAA
CTACAGTGGATTCCATAAGGTTCCTGCCTACCATCGGCTTTAAAG
AAAAAAATAGGCCCGGACGGGTTAGCAACAAGCGACTGCCGGTGC
CAACAGCGCAATCCACAATCTACAATGGATCCCACTGATCTGAGC
TTCTCCCCAGATGAGATCAATAAGCTCATAGAGACAGGCCTGAAT
ACTGTAGAGTATTTTACTTCCCAACAAGTCACAGGAACATCCTCT
CTTGGAAAGAATACAATACCACCAGGGGTCACAGGACTACTAACC
AATGCTGCAGAGGCAAAGATCCAAGAGTCAACTAACCATCAGAAG
GGCTCAGTTGGTGGGGGTGCAAAACCAAAGAAACCGCGACCAAAA
ATTGCCATTGTGCCAGCAGATGACAAAACAGTGCCCGGAAAGCCG
ATCCCAAACCCTCTATTAGGTCTGGACTCCACCCCGAGCACCCAA
ACTGTGCTTGATCTAAGTGGGAAAACATTACCATCAGGATCCTAT
AAGGGGGTTAAGCTTGCGAAATTTGGAAAAGAAAATCTGATGACA
CGGTTCATCGAGGAACCCAGAGAGAATCCTATCGCAACCAGTTtC
CCCATCGATTTTAAGAGGGGCAGGGATACCGGCGGGTTCCATAGA
AGGGAGTACTCAATCGGATGGGTGGGAGATGAAGTCAAGGTCACT
GAGTGGTGCAATCCATCCTGTTCTCCAATCACCGCTGCAGCAAGG
CGATTTGAATGCACTTGTCACCAGTGTCCAGTCACTTGCTCTGAA
TGTGAACGAGATACTTAATACAGTGAGAAATTTGGACTCTCGGAT
GAATCAACTGGAGACAAAAGTAGATCGCATTCTCTCATCTCAGTC
TCTAATCCAGACCATCAAGAATGACATAGTTGGACTTAAAGCAGG
GATGGCTACTTTAGAAGGAATGATTACAACTGTGAAAATCATGGA
CCCGGGAGTTCCCAGTAATGTTACTGTGGAAGATGTACGCAAGAC
ACTAAGTAACCATGCTGTTGTTGTGCCAGAATCATTCAATGATAG
TTTCTTGACTCAATCTGAAGATGTAATTTCACTTGATGAGTTGGC
TCGACCAACTGCAACAAGTGTTAAGAAGATTGTCAGGAAGGTTCC
TCCTCAGAAGGATCTGACTGGATTGAAGATTACACTAGAGCAATT
GGCAAAGGATTGCATCAGCAAACCGAAGATGAGGGAAGAGTATCT
CCTCAAAATCAACCAGGCTTCCAGTGAGGCTCAGCTAATTGACCT
CAAGAAAGCAATCATCCGCAGTGCAATTTGATCAAGAAACACCCA
ATTACACTACACTGGTATGACACTGTACTAACCCTGAGGGTTTTA
GAAAAAACGATTAACGATAAATAAGCCCGAACACTACACACTACC
TGAGGCAGCCATGCCATCCATCAGCATTCCCGCAGACCCCACCAA
TCCACGTCAATCAATAAAAGCGTTCCCAATTGTGATCAACAGTGA
TGGGGGTGAGAAAGGCCGCTTGGTTAAACAACTACGCACAACCTA
CTTGAATGACCTAGATACTCATGAGCCACTGGTGACATTCATAAA
TACCTATGGATTCATCTACGAACAGGATCGGGGGAATACCATTGT
CGGAGAGGATCAACTTGGGAAGAAAAGAGAGGCTGTGACCGCTGC
AATGGTTACCCTTGGATGTGGGCCTAATCTACCATCATTAGGGAA
TGTCCTGGGACAACTGAGGGAATTCCAGGTCACTGTTAGGAAGAC
ATCCAGCAAAGCGGAAGAGATGGTCTTTGAAATTGTTAAGTATCC
GAGAATATTTCGGGGTCATACATTAATCCAGAAAGGACTAGTCTG
TGTCTCCGCAGAAAAATTTGTTAAGTCACCAGGGAAAATACAATC
TGGAATGGACTATCTCTTCATTCCGACATTTCTGTCAGTGACTTA
CTGTCCAGCTGCAATCAAATTTCAGGTACCTGGCCCCATGTTGAA
AATGAGATCAAGATACACTCAGAGCTTACAACTTGAACTAATGAT
AAGAATCCTGTGTAAGCCCGATTCGCCACTTATGAAGGTCCATAC
CCCTGACAAGGAGGGAAGAGGATGTCTTGTATCAGTATGGCTGCA
TGTATGCAACATCTTCAAATCAGGAAACAAGAATGGCAGTGAGTG
GCAGGAATACTGGATGAGAAAGTGTGCTAACATGCAACTTGAAGT
GTCGATTGCAGATATGTGGGGACCAACTATCATAATTCATGCCAG
AGGTCACATTCCCAAAAGTGCTAAGTTGTTTTTTGGAAAGGGTGG
ATGGAGCTGCCATCCACTTCACGAAGTTGTTCCAAGTGTCACTAA
AACACTATGGTCCGTGGGCTGTGAGATTACAAAGGCGAAGGCAAT
AATACAAGAGAGTAGCATCTCTCTTCTCGTGGAGACTACTGACAT
CATAAGTCCAAAAGTCAAAATTTCATCTAAGCATCGCCGCTTTGG
GAAATCAAATTGGGGTCTGTTCAAGAAAACTAAATCACTGCCTAA
CCTGACGGAGCTGGAATGACTGACCTCTAATCGAGACTACACCGC
CGCAAACTATAGGTGGGTGGTACCTCAGTGATTAATCTTGTAAGC
ACTGATCGTAGGCTACAACACACTAATATTATCCAGATTAGAGAG
CTTAATTAGCTCTGTATTAATAATAACACTACTATTCCAATAACT
GGAATCACCAGCTTGATTTATCTCCAAAATGATTCAAAGAAAACA
AATCATATTAAGACTATCCTAAGCACGAACCCATATCGTCCTTCA
AATCATGGGTACTATAATTCAATTTCTGGTGGTCTCCTGTCTATT
GGCAGGAGCAGGCAGCCTTGATCCAGCAGCCCTCATGCAAATCGG
TGTCATTCCAACAAATGTCCGGCAACTTATGTATTATACTGAAGC
TTCATCAGCATTCATTGTTGTGAAGTTAATGCCTACAATTGACTC
GCCGATTAGTGGATGTAATATAACATCAATTTCAAGCTATAATGC
AACAGTGACAAAACTCCTACAGCCGATCGGTGAGAATTTGGAGAC
GATTAGGAACCAGTTGATTCCAACTCGGAGGAGACGCCGGTTTGC
AGGGGTGGTGATTGGATTAGCTGCATTAGGAGTAGCTACTGCCGC
ACAGGTCACTGCCGCAGTGGCACTAGTAAAGGCAAATGAAAATGC
TGCGGCTATACTCAATCTCAAAAATGCAATCCAAAAAACAAATGC
AGCAGTTGCAGATGTGGTCCAGGCCACACAATCACTAGGAACGGC
AGTTCAAGCAGTTCAAGATCACATAAACAGTGTGGTAAGTCCAGC
AATTACAGCAGCCAATTGTAAGGCCCAAGATGCTATCATTGGCTC
AATCCTCAATCTCTATTTGACCGAGTTGACAACCATCTTCCACAA
TCAAATTACAAACCCTGCATTGAGTCCCATTACAATTCAAGCTTT
AAGGATCCTACTGGGGAGTACCTTGCCGACTGTGGTCGAAAAATC
TTTCAATACCCAGATAAGTGCAGCTGAGCTTCTCTCATCAGGGTT
ATTGACAGGCCAGATTGTGGGATTAGATTTGACCTATATGCAGAT
GGTCATAAAAATTGAGCTGCCAACTTTAACTGTACAACCTGCAAC
CCAGATCATAGATCTGGCCACCATTTCTGCATTCATTAACAATCA
AGAAGTCATGGCCCAATTACCAACACGTGTTATGGTGACTGGCAG
CTTGATCCAAGCCTATCCCGCATCGCAATGCACCATTACACCCAA
CACTGTGTACTGTAGGTATAATGATGCCCAAGTACTCTCAGATGA
TACTATGGCTTGCCTCCAAGGTAACTTGACAAGATGCACCTTCTC
TCCAGTGGTTGGGAGCTTTCTCACTCGATTCGTGCTGTTCGATGG
AATAGTTTATGCAAATTGCAGGTCGATGTTGTGCAAGTGCATGCA
ACCTGCTGCTGTGATCCTACAGCCGAGTTCATCCCCTGTAACTGT
CATTGACATGTACAAATGTGTGAGTCTGCAGCTTGACAATCTCAG
ATTCACCATCACTCAATTGGCCAATGTAACCTACAATAGCACCAT
CAAGCTTGAATCATCCCAGATCTTGTCTATTGATCCGTTGGATAT
ATCCCAGAATCTAGCTGCGGTGAATAAGAGTCTAAGTGATGCACT
ACAACACTTAGCACAAAGTGACACATATCTTTCTGCAATCACATC
AGCTACGACTACAAGTGTATTATCCATAATAGCAATCTGTCTTGG
ATCGTTAGGTTTAATATTAATAATCTTGCTCAGTGTAGTTGTGTG
GAAGTTATTGACCATTGTCGTTGCTAATCGAAATAGAATGGAGAA
TTTTGTTTATCATAAATAAGCATTCCACCACTCACGATCTGATCT
CAGTGAGAAAAATCAACCTGCAACTCTTGGAACAAGATAAGACAG
TCATCCATTAGTAATTTTTAAGAAAAAAACGATAGGACCGAACCT
AGTATTGAAAGAACCGTCTCGGTCAATCTAGGTAATCGAGCTGAT
ACCGTCTCGGAAAGCTCAAATATGGTTGCAGAAGATGCCCCTGTT
AGGGCCACTTGCCGAGTATTATTTCGAACAACAACTTTAATCTTT
CTATGCACACTACTAGCATTAAGCATCTCTATCCTTTATGAGAGT
TTAATAACCCAAAAGCAAATCATGAGCCAAGCAGGCTCAACTGGA
TCTAATTCTGGATTAGGAAGTATCACTGATCTTCTTAATAATATT
CTCTCTGTCGCAAATCAGATTATATATAACTCTGCAGTCGCTCTA
CCTCTACAATTGGACACTCTTGAATCAACACTCCTTACAGCCATT
AAGTCTCTTCAAACCAGTGACAAGCTAGAACAGAACTGCTCGTGG
AGTGCTGCACTGATTAATGATAATAGATACATTAATGGCATCAAT
CAGTTCTATTTTTCAATTGCTGAGGGTCGCAATCTGACACTTGGC
CCACTTCTTAATATGCCTAGTTTCATTCCAACTGCCACGACACCA
GAGGGCTGCACCAGGATCCCATCATTCTCGCTCACTAAGACACAC
TGGTGTTATACACACAATGTTATCCTGAATGGATGCCAGGATCAT
GTATCCTCAAATCAATTTGTTTCTATGGGAATCATTGAACCCACT
TCTGCCGGGTTTCCATTCTTTCGAACCCTAAAGACTCTATATCTC
AGCGATGGGGTCAATCGTAAGAGCTGCTCTATCAGTACAGTTCCG
GGGGGTTGTATGATGTACTGTTTTGTTTCTACTCAACCAGAGAGG
GATGACTACTTTTCTGCCGCTCCTCCAGAACAACGAATTATTATA
ATGTACTATAATGATACAATCGTGGAGCGCATAATTAATCCACCC
GGGGTACTAGATGTATGGGCAACATTGAACCCAGGAACAGGAAGC
GGGGTATATTATTTAGGTTGGGTGCTCTTTCCAATATATGGCGGC
GTGATTAAAGGTACGAGTTTATGGAATAATCAAGCAAATAAATAC
TTTATCCCCCAGATGGTTGCTGCTCTCTGCTCACAAAACCAGGCA
ACTCAAGTCCAAAATGCTAAGTCATCATACTATAGCAGCTGGTTT
GGCAATCGAATGATTCAGTCTGGGATCCTGGCATGTCCTCTTCGA
CAGGATCTAACCAATGAGTGTTTAGTTCTGCCCTTTTCTAATGAT
CAGGTGCTTATGGGTGCTGAAGGGAGATTATACATGTATGGTGAC
TCGGTGTATTACTATCAAAGAAGCAATAGTTGGTGGCCTATGACC
ATGCTGTATAAGGTAACCATAACATTCACTAATGGTCAGCCATCT
GCTATATCAGCTCAGAATGTGCCCACACAGCAGGTCCCTAGACCT
GGGACAGGAGACTGCTCTGCAACCAATAGATGTCCCGGTTTTTGC
TTGACAGGAGTGTATGCCGATGCCTGGTTACTGACCAACCCTTCG
TCTACCAGTACATTTGGATCAGAAGCAACCTTCACTGGTTCTTAT
CTCAACACAGCAACTCAGCGTATCAATCCGACGATGTATATCGCG
AACAACACACAGATCATAAGCTCACAGCAATTTGGATCAAGCGGT
CAAGAAGCAGCATATGGCCACACAACTTGTTTTAGGGACACAGGC
TCTGTTATGGTATACTGTATCTATATTATTGAATTGTCCTCATCT
CTCTTAGGACAATTTCAGATTGTCCCATTTATCCGTCAGGTGACA
CTATCCTAAAGGCAGAAGCCTTCAGGTCTGACCCAGCCAATCAAA
GCATTATACCAGACCATGGCCTACCATCGGCTTTAAAGAAAAAAA
TAGGCCCGGACGGGTTAGCAACAAGCGGCGGCCGCCatgttcgtc
ttcctggtcctgctgcctctggtctcctcacagtgcgtcaatctg
acaactcggactcagctgccacctgcttatactaatagcttcacc
agaggcgtgtactatcctgacaaggtgtttagaagctccgtgctg
cactctacacaggatctgtttctgccattctttagcaacgtgacc
tggttccacgccatccacgtgagcggcaccaatggcacaaagcgg
ttcgacaatcccgtgctgccttttaacgatggcgtgtacttcgcc
tctaccgagaagagcaacatcatcagaggctggatctttggcacc
acactggactccaagacacagtctctgctgatcgtgaacaatgcc
accaacgtggtcatcaaggtgtgcgagttccagttttgtaatgat
cccttcctgggcgtgtactatcacaagaacaataagagctggatg
gagtccgagtttagagtgtattctagcgccaacaactgcacattt
gagtacgtgagccagcctttcctgatggacctggagggcaagcag
ggcaatttcaagaacctgagggagttcgtgtttaagaatatcgac
ggctacttcaaaatctactctaagcacacccccatcaacctggtg
cgcgacctgcctcagggcttcagcgccctggagcccctggtggat
ctgcctatcggcatcaacatcacccggtttcagacactgctggcc
ctgcacagaagctacctgacacccggcgactcctctagcggatgg
accgccggcgctgccgcctactatgtgggctacctccagccccgg
accttcctgctgaagtacaacgagaatggcaccatcacagacgca
gtggattgcgccctggaccccctgagcgagacaaagtgtacactg
aagtcctttaccgtggagaagggcatctatcagacatccaatttc
agggtgcagccaaccgagtctatcgtgcgctttcctaatatcaca
aacctgtgcccatttggcgaggtgttcaacgcaacccgcttcgcc
agcgtgtacgcctggaataggaagcggatcagcaactgcgtggcc
gactatagcgtgctgtacaactccgcctctttcagcacctttaag
tgctatggcgtgtcccccacaaagctgaatgacctgtgctttacc
aacgtctacgccgattctttcgtgatcaggggcgacgaggtgcgc
cagatcgcccccggccagacaggcaagatcgcagactacaattat
aagctgccagacgatttcaccggctgcgtgatcgcctggaacagc
aacaatctggattccaaagtgggcggcaactacaattatctgtac
cggctgtttagaaagagcaatctgaagcccttcgagagggacatc
tctacagaaatctaccaggccggcagcaccccttgcaatggcgtg
gagggctttaactgttatttcccactccagtcctacggcttccag
cccacaaacggcgtgggctatcagccttaccgcgtggtggtgctg
agctttgagctgctgcacgccccagcaacagtgtgcggccccaag
aagtccaccaatctggtgaagaacaagtgcgtgaacttcaacttc
aacggcctgaccggcacaggcgtgctgaccgagtccaacaagaag
ttcctgccatttcagcagttcggcagggacatcgcagataccaca
gacgccgtgcgcgacccacagaccctggagatcctggacatcaca
ccctgctctttcggcggcgtgagcgtgatcacacccggcaccaat
acaagcaaccaggtggccgtgctgtatcaggacgtgaattgtacc
gaggtgcccgtggctatccacgccgatcagctgaccccaacatgg
cgggtgtacagcaccggctccaacgtcttccagacaagagccgga
tgcctgatcggagcagagcacgtgaacaattcctatgagtgcgac
atcccaatcggcgccggcatctgtgcctcttaccagacccagaca
aactctcccagaagagcccggagcgtggcctcccagtctatcatc
gcctataccatgtccctgggcgccgagaacagcgtggcctactct
aacaatagcatcgccatcccaaccaacttcacaatctctgtgacc
acagagatcctgcccgtgtccatgaccaagacatctgtggactgc
acaatgtatatctgtggcgattctaccgagtgcagcaacctgctg
ctccagtacggcagcttttgtacccagctgaatagagccctgaca
ggcatcgccgtggagcaggataagaacacacaggaggtgttcgcc
caggtgaagcaaatctacaagaccccccctatcaaggactttggc
ggcttcaatttttcccagatcctgcctgatccatccaagccttct
aagcggagctttatcgaggacctgctgttcaacaaggtgaccctg
gccgatgccggcttcatcaagcagtatggcgattgcctgggcgac
atcgcagccagggacctgatctgcgcccagaagtttaatggcctg
accgtgctgccacccctgctgacagatgagatgatcgcacagtac
acaagcgccctgctggccggcaccatcacatccggatggaccttc
ggcgcaggagccgccctccagatcccctttgccatgcagatggcc
tataggttcaacggcatcggcgtgacccagaatgtgctgtacgag
aaccagaagctgatcgccaatcagtttaactccgccatcggcaag
atccaggacagcctgtcctctacagccagcgccctgggcaagctc
caggatgtggtgaatcagaacgcccaggccctgaataccctggtg
aagcagctgagcagcaacttcggcgccatctctagcgtgctgaat
gacatcctgagccggctggacaaggtggaggcagaggtgcagatc
gaccggctgatcaccggccggctccagagcctccagacctatgtg
acacagcagctgatcagggccgccgagatcagggccagcgccaat
ctggcagcaaccaagatgtccgagtgcgtgctgggccagtctaag
agagtggacttttgtggcaagggctatcacctgatgtccttccct
cagtctgccccacacggcgtggtgtttctgcacgtgacctacgtg
cccgcccaggagaagaacttcaccacagcccctgccatctgccac
gatggcaaggcccactttccaagggagggcgtgttcgtgtccaac
ggcacccactggtttgtgacacagcgcaatttctacgagccccag
atcatcaccacagacaacaccttcgtgagcggcaactgtgacgtg
gtcatcggcatcgtgaacaataccgtgtatgatccactccagccc
gagctggacagctttaaggaggagctggataagtatttcaagaat
cacacctcccctgacgtggatctgggcgacatcagcggcatcaat
gcctccgtggtgaacatccagaaggagatcgaccgcctgaacgag
gtggctaagaatctgaacgagagcctgatcgacctccaggagctg
ggcaagtatgagcagtacatcaagtggccctggtacatctggctg
ggcttcatcgccggcctgatcgccatcgtgatggtgaccatcatg
ctgtgctgtatgacatcctgctgttcttgcctgaagggctgctgt
agctgtggctcctgctgtaagttattgaccattgtcgttgctaat
cgaaatagaatggagaattttgtttatcataaatgaAGTCGACTC
ATGGAATGCATACCAAACATTATTGACACTAATGACACACAAAAT
TGGTTTTAAGAAAAACCAAGAGAACAATAGGCCAGAATGGCTGGG
TCTCGGGAGATATTACTCCCTGAAGTCCATCTCAATTCACCAATT
GTAAAGCATAAGCTATACTATTACATTCTACTTGGAAACCTCCCA
AATGAGATCGACCTTGACGATTTAGGTCCATTACATAATCAAAAT
TGGAATCAGATAGCACATGAAGAGTCTAACTTAGCTCAACGCTTG
GTAAATGTAAGAAATTTTCTAATTACCCACATCCCTGATCTTAGA
AAGGGCCATTGGCAAGAGTATGTCAATGTAATACTGTGGCCGCGA
ATTCTTCCCTTGATCCCGGATTTTAAAATCAATGACCAATTGCCT
CTGCTCAAAAATTGGGACAAGTTAGTTAAAGAATCATGTTCAGTA
ATCAATGCAGGTACTTCCCAGTGCATTCAGAATCTCAGCTATGGA
CTGACAGGTCGTGGGAACCTCTTTACACGATCACGTGAACTCTCT
GGTGACCGCAGGGATATTGATCTTAAGACAGTTGTGGCAGCATGG
CATGACTCAGACTGGAAAAGAATAAGTGATTTTTGGATTATGATC
AAATTCCAGATGAGACAATTAATTGTTAGGCAAACAGATCATAAT
GATTCTGATTTAATCACGTATATCGAAAATAGAGAAGGCATAATC
ATCATAACCCCTGAACTGGTAGCATTATTTAACACTGAGAATCAT
ACACTAACATACATGACCTTTGAAATTGTACTGATGGTTTCAGAT
ATGTACGAAGGTCGTCACAACATTTTATCACTATGCACAGTTAGC
ACTTACCTGAATCCTCTGAAGAAAAGAATAACATATTTATTGAGC
CTTGTAGATAACTTAGCTTTTCAGATAGGTGATGCTGTATATAAC
ATAATTGCTTTGCTAGAATCCTTTGTATATGCACAGTTGCAAATG
TCAGATCCCATCCCAGAACTCAGAGGACAATTCCATGCATTCGTA
TGTTCTGAGATTCTTGATGCACTAAGAGGAACTAATAGTTTCACC
CAGGATGAATTAAGAACTGTGACAACTAATTTGATATCCCCATTC
CAAGATCTGACCCCAGATCTTACGGCTGAATTGCTCTGTATAATG
AGGCTTTGGGGACACCCCATGCTCACTGCCAGTCAAGCTGCAGGA
AAGGTACGCGAGTCTATGTGTGCTGGAAAAGTATTAGACTTTCCC
ACCATTATGAAAACACTAGCCTTTTTCCATACTATTCTGATCAAT
GGATACAGGAGGAAGCATCATGGAGTATGGCCACCCTTAAACTTA
CCGGGTAATGCTTCAAAGGGTCTCACGGAACTTATGAATGACAAT
ACTGAAATAAGCTATGAATTCACACTTAAGCATTGGAAGGAAGTC
TCTCTTATAAAATTCAAGAAATGTTTTGATGCAGACGCAGGTGAG
GAACTCAGTATATTTATGAAAGATAAGGCAATTAGTGCCCCAAAA
CAAGACTGGATGAGTGTGTTTAGAAGAAGCCTAATCAAACAGCGC
CATCAGCATCATCAGGTCCCCCTACCAAATCCATTCAATCGACGG
CTGTTGCTAAACTTTCTCGGAGATGACAAATTCGACCCGAATGTG
GAGCTACAGTATGTAACATCAGGTGAGTATCTACATGATGACACG
TTTTGTGCATCATATTCACTAAAAGAGAAGGAAATTAAACCTGAT
GGTCGAATTTTTGCAAAGTTGACTAAGAGAATGAGATCATGTCAA
GTTATAGCAGAATCTCTTTTAGCGAACCATGCTGGGAAGTTAATG
AAAGAGAATGGTGTTGTGATGAATCAGCTATCATTAACAAAATCA
CTATTAACAATGAGTCAGATTGGAATAATATCCGAGAAAGCTAGA
AAGTCAACTCGAGATAACATAAATCAACCTGGTTTCCAGAATATC
CAGAGAAATAAATCACATCACTCCAAGCAAGTCAATCAGCGAGAT
CCAAGTGATGACTTTGAATTGGCAGCATCTTTTTTAACTACTGAT
CTCAAAAAATATTGTTTACAATGGAGGTACCAGACAATTATCCCA
TTTGCTCAATCATTAAACAGAATGTATGGTTATCCTCATCTCTTT
GAGTGGATTCACTTACGGCTAATGCGTAGTACACTTTACGTGGGG
GATCCCTTCAACCCACCAGCAGATACCAGTCAATTTGATCTAGAT
AAAGTAATTAATGGAGATATCTTCATTGTATCACCCAGAGGTGGA
ATTGAAGGGCTGTGTCAAAAGGCTTGGACAATGATATCTATCGCT
GTGATAATTCTATCTGCCACAGAGTCTGGCACACGAGTAATGAGT
ATGGTGCAGGGAGATAATCAAGCAATTGCTGTCACCACACGAGTA
CCAAGGAGCCTGCCGACTCTTGAGAAAAAGACTATTGCTTTTAGA
TCTTGTAATCTATTCTTTGAGAGGTTAAAATGTAATAATTTTGGA
TTAGGTCACCATTTGAAAGAACAAGAGACTATCATTAGTTCTCAC
TTCTTTGTTTATAGCAAGAGAATATTCTATCAGGGGAGGATTCTA
ACGCAAGCCTTAAAAAATGCTAGTAAGCTCTGCTTGACAGCTGAT
GTCCTAGGAGAATGCACCCAATCATCATGTTCTAATCTTGCAACT
ACTGTCATGAGGTTAACTGAGAATGGTGTTGAAAAAGATATCTGT
TTCTACTTGAATATCTATATGACCATCAAACAGCTCTCCTATGAT
ATCATCTTCCCTCAAGTGTCAATTCCTGGAGATCAGATCACATTA
GAATACATAAATAATCCACACCTGGTATCACGATTGGCTCTTTTG
CCATCCCAGTTAGGAGGTCTAAACTACCTGTCATGCAGTAGGCTG
TTCAATCGAAACATAGGCGACCCGGTGGTTTCCGCAGTTGCAGAT
CTTAAGAGATTAATTAAATCAGGATGTATGGATTACTGGATCCTT
TATAACTTATTAGGGAGAAAACCGGGAAACGGCTCATGGGCTACT
TTAGCAGCTGACCCGTACTCAATCAATATAGAGTATCAATACCCT
CCAACTACAGCTCTTAAGAGGCACACCCAACAAGCTCTGATGGAA
CTCAGTACGAATCCAATGTTACGTGGCATATTCTCTGACAATGCA
CAGGCAGAAGAAAATAACCTTGCTAGGTTTCTCCTGGATAGGGAG
GTGATCTTTCCGCGTGTAGCTCACATCATCATTGAGCAAACCAGT
GTCGGGAGGAGAAAACAGATTCAAGGATATTTGGATTCAACTAGA
TCGATAATGAGGAAATCACTAGAAATTAAGCCCTTATCCAATAGG
AAGCTTAATGAAATACTGGATTACAACATCAATTACCTAGCTTAC
AATTTGGCATTACTCAAGAATGCTATTGAACCTCCGACTTATTTG
AAGGCAATGACACTTGAAACATGTAGCATCGACATTGCAAGGAAC
CTCCGGAAGCTCTCCTGGGCCCCACTCTTGGGTGGGAGAAATCTT
GAAGGATTAGAGACGCCAGATCCCATTGAAATTACTGCAGGAGCA
TTAATTGTTGGATCGGGCTACTGTGAACAGTGTGCTGCAGGAGAC
AATCGATTCACATGGTTTTTCTTGCCATCTGGTATCGAGATAGGA
GGGGATCCCCGTGATAATCCTCCTATCCGTGTACCGTACATTGGC
TCCAGGACTGATGAGAGGAGGGTAGCCTCAATGGCATACATCAGG
GGTGCCTCGAGTAGCTTAAAAGCAGTTCTTAGACTGGCGGGAGTG
TACATCTGGGCATTCGGAGATACTCTGGAGAATTGGATAGATGCA
CTGGATTTGTCTCACACTAGAGTTAACATCACACTTGAACAGCTG
CAATCCCTCACCCCACTTCCAACCTCTGCCAATCTAACCCATCGG
TTGGATGATGGCACAACTACCCTAAAGTTTACTCCTGCGAGCTCT
TACACCTTTTCAAGTTTCACTCATATATCAAATGATGAGCAATAC
CTGACAATTAATGACAAAACTGCAGATTCAAATATAATCTACCAA
CAGTTAATGATCACTGGACTCGGAATCTTAGAAACATGGAATAAT
CCCCCAATCAATAGAACATTCGAAGAATCTACCCTACATTTGCAC
ACTGGTGCATCATGTTGTGTCCGACCTGTGGACTCCTGCATTCTC
TCAGAAGCATTAACAGTCAAGCCACATATTACAGTACCGTACAGC
AATAAATTTGTATTTGATGAGGACCCGCTATCTGAATATGAAACT
GCAAAACTGGAATCGTTATCATTCCAAGCCCAATTAGGCAACATT
GATGCTGTAGATATGACAGGTAAATTAACATTATTGTCCCAATTC
ACTGCAAGGCAGATTATCAATGCAATCACTGGACTCGATGAGTCT
GTCTCTCTTACTAATGATGCCATTGTTGCATCAGACTATGTCTCC
AATTGGATTAGTGAATGCATGTATACCAAATTAGATGAATTATTT
ATGTATTGTGGGTGGGAACTACTATTGGAACTATCCTATCAAATG
TATTATCTGAGGGTAGTTGGGTGGAGTAATATAGTGGATTATTCT
TACATGATCTTGAGAAGAATCCCGGGTGCAGCATTAAACAATCTG
GCATCTACATTAAGTCATCCAAAACTTTTCCGACGAGCTATCAAC
CTAGATATAGTTGCCCCCTTAAATGCTCCTCATTTTGCATCTCTG
GACTACATCAAGATGAGTGTGGATGCAATACTCTGGGGCTGTAAA
AGAGTCATCAATGTGCTCTCCAATGGAGGGGACTTAGAATTAGTT
GTGACATCTGAAGATAGCCTTATTCTCAGTGACCGATCCATGAAT
CTCATTGCAAGGAAATTAACTTTATTATCACTGATTCACCATAAT
GGTTTGGAACTACCAAAGATTAAGGGGTTCTCTCCTGATGAGAAG
TGTTTCGCTTTGACAGAATTTTTGAGGAAAGTGGTGAACTCAGGG
TTGAGTTCAATAGAGAACCTATCAAATTTTATGTACAATGTGGAG
AACCCACGGCTTGCAGCATTCGCCAGCAACAATTACTACCTGACC
AGAAAATTATTGAATTCAATACGAGATACTGAGTCGGGTCAAGTA
GCAGTCACCTCATATTATGAATCATTAGAATATATTGATAGTCTT
AAGCTAACCCCACATGTGCCTGGCACCTCATGCATTGAGGATGAT
AGTCTATGTACAAATGATTACATAATCTGGATCATAGAGTCTAAT
GCAAACTTGGAGAAGTATCCAATTCCAAATAGCCCTGAGGATGAT
TCCAATTTCCATAACTTTAAGTTGAATGCTCCATCGCACCATACC
TTACGCCCATTAGGGTTGTCATCAACTGCTTGGTATAAGGGTATA
AGCTGCTGCAGGTACCTTGAGCGATTAAAGCTACCACAAGGTGAT
CATTTATATATTGCAGAAGGTAGTGGTGCCAGTATGACAATCATA
GAATACCTATTCCCAGGAAGAAAGATATATTACAATTCTTTATTT
AGTAGTGGTGACAATCCCCCACAAAGAAATTATGCACCAATGCCT
ACTCAGTTCATTGAGAGTGTCCCATACAAGCTCTGGCAAGCACAC
ACAGATCAATATCCCGAGATTTTTGAGGACTTCATCCCTCTATGG
AACGGAAACGCCGCCATGACTGACATAGGAATGACAGCTTGTGTA
GAATTCATCATCAATCGAGTCGGCCCAAGGACTTGCAGTTTAGTA
CATGTAGATTTGGAATCAAGTGCAAGCTTAAATCAACAATGCCTG
TCAAAGCCGATAATTAATGCTATCATCACTGCTACAACTGTTTTG
TGCCCTCATGGGGTGCTTATTCTGAAATATAGTTGGTTGCCATTT
ACTAGATTTAGTACTTTGATCACTTTCTTATGGTGCTACTTTGAG
AGAATCACTGTTCTTAGGAGCACATATTCTGATCCAGCTAATCAT
GAGGTTTATTTAATTTGTATCCTTGCCAACAACTTTGCATTCCAG
ACTGTCTCGCAGGCAACAGGAATGGCGATGACTTTAACTGATCAA
GGGTTTACTTTGATATCACCTGAAAGAATAAATCAGTATTGGGAT
GGTCACTTGAAGCAAGAACGTATCGTAGCAGAAGCAATTGATAAG
GTGGTTCTAGGAGAAAATGCTCTATTTAATTCGAGTGATAATGAA
TTAATTCTCAAATGTGGAGGGACACCAAATGCACGGAATCTCATC
GATATCGAGCCAGTCGCAACTTTCATAGAATTTGAACAATTGATC
TGCACAATGTTGACAACCCACTTGAAGGAAATAATTGATATAACA
AGGTCTGGAACCCAGGATTATGAAAGTTTATTACTCACTCCTTAC
AATTTAGGTCTTCTTGGTAAAATCAGTACGATAGTGAGATTATTA
ACAGAAAGGATTCTAAATCATACTATCAGGAATTGGTTGATCCTC
CCACCTTCGCTCCGGATGATCGTGAAGCAGGACTTGGAATTCGGC
ATATTCAGGATTACTTCCATCCTCAATTCTGATCGGTTCCTGAAG
CTTTCTCCAAATAGGAAATACTTGATTGCACAATTAACTGCAGGC
TACATTAGGAAATTGATTGAGGGGGATTGCAATATCGATCTAACC
AGACCTATCCAAAAGCAAATCTGGAAAGCATTAGGTTGTGTAGTC
TATTGTCACGATCCAATGGATCAAAGGGAGTCAACAGAGTTTATT
GATATAAATATTAATGAAGAAATAGACCGCGGGATCGATGGCGAG
GAAATCTAAACATATCAAGAATCAGAATTAGTTTAAGAAAAAAGA
AGAGGATTAATCTTGGTTTTCCCCTTGGTGGGTCGGCATGGCATC
TCCACCTCCTCGCGGTCCGACCTGGGCATCCGAAGGAGGACGCAC
GTCCACTCGGATGGCTAAGGGAGCGGCCGGGGATCCGGCTGCTAA
CAAAGCCCGAAAGGAAGCTGAGTTGGCTGCTGCCACCGCTGAGCA
ATAACTAGCATAACCCCTTGGGGCCTCTAAACGGGTCTTGAGGGG
TTTTTTGCTGAAAGGAGGAACTATATCCGGATCATGTGAGCAAAA
GGCCAGCAAAAGGCCAGGAACCGTAAAAAGGCCGCGTTGCTGGCG
TTTTTCCATAGGCTCCGCCCCCCTGACGAGCATCACAAAAATCGA
CGCTCAAGTCAGAGGTGGCGAAACCCGACAGGACTATAAAGATAC
CAGGCGTTTCCCCCTGGAAGCTCCCTCGTGCGCTCTCCTGTTCCG
ACCCTGCCGCTTACCGGATACCTGTCCGCCTTTCTCCCTTCGGGA
AGCGTGGCGCTTTCTCATAGCTCACGCTGTAGGTATCTCAGTTCG
GTGTAGGTCGTTCGCTCCAAGCTGGGCTGTGTGCACGAACCCCCC
GTTCAGCCCGACCGCTGCGCCTTATCCGGTAACTATCGTCTTGAG
TCCAACCCGGTAAGACACGACTTATCGCCACTGGCAGCAGCCACT
GGTAACAGGATTAGCAGAGCGAGGTATGTAGGCGGTGCTACAGAG
TTCTTGAAGTGGTGGCCTAACTACGGCTACACTAGAAGAACAGTA
TTTGGTATCTGCGCTCTGCTGAAGCCAGTTACCTTCGGAAAAAGA
GTTGGTAGCTCTTGATCCGGCAAACAAACCACCGCTGGTAGCGGT
GGTTTTTTTGTTTGCAAGCAGCAGATTACGCGCAGAAAAAAAGGA
TCTCAAGAAGATCCTTTGATCTTTTCTACGGGGTCTGACGCTCAG
TGGAACGAAAACTCACGTTAAGGGATTTTGGTCATGAGATTATCA
AAAAGGATCTTCACCTAGATCCTTTTAAATTAAAAATGAAGTTTT
AAATCAATCTAAAGTATATATGAGTAAACTTGGTCTGACAGTTAC
CAATGCTTAATCAGTGAGGCACCTATCTCAGCGATCTGTCTATTT
CGTTCATCCATAGTTGCCTGACTCCCCGTCGTGTAGATAACTACG
ATACGGGAGGGCTTACCATCTGGCCCCAGTGCTGCAATGATACCG
CGAGACCCACGCTCACCGGCTCCAGATTTATCAGCAATAAACCAG
CCAGCCGGAAGGGCCGAGCGCAGAAGTGGTCCTGCAACTTTATCC
GCCTCCATCCAGTCTATTAATTGTTGCCGGGAAGCTAGAGTAAGT
AGTTCGCCAGTTAATAGTTTGCGCAACGTTGTTGCCATTGCTACA
GGCATCGTGGTGTCACGCTCGTCGTTTGGTATGGCTTCATTCAGC
TCCGGTTCCCAACGATCAAGGCGAGTTACATGATCCCCCATGTTG
TGCAAAAAAGCGGTTAGCTCCTTCGGTCCTCCGATCGTTGTCAGA
AGTAAGTTGGCCGCAGTGTTATCACTCATGGTTATGGCAGCACTG
CATAATTCTCTTACTGTCATGCCATCCGTAAGATGCTTTTCTGTG
ACTGGTGAGTACTCAACCAAGTCATTCTGAGAATAGTGTATGCGG
CGACCGAGTTGCTCTTGCCCGGCGTCAATACGGGATAATACCGCG
CCACATAGCAGAACTTTAAAAGTGCTCATCATTGGAAAACGTTCT
TCGGGGCGAAAACTCTCAAGGATCTTACCGCTGTTGAGATCCAGT
TCGATGTAACCCACTCGTGCACCCAACTGATCTTCAGCATCTTTT
ACTTTCACCAGCGTTTCTGGGTGAGCAAAAACAGGAAGGCAAAAT
GCCGCAAAAAAGGGAATAAGGGCGACACGGAAATGTTGAATACTC
ATACTCTTCCTTTTTCAATATTATTGAAGCATTTATCAGGGTTAT
TGTCTCATGAGCGGATACATATTTGAATGTATTTAGAAAAATAAA
CAAATAGGGGTTCCGCGCACATTTCCCCGAAAAGT.

iii. CVL85

The nucleic acid sequence for CVL85 is:

(SEQ ID NO: 5)
GCCACCTGACGTCTAAGAAACCATTATTATCATGACATTAACCTA
TAAAAATAGGCGTATCACGAGGCCCTTTCGTCTCGCGCGTTTCGG
TGATGACGGTGAAAACCTCTGACACATGCAGCTCCCGGAGACGGT
CACAGCTTGTCTGTAAGCGGATGCCGGGAGCAGACAAGCCCGTCA
GGGCGCGTCAGCGGGTGTTGGCGGGTGTCGGGGCTGGCTTAACTA
TGCGGCATCAGAGCAGATTGTACTGAGAGTGCACCATATATGCGG
TGTGAAATACCGCACAGATGCGTAAGGAGAAAATACCGCATCAGG
CTAATACGACTCACTATAGGGACCAAGGGGAAAATGAAGTGGTGA
CTCAAATCATCGAAGACCCTCGAGATTACATAGGTCCGGAACCTA
TGGCCTTCGTGACCGACCTCGAGTCAGAGTAGTTCAATAAGGACC
TATCAAGTTTGGGCAATTTTTCGTCCCCGACACAAAAATGTCATC
CGTGCTTAAAGCATATGAGCGATTCACGCTCACTCAAGAACTGCA
AGATCAGAGTGAGGAAGGTACAATCCCACCTACAACACTAAAACC
GGTAATCAGGGTATTTATACTAACCTCTAATAACCCAGAGCTAAG
ATCCCGGCTTCTTCTATTCTGCCTACGGATTGTTCTCAGTAATGG
TGCAAGGGATTCCCATCGCTTTGGAGCATTACTCACAATGTTTTC
GCTACCATCAGCCACAATGCTCAATCATGTCAAATTAGCTGACCA
GTCACCAGAAGCTGATATCGAAAGGGTAGAGATCGATGGCTTTGA
GGAGGGATCATTCCGCTTAATCCCCAATGCTCGTTCAGGTATGAG
CCGTGGAGAGATCAATGCCTATGCTGCACTTGCAGAAGATCTACC
TGACACACTAAACCATGCAACACCTTTCGTTGATTCCGAAGTCGA
GGGAACTGCATGGGATGAGATTGAGACTTTCTTAGATATGTGTTA
CAGTGTCCTAATGCAGGCATGGATAGTGACTTGCAAGTGCATGAC
TGCGCCAGACCAACCTGCTGCTTCTATTGAGAAACGCCTGCAAAA
ATATCGTCAGCAAGGCAGGATCAACCCGAGATATCTCCTGCAACC
GGAGGCTCGACGAATAATCCAGAATGTAATCCGGAAGGGAATGGT
GGTCAGACATTTCCTCACCTTTGAACTGCAGCTTGCCCGAGCACA
AAGCCTTGTATCAAATAGGTATTATGCTATGGTAGGGGATGTTGG
AAAGTATATAGAGAATTGTGGAATGGGAGGCTTCTTTTTGACACT
AAAATATGCATTAGGAACTAGATGGCCCACACTTGCTTTAGCTGC
ATTTTCAGGAGAGCTAACAAAGCTAAAGTCCCTCATGGCATTATA
CCAGACCCTTGGTGAGCAGGCCCGATATTTGGCCCTATTGGAGTC
ACCACATTTGATGGATTTTGCTGCAGCAAACTACCCACTGCTATA
TAGCTATGCTATGGGAATAGGCTATGTGTTAGATGTCAACATGAG
GAACTACGCTTTCTCCAGATCATACATGAACAAGACATATTTCCA
ATTGGGAATGGAAACTGCAAGAAAACAACAGGGTGCAGTTGACAT
GAGGATGGCAGAAGATCTCGGTCTAACTCAAGCCGAACGCACCGA
GATGGCAAATACACTTGCCAAATTGACCACAGCAAATCGAGGGGC
AGACACCAGGGGAGGAGTCAACCCGTTCTCATCTGTCACTGGGAC
AACTCAGGTGCCCGCTGCAGCAACAGGTGACACACTCGAGAGTTA
CATGGCAGCGGATCGACTGAGGCAGAGATATGCTGATGCAGGCAC
CCATGATGATGAGATGCCACCATTGGAAGAGGAGGAAGAGGACGA
CACATCTGCAGGTCCACGCACTGGACCAACTCTTGAACAAGTGGC
CTTGGACATCCAGAACGCAGCAGTTGGAGCTCCCATCCATACAGA
TGACCTGAATGCCGCACTGGGTGATCTTGACATCTAGACAATTCA
GATCCCAATCTAAAATTGACATACCTAATTGATTAGTTAGATGGA
ACTACAGTGGATTCCATAAGGTTCCTGCCTACCATCGGCTTTAAA
GAAAAAAATAGGCCCGGACGGGTTAGCAACAAGCGACTGCCGGTG
CCAACAGCGCAATCCACAATCTACAATGGATCCCACTGATCTGAG
CTTCTCCCCAGATGAGATCAATAAGCTCATAGAGACAGGCCTGAA
TACTGTAGAGTATTTTACTTCCCAACAAGTCACAGGAACATCCTC
TCTTGGAAAGAATACAATACCACCAGGGGTCACAGGACTACTAAC
CAATGCTGCAGAGGCAAAGATCCAAGAGTCAACTAACCATCAGAA
GGGCTCAGTTGGTGGGGGTGCAAAACCAAAGAAACCGCGACCAAA
AATTGCCATTGTGCCAGCAGATGACAAAACAGTGCCCGGAAAGCC
GATCCCAAACCCTCTATTAGGTCTGGACTCCACCCCGAGCACCCA
AACTGTGCTTGATCTAAGTGGGAAAACATTACCATCAGGATCCTA
TAAGGGGGTTAAGCTTGCGAAATTTGGAAAAGAAAATCTGATGAC
ACGGTTCATCGAGGAACCCAGAGAGAATCCTATCGCAACCAGTTt
CCCCATCGATTTTAAGAGGGGCAGGGATACCGGCGGGTTCCATAG
AAGGGAGTACTCAATCGGATGGGTGGGAGATGAAGTCAAGGTCAC
TGAGTGGTGCAATCCATCCTGTTCTCCAATCACCGCTGCAGCAAG
GCGATTTGAATGCACTTGTCACCAGTGTCCAGTCACTTGCTCTGA
ATGTGAACGAGATACTTAATACAGTGAGAAATTTGGACTCTCGGA
TGAATCAACTGGAGACAAAAGTAGATCGCATTCTCTCATCTCAGT
CTCTAATCCAGACCATCAAGAATGACATAGTTGGACTTAAAGCAG
GGATGGCTACTTTAGAAGGAATGATTACAACTGTGAAAATCATGG
ACCCGGGAGTTCCCAGTAATGTTACTGTGGAAGATGTACGCAAGA
CACTAAGTAACCATGCTGTTGTTGTGCCAGAATCATTCAATGATA
GTTTCTTGACTCAATCTGAAGATGTAATTTCACTTGATGAGTTGG
CTCGACCAACTGCAACAAGTGTTAAGAAGATTGTCAGGAAGGTTC
CTCCTCAGAAGGATCTGACTGGATTGAAGATTACACTAGAGCAAT
TGGCAAAGGATTGCATCAGCAAACCGAAGATGAGGGAAGAGTATC
TCCTCAAAATCAACCAGGCTTCCAGTGAGGCTCAGCTAATTGACC
TCAAGAAAGCAATCATCCGCAGTGCAATTTGATCAAGAAACACCC
AATTACACTACACTGGTATGACACTGTACTAACCCTGAGGGTTTT
AGAAAAAACGATTAACGATAAATAAGCCCGAACACTACACACTAC
CTGAGGCAGCCATGCCATCCATCAGCATTCCCGCAGACCCCACCA
ATCCACGTCAATCAATAAAAGCGTTCCCAATTGTGATCAACAGTG
ATGGGGGTGAGAAAGGCCGCTTGGTTAAACAACTACGCACAACCT
ACTTGAATGACCTAGATACTCATGAGCCACTGGTGACATTCATAA
ATACCTATGGATTCATCTACGAACAGGATCGGGGGAATACCATTG
TCGGAGAGGATCAACTTGGGAAGAAAAGAGAGGCTGTGACCGCTG
CAATGGTTACCCTTGGATGTGGGCCTAATCTACCATCATTAGGGA
ATGTCCTGGGACAACTGAGGGAATTCCAGGTCACTGTTAGGAAGA
CATCCAGCAAAGCGGAAGAGATGGTCTTTGAAATTGTTAAGTATC
CGAGAATATTTCGGGGTCATACATTAATCCAGAAAGGACTAGTCT
GTGTCTCCGCAGAAAAATTTGTTAAGTCACCAGGGAAAATACAAT
CTGGAATGGACTATCTCTTCATTCCGACATTTCTGTCAGTGACTT
ACTGTCCAGCTGCAATCAAATTTCAGGTACCTGGCCCCATGTTGA
AAATGAGATCAAGATACACTCAGAGCTTACAACTTGAACTAATGA
TAAGAATCCTGTGTAAGCCCGATTCGCCACTTATGAAGGTCCATA
CCCCTGACAAGGAGGGAAGAGGATGTCTTGTATCAGTATGGCTGC
ATGTATGCAACATCTTCAAATCAGGAAACAAGAATGGCAGTGAGT
GGCAGGAATACTGGATGAGAAAGTGTGCTAACATGCAACTTGAAG
TGTCGATTGCAGATATGTGGGGACCAACTATCATAATTCATGCCA
GAGGTCACATTCCCAAAAGTGCTAAGTTGTTTTTTGGAAAGGGTG
GATGGAGCTGCCATCCACTTCACGAAGTTGTTCCAAGTGTCACTA
AAACACTATGGTCCGTGGGCTGTGAGATTACAAAGGCGAAGGCAA
TAATACAAGAGAGTAGCATCTCTCTTCTCGTGGAGACTACTGACA
TCATAAGTCCAAAAGTCAAAATTTCATCTAAGCATCGCCGCTTTG
GGAAATCAAATTGGGGTCTGTTCAAGAAAACTAAATCACTGCCTA
ACCTGACGGAGCTGGAATGACTGACCTCTAATCGAGACTACACCG
CCGCAAACTATAGGTGGGTGGTACCTCAGTGATTAATCTTGTAAG
CACTGATCGTAGGCTACAACACACTAATATTATCCAGATTAGAGA
GCTTAATTAGCTCTGTATTAATAATAACACTACTATTCCAATAAC
TGGAATCACCAGCTTGATTTATCTCCAAAATGATTCAAAGAAAAC
AAATCATATTAAGACTATCCTAAGCACGAACCCATATCGTCCTTC
AAATCATGGGTACTATAATTCAATTTCTGGTGGTCTCCTGTCTAT
TGGCAGGAGCAGGCAGCCTTGATCCAGCAGCCCTCATGCAAATCG
GTGTCATTCCAACAAATGTCCGGCAACTTATGTATTATACTGAAG
CTTCATCAGCATTCATTGTTGTGAAGTTAATGCCTACAATTGACT
CGCCGATTAGTGGATGTAATATAACATCAATTTCAAGCTATAATG
CAACAGTGACAAAACTCCTACAGCCGATCGGTGAGAATTTGGAGA
CGATTAGGAACCAGTTGATTCCAACTCGGAGGAGACGCCGGTTTG
CAGGGGTGGTGATTGGATTAGCTGCATTAGGAGTAGCTACTGCCG
CACAGGTCACTGCCGCAGTGGCACTAGTAAAGGCAAATGAAAATG
CTGCGGCTATACTCAATCTCAAAAATGCAATCCAAAAAACAAATG
CAGCAGTTGCAGATGTGGTCCAGGCCACACAATCACTAGGAACGG
CAGTTCAAGCAGTTCAAGATCACATAAACAGTGTGGTAAGTCCAG
CAATTACAGCAGCCAATTGTAAGGCCCAAGATGCTATCATTGGCT
CAATCCTCAATCTCTATTTGACCGAGTTGACAACCATCTTCCACA
ATCAAATTACAAACCCTGCATTGAGTCCCATTACAATTCAAGCTT
TAAGGATCCTACTGGGGAGTACCTTGCCGACTGTGGTCGAAAAAT
CTTTCAATACCCAGATAAGTGCAGCTGAGCTTCTCTCATCAGGGT
TATTGACAGGCCAGATTGTGGGATTAGATTTGACCTATATGCAGA
TGGTCATAAAAATTGAGCTGCCAACTTTAACTGTACAACCTGCAA
CCCAGATCATAGATCTGGCCACCATTTCTGCATTCATTAACAATC
AAGAAGTCATGGCCCAATTACCAACACGTGTTATGGTGACTGGCA
GCTTGATCCAAGCCTATCCCGCATCGCAATGCACCATTACACCCA
ACACTGTGTACTGTAGGTATAATGATGCCCAAGTACTCTCAGATG
ATACTATGGCTTGCCTCCAAGGTAACTTGACAAGATGCACCTTCT
CTCCAGTGGTTGGGAGCTTTCTCACTCGATTCGTGCTGTTCGATG
GAATAGTTTATGCAAATTGCAGGTCGATGTTGTGCAAGTGCATGC
AACCTGCTGCTGTGATCCTACAGCCGAGTTCATCCCCTGTAACTG
TCATTGACATGTACAAATGTGTGAGTCTGCAGCTTGACAATCTCA
GATTCACCATCACTCAATTGGCCAATGTAACCTACAATAGCACCA
TCAAGCTTGAATCATCCCAGATCTTGTCTATTGATCCGTTGGATA
TATCCCAGAATCTAGCTGCGGTGAATAAGAGTCTAAGTGATGCAC
TACAACACTTAGCACAAAGTGACACATATCTTTCTGCAATCACAT
CAGCTACGACTACAAGTGTATTATCCATAATAGCAATCTGTCTTG
GATCGTTAGGTTTAATATTAATAATCTTGCTCAGTGTAGTTGTGT
GGAAGTTATTGACCATTGTCGTTGCTAATCGAAATAGAATGGAGA
ATTTTGTTTATCATAAATAAGCATTCCACCACTCACGATCTGATC
TCAGTGAGAAAAATCAACCTGCAACTCTTGGAACAAGATAAGACA
GTCATCCATTAGTAATTTTTAAGAAAAAAACGATAGGACCGAACC
TAGTATTGAAAGAACCGTCTCGGTCAATCTAGGTAATCGAGCTGA
TACCGTCTCGGAAAGCTCAAATCGCGCGCCACCatgtcagataac
ggaccacagaaccagaggaacgcacccaggattactttcggagga
ccaagcgatagcaccgggagcaaccagaatggagagcggagcgga
gcaagatccaagcagagacggccccagggcctgccaaacaatacc
gcatcctggttcaccgccctgacacagcacggcaaggaggacctg
aagtttccaaggggacagggagtgcctatcaacaccaatagctcc
cctgacgatcagatcggctactataggagggcaacaaggagaatc
aggggaggcgacggcaagatgaaggatctgagcccacgctggtac
ttctactatctgggaaccggacctgaggcaggcctgccatatggc
gccaacaaggacggaatcatctgggtggcaaccgagggcgccctg
aacacaccaaaggatcacatcggcacaagaaatcccgccaacaat
gcagcaatcgtgctgcagctgccacagggaaccacactgcccaag
ggcttttacgcagagggctctcggggaggcagccaggcatctagc
agatcctctagccggagcagaaactcctctaggaattccacccca
ggaagctccaggggcacatcccctgcccgcatggcaggaaacgga
ggcgacgccgccctggccctgctgctgctggatcgcctgaatcag
ctggagtccaagatgtctggcaagggacagcagcagcagggacag
accgtgacaaagaagtccgccgccgaggcctctaagaagccaagg
cagaagcgcaccgccacaaaggcctacaacgtgacccaggccttc
ggcaggcgcggaccagagcagacacagggcaattttggcgaccag
gagctgatcaggcagggaaccgattataagcactggcctcagatc
gcccagttogccccatctgccagcgccttctttggcatgtctaga
atcggcatggaggtgacccccagcggcacatggctgacctacaca
ggcgccatcaagctggacgataaggaccctaacttcaaggatcag
gtcatcctgctgaacaagcacatcgacgcctataagacctttccc
cctacagagcccaagaaggacaagaagaagaaggccgatgagaca
caggccctgcctcagaggcagaagaagcagcagaccgtgacactg
ctgccagccgccgatctggacgatttctccaaacagctgcagcag
agcatgtccagtgccgactccacccaggcttgaCGTACGACCTGC
TATAGGCTATCCACTGCATCATCTCTCCTGCCATACTTCCTACTC
ACATCATATCTATTTTAAAGAAAAAATAGGCCCGAACACTAATCG
TGCCGGCAGTGCCACTGCACACACAACACTACACATACAATACAC
TACAATGGTTGCAGAAGATGCCCCTGTTAGGGCCACTTGCCGAGT
ATTATTTCGAACAACAACTTTAATCTTTCTATGCACACTACTAGC
ATTAAGCATCTCTATCCTTTATGAGAGTTTAATAACCCAAAAGCA
AATCATGAGCCAAGCAGGCTCAACTGGATCTAATTCTGGATTAGG
AAGTATCACTGATCTTCTTAATAATATTCTCTCTGTCGCAAATCA
GATTATATATAACTCTGCAGTCGCTCTACCTCTACAATTGGACAC
TCTTGAATCAACACTCCTTACAGCCATTAAGTCTCTTCAAACCAG
TGACAAGCTAGAACAGAACTGCTCGTGGAGTGCTGCACTGATTAA
TGATAATAGATACATTAATGGCATCAATCAGTTCTATTTTTCAAT
TGCTGAGGGTCGCAATCTGACACTTGGCCCACTTCTTAATATGCC
TAGTTTCATTCCAACTGCCACGACACCAGAGGGCTGCACCAGGAT
CCCATCATTCTCGCTCACTAAGACACACTGGTGTTATACACACAA
TGTTATCCTGAATGGATGCCAGGATCATGTATCCTCAAATCAATT
TGTTTCTATGGGAATCATTGAACCCACTTCTGCCGGGTTTCCATT
CTTTCGAACCCTAAAGACTCTATATCTCAGCGATGGGGTCAATCG
TAAGAGCTGCTCTATCAGTACAGTTCCGGGGGGTTGTATGATGTA
CTGTTTTGTTTCTACTCAACCAGAGAGGGATGACTACTTTTCTGC
CGCTCCTCCAGAACAACGAATTATTATAATGTACTATAATGATAC
AATCGTGGAGCGCATAATTAATCCACCCGGGGTACTAGATGTATG
GGCAACATTGAACCCAGGAACAGGAAGCGGGGTATATTATTTAGG
TTGGGTGCTCTTTCCAATATATGGCGGCGTGATTAAAGGTACGAG
TTTATGGAATAATCAAGCAAATAAATACTTTATCCCCCAGATGGT
TGCTGCTCTCTGCTCACAAAACCAGGCAACTCAAGTCCAAAATGC
TAAGTCATCATACTATAGCAGCTGGTTTGGCAATCGAATGATTCA
GTCTGGGATCCTGGCATGTCCTCTTCGACAGGATCTAACCAATGA
GTGTTTAGTTCTGCCCTTTTCTAATGATCAGGTGCTTATGGGTGC
TGAAGGGAGATTATACATGTATGGTGACTCGGTGTATTACTATCA
AAGAAGCAATAGTTGGTGGCCTATGACCATGCTGTATAAGGTAAC
CATAACATTCACTAATGGTCAGCCATCTGCTATATCAGCTCAGAA
TGTGCCCACACAGCAGGTCCCTAGACCTGGGACAGGAGACTGCTC
TGCAACCAATAGATGTCCCGGTTTTTGCTTGACAGGAGTGTATGC
CGATGCCTGGTTACTGACCAACCCTTCGTCTACCAGTACATTTGG
ATCAGAAGCAACCTTCACTGGTTCTTATCTCAACACAGCAACTCA
GCGTATCAATCCGACGATGTATATCGCGAACAACACACAGATCAT
AAGCTCACAGCAATTTGGATCAAGCGGTCAAGAAGCAGCATATGG
CCACACAACTTGTTTTAGGGACACAGGCTCTGTTATGGTATACTG
TATCTATATTATTGAATTGTCCTCATCTCTCTTAGGACAATTTCA
GATTGTCCCATTTATCCGTCAGGTGACACTATCCTAAAGGCAGAA
GCCTTCAGGTCTGACCCAGCCAATCAAAGCATTATACCAGACCAT
GGCCTACCATCGGCTTTAAAGAAAAAAATAGGCCCGGACGGGTTA
GCAACAAGCGGCGGCCGCCatgttcgtcttcctggtcctgctgcc
tctggtctcctcacagtgcgtcaatctgacaactcggactcagct
gccacctgcttatactaatagcttcaccagaggcgtgtactatcc
tgacaaggtgtttagaagctccgtgctgcactctacacaggatct
gtttctgccattctttagcaacgtgacctggttccacgccatcca
cgtgagcggcaccaatggcacaaagcggttcgacaatcccgtgct
gccttttaacgatggcgtgtacttcgcctctaccgagaagagcaa
catcatcagaggctggatctttggcaccacactggactccaagac
acagtctctgctgatcgtgaacaatgccaccaacgtggtcatcaa
ggtgtgcgagttccagttttgtaatgatcccttcctgggcgtgta
ctatcacaagaacaataagagctggatggagtccgagtttagagt
gtattctagcgccaacaactgcacatttgagtacgtgagccagcc
tttcctgatggacctggagggcaagcagggcaatttcaagaacct
gagggagttcgtgtttaagaatatcgacggctacttcaaaatcta
ctctaagcacacccccatcaacctggtgcgcgacctgcctcaggg
cttcagcgccctggagcccctggtggatctgcctatcggcatcaa
catcacccggtttcagacactgctggccctgcacagaagctacct
gacacccggcgactcctctagcggatggaccgccggcgctgccgc
ctactatgtgggctacctccagccccggaccttcctgctgaagta
caacgagaatggcaccatcacagacgcagtggattgcgccctgga
ccccctgagcgagacaaagtgtacactgaagtcctttaccgtgga
gaagggcatctatcagacatccaatttcagggtgcagccaaccga
gtctatcgtgcgctttcctaatatcacaaacctgtgcccatttgg
cgaggtgttcaacgcaacccgcttcgccagcgtgtacgcctggaa
taggaagcggatcagcaactgcgtggccgactatagcgtgctgta
caactccgcctctttcagcacctttaagtgctatggcgtgtcccc
cacaaagctgaatgacctgtgctttaccaacgtctacgccgattc
tttcgtgatcaggggcgacgaggtgcgccagatcgcccccggcca
gacaggcaagatcgcagactacaattataagctgccagacgattt
caccggctgcgtgatcgcctggaacagcaacaatctggattccaa
agtgggcggcaactacaattatctgtaccggctgtttagaaagag
caatctgaagcccttcgagagggacatctctacagaaatctacca
ggccggcagcaccccttgcaatggcgtggagggctttaactgtta
tttcccactccagtcctacggcttccagcccacaaacggcgtggg
ctatcagccttaccgcgtggtggtgctgagctttgagctgctgca
cgccccagcaacagtgtgcggccccaagaagtccaccaatctggt
gaagaacaagtgcgtgaacttcaacttcaacggcctgaccggcac
aggcgtgctgaccgagtccaacaagaagttcctgccatttcagca
gttcggcagggacatcgcagataccacagacgccgtgcgcgaccc
acagaccctggagatcctggacatcacaccctgctctttcggcgg
cgtgagcgtgatcacacccggcaccaatacaagcaaccaggtggc
cgtgctgtatcaggacgtgaattgtaccgaggtgcccgtggctat
ccacgccgatcagctgaccccaacatggcgggtgtacagcaccgg
ctccaacgtcttccagacaagagccggatgcctgatcggagcaga
gcacgtgaacaattcctatgagtgcgacatcccaatcggcgccgg
catctgtgcctcttaccagacccagacaaactctcccagaagagc
ccggagcgtggcctcccagtctatcatcgcctataccatgtccct
gggcgccgagaacagcgtggcctactctaacaatagcatogccat
cccaaccaacttcacaatctctgtgaccacagagatcctgcccgt
gtccatgaccaagacatctgtggactgcacaatgtatatctgtgg
cgattctaccgagtgcagcaacctgctgctccagtacggcagctt
ttgtacccagctgaatagagccctgacaggcatcgccgtggagca
ggataagaacacacaggaggtgttcgcccaggtgaagcaaatcta
caagaccccccctatcaaggactttggcggcttcaatttttccca
gatcctgcctgatccatccaagccttctaagcggagctttatcga
ggacctgctgttcaacaaggtgaccctggccgatgccggcttcat
caagcagtatggcgattgcctgggcgacatcgcagccagggacct
gatctgcgcccagaagtttaatggcctgaccgtgctgccacccct
gctgacagatgagatgatcgcacagtacacaagcgccctgctggc
cggcaccatcacatccggatggaccttcggcgcaggagccgccct
ccagatcccctttgccatgcagatggcctataggttcaacggcat
cggcgtgacccagaatgtgctgtacgagaaccagaagctgatcgc
caatcagtttaactccgccatcggcaagatccaggacagcctgtc
ctctacagccagcgccctgggcaagctccaggatgtggtgaatca
gaacgcccaggccctgaataccctggtgaagcagctgagcagcaa
cttcggcgccatctctagcgtgctgaatgacatcctgagccggct
ggacaaggtggaggcagaggtgcagatcgaccggctgatcaccgg
ccggctccagagcctccagacctatgtgacacagcagctgatcag
ggccgccgagatcagggccagcgccaatctggcagcaaccaagat
gtccgagtgcgtgctgggccagtctaagagagtggacttttgtgg
caagggctatcacctgatgtccttccctcagtctgccccacacgg
cgtggtgtttctgcacgtgacctacgtgcccgcccaggagaagaa
cttcaccacagcccctgccatctgccacgatggcaaggcccactt
tccaagggagggcgtgttcgtgtccaacggcacccactggtttgt
gacacagcgcaatttctacgagccccagatcatcaccacagacaa
caccttcgtgagcggcaactgtgacgtggtcatcggcatcgtgaa
caataccgtgtatgatccactccagcccgagctggacagctttaa
ggaggagctggataagtatttcaagaatcacacctcccctgacgt
ggatctgggcgacatcagcggcatcaatgcctccgtggtgaacat
ccagaaggagatcgaccgcctgaacgaggtggctaagaatctgaa
cgagagcctgatcgacctccaggagctgggcaagtatgagcagta
catcaagtggccctggtacatctggctgggcttcatcgccggcct
gatcgccatcgtgatggtgaccatcatgctgtgctgtatgacatc
ctgctgttcttgcctgaagggctgctgtagctgtggctcctgctg
taagttattgaccattgtcgttgctaatcgaaatagaatggagaa
ttttgtttatcataaatgaAGTCGACTCATGGAATGCATACCAAA
CATTATTGACACTAATGACACACAAAATTGGTTTTAAGAAAAACC
AAGAGAACAATAGGCCAGAATGGCTGGGTCTCGGGAGATATTACT
CCCTGAAGTCCATCTCAATTCACCAATTGTAAAGCATAAGCTATA
CTATTACATTCTACTTGGAAACCTCCCAAATGAGATCGACCTTGA
CGATTTAGGTCCATTACATAATCAAAATTGGAATCAGATAGCACA
TGAAGAGTCTAACTTAGCTCAACGCTTGGTAAATGTAAGAAATTT
TCTAATTACCCACATCCCTGATCTTAGAAAGGGCCATTGGCAAGA
GTATGTCAATGTAATACTGTGGCCGCGAATTCTTCCCTTGATCCC
GGATTTTAAAATCAATGACCAATTGCCTCTGCTCAAAAATTGGGA
CAAGTTAGTTAAAGAATCATGTTCAGTAATCAATGCAGGTACTTC
CCAGTGCATTCAGAATCTCAGCTATGGACTGACAGGTCGTGGGAA
CCTCTTTACACGATCACGTGAACTCTCTGGTGACCGCAGGGATAT
TGATCTTAAGACAGTTGTGGCAGCATGGCATGACTCAGACTGGAA
AAGAATAAGTGATTTTTGGATTATGATCAAATTCCAGATGAGACA
ATTAATTGTTAGGCAAACAGATCATAATGATTCTGATTTAATCAC
GTATATCGAAAATAGAGAAGGCATAATCATCATAACCCCTGAACT
GGTAGCATTATTTAACACTGAGAATCATACACTAACATACATGAC
CTTTGAAATTGTACTGATGGTTTCAGATATGTACGAAGGTCGTCA
CAACATTTTATCACTATGCACAGTTAGCACTTACCTGAATCCTCT
GAAGAAAAGAATAACATATTTATTGAGCCTTGTAGATAACTTAGC
TTTTCAGATAGGTGATGCTGTATATAACATAATTGCTTTGCTAGA
ATCCTTTGTATATGCACAGTTGCAAATGTCAGATCCCATCCCAGA
ACTCAGAGGACAATTCCATGCATTCGTATGTTCTGAGATTCTTGA
TGCACTAAGAGGAACTAATAGTTTCACCCAGGATGAATTAAGAAC
TGTGACAACTAATTTGATATCCCCATTCCAAGATCTGACCCCAGA
TCTTACGGCTGAATTGCTCTGTATAATGAGGCTTTGGGGACACCC
CATGCTCACTGCCAGTCAAGCTGCAGGAAAGGTACGCGAGTCTAT
GTGTGCTGGAAAAGTATTAGACTTTCCCACCATTATGAAAACACT
AGCCTTTTTCCATACTATTCTGATCAATGGATACAGGAGGAAGCA
TCATGGAGTATGGCCACCCTTAAACTTACCGGGTAATGCTTCAAA
GGGTCTCACGGAACTTATGAATGACAATACTGAAATAAGCTATGA
ATTCACACTTAAGCATTGGAAGGAAGTCTCTCTTATAAAATTCAA
GAAATGTTTTGATGCAGACGCAGGTGAGGAACTCAGTATATTTAT
GAAAGATAAGGCAATTAGTGCCCCAAAACAAGACTGGATGAGTGT
GTTTAGAAGAAGCCTAATCAAACAGCGCCATCAGCATCATCAGGT
CCCCCTACCAAATCCATTCAATCGACGGCTGTTGCTAAACTTTCT
CGGAGATGACAAATTCGACCCGAATGTGGAGCTACAGTATGTAAC
ATCAGGTGAGTATCTACATGATGACACGTTTTGTGCATCATATTC
ACTAAAAGAGAAGGAAATTAAACCTGATGGTCGAATTTTTGCAAA
GTTGACTAAGAGAATGAGATCATGTCAAGTTATAGCAGAATCTCT
TTTAGCGAACCATGCTGGGAAGTTAATGAAAGAGAATGGTGTTGT
GATGAATCAGCTATCATTAACAAAATCACTATTAACAATGAGTCA
GATTGGAATAATATCCGAGAAAGCTAGAAAGTCAACTCGAGATAA
CATAAATCAACCTGGTTTCCAGAATATCCAGAGAAATAAATCACA
TCACTCCAAGCAAGTCAATCAGCGAGATCCAAGTGATGACTTTGA
ATTGGCAGCATCTTTTTTAACTACTGATCTCAAAAAATATTGTTT
ACAATGGAGGTACCAGACAATTATCCCATTTGCTCAATCATTAAA
CAGAATGTATGGTTATCCTCATCTCTTTGAGTGGATTCACTTACG
GCTAATGCGTAGTACACTTTACGTGGGGGATCCCTTCAACCCACC
AGCAGATACCAGTCAATTTGATCTAGATAAAGTAATTAATGGAGA
TATCTTCATTGTATCACCCAGAGGTGGAATTGAAGGGCTGTGTCA
AAAGGCTTGGACAATGATATCTATCGCTGTGATAATTCTATCTGC
CACAGAGTCTGGCACACGAGTAATGAGTATGGTGCAGGGAGATAA
TCAAGCAATTGCTGTCACCACACGAGTACCAAGGAGCCTGCCGAC
TCTTGAGAAAAAGACTATTGCTTTTAGATCTTGTAATCTATTCTT
TGAGAGGTTAAAATGTAATAATTTTGGATTAGGTCACCATTTGAA
AGAACAAGAGACTATCATTAGTTCTCACTTCTTTGTTTATAGCAA
GAGAATATTCTATCAGGGGAGGATTCTAACGCAAGCCTTAAAAAA
TGCTAGTAAGCTCTGCTTGACAGCTGATGTCCTAGGAGAATGCAC
CCAATCATCATGTTCTAATCTTGCAACTACTGTCATGAGGTTAAC
TGAGAATGGTGTTGAAAAAGATATCTGTTTCTACTTGAATATCTA
TATGACCATCAAACAGCTCTCCTATGATATCATCTTCCCTCAAGT
GTCAATTCCTGGAGATCAGATCACATTAGAATACATAAATAATCC
ACACCTGGTATCACGATTGGCTCTTTTGCCATCCCAGTTAGGAGG
TCTAAACTACCTGTCATGCAGTAGGCTGTTCAATCGAAACATAGG
CGACCCGGTGGTTTCCGCAGTTGCAGATCTTAAGAGATTAATTAA
ATCAGGATGTATGGATTACTGGATCCTTTATAACTTATTAGGGAG
AAAACCGGGAAACGGCTCATGGGCTACTTTAGCAGCTGACCCGTA
CTCAATCAATATAGAGTATCAATACCCTCCAACTACAGCTCTTAA
GAGGCACACCCAACAAGCTCTGATGGAACTCAGTACGAATCCAAT
GTTACGTGGCATATTCTCTGACAATGCACAGGCAGAAGAAAATAA
CCTTGCTAGGTTTCTCCTGGATAGGGAGGTGATCTTTCCGCGTGT
AGCTCACATCATCATTGAGCAAACCAGTGTCGGGAGGAGAAAACA
GATTCAAGGATATTTGGATTCAACTAGATCGATAATGAGGAAATC
ACTAGAAATTAAGCCCTTATCCAATAGGAAGCTTAATGAAATACT
GGATTACAACATCAATTACCTAGCTTACAATTTGGCATTACTCAA
GAATGCTATTGAACCTCCGACTTATTTGAAGGCAATGACACTTGA
AACATGTAGCATCGACATTGCAAGGAACCTCCGGAAGCTCTCCTG
GGCCCCACTCTTGGGTGGGAGAAATCTTGAAGGATTAGAGACGCC
AGATCCCATTGAAATTACTGCAGGAGCATTAATTGTTGGATCGGG
CTACTGTGAACAGTGTGCTGCAGGAGACAATCGATTCACATGGTT
TTTCTTGCCATCTGGTATCGAGATAGGAGGGGATCCCCGTGATAA
TCCTCCTATCCGTGTACCGTACATTGGCTCCAGGACTGATGAGAG
GAGGGTAGCCTCAATGGCATACATCAGGGGTGCCTCGAGTAGCTT
AAAAGCAGTTCTTAGACTGGCGGGAGTGTACATCTGGGCATTCGG
AGATACTCTGGAGAATTGGATAGATGCACTGGATTTGTCTCACAC
TAGAGTTAACATCACACTTGAACAGCTGCAATCCCTCACCCCACT
TCCAACCTCTGCCAATCTAACCCATCGGTTGGATGATGGCACAAC
TACCCTAAAGTTTACTCCTGCGAGCTCTTACACCTTTTCAAGTTT
CACTCATATATCAAATGATGAGCAATACCTGACAATTAATGACAA
AACTGCAGATTCAAATATAATCTACCAACAGTTAATGATCACTGG
ACTCGGAATCTTAGAAACATGGAATAATCCCCCAATCAATAGAAC
ATTCGAAGAATCTACCCTACATTTGCACACTGGTGCATCATGTTG
TGTCCGACCTGTGGACTCCTGCATTCTCTCAGAAGCATTAACAGT
CAAGCCACATATTACAGTACCGTACAGCAATAAATTTGTATTTGA
TGAGGACCCGCTATCTGAATATGAAACTGCAAAACTGGAATCGTT
ATCATTCCAAGCCCAATTAGGCAACATTGATGCTGTAGATATGAC
AGGTAAATTAACATTATTGTCCCAATTCACTGCAAGGCAGATTAT
CAATGCAATCACTGGACTCGATGAGTCTGTCTCTCTTACTAATGA
TGCCATTGTTGCATCAGACTATGTCTCCAATTGGATTAGTGAATG
CATGTATACCAAATTAGATGAATTATTTATGTATTGTGGGTGGGA
ACTACTATTGGAACTATCCTATCAAATGTATTATCTGAGGGTAGT
TGGGTGGAGTAATATAGTGGATTATTCTTACATGATCTTGAGAAG
AATCCCGGGTGCAGCATTAAACAATCTGGCATCTACATTAAGTCA
TCCAAAACTTTTCCGACGAGCTATCAACCTAGATATAGTTGCCCC
CTTAAATGCTCCTCATTTTGCATCTCTGGACTACATCAAGATGAG
TGTGGATGCAATACTCTGGGGCTGTAAAAGAGTCATCAATGTGCT
CTCCAATGGAGGGGACTTAGAATTAGTTGTGACATCTGAAGATAG
CCTTATTCTCAGTGACCGATCCATGAATCTCATTGCAAGGAAATT
AACTTTATTATCACTGATTCACCATAATGGTTTGGAACTACCAAA
GATTAAGGGGTTCTCTCCTGATGAGAAGTGTTTCGCTTTGACAGA
ATTTTTGAGGAAAGTGGTGAACTCAGGGTTGAGTTCAATAGAGAA
CCTATCAAATTTTATGTACAATGTGGAGAACCCACGGCTTGCAGC
ATTCGCCAGCAACAATTACTACCTGACCAGAAAATTATTGAATTC
AATACGAGATACTGAGTCGGGTCAAGTAGCAGTCACCTCATATTA
TGAATCATTAGAATATATTGATAGTCTTAAGCTAACCCCACATGT
GCCTGGCACCTCATGCATTGAGGATGATAGTCTATGTACAAATGA
TTACATAATCTGGATCATAGAGTCTAATGCAAACTTGGAGAAGTA
TCCAATTCCAAATAGCCCTGAGGATGATTCCAATTTCCATAACTT
TAAGTTGAATGCTCCATCGCACCATACCTTACGCCCATTAGGGTT
GTCATCAACTGCTTGGTATAAGGGTATAAGCTGCTGCAGGTACCT
TGAGCGATTAAAGCTACCACAAGGTGATCATTTATATATTGCAGA
AGGTAGTGGTGCCAGTATGACAATCATAGAATACCTATTCCCAGG
AAGAAAGATATATTACAATTCTTTATTTAGTAGTGGTGACAATCC
CCCACAAAGAAATTATGCACCAATGCCTACTCAGTTCATTGAGAG
TGTCCCATACAAGCTCTGGCAAGCACACACAGATCAATATCCCGA
GATTTTTGAGGACTTCATCCCTCTATGGAACGGAAACGCCGCCAT
GACTGACATAGGAATGACAGCTTGTGTAGAATTCATCATCAATCG
AGTCGGCCCAAGGACTTGCAGTTTAGTACATGTAGATTTGGAATC
AAGTGCAAGCTTAAATCAACAATGCCTGTCAAAGCCGATAATTAA
TGCTATCATCACTGCTACAACTGTTTTGTGCCCTCATGGGGTGCT
TATTCTGAAATATAGTTGGTTGCCATTTACTAGATTTAGTACTTT
GATCACTTTCTTATGGTGCTACTTTGAGAGAATCACTGTTCTTAG
GAGCACATATTCTGATCCAGCTAATCATGAGGTTTATTTAATTTG
TATCCTTGCCAACAACTTTGCATTCCAGACTGTCTCGCAGGCAAC
AGGAATGGCGATGACTTTAACTGATCAAGGGTTTACTTTGATATC
ACCTGAAAGAATAAATCAGTATTGGGATGGTCACTTGAAGCAAGA
ACGTATCGTAGCAGAAGCAATTGATAAGGTGGTTCTAGGAGAAAA
TGCTCTATTTAATTCGAGTGATAATGAATTAATTCTCAAATGTGG
AGGGACACCAAATGCACGGAATCTCATCGATATCGAGCCAGTCGC
AACTTTCATAGAATTTGAACAATTGATCTGCACAATGTTGACAAC
CCACTTGAAGGAAATAATTGATATAACAAGGTCTGGAACCCAGGA
TTATGAAAGTTTATTACTCACTCCTTACAATTTAGGTCTTCTTGG
TAAAATCAGTACGATAGTGAGATTATTAACAGAAAGGATTCTAAA
TCATACTATCAGGAATTGGTTGATCCTCCCACCTTCGCTCCGGAT
GATCGTGAAGCAGGACTTGGAATTCGGCATATTCAGGATTACTTC
CATCCTCAATTCTGATCGGTTCCTGAAGCTTTCTCCAAATAGGAA
ATACTTGATTGCACAATTAACTGCAGGCTACATTAGGAAATTGAT
TGAGGGGGATTGCAATATCGATCTAACCAGACCTATCCAAAAGCA
AATCTGGAAAGCATTAGGTTGTGTAGTCTATTGTCACGATCCAAT
GGATCAAAGGGAGTCAACAGAGTTTATTGATATAAATATTAATGA
AGAAATAGACCGCGGGATCGATGGCGAGGAAATCTAAACATATCA
AGAATCAGAATTAGTTTAAGAAAAAAGAAGAGGATTAATCTTGGT
TTTCCCCTTGGTGGGTCGGCATGGCATCTCCACCTCCTCGCGGTC
CGACCTGGGCATCCGAAGGAGGACGCACGTCCACTCGGATGGCTA
AGGGAGCGGCCGGGGATCCGGCTGCTAACAAAGCCCGAAAGGAAG
CTGAGTTGGCTGCTGCCACCGCTGAGCAATAACTAGCATAACCCC
TTGGGGCCTCTAAACGGGTCTTGAGGGGTTTTTTGCTGAAAGGAG
GAACTATATCCGGATCATGTGAGCAAAAGGCCAGCAAAAGGCCAG
GAACCGTAAAAAGGCCGCGTTGCTGGCGTTTTTCCATAGGCTCCG
CCCCCCTGACGAGCATCACAAAAATCGACGCTCAAGTCAGAGGTG
GCGAAACCCGACAGGACTATAAAGATACCAGGCGTTTCCCCCTGG
AAGCTCCCTCGTGCGCTCTCCTGTTCCGACCCTGCCGCTTACCGG
ATACCTGTCCGCCTTTCTCCCTTCGGGAAGCGTGGCGCTTTCTCA
TAGCTCACGCTGTAGGTATCTCAGTTCGGTGTAGGTCGTTCGCTC
CAAGCTGGGCTGTGTGCACGAACCCCCCGTTCAGCCCGACCGCTG
CGCCTTATCCGGTAACTATCGTCTTGAGTCCAACCCGGTAAGACA
CGACTTATCGCCACTGGCAGCAGCCACTGGTAACAGGATTAGCAG
AGCGAGGTATGTAGGCGGTGCTACAGAGTTCTTGAAGTGGTGGCC
TAACTACGGCTACACTAGAAGAACAGTATTTGGTATCTGCGCTCT
GCTGAAGCCAGTTACCTTCGGAAAAAGAGTTGGTAGCTCTTGATC
CGGCAAACAAACCACCGCTGGTAGCGGTGGTTTTTTTGTTTGCAA
GCAGCAGATTACGCGCAGAAAAAAAGGATCTCAAGAAGATCCTTT
GATCTTTTCTACGGGGTCTGACGCTCAGTGGAACGAAAACTCACG
TTAAGGGATTTTGGTCATGAGATTATCAAAAAGGATCTTCACCTA
GATCCTTTTAAATTAAAAATGAAGTTTTAAATCAATCTAAAGTAT
ATATGAGTAAACTTGGTCTGACAGTTACCAATGCTTAATCAGTGA
GGCACCTATCTCAGCGATCTGTCTATTTCGTTCATCCATAGTTGC
CTGACTCCCCGTCGTGTAGATAACTACGATACGGGAGGGCTTACC
ATCTGGCCCCAGTGCTGCAATGATACCGCGAGACCCACGCTCACC
GGCTCCAGATTTATCAGCAATAAACCAGCCAGCCGGAAGGGCCGA
GCGCAGAAGTGGTCCTGCAACTTTATCCGCCTCCATCCAGTCTAT
TAATTGTTGCCGGGAAGCTAGAGTAAGTAGTTCGCCAGTTAATAG
TTTGCGCAACGTTGTTGCCATTGCTACAGGCATCGTGGTGTCACG
CTCGTCGTTTGGTATGGCTTCATTCAGCTCCGGTTCCCAACGATC
AAGGCGAGTTACATGATCCCCCATGTTGTGCAAAAAAGCGGTTAG
CTCCTTCGGTCCTCCGATCGTTGTCAGAAGTAAGTTGGCCGCAGT
GTTATCACTCATGGTTATGGCAGCACTGCATAATTCTCTTACTGT
CATGCCATCCGTAAGATGCTTTTCTGTGACTGGTGAGTACTCAAC
CAAGTCATTCTGAGAATAGTGTATGCGGCGACCGAGTTGCTCTTG
CCCGGCGTCAATACGGGATAATACCGCGCCACATAGCAGAACTTT
AAAAGTGCTCATCATTGGAAAACGTTCTTCGGGGCGAAAACTCTC
AAGGATCTTACCGCTGTTGAGATCCAGTTCGATGTAACCCACTCG
TGCACCCAACTGATCTTCAGCATCTTTTACTTTCACCAGCGTTTC
TGGGTGAGCAAAAACAGGAAGGCAAAATGCCGCAAAAAAGGGAAT
AAGGGCGACACGGAAATGTTGAATACTCATACTCTTCCTTTTTCA
ATATTATTGAAGCATTTATCAGGGTTATTGTCTCATGAGCGGATA
CATATTTGAATGTATTTAGAAAAATAAACAAATAGGGGTTCCGCG
CACATTTCCCCGAAAAGT.

iv. CVL83

The nucleic acid sequence for CVL83 is:

(SEQ ID NO: 6)
GCCACCTGACGTCTAAGAAACCATTATTATCATGACATTAACCTATAAAA
ATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAA
GCGGATGCCGGGAGCAGACAAGCCCGTCAGGGCGCGTCAGCGGGTGTTG
GCGGGTGTCGGGGCTGGCTTAACTATGCGGCATCAGAGCAGATTGTACTG
AGAGTGCACCATATATGCGGTGTGAAATACCGCACAGATGCGTAAGGAGA
AAATACCGCATCAGGCTAATACGACTCACTATAGGGACCAAGGGGAAAAT
GAAGTGGTGACTCAAATCATCGAAGACCCTCGAGATTACATAGGTCCGGA
ACCTATGGCCTTCGTGACCGACCTCGAGTCAGAGTAGTTCAATAAGGACC
TATCAAGTTTGGGCAATTTTTCGTCCCCGACACAAAAATGTCATCCGTGCT
TAAAGCATATGAGCGATTCACGCTCACTCAAGAACTGCAAGATCAGAGTG
AGGAAGGTACAATCCCACCTACAACACTAAAACCGGTAATCAGGGTATTT
ATACTAACCTCTAATAACCCAGAGCTAAGATCCCGGCTTCTTCTATTCTGC
CTACGGATTGTTCTCAGTAATGGTGCAAGGGATTCCCATCGCTTTGGAGCA
TTACTCACAATGTTTTCGCTACCATCAGCCACAATGCTCAATCATGTCAAA
TTAGCTGACCAGTCACCAGAAGCTGATATCGAAAGGGTAGAGATCGATGG
CTTTGAGGAGGGATCATTCCGCTTAATCCCCAATGCTCGTTCAGGTATGAG
CCGTGGAGAGATCAATGCCTATGCTGCACTTGCAGAAGATCTACCTGACA
CACTAAACCATGCAACACCTTTCGTTGATTCCGAAGTCGAGGGAACTGCA
TGGGATGAGATTGAGACTTTCTTAGATATGTGTTACAGTGTCCTAATGCAG
GCATGGATAGTGACTTGCAAGTGCATGACTGCGCCAGACCAACCTGCTGC
TTCTATTGAGAAACGCCTGCAAAAATATCGTCAGCAAGGCAGGATCAACC
CGAGATATCTCCTGCAACCGGAGGCTCGACGAATAATCCAGAATGTAATC
CGGAAGGGAATGGTGGTCAGACATTTCCTCACCTTTGAACTGCAGCTTGC
CCGAGCACAAAGCCTTGTATCAAATAGGTATTATGCTATGGTAGGGGATG
TTGGAAAGTATATAGAGAATTGTGGAATGGGAGGCTTCTTTTTGACACTA
AAATATGCATTAGGAACTAGATGGCCCACACTTGCTTTAGCTGCATTTTCA
GGAGAGCTAACAAAGCTAAAGTCCCTCATGGCATTATACCAGACCCTTGG
TGAGCAGGCCCGATATTTGGCCCTATTGGAGTCACCACATTTGATGGATTT
TGCTGCAGCAAACTACCCACTGCTATATAGCTATGCTATGGGAATAGGCT
ATGTGTTAGATGTCAACATGAGGAACTACGCTTTCTCCAGATCATACATGA
ACAAGACATATTTCCAATTGGGAATGGAAACTGCAAGAAAACAACAGGG
TGCAGTTGACATGAGGATGGCAGAAGATCTCGGTCTAACTCAAGCCGAAC
GCACCGAGATGGCAAATACACTTGCCAAATTGACCACAGCAAATCGAGGG
GCAGACACCAGGGGAGGAGTCAACCCGTTCTCATCTGTCACTGGGACAAC
TCAGGTGCCCGCTGCAGCAACAGGTGACACACTCGAGAGTTACATGGCAG
CGGATCGACTGAGGCAGAGATATGCTGATGCAGGCACCCATGATGATGAG
ATGCCACCATTGGAAGAGGAGGAAGAGGACGACACATCTGCAGGTCCAC
GCACTGGACCAACTCTTGAACAAGTGGCCTTGGACATCCAGAACGCAGCA
GTTGGAGCTCCCATCCATACAGATGACCTGAATGCCGCACTGGGTGATCTT
GACATCTAGACAATTCAGATCCCAATCTAAAATTGACATACCTAATTGATT
AGTTAGATGGAACTACAGTGGATTCCATAAGGTTCCTGCCTACCATCGGCT
TTAAAGAAAAAAATAGGCCCGGACGGGTTAGCAACAAGCGACTGCCGGT
GCCAACAGCGCAATCCACAATCTACAATGGATCCCACTGATCTGAGCTTC
TCCCCAGATGAGATCAATAAGCTCATAGAGACAGGCCTGAATACTGTAGA
GTATTTTACTTCCCAACAAGTCACAGGAACATCCTCTCTTGGAAAGAATAC
AATACCACCAGGGGTCACAGGACTACTAACCAATGCTGCAGAGGCAAAG
ATCCAAGAGTCAACTAACCATCAGAAGGGCTCAGTTGGTGGGGGTGCAAA
ACCAAAGAAACCGCGACCAAAAATTGCCATTGTGCCAGCAGATGACAAA
ACAGTGCCCGGAAAGCCGATCCCAAACCCTCTATTAGGTCTGGACTCCAC
CCCGAGCACCCAAACTGTGCTTGATCTAAGTGGGAAAACATTACCATCAG
GATCCTATAAGGGGGTTAAGCTTGCGAAATTTGGAAAAGAAAATCTGATG
ACACGGTTCATCGAGGAACCCAGAGAGAATCCTATCGCAACCAGTTtCCCC
ATCGATTTTAAGAGGGGCAGGGATACCGGCGGGTTCCATAGAAGGGAGTA
CTCAATCGGATGGGTGGGAGATGAAGTCAAGGTCACTGAGTGGTGCAATC
CATCCTGTTCTCCAATCACCGCTGCAGCAAGGCGATTTGAATGCACTTGTC
ACCAGTGTCCAGTCACTTGCTCTGAATGTGAACGAGATACTTAATACAGT
GAGAAATTTGGACTCTCGGATGAATCAACTGGAGACAAAAGTAGATCGCA
TTCTCTCATCTCAGTCTCTAATCCAGACCATCAAGAATGACATAGTTGGAC
TTAAAGCAGGGATGGCTACTTTAGAAGGAATGATTACAACTGTGAAAATC
ATGGACCCGGGAGTTCCCAGTAATGTTACTGTGGAAGATGTACGCAAGAC
ACTAAGTAACCATGCTGTTGTTGTGCCAGAATCATTCAATGATAGTTTCTT
GACTCAATCTGAAGATGTAATTTCACTTGATGAGTTGGCTCGACCAACTGC
AACAAGTGTTAAGAAGATTGTCAGGAAGGTTCCTCCTCAGAAGGATCTGA
CTGGATTGAAGATTACACTAGAGCAATTGGCAAAGGATTGCATCAGCAAA
CCGAAGATGAGGGAAGAGTATCTCCTCAAAATCAACCAGGCTTCCAGTGA
GGCTCAGCTAATTGACCTCAAGAAAGCAATCATCCGCAGTGCAATTTGAT
CAAGAAACACCCAATTACACTACACTGGTATGACACTGTACTAACCCTGA
GGGTTTTAGAAAAAACGATTAACGATAAATAAGCCCGAACACTACACACT
ACCTGAGGCAGCCATGCCATCCATCAGCATTCCCGCAGACCCCACCAATC
CACGTCAATCAATAAAAGCGTTCCCAATTGTGATCAACAGTGATGGGGGT
GAGAAAGGCCGCTTGGTTAAACAACTACGCACAACCTACTTGAATGACCT
AGATACTCATGAGCCACTGGTGACATTCATAAATACCTATGGATTCATCTA
CGAACAGGATCGGGGGAATACCATTGTCGGAGAGGATCAACTTGGGAAG
AAAAGAGAGGCTGTGACCGCTGCAATGGTTACCCTTGGATGTGGGCCTAA
TCTACCATCATTAGGGAATGTCCTGGGACAACTGAGGGAATTCCAGGTCA
CTGTTAGGAAGACATCCAGCAAAGCGGAAGAGATGGTCTTTGAAATTGTT
AAGTATCCGAGAATATTTCGGGGTCATACATTAATCCAGAAAGGACTAGT
CTGTGTCTCCGCAGAAAAATTTGTTAAGTCACCAGGGAAAATACAATCTG
GAATGGACTATCTCTTCATTCCGACATTTCTGTCAGTGACTTACTGTCCAG
CTGCAATCAAATTTCAGGTACCTGGCCCCATGTTGAAAATGAGATCAAGA
TACACTCAGAGCTTACAACTTGAACTAATGATAAGAATCCTGTGTAAGCC
CGATTCGCCACTTATGAAGGTCCATACCCCTGACAAGGAGGGAAGAGGAT
GTCTTGTATCAGTATGGCTGCATGTATGCAACATCTTCAAATCAGGAAACA
AGAATGGCAGTGAGTGGCAGGAATACTGGATGAGAAAGTGTGCTAACAT
GCAACTTGAAGTGTCGATTGCAGATATGTGGGGACCAACTATCATAATTC
ATGCCAGAGGTCACATTCCCAAAAGTGCTAAGTTGTTTTTTGGAAAGGGT
GGATGGAGCTGCCATCCACTTCACGAAGTTGTTCCAAGTGTCACTAAAAC
ACTATGGTCCGTGGGCTGTGAGATTACAAAGGCGAAGGCAATAATACAAG
AGAGTAGCATCTCTCTTCTCGTGGAGACTACTGACATCATAAGTCCAAAA
GTCAAAATTTCATCTAAGCATCGCCGCTTTGGGAAATCAAATTGGGGTCTG
TTCAAGAAAACTAAATCACTGCCTAACCTGACGGAGCTGGAATGACTGAC
CTCTAATCGAGACTACACCGCCGCAAACTATAGGTGGGTGGTACCTCAGT
GATTAATCTTGTAAGCACTGATCGTAGGCTACAACACACTAATATTATCCA
GATTAGAGAGCTTAATTAGCTCTGTATTAATAATAACACTACTATTCCAAT
AACTGGAATCACCAGCTTGATTTATCTCCAAAATGATTCAAAGAAAACAA
ATCATATTAAGACTATCCTAAGCACGAACCCATATCGTCCTTCAAATCATG
GGTACTATAATTCAATTTCTGGTGGTCTCCTGTCTATTGGCAGGAGCAGGC
AGCCTTGATCCAGCAGCCCTCATGCAAATCGGTGTCATTCCAACAAATGTC
CGGCAACTTATGTATTATACTGAAGCTTCATCAGCATTCATTGTTGTGAAG
TTAATGCCTACAATTGACTCGCCGATTAGTGGATGTAATATAACATCAATT
TCAAGCTATAATGCAACAGTGACAAAACTCCTACAGCCGATCGGTGAGAA
TTTGGAGACGATTAGGAACCAGTTGATTCCAACTCGGAGGAGACGCCGGT
TTGCAGGGGTGGTGATTGGATTAGCTGCATTAGGAGTAGCTACTGCCGCA
CAGGTCACTGCCGCAGTGGCACTAGTAAAGGCAAATGAAAATGCTGCGGC
TATACTCAATCTCAAAAATGCAATCCAAAAAACAAATGCAGCAGTTGCAG
ATGTGGTCCAGGCCACACAATCACTAGGAACGGCAGTTCAAGCAGTTCAA
GATCACATAAACAGTGTGGTAAGTCCAGCAATTACAGCAGCCAATTGTAA
GGCCCAAGATGCTATCATTGGCTCAATCCTCAATCTCTATTTGACCGAGTT
GACAACCATCTTCCACAATCAAATTACAAACCCTGCATTGAGTCCCATTAC
AATTCAAGCTTTAAGGATCCTACTGGGGAGTACCTTGCCGACTGTGGTCG
AAAAATCTTTCAATACCCAGATAAGTGCAGCTGAGCTTCTCTCATCAGGGT
TATTGACAGGCCAGATTGTGGGATTAGATTTGACCTATATGCAGATGGTC
ATAAAAATTGAGCTGCCAACTTTAACTGTACAACCTGCAACCCAGATCAT
AGATCTGGCCACCATTTCTGCATTCATTAACAATCAAGAAGTCATGGCCCA
ATTACCAACACGTGTTATGGTGACTGGCAGCTTGATCCAAGCCTATCCCGC
ATCGCAATGCACCATTACACCCAACACTGTGTACTGTAGGTATAATGATG
CCCAAGTACTCTCAGATGATACTATGGCTTGCCTCCAAGGTAACTTGACAA
GATGCACCTTCTCTCCAGTGGTTGGGAGCTTTCTCACTCGATTCGTGCTGT
TCGATGGAATAGTTTATGCAAATTGCAGGTCGATGTTGTGCAAGTGCATG
CAACCTGCTGCTGTGATCCTACAGCCGAGTTCATCCCCTGTAACTGTCATT
GACATGTACAAATGTGTGAGTCTGCAGCTTGACAATCTCAGATTCACCATC
ACTCAATTGGCCAATGTAACCTACAATAGCACCATCAAGCTTGAATCATC
CCAGATCTTGTCTATTGATCCGTTGGATATATCCCAGAATCTAGCTGCGGT
GAATAAGAGTCTAAGTGATGCACTACAACACTTAGCACAAAGTGACACAT
ATCTTTCTGCAATCACATCAGCTACGACTACAAGTGTATTATCCATAATAG
CAATCTGTCTTGGATCGTTAGGTTTAATATTAATAATCTTGCTCAGTGTAG
TTGTGTGGAAGTTATTGACCATTGTCGTTGCTAATCGAAATAGAATGGAG
AATTTTGTTTATCATAAATAAGCATTCCACCACTCACGATCTGATCTCAGT
GAGAAAAATCAACCTGCAACTCTTGGAACAAGATAAGACAGTCATCCATT
AGTAATTTTTAAGAAAAAAACGATAGGACCGAACCTAGTATTGAAAGAAC
CGTCTCGGTCAATCTAGGTAATCGAGCTGATACCGTCTCGGAAAGCTCAA
ATCGCGCGCCACCatgtcagataacggaccacagaaccagaggaacgcacccaggattactttcggaggacc
aagcgatagcaccgggagcaaccagaatggagagcggagcggagcaagatccaagcagagacggccccagggcct
gccaaacaataccgcatcctggttcaccgccctgacacagcacggcaaggaggacctgaagtttccaaggggacaggg
agtgcctatcaacaccaatagctcccctgacgatcagatcggctactataggagggcaacaaggagaatcaggggaggc
gacggcaagatgaaggatctgagcccacgctggtacttctactatctgggaaccggacctgaggcaggcctgccatatgg
cgccaacaaggacggaatcatctgggtggcaaccgagggcgccctgaacacaccaaaggatcacatcggcacaagaa
atcccgccaacaatgcagcaatcgtgctgcagctgccacagggaaccacactgcccaagggcttttacgcagagggctct
cggggaggcagccaggcatctagcagatcctctagccggagcagaaactcctctaggaattccaccccaggaagctcca
ggggcacatcccctgcccgcatggcaggaaacggaggcgacgccgccctggccctgctgctgctggatcgcctgaatc
agctggagtccaagatgtctggcaagggacagcagcagcagggacagaccgtgacaaagaagtccgccgccgaggcc
tctaagaagccaaggcagaagcgcaccgccacaaaggcctacaacgtgacccaggccttcggcaggcgcggaccaga
gcagacacagggcaattttggcgaccaggagctgatcaggcagggaaccgattataagcactggcctcagatcgcccag
ttcgccccatctgccagcgccttctttggcatgtctagaatcggcatggaggtgacccccagcggcacatggctgacctaca
caggcgccatcaagctggacgataaggaccctaacttcaaggatcaggtcatcctgctgaacaagcacatcgacgcctat
aagacctttccccctacagagcccaagaaggacaagaagaagaaggccgatgagacacaggccctgcctcagaggca
gaagaagcagcagaccgtgacactgctgccagccgccgatctggacgatttctccaaacagctgcagcagagcatgtcc
agtgccgactccacccaggcttgaCGTACGACCTGCTATAGGCTATCCACTGCATCATCT
CTCCTGCCATACTTCCTACTCACATCATATCTATTTTAAAGAAAAAATAGG
CCCGAACACTAATCGTGCCGGCAGTGCCACTGCACACACAACACTACACA
TACAATACACTACAATGGTTGCAGAAGATGCCCCTGTTAGGGCCACTTGC
CGAGTATTATTTCGAACAACAACTTTAATCTTTCTATGCACACTACTAGCA
TTAAGCATCTCTATCCTTTATGAGAGTTTAATAACCCAAAAGCAAATCATG
AGCCAAGCAGGCTCAACTGGATCTAATTCTGGATTAGGAAGTATCACTGA
TCTTCTTAATAATATTCTCTCTGTCGCAAATCAGATTATATATAACTCTGCA
GTCGCTCTACCTCTACAATTGGACACTCTTGAATCAACACTCCTTACAGCC
ATTAAGTCTCTTCAAACCAGTGACAAGCTAGAACAGAACTGCTCGTGGAG
TGCTGCACTGATTAATGATAATAGATACATTAATGGCATCAATCAGTTCTA
TTTTTCAATTGCTGAGGGTCGCAATCTGACACTTGGCCCACTTCTTAATAT
GCCTAGTTTCATTCCAACTGCCACGACACCAGAGGGCTGCACCAGGATCC
CATCATTCTCGCTCACTAAGACACACTGGTGTTATACACACAATGTTATCC
TGAATGGATGCCAGGATCATGTATCCTCAAATCAATTTGTTTCTATGGGAA
TCATTGAACCCACTTCTGCCGGGTTTCCATTCTTTCGAACCCTAAAGACTC
TATATCTCAGCGATGGGGTCAATCGTAAGAGCTGCTCTATCAGTACAGTTC
CGGGGGGTTGTATGATGTACTGTTTTGTTTCTACTCAACCAGAGAGGGATG
ACTACTTTTCTGCCGCTCCTCCAGAACAACGAATTATTATAATGTACTATA
ATGATACAATCGTGGAGCGCATAATTAATCCACCCGGGGTACTAGATGTA
TGGGCAACATTGAACCCAGGAACAGGAAGCGGGGTATATTATTTAGGTTG
GGTGCTCTTTCCAATATATGGCGGCGTGATTAAAGGTACGAGTTTATGGA
ATAATCAAGCAAATAAATACTTTATCCCCCAGATGGTTGCTGCTCTCTGCT
CACAAAACCAGGCAACTCAAGTCCAAAATGCTAAGTCATCATACTATAGC
AGCTGGTTTGGCAATCGAATGATTCAGTCTGGGATCCTGGCATGTCCTCTT
CGACAGGATCTAACCAATGAGTGTTTAGTTCTGCCCTTTTCTAATGATCAG
GTGCTTATGGGTGCTGAAGGGAGATTATACATGTATGGTGACTCGGTGTA
TTACTATCAAAGAAGCAATAGTTGGTGGCCTATGACCATGCTGTATAAGG
TAACCATAACATTCACTAATGGTCAGCCATCTGCTATATCAGCTCAGAATG
TGCCCACACAGCAGGTCCCTAGACCTGGGACAGGAGACTGCTCTGCAACC
AATAGATGTCCCGGTTTTTGCTTGACAGGAGTGTATGCCGATGCCTGGTTA
CTGACCAACCCTTCGTCTACCAGTACATTTGGATCAGAAGCAACCTTCACT
GGTTCTTATCTCAACACAGCAACTCAGCGTATCAATCCGACGATGTATATC
GCGAACAACACACAGATCATAAGCTCACAGCAATTTGGATCAAGCGGTCA
AGAAGCAGCATATGGCCACACAACTTGTTTTAGGGACACAGGCTCTGTTA
TGGTATACTGTATCTATATTATTGAATTGTCCTCATCTCTCTTAGGACAATT
TCAGATTGTCCCATTTATCCGTCAGGTGACACTATCCTAAAGGCAGAAGCC
TTCAGGTCTGACCCAGCCAATCAAAGCATTATACCAGACCATGGCCTACC
ATCGGCTTTAAAGAAAAAAATAGGCCCGGACGGGTTAGCAACAAGCGGC
GGCCGCCACCatgttcgtcttcctggtcctgctgcctctggtctcctcacagtgcgtcaatctgacaactcggactca
gctgccacctgcttatactaatagcttcaccagaggcgtgtactatcctgacaaggtgtttagaagctccgtgctgcactctac
acaggatctgtttctgccattctttagcaacgtgacctggttccacGTGatcagcggcaccaatggcacaaagcggttcga
caatcccgtgctgccttttaacgatggcgtgtacttcgcctctATCgagaagagcaacatcatcagaggctggatctttgg
caccacactggactccaagacacagtctctgctgatgtgaacaatgccaccaacgtggtcatcaaggtgtgcgagttcca
gttttgtaatgatcccttcctgGATcacaagaacaataagagctggatggagtccgagtttagagtgtattctagcgccaac
aactgcacatttgagtacgtgagccagcctttcctgatggacctggagggcaagcagggcaatttcaagaacctgaggga
gttcgtgtttaagaatatcgacggctacttcaaaatctactctaagcacacccccATCATCgtgcgcGAGCCTGA
Ggacctgcctcagggcttcagcgccctggagcccctggtggatctgcctatcggcatcaacatcacccggtttcagacact
gctggccctgcacagaagctacctgacacccggcgactcctctagcggatggaccgccggcgctgccgcctactatgtg
ggctacctccagccccggaccttcctgctgaagtacaacgagaatggcaccatcacagacgcagtggattgcgccctgga
ccccctgagcgagacaaagtgtacactgaagtcctttaccgtggagaagggcatctatcagacatccaatttcagggtgca
gccaaccgagtctatcgtgcgctttcctaatatcacaaacctgtgcccatttGACgaggtgttcaacgcaacccgcttcgc
cagcgtgtacgcctggaataggaagcggatcagcaactgcgtggccgactatagcgtgctgtacaacCTGgccCCCt
tcTTCacctttaagtgctatggcgtgtcccccacaaagctgaatgacctgtgctttaccaacgtctacgccgattctttcgtg
atcaggggcgacgaggtgcgccagatcgcccccggccagacaggcAATatcgcagactacaattataagctgccag
acgatttcaccggctgcgtgatcgcctggaacagcaacAAGctggattccaaagtgTCCggcaactacaattatctgt
accggctgtttagaaagagcaatctgaagcccttcgagagggacatctctacagaaatctaccaggccggcAATAAG
ccttgcaatggcgtgGCCggctttaactgttatttcccactcAAGtcctacTCCttcCGCcccacaTATggcgtg
ggcCACcagccttaccgcgtggtggtgctgagctttgagctgctgcacgccccagcaacagtgtgcggccccaagaag
tccaccaatctggtgaagaacaagtgcgtgaacttcaacttcaacggcctgAAGggcacaggcgtgctgaccgagtcc
aacaagaagttcctgccatttcagcagttcggcagggacatcgcagataccacagacgccgtgcgcgacccacagaccct
ggagatcctggacatcacaccctgctctttcggcggcgtgagcgtgatcacacccggcaccaatacaagcaaccaggtgg
ccgtgctgtatcagGGCgtgaattgtaccgaggtgcccgtggctatccacgccgatcagctgaccccaacatggcgggt
gtacagcaccggctccaacgtcttccagacaagagccggatgcctgatcggagcagagTACgtgaacaattcctatga
gtgcgacatcccaatcggcgccggcatctgtgcctcttaccagacccagacaAAGtctCACagaagagcccggagc
gtggcctcccagtctatcatcgcctataccatgtccctgggcgccgagaacagcgtggcctactctaacaatagcatcgcca
tcccaaccaacttcacaatctctgtgaccacagagatcctgcccgtgtccatgaccaagacatctgtggactgcacaatgtat
atctgtggcgattctaccgagtgcagcaacctgctgctccagtacggcagcttttgtacccagctgAAGagagccctgac
aggcatcgccgtggagcaggataagaacacacaggaggtgttcgcccaggtgaagcaaatctacaagaccccccctatc
aagTACtttggcggcttcaatttttcccagatcctgcctgatccatccaagccttctaagcggagctttatcgaggacctgct
gttcaacaaggtgaccctggccgatgccggcttcatcaagcagtatggcgattgcctgggcgacatcgcagccagggacc
tgatctgcgcccagaagtttAAGggcctgaccgtgctgccacccctgctgacagatgagatgatcgcacagtacacaag
cgccctgctggccggcaccatcacatccggatggaccttcggcgcaggagccgccctccagatcccctttgccatgcaga
tggcctataggttcaacggcatcggcgtgacccagaatgtgctgtacgagaaccagaagctgatcgccaatcagtttaactc
cgccatcggcaagatccaggacagcctgtcctctacagccagcgccctgggcaagctccaggatgtggtgaatCACaa
cgcccaggccctgaataccctggtgaagcagctgagcagcAAGttcggcgccatctctagcgtgctgaatgacatcTT
Tagccggctggacaaggtggaggcagaggtgcagatcgaccggctgatcaccggccggctccagagcctccagacct
atgtgacacagcagctgatcagggccgccgagatcagggccagcgccaatctggcagcaaccaagatgtccgagtgcg
tgctgggccagtctaagagagtggacttttgtggcaagggctatcacctgatgtccttccctcagtctgccccacacggcgt
ggtgtttctgcacgtgacctacgtgcccgcccaggagaagaacttcaccacagcccctgccatctgccacgatggcaagg
cccactttccaagggagggcgtgttcgtgtccaacggcacccactggtttgtgacacagcgcaatttctacgagccccagat
catcaccacagacaacaccttcgtgagcggcaactgtgacgtggtcatcggcatcgtgaacaataccgtgtatgatccactc
cagcccgagctggacagctttaaggaggagctggataagtatttcaagaatcacacctcccctgacgtggatctgggcgac
atcagcggcatcaatgcctccgtggtgaacatccagaaggagatcgaccgcctgaacgaggtggctaagaatctgaacg
agagcctgatcgacctccaggagctgggcaagtatgagcagtacatcaagtggccctggtacatctggctgggcttcatcg
ccggcctgatcgccatcgtgatggtgaccatcatgctgtgctgtatgacatcctgctgttcttgcctgaagggctgctgtagct
gtggctcctgctgtaagttattgaccattgtcgttgctaatcgaaatagaatggagaattttgtttatcataaatgaAGTCG
ACTCATGGAATGCATACCAAACATTATTGACACTAATGACACACAAAATT
GGTTTTAAGAAAAACCAAGAGAACAATAGGCCAGAATGGCTGGGTCTCG
GGAGATATTACTCCCTGAAGTCCATCTCAATTCACCAATTGTAAAGCATAA
GCTATACTATTACATTCTACTTGGAAACCTCCCAAATGAGATCGACCTTGA
CGATTTAGGTCCATTACATAATCAAAATTGGAATCAGATAGCACATGAAG
AGTCTAACTTAGCTCAACGCTTGGTAAATGTAAGAAATTTTCTAATTACCC
ACATCCCTGATCTTAGAAAGGGCCATTGGCAAGAGTATGTCAATGTAATA
CTGTGGCCGCGAATTCTTCCCTTGATCCCGGATTTTAAAATCAATGACCAA
TTGCCTCTGCTCAAAAATTGGGACAAGTTAGTTAAAGAATCATGTTCAGTA
ATCAATGCAGGTACTTCCCAGTGCATTCAGAATCTCAGCTATGGACTGAC
AGGTCGTGGGAACCTCTTTACACGATCACGTGAACTCTCTGGTGACCGCA
GGGATATTGATCTTAAGACAGTTGTGGCAGCATGGCATGACTCAGACTGG
AAAAGAATAAGTGATTTTTGGATTATGATCAAATTCCAGATGAGACAATT
AATTGTTAGGCAAACAGATCATAATGATTCTGATTTAATCACGTATATCGA
AAATAGAGAAGGCATAATCATCATAACCCCTGAACTGGTAGCATTATTTA
ACACTGAGAATCATACACTAACATACATGACCTTTGAAATTGTACTGATG
GTTTCAGATATGTACGAAGGTCGTCACAACATTTTATCACTATGCACAGTT
AGCACTTACCTGAATCCTCTGAAGAAAAGAATAACATATTTATTGAGCCTT
GTAGATAACTTAGCTTTTCAGATAGGTGATGCTGTATATAACATAATTGCT
TTGCTAGAATCCTTTGTATATGCACAGTTGCAAATGTCAGATCCCATCCCA
GAACTCAGAGGACAATTCCATGCATTCGTATGTTCTGAGATTCTTGATGCA
CTAAGAGGAACTAATAGTTTCACCCAGGATGAATTAAGAACTGTGACAAC
TAATTTGATATCCCCATTCCAAGATCTGACCCCAGATCTTACGGCTGAATT
GCTCTGTATAATGAGGCTTTGGGGACACCCCATGCTCACTGCCAGTCAAG
CTGCAGGAAAGGTACGCGAGTCTATGTGTGCTGGAAAAGTATTAGACTTT
CCCACCATTATGAAAACACTAGCCTTTTTCCATACTATTCTGATCAATGGA
TACAGGAGGAAGCATCATGGAGTATGGCCACCCTTAAACTTACCGGGTAA
TGCTTCAAAGGGTCTCACGGAACTTATGAATGACAATACTGAAATAAGCT
ATGAATTCACACTTAAGCATTGGAAGGAAGTCTCTCTTATAAAATTCAAG
AAATGTTTTGATGCAGACGCAGGTGAGGAACTCAGTATATTTATGAAAGA
TAAGGCAATTAGTGCCCCAAAACAAGACTGGATGAGTGTGTTTAGAAGAA
GCCTAATCAAACAGCGCCATCAGCATCATCAGGTCCCCCTACCAAATCCA
TTCAATCGACGGCTGTTGCTAAACTTTCTCGGAGATGACAAATTCGACCCG
AATGTGGAGCTACAGTATGTAACATCAGGTGAGTATCTACATGATGACAC
GTTTTGTGCATCATATTCACTAAAAGAGAAGGAAATTAAACCTGATGGTC
GAATTTTTGCAAAGTTGACTAAGAGAATGAGATCATGTCAAGTTATAGCA
GAATCTCTTTTAGCGAACCATGCTGGGAAGTTAATGAAAGAGAATGGTGT
TGTGATGAATCAGCTATCATTAACAAAATCACTATTAACAATGAGTCAGA
TTGGAATAATATCCGAGAAAGCTAGAAAGTCAACTCGAGATAACATAAAT
CAACCTGGTTTCCAGAATATCCAGAGAAATAAATCACATCACTCCAAGCA
AGTCAATCAGCGAGATCCAAGTGATGACTTTGAATTGGCAGCATCTTTTTT
AACTACTGATCTCAAAAAATATTGTTTACAATGGAGGTACCAGACAATTA
TCCCATTTGCTCAATCATTAAACAGAATGTATGGTTATCCTCATCTCTTTG
AGTGGATTCACTTACGGCTAATGCGTAGTACACTTTACGTGGGGGATCCCT
TCAACCCACCAGCAGATACCAGTCAATTTGATCTAGATAAAGTAATTAAT
GGAGATATCTTCATTGTATCACCCAGAGGTGGAATTGAAGGGCTGTGTCA
AAAGGCTTGGACAATGATATCTATCGCTGTGATAATTCTATCTGCCACAGA
GTCTGGCACACGAGTAATGAGTATGGTGCAGGGAGATAATCAAGCAATTG
CTGTCACCACACGAGTACCAAGGAGCCTGCCGACTCTTGAGAAAAAGACT
ATTGCTTTTAGATCTTGTAATCTATTCTTTGAGAGGTTAAAATGTAATAAT
TTTGGATTAGGTCACCATTTGAAAGAACAAGAGACTATCATTAGTTCTCAC
TTCTTTGTTTATAGCAAGAGAATATTCTATCAGGGGAGGATTCTAACGCAA
GCCTTAAAAAATGCTAGTAAGCTCTGCTTGACAGCTGATGTCCTAGGAGA
ATGCACCCAATCATCATGTTCTAATCTTGCAACTACTGTCATGAGGTTAAC
TGAGAATGGTGTTGAAAAAGATATCTGTTTCTACTTGAATATCTATATGAC
CATCAAACAGCTCTCCTATGATATCATCTTCCCTCAAGTGTCAATTCCTGG
AGATCAGATCACATTAGAATACATAAATAATCCACACCTGGTATCACGAT
TGGCTCTTTTGCCATCCCAGTTAGGAGGTCTAAACTACCTGTCATGCAGTA
GGCTGTTCAATCGAAACATAGGCGACCCGGTGGTTTCCGCAGTTGCAGAT
CTTAAGAGATTAATTAAATCAGGATGTATGGATTACTGGATCCTTTATAAC
TTATTAGGGAGAAAACCGGGAAACGGCTCATGGGCTACTTTAGCAGCTGA
CCCGTACTCAATCAATATAGAGTATCAATACCCTCCAACTACAGCTCTTAA
GAGGCACACCCAACAAGCTCTGATGGAACTCAGTACGAATCCAATGTTAC
GTGGCATATTCTCTGACAATGCACAGGCAGAAGAAAATAACCTTGCTAGG
TTTCTCCTGGATAGGGAGGTGATCTTTCCGCGTGTAGCTCACATCATCATT
GAGCAAACCAGTGTCGGGAGGAGAAAACAGATTCAAGGATATTTGGATTC
AACTAGATCGATAATGAGGAAATCACTAGAAATTAAGCCCTTATCCAATA
GGAAGCTTAATGAAATACTGGATTACAACATCAATTACCTAGCTTACAAT
TTGGCATTACTCAAGAATGCTATTGAACCTCCGACTTATTTGAAGGCAATG
ACACTTGAAACATGTAGCATCGACATTGCAAGGAACCTCCGGAAGCTCTC
CTGGGCCCCACTCTTGGGTGGGAGAAATCTTGAAGGATTAGAGACGCCAG
ATCCCATTGAAATTACTGCAGGAGCATTAATTGTTGGATCGGGCTACTGTG
AACAGTGTGCTGCAGGAGACAATCGATTCACATGGTTTTTCTTGCCATCTG
GTATCGAGATAGGAGGGGATCCCCGTGATAATCCTCCTATCCGTGTACCG
TACATTGGCTCCAGGACTGATGAGAGGAGGGTAGCCTCAATGGCATACAT
CAGGGGTGCCTCGAGTAGCTTAAAAGCAGTTCTTAGACTGGCGGGAGTGT
ACATCTGGGCATTCGGAGATACTCTGGAGAATTGGATAGATGCACTGGAT
TTGTCTCACACTAGAGTTAACATCACACTTGAACAGCTGCAATCCCTCACC
CCACTTCCAACCTCTGCCAATCTAACCCATCGGTTGGATGATGGCACAACT
ACCCTAAAGTTTACTCCTGCGAGCTCTTACACCTTTTCAAGTTTCACTCAT
ATATCAAATGATGAGCAATACCTGACAATTAATGACAAAACTGCAGATTC
AAATATAATCTACCAACAGTTAATGATCACTGGACTCGGAATCTTAGAAA
CATGGAATAATCCCCCAATCAATAGAACATTCGAAGAATCTACCCTACAT
TTGCACACTGGTGCATCATGTTGTGTCCGACCTGTGGACTCCTGCATTCTC
TCAGAAGCATTAACAGTCAAGCCACATATTACAGTACCGTACAGCAATAA
ATTTGTATTTGATGAGGACCCGCTATCTGAATATGAAACTGCAAAACTGG
AATCGTTATCATTCCAAGCCCAATTAGGCAACATTGATGCTGTAGATATGA
CAGGTAAATTAACATTATTGTCCCAATTCACTGCAAGGCAGATTATCAATG
CAATCACTGGACTCGATGAGTCTGTCTCTCTTACTAATGATGCCATTGTTG
CATCAGACTATGTCTCCAATTGGATTAGTGAATGCATGTATACCAAATTAG
ATGAATTATTTATGTATTGTGGGTGGGAACTACTATTGGAACTATCCTATC
AAATGTATTATCTGAGGGTAGTTGGGTGGAGTAATATAGTGGATTATTCTT
ACATGATCTTGAGAAGAATCCCGGGTGCAGCATTAAACAATCTGGCATCT
ACATTAAGTCATCCAAAACTTTTCCGACGAGCTATCAACCTAGATATAGTT
GCCCCCTTAAATGCTCCTCATTTTGCATCTCTGGACTACATCAAGATGAGT
GTGGATGCAATACTCTGGGGCTGTAAAAGAGTCATCAATGTGCTCTCCAA
TGGAGGGGACTTAGAATTAGTTGTGACATCTGAAGATAGCCTTATTCTCA
GTGACCGATCCATGAATCTCATTGCAAGGAAATTAACTTTATTATCACTGA
TTCACCATAATGGTTTGGAACTACCAAAGATTAAGGGGTTCTCTCCTGATG
AGAAGTGTTTCGCTTTGACAGAATTTTTGAGGAAAGTGGTGAACTCAGGG
TTGAGTTCAATAGAGAACCTATCAAATTTTATGTACAATGTGGAGAACCC
ACGGCTTGCAGCATTCGCCAGCAACAATTACTACCTGACCAGAAAATTAT
TGAATTCAATACGAGATACTGAGTCGGGTCAAGTAGCAGTCACCTCATAT
TATGAATCATTAGAATATATTGATAGTCTTAAGCTAACCCCACATGTGCCT
GGCACCTCATGCATTGAGGATGATAGTCTATGTACAAATGATTACATAAT
CTGGATCATAGAGTCTAATGCAAACTTGGAGAAGTATCCAATTCCAAATA
GCCCTGAGGATGATTCCAATTTCCATAACTTTAAGTTGAATGCTCCATCGC
ACCATACCTTACGCCCATTAGGGTTGTCATCAACTGCTTGGTATAAGGGTA
TAAGCTGCTGCAGGTACCTTGAGCGATTAAAGCTACCACAAGGTGATCAT
TTATATATTGCAGAAGGTAGTGGTGCCAGTATGACAATCATAGAATACCT
ATTCCCAGGAAGAAAGATATATTACAATTCTTTATTTAGTAGTGGTGACAA
TCCCCCACAAAGAAATTATGCACCAATGCCTACTCAGTTCATTGAGAGTGT
CCCATACAAGCTCTGGCAAGCACACACAGATCAATATCCCGAGATTTTTG
AGGACTTCATCCCTCTATGGAACGGAAACGCCGCCATGACTGACATAGGA
ATGACAGCTTGTGTAGAATTCATCATCAATCGAGTCGGCCCAAGGACTTG
CAGTTTAGTACATGTAGATTTGGAATCAAGTGCAAGCTTAAATCAACAAT
GCCTGTCAAAGCCGATAATTAATGCTATCATCACTGCTACAACTGTTTTGT
GCCCTCATGGGGTGCTTATTCTGAAATATAGTTGGTTGCCATTTACTAGAT
TTAGTACTTTGATCACTTTCTTATGGTGCTACTTTGAGAGAATCACTGTTCT
TAGGAGCACATATTCTGATCCAGCTAATCATGAGGTTTATTTAATTTGTAT
CCTTGCCAACAACTTTGCATTCCAGACTGTCTCGCAGGCAACAGGAATGG
CGATGACTTTAACTGATCAAGGGTTTACTTTGATATCACCTGAAAGAATAA
ATCAGTATTGGGATGGTCACTTGAAGCAAGAACGTATCGTAGCAGAAGCA
ATTGATAAGGTGGTTCTAGGAGAAAATGCTCTATTTAATTCGAGTGATAAT
GAATTAATTCTCAAATGTGGAGGGACACCAAATGCACGGAATCTCATCGA
TATCGAGCCAGTCGCAACTTTCATAGAATTTGAACAATTGATCTGCACAAT
GTTGACAACCCACTTGAAGGAAATAATTGATATAACAAGGTCTGGAACCC
AGGATTATGAAAGTTTATTACTCACTCCTTACAATTTAGGTCTTCTTGGTA
AAATCAGTACGATAGTGAGATTATTAACAGAAAGGATTCTAAATCATACT
ATCAGGAATTGGTTGATCCTCCCACCTTCGCTCCGGATGATCGTGAAGCAG
GACTTGGAATTCGGCATATTCAGGATTACTTCCATCCTCAATTCTGATCGG
TTCCTGAAGCTTTCTCCAAATAGGAAATACTTGATTGCACAATTAACTGCA
GGCTACATTAGGAAATTGATTGAGGGGGATTGCAATATCGATCTAACCAG
ACCTATCCAAAAGCAAATCTGGAAAGCATTAGGTTGTGTAGTCTATTGTC
ACGATCCAATGGATCAAAGGGAGTCAACAGAGTTTATTGATATAAATATT
AATGAAGAAATAGACCGCGGGATCGATGGCGAGGAAATCTAAACATATC
AAGAATCAGAATTAGTTTAAGAAAAAAGAAGAGGATTAATCTTGGTTTTC
CCCTTGGTGGGTCGGCATGGCATCTCCACCTCCTCGCGGTCCGACCTGGGC
ATCCGAAGGAGGACGCACGTCCACTCGGATGGCTAAGGGAGCGGCCGGG
GATCCGGCTGCTAACAAAGCCCGAAAGGAAGCTGAGTTGGCTGCTGCCAC
CGCTGAGCAATAACTAGCATAACCCCTTGGGGCCTCTAAACGGGTCTTGA
GGGGTTTTTTGCTGAAAGGAGGAACTATATCCGGATCATGTGAGCAAAAG
GCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCA
GAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTC
TTGAGTCCAACCCGGTAAGACACGACTTATCGCCACTGGCAGCAGCCACT
GGTAACAGGATTAGCAGAGCGAGGTATGTAGGCGGTGCTACAGAGTTCTT
GAAGTGGTGGCCTAACTACGGCTACACTAGAAGAACAGTATTTGGTATCT
GCGCTCTGCTGAAGCCAGTTACCTTCGGAAAAAGAGTTGGTAGCTCTTGA
TCCGGCAAACAAACCACCGCTGGTAGCGGTGGTTTTTTTGTTTGCAAGCAG
CAGATTACGCGCAGAAAAAAAGGATCTCAAGAAGATCCTTTGATCTTTTC
TACGGGGTCTGACGCTCAGTGGAACGAAAACTCACGTTAAGGGATTTTGG
TCATGAGATTATCAAAAAGGATCTTCACCTAGATCCTTTTAAATTAAAAAT
GAAGTTTTAAATCAATCTAAAGTATATATGAGTAAACTTGGTCTGACAGTT
ACCAATGCTTAATCAGTGAGGCACCTATCTCAGCGATCTGTCTATTTCGTT
CATCCATAGTTGCCTGACTCCCCGTCGTGTAGATAACTACGATACGGGAG
GGCTTACCATCTGGCCCCAGTGCTGCAATGATACCGCGAGACCCACGCTC
ACCGGCTCCAGATTTATCAGCAATAAACCAGCCAGCCGGAAGGGCCGAGC
GCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCT
TCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCT
CTTACTGTCATGCCATCCGTAAGATGCTTTTCTGTGACTGGTGAGTACTCA
ACCAAGTCATTCTGAGAATAGTGTATGCGGCGACCGAGTTGCTCTTGCCC
GGCGTCAATACGGGATAATACCGCGCCACATAGCAGAACTTTAAAAGTGC
TCATCATTGGAAAACGTTCTTCGGGGCGAAAACTCTCAAGGATCTTACCG
CTGTTGAGATCCAGTTCGATGTAACCCACTCGTGCACCCAACTGATCTTCA
GCATCTTTTACTTTCACCAGCGTTTCTGGGTGAGCAAAAACAGGAAGGCA
AAATGCCGCAAAAAAGGGAATAAGGGCGACACGGAAATGTTGAATACTC
ATACTCTTCCTTTTTCAATATTATTGAAGCATTTATCAGGGTTATTGTCTCA
TGAGCGGATACATATTTGAATGTATTTAGAAAAATAAACAAATAGGGGTT
CCGCGCACATTTCCCCGAAAAGT.

v. CVL109

The nucleic acid sequence for CVL109 is:

(SEQ ID NO: 7)
GCCACCTGACGTCTAAGAAACCATTATTATCATGACATTAACCTATAAAA
ATAGGCGTATCACGAGGCCCTTTCGTCTCGCGCGTTTCGGTGATGACGGTG
AAAACCTCTGACACATGCAGCTCCCGGAGACGGTCACAGCTTGTCTGTAA
GCGGATGCCGGGAGCAGACAAGCCCGTCAGGGCGCGTCAGCGGGTGTTG
GCGGGTGTCGGGGCTGGCTTAACTATGCGGCATCAGAGCAGATTGTACTG
AGAGTGCACCATATATGCGGTGTGAAATACCGCACAGATGCGTAAGGAGA
AAATACCGCATCAGGCTAATACGACTCACTATAGGGACCAAGGGGAAAAT
GAAGTGGTGACTCAAATCATCGAAGACCCTCGAGATTACATAGGTCCGGA
ACCTATGGCCTTCGTGACCGACCTCGAGTCAGAGTAGTTCAATAAGGACC
TATCAAGTTTGGGCAATTTTTCGTCCCCGACACAAAAATGTCATCCGTGCT
TAAAGCATATGAGCGATTCACGCTCACTCAAGAACTGCAAGATCAGAGTG
AGGAAGGTACAATCCCACCTACAACACTAAAACCGGTAATCAGGGTATTT
ATACTAACCTCTAATAACCCAGAGCTAAGATCCCGGCTTCTTCTATTCTGC
CTACGGATTGTTCTCAGTAATGGTGCAAGGGATTCCCATCGCTTTGGAGCA
TTACTCACAATGTTTTCGCTACCATCAGCCACAATGCTCAATCATGTCAAA
TTAGCTGACCAGTCACCAGAAGCTGATATCGAAAGGGTAGAGATCGATGG
CTTTGAGGAGGGATCATTCCGCTTAATCCCCAATGCTCGTTCAGGTATGAG
CCGTGGAGAGATCAATGCCTATGCTGCACTTGCAGAAGATCTACCTGACA
CACTAAACCATGCAACACCTTTCGTTGATTCCGAAGTCGAGGGAACTGCA
TGGGATGAGATTGAGACTTTCTTAGATATGTGTTACAGTGTCCTAATGCAG
GCATGGATAGTGACTTGCAAGTGCATGACTGCGCCAGACCAACCTGCTGC
TTCTATTGAGAAACGCCTGCAAAAATATCGTCAGCAAGGCAGGATCAACC
CGAGATATCTCCTGCAACCGGAGGCTCGACGAATAATCCAGAATGTAATC
CGGAAGGGAATGGTGGTCAGACATTTCCTCACCTTTGAACTGCAGCTTGC
CCGAGCACAAAGCCTTGTATCAAATAGGTATTATGCTATGGTAGGGGATG
TTGGAAAGTATATAGAGAATTGTGGAATGGGAGGCTTCTTTTTGACACTA
AAATATGCATTAGGAACTAGATGGCCCACACTTGCTTTAGCTGCATTTTCA
GGAGAGCTAACAAAGCTAAAGTCCCTCATGGCATTATACCAGACCCTTGG
TGAGCAGGCCCGATATTTGGCCCTATTGGAGTCACCACATTTGATGGATTT
TGCTGCAGCAAACTACCCACTGCTATATAGCTATGCTATGGGAATAGGCT
ATGTGTTAGATGTCAACATGAGGAACTACGCTTTCTCCAGATCATACATGA
ACAAGACATATTTCCAATTGGGAATGGAAACTGCAAGAAAACAACAGGG
TGCAGTTGACATGAGGATGGCAGAAGATCTCGGTCTAACTCAAGCCGAAC
GCACCGAGATGGCAAATACACTTGCCAAATTGACCACAGCAAATCGAGGG
GCAGACACCAGGGGAGGAGTCAACCCGTTCTCATCTGTCACTGGGACAAC
TCAGGTGCCCGCTGCAGCAACAGGTGACACACTCGAGAGTTACATGGCAG
CGGATCGACTGAGGCAGAGATATGCTGATGCAGGCACCCATGATGATGAG
ATGCCACCATTGGAAGAGGAGGAAGAGGACGACACATCTGCAGGTCCAC
GCACTGGACCAACTCTTGAACAAGTGGCCTTGGACATCCAGAACGCAGCA
GTTGGAGCTCCCATCCATACAGATGACCTGAATGCCGCACTGGGTGATCTT
GACATCTAGACAATTCAGATCCCAATCTAAAATTGACATACCTAATTGATT
AGTTAGATGGAACTACAGTGGATTCCATAAGGTTCCTGCCTACCATCGGCT
TTAAAGAAAAAAATAGGCCCGGACGGGTTAGCAACAAGCGACTGCCGGT
GCCAACAGCGCAATCCACAATCTACAATGGATCCCACTGATCTGAGCTTC
TCCCCAGATGAGATCAATAAGCTCATAGAGACAGGCCTGAATACTGTAGA
GTATTTTACTTCCCAACAAGTCACAGGAACATCCTCTCTTGGAAAGAATAC
AATACCACCAGGGGTCACAGGACTACTAACCAATGCTGCAGAGGCAAAG
ATCCAAGAGTCAACTAACCATCAGAAGGGCTCAGTTGGTGGGGGTGCAAA
ACCAAAGAAACCGCGACCAAAAATTGCCATTGTGCCAGCAGATGACAAA
ACAGTGCCCGGAAAGCCGATCCCAAACCCTCTATTAGGTCTGGACTCCAC
CCCGAGCACCCAAACTGTGCTTGATCTAAGTGGGAAAACATTACCATCAG
GATCCTATAAGGGGGTTAAGCTTGCGAAATTTGGAAAAGAAAATCTGATG
ACACGGTTCATCGAGGAACCCAGAGAGAATCCTATCGCAACCAGTTtCCCC
ATCGATTTTAAGAGGGGCAGGGATACCGGCGGGTTCCATAGAAGGGAGTA
CTCAATCGGATGGGTGGGAGATGAAGTCAAGGTCACTGAGTGGTGCAATC
CATCCTGTTCTCCAATCACCGCTGCAGCAAGGCGATTTGAATGCACTTGTC
ACCAGTGTCCAGTCACTTGCTCTGAATGTGAACGAGATACTTAATACAGT
GAGAAATTTGGACTCTCGGATGAATCAACTGGAGACAAAAGTAGATCGCA
TTCTCTCATCTCAGTCTCTAATCCAGACCATCAAGAATGACATAGTTGGAC
TTAAAGCAGGGATGGCTACTTTAGAAGGAATGATTACAACTGTGAAAATC
ATGGACCCGGGAGTTCCCAGTAATGTTACTGTGGAAGATGTACGCAAGAC
ACTAAGTAACCATGCTGTTGTTGTGCCAGAATCATTCAATGATAGTTTCTT
GACTCAATCTGAAGATGTAATTTCACTTGATGAGTTGGCTCGACCAACTGC
AACAAGTGTTAAGAAGATTGTCAGGAAGGTTCCTCCTCAGAAGGATCTGA
CTGGATTGAAGATTACACTAGAGCAATTGGCAAAGGATTGCATCAGCAAA
CCGAAGATGAGGGAAGAGTATCTCCTCAAAATCAACCAGGCTTCCAGTGA
GGCTCAGCTAATTGACCTCAAGAAAGCAATCATCCGCAGTGCAATTTGAT
CAAGAAACACCCAATTACACTACACTGGTATGACACTGTACTAACCCTGA
GGGTTTTAGAAAAAACGATTAACGATAAATAAGCCCGAACACTACACACT
ACCTGAGGCAGCCATGCCATCCATCAGCATTCCCGCAGACCCCACCAATC
CACGTCAATCAATAAAAGCGTTCCCAATTGTGATCAACAGTGATGGGGGT
GAGAAAGGCCGCTTGGTTAAACAACTACGCACAACCTACTTGAATGACCT
AGATACTCATGAGCCACTGGTGACATTCATAAATACCTATGGATTCATCTA
CGAACAGGATCGGGGGAATACCATTGTCGGAGAGGATCAACTTGGGAAG
AAAAGAGAGGCTGTGACCGCTGCAATGGTTACCCTTGGATGTGGGCCTAA
TCTACCATCATTAGGGAATGTCCTGGGACAACTGAGGGAATTCCAGGTCA
CTGTTAGGAAGACATCCAGCAAAGCGGAAGAGATGGTCTTTGAAATTGTT
AAGTATCCGAGAATATTTCGGGGTCATACATTAATCCAGAAAGGACTAGT
CTGTGTCTCCGCAGAAAAATTTGTTAAGTCACCAGGGAAAATACAATCTG
GAATGGACTATCTCTTCATTCCGACATTTCTGTCAGTGACTTACTGTCCAG
CTGCAATCAAATTTCAGGTACCTGGCCCCATGTTGAAAATGAGATCAAGA
TACACTCAGAGCTTACAACTTGAACTAATGATAAGAATCCTGTGTAAGCC
CGATTCGCCACTTATGAAGGTCCATACCCCTGACAAGGAGGGAAGAGGAT
GTCTTGTATCAGTATGGCTGCATGTATGCAACATCTTCAAATCAGGAAACA
AGAATGGCAGTGAGTGGCAGGAATACTGGATGAGAAAGTGTGCTAACAT
GCAACTTGAAGTGTCGATTGCAGATATGTGGGGACCAACTATCATAATTC
ATGCCAGAGGTCACATTCCCAAAAGTGCTAAGTTGTTTTTTGGAAAGGGT
GGATGGAGCTGCCATCCACTTCACGAAGTTGTTCCAAGTGTCACTAAAAC
ACTATGGTCCGTGGGCTGTGAGATTACAAAGGCGAAGGCAATAATACAAG
AGAGTAGCATCTCTCTTCTCGTGGAGACTACTGACATCATAAGTCCAAAA
GTCAAAATTTCATCTAAGCATCGCCGCTTTGGGAAATCAAATTGGGGTCTG
TTCAAGAAAACTAAATCACTGCCTAACCTGACGGAGCTGGAATGACTGAC
CTCTAATCGAGACTACACCGCCGCAAACTATAGGTGGGTGGTACCTCAGT
GATTAATCTTGTAAGCACTGATCGTAGGCTACAACACACTAATATTATCCA
GATTAGAGAGCTTAATTAGCTCTGTATTAATAATAACACTACTATTCCAAT
AACTGGAATCACCAGCTTGATTTATCTCCAAAATGATTCAAAGAAAACAA
ATCATATTAAGACTATCCTAAGCACGAACCCATATCGTCCTTCAAATCATG
GGTACTATAATTCAATTTCTGGTGGTCTCCTGTCTATTGGCAGGAGCAGGC
AGCCTTGATCCAGCAGCCCTCATGCAAATCGGTGTCATTCCAACAAATGTC
CGGCAACTTATGTATTATACTGAAGCTTCATCAGCATTCATTGTTGTGAAG
TTAATGCCTACAATTGACTCGCCGATTAGTGGATGTAATATAACATCAATT
TCAAGCTATAATGCAACAGTGACAAAACTCCTACAGCCGATCGGTGAGAA
TTTGGAGACGATTAGGAACCAGTTGATTCCAACTCGGAGGAGACGCCGGT
TTGCAGGGGTGGTGATTGGATTAGCTGCATTAGGAGTAGCTACTGCCGCA
CAGGTCACTGCCGCAGTGGCACTAGTAAAGGCAAATGAAAATGCTGCGGC
TATACTCAATCTCAAAAATGCAATCCAAAAAACAAATGCAGCAGTTGCAG
ATGTGGTCCAGGCCACACAATCACTAGGAACGGCAGTTCAAGCAGTTCAA
GATCACATAAACAGTGTGGTAAGTCCAGCAATTACAGCAGCCAATTGTAA
GGCCCAAGATGCTATCATTGGCTCAATCCTCAATCTCTATTTGACCGAGTT
GACAACCATCTTCCACAATCAAATTACAAACCCTGCATTGAGTCCCATTAC
AATTCAAGCTTTAAGGATCCTACTGGGGAGTACCTTGCCGACTGTGGTCG
AAAAATCTTTCAATACCCAGATAAGTGCAGCTGAGCTTCTCTCATCAGGGT
TATTGACAGGCCAGATTGTGGGATTAGATTTGACCTATATGCAGATGGTC
ATAAAAATTGAGCTGCCAACTTTAACTGTACAACCTGCAACCCAGATCAT
AGATCTGGCCACCATTTCTGCATTCATTAACAATCAAGAAGTCATGGCCCA
ATTACCAACACGTGTTATGGTGACTGGCAGCTTGATCCAAGCCTATCCCGC
ATCGCAATGCACCATTACACCCAACACTGTGTACTGTAGGTATAATGATG
CCCAAGTACTCTCAGATGATACTATGGCTTGCCTCCAAGGTAACTTGACAA
GATGCACCTTCTCTCCAGTGGTTGGGAGCTTTCTCACTCGATTCGTGCTGT
TCGATGGAATAGTTTATGCAAATTGCAGGTCGATGTTGTGCAAGTGCATG
CAACCTGCTGCTGTGATCCTACAGCCGAGTTCATCCCCTGTAACTGTCATT
GACATGTACAAATGTGTGAGTCTGCAGCTTGACAATCTCAGATTCACCATC
ACTCAATTGGCCAATGTAACCTACAATAGCACCATCAAGCTTGAATCATC
CCAGATCTTGTCTATTGATCCGTTGGATATATCCCAGAATCTAGCTGCGGT
GAATAAGAGTCTAAGTGATGCACTACAACACTTAGCACAAAGTGACACAT
ATCTTTCTGCAATCACATCAGCTACGACTACAAGTGTATTATCCATAATAG
CAATCTGTCTTGGATCGTTAGGTTTAATATTAATAATCTTGCTCAGTGTAG
TTGTGTGGAAGTTATTGACCATTGTCGTTGCTAATCGAAATAGAATGGAG
AATTTTGTTTATCATAAATAAGCATTCCACCACTCACGATCTGATCTCAGT
GAGAAAAATCAACCTGCAACTCTTGGAACAAGATAAGACAGTCATCCATT
AGTAATTTTTAAGAAAAAAACGATAGGACCGAACCTAGTATTGAAAGAAC
CGTCTCGGTCAATCTAGGTAATCGAGCTGATACCGTCTCGGAAAGCTCAA
ATCGCGCGCCACCatgtcagataacggaccacagaaccagaggaacgcacccaggattactttcggaggacc
aagcgatagcaccgggagcaaccagaatggagagcggagcggagcaagatccaagcagagacggccccagggcct
gccaaacaataccgcatcctggttcaccgccctgacacagcacggcaaggaggacctgaagtttccaaggggacaggg
agtgcctatcaacaccaatagctcccctgacgatcagatcggctactataggagggcaacaaggagaatcaggggaggc
gacggcaagatgaaggatctgagcccacgctggtacttctactatctgggaaccggacctgaggcaggcctgccatatgg
cgccaacaaggacggaatcatctgggtggcaaccgagggcgccctgaacacaccaaaggatcacatcggcacaagaa
atcccgccaacaatgcagcaatcgtgctgcagctgccacagggaaccacactgcccaagggcttttacgcagagggctct
cggggaggcagccaggcatctagcagatcctctagccggagcagaaactcctctaggaattccaccccaggaagctcca
ggggcacatcccctgcccgcatggcaggaaacggaggcgacgccgccctggccctgctgctgctggatcgcctgaatc
agctggagtccaagatgtctggcaagggacagcagcagcagggacagaccgtgacaaagaagtccgccgccgaggcc
tctaagaagccaaggcagaagcgcaccgccacaaaggcctacaacgtgacccaggccttcggcaggcgcggaccaga
gcagacacagggcaattttggcgaccaggagctgatcaggcagggaaccgattataagcactggcctcagatcgcccag
ttcgccccatctgccagcgccttctttggcatgtctagaatcggcatggaggtgacccccagcggcacatggctgacctaca
caggcgccatcaagctggacgataaggaccctaacttcaaggatcaggtcatcctgctgaacaagcacatcgacgcctat
aagacctttccccctacagagcccaagaaggacaagaagaagaaggccgatgagacacaggccctgcctcagaggca
gaagaagcagcagaccgtgacactgctgccagccgccgatctggacgatttctccaaacagctgcagcagagcatgtcc
agtgccgactccacccaggcttgaCGTACGACCTGCTATAGGCTATCCACTGCATCATCT
CTCCTGCCATACTTCCTACTCACATCATATCTATTTTAAAGAAAAAATAGG
CCCGAACACTAATCGTGCCGGCAGTGCCACTGCACACACAACACTACACA
TACAATACACTACAATGGTTGCAGAAGATGCCCCTGTTAGGGCCACTTGC
CGAGTATTATTTCGAACAACAACTTTAATCTTTCTATGCACACTACTAGCA
TTAAGCATCTCTATCCTTTATGAGAGTTTAATAACCCAAAAGCAAATCATG
AGCCAAGCAGGCTCAACTGGATCTAATTCTGGATTAGGAAGTATCACTGA
TCTTCTTAATAATATTCTCTCTGTCGCAAATCAGATTATATATAACTCTGCA
GTCGCTCTACCTCTACAATTGGACACTCTTGAATCAACACTCCTTACAGCC
ATTAAGTCTCTTCAAACCAGTGACAAGCTAGAACAGAACTGCTCGTGGAG
TGCTGCACTGATTAATGATAATAGATACATTAATGGCATCAATCAGTTCTA
TTTTTCAATTGCTGAGGGTCGCAATCTGACACTTGGCCCACTTCTTAATAT
GCCTAGTTTCATTCCAACTGCCACGACACCAGAGGGCTGCACCAGGATCC
CATCATTCTCGCTCACTAAGACACACTGGTGTTATACACACAATGTTATCC
TGAATGGATGCCAGGATCATGTATCCTCAAATCAATTTGTTTCTATGGGAA
TCATTGAACCCACTTCTGCCGGGTTTCCATTCTTTCGAACCCTAAAGACTC
TATATCTCAGCGATGGGGTCAATCGTAAGAGCTGCTCTATCAGTACAGTTC
CGGGGGGTTGTATGATGTACTGTTTTGTTTCTACTCAACCAGAGAGGGATG
ACTACTTTTCTGCCGCTCCTCCAGAACAACGAATTATTATAATGTACTATA
ATGATACAATCGTGGAGCGCATAATTAATCCACCCGGGGTACTAGATGTA
TGGGCAACATTGAACCCAGGAACAGGAAGCGGGGTATATTATTTAGGTTG
GGTGCTCTTTCCAATATATGGCGGCGTGATTAAAGGTACGAGTTTATGGA
ATAATCAAGCAAATAAATACTTTATCCCCCAGATGGTTGCTGCTCTCTGCT
CACAAAACCAGGCAACTCAAGTCCAAAATGCTAAGTCATCATACTATAGC
AGCTGGTTTGGCAATCGAATGATTCAGTCTGGGATCCTGGCATGTCCTCTT
CGACAGGATCTAACCAATGAGTGTTTAGTTCTGCCCTTTTCTAATGATCAG
GTGCTTATGGGTGCTGAAGGGAGATTATACATGTATGGTGACTCGGTGTA
TTACTATCAAAGAAGCAATAGTTGGTGGCCTATGACCATGCTGTATAAGG
TAACCATAACATTCACTAATGGTCAGCCATCTGCTATATCAGCTCAGAATG
TGCCCACACAGCAGGTCCCTAGACCTGGGACAGGAGACTGCTCTGCAACC
AATAGATGTCCCGGTTTTTGCTTGACAGGAGTGTATGCCGATGCCTGGTTA
CTGACCAACCCTTCGTCTACCAGTACATTTGGATCAGAAGCAACCTTCACT
GGTTCTTATCTCAACACAGCAACTCAGCGTATCAATCCGACGATGTATATC
GCGAACAACACACAGATCATAAGCTCACAGCAATTTGGATCAAGCGGTCA
AGAAGCAGCATATGGCCACACAACTTGTTTTAGGGACACAGGCTCTGTTA
TGGTATACTGTATCTATATTATTGAATTGTCCTCATCTCTCTTAGGACAATT
TCAGATTGTCCCATTTATCCGTCAGGTGACACTATCCTAAAGGCAGAAGCC
TTCAGGTCTGACCCAGCCAATCAAAGCATTATACCAGACCATGGCCTACC
ATCGGCTTTAAAGAAAAAAATAGGCCCGGACGGGTTAGCAACAAGCGGC
GGCCGCCACCatgttcgtcttcctggtcctgctgcctctggtctcctcacagtgcgtcaatctgatcactcggactca
gtcctatactaatagcttcaccagaggcgtgtactatcctgacaaggtgtttagaagctccgtgctgcactctacacaggatct
gtttctgccattctttagcaacgtgacctggttccacgccatcagcggcaccaatggcacaaagcggttcgacaatcccgtg
ctgccttttaacgatggcgtgtacttcgcctctaccgagaagagcaacatcatcagaggctggatctttggcaccacactgg
actccaagacacagtctctgctgatcgtgaacaatgccaccaacgtggtcatcaaggtgtgcgagttccagttttgtaatgat
cccttcctggacgtgtactatcacaagaacaataagagctggatggagtccgagtttagagtgtattctagcgccaacaact
gcacatttgagtacgtgagccagcctttcctgatggacctggagggcaagcagggcaatttcaagaacctgagggagttcg
tgtttaagaatatcgacggctacttcaaaatctactctaagcacacccccatcaacctgggccgcgacctgcctcagggcttc
agcgccctggagcccctggtggatctgcctatcggcatcaacatcacccggtttcagacactgctggccctgcacagaag
ctacctgacacccggcgactcctctagcggatggaccgccggcgctgccgcctactatgtgggctacctccagccccgga
ccttcctgctgaagtacaacgagaatggcaccatcacagacgcagtggattgcgccctggaccccctgagcgagacaaa
gtgtacactgaagtcctttaccgtggagaagggcatctatcagacatccaatttcagggtgcagccaaccgagtctatcgtg
cgctttcctaatatcacaaacctgtgcccatttgacgaggtgttcaacgcaacccgcttcgccagcgtgtacgcctggaatag
gaagcggatcagcaactgcgtggccgactatagcgtgctgtacaacctggcccccttcttcgcctttaagtgctatggcgtg
tcccccacaaagctgaatgacctgtgctttaccaacgtctacgccgattctttcgtgatcaggggcaacgaggtgtcccagat
cgcccccggccagacaggcaatatcgcagactacaattataagctgccagacgatttcaccggctgcgtgatcgcctgga
acagcaacaagctggattccaaagtgggcggcaactacaattatagataccggctgtttagaaagagcaatctgaagccct
tcgagagggacatctctacagaaatctaccaggccggcaataagccttgcaatggcgtggccggcgtgaactgttatttcc
cactccagtcctacggcttccgccccacatatggcgtgggccaccagccttaccgcgtggtggtgctgagctttgagctgct
gcacgccccagcaacagtgtgcggccccaagaagtccaccaatctggtgaagaacaagtgcgtgaacttcaacttcaacg
gcctgaccggcacaggcgtgctgaccgagtccaacaagaagttcctgccatttcagcagttcggcagggacatcgcagat
accacagacgccgtgcgcgacccacagaccctggagatcctggacatcacaccctgctctttcggcggcgtgagcgtgat
cacacccggcaccaatacaagcaaccaggtggccgtgctgtatcagggcgtgaattgtaccgaggtgcccgtggctatcc
acgccgatcagctgaccccaacatggcgggtgtacagcaccggctccaacgtcttccagacaagagccggatgcctgat
cggagcagagtacgtgaacaattcctatgagtgcgacatcccaatcggcgccggcatctgtgcctcttaccagacccagac
aaagtctcacagaagagcccggagcgtggcctcccagtctatcatcgcctataccatgtccctgggcgccgagaacagcg
tggcctactctaacaatagcatcgccatcccaaccaacttcacaatctctgtgaccacagagatcctgcccgtgtccatgacc
aagacatctgtggactgcacaatgtatatctgtggcgattctaccgagtgcagcaacctgctgctccagtacggcagcttttg
tacccagctgaagagagccctgacaggcatcgccgtggagcaggataagaacacacaggaggtgttcgcccaggtgaa
gcaaatctacaagaccccccctatcaagtactttggcggcttcaatttttcccagatcctgcctgatccatccaagccttctaag
cggagctttatcgaggacctgctgttcaacaaggtgaccctggccgatgccggcttcatcaagcagtatggcgattgcctg
ggcgacatcgcagccagggacctgatctgcgcccagaagtttaatggcctgaccgtgctgccacccctgctgacagatga
gatgatcgcacagtacacaagcgccctgctggccggcaccatcacatccggatggaccttcggcgcaggagccgccctc
cagatcccctttgccatgcagatggcctataggttcaacggcatcggcgtgacccagaatgtgctgtacgagaaccagaag
ctgatcgccaatcagtttaactccgccatcggcaagatccaggacagcctgtcctctacagccagcgccctgggcaagctc
caggatgtggtgaatcacaacgcccaggccctgaataccctggtgaagcagctgagcagcaagttcggcgccatctctag
cgtgctgaatgacatcctgagccggctggacaaggtggaggcagaggtgcagatcgaccggctgatcaccggccggct
ccagagcctccagacctatgtgacacagcagctgatcagggccgccgagatcagggccagcgccaatctggcagcaac
caagatgtccgagtgcgtgctgggccagtctaagagagtggacttttgtggcaagggctatcacctgatgtccttccctcagt
ctgccccacacggcgtggtgtttctgcacgtgacctacgtgcccgcccaggagaagaacttcaccacagcccctgccatct
gccacgatggcaaggcccactttccaagggagggcgtgttcgtgtccaacggcacccactggtttgtgacacagcgcaat
ttctacgagccccagatcatcaccacagacaacaccttcgtgagcggcaactgtgacgtggtcatcggcatcgtgaacaat
accgtgtatgatccactccagcccgagctggacagctttaaggaggagctggataagtatttcaagaatcacacctcccctg
acgtggatctgggcgacatcagcggcatcaatgcctccgtggtgaacatccagaaggagatcgaccgcctgaacgaggt
ggctaagaatctgaacgagagcctgatcgacctccaggagctgggcaagtatgagcagtacatcaagtggccctggtaca
tctggctgggcttcatcgccggcctgatcgccatcgtgatggtgaccatcatgctgtgctgtatgacatcctgctgttcttgcct
gaagggctgctgtagctgtggctcctgctgtaagttattgaccattgtcgttgctaatcgaaatagaatggagaattttgtttatc
ataaatgaAGTCGACTCATGGAATGCATACCAAACATTATTGACACTAATGAC
ACACAAAATTGGTTTTAAGAAAAACCAAGAGAACAATAGGCCAGAATGG
CTGGGTCTCGGGAGATATTACTCCCTGAAGTCCATCTCAATTCACCAATTG
TAAAGCATAAGCTATACTATTACATTCTACTTGGAAACCTCCCAAATGAG
ATCGACCTTGACGATTTAGGTCCATTACATAATCAAAATTGGAATCAGAT
AGCACATGAAGAGTCTAACTTAGCTCAACGCTTGGTAAATGTAAGAAATT
TTCTAATTACCCACATCCCTGATCTTAGAAAGGGCCATTGGCAAGAGTATG
TCAATGTAATACTGTGGCCGCGAATTCTTCCCTTGATCCCGGATTTTAAAA
TCAATGACCAATTGCCTCTGCTCAAAAATTGGGACAAGTTAGTTAAAGAA
TCATGTTCAGTAATCAATGCAGGTACTTCCCAGTGCATTCAGAATCTCAGC
TATGGACTGACAGGTCGTGGGAACCTCTTTACACGATCACGTGAACTCTCT
GGTGACCGCAGGGATATTGATCTTAAGACAGTTGTGGCAGCATGGCATGA
CTCAGACTGGAAAAGAATAAGTGATTTTTGGATTATGATCAAATTCCAGA
TGAGACAATTAATTGTTAGGCAAACAGATCATAATGATTCTGATTTAATCA
CGTATATCGAAAATAGAGAAGGCATAATCATCATAACCCCTGAACTGGTA
GCATTATTTAACACTGAGAATCATACACTAACATACATGACCTTTGAAATT
GTACTGATGGTTTCAGATATGTACGAAGGTCGTCACAACATTTTATCACTA
TGCACAGTTAGCACTTACCTGAATCCTCTGAAGAAAAGAATAACATATTT
ATTGAGCCTTGTAGATAACTTAGCTTTTCAGATAGGTGATGCTGTATATAA
CATAATTGCTTTGCTAGAATCCTTTGTATATGCACAGTTGCAAATGTCAGA
TCCCATCCCAGAACTCAGAGGACAATTCCATGCATTCGTATGTTCTGAGAT
TCTTGATGCACTAAGAGGAACTAATAGTTTCACCCAGGATGAATTAAGAA
CTGTGACAACTAATTTGATATCCCCATTCCAAGATCTGACCCCAGATCTTA
CGGCTGAATTGCTCTGTATAATGAGGCTTTGGGGACACCCCATGCTCACTG
CCAGTCAAGCTGCAGGAAAGGTACGCGAGTCTATGTGTGCTGGAAAAGTA
TTAGACTTTCCCACCATTATGAAAACACTAGCCTTTTTCCATACTATTCTG
ATCAATGGATACAGGAGGAAGCATCATGGAGTATGGCCACCCTTAAACTT
ACCGGGTAATGCTTCAAAGGGTCTCACGGAACTTATGAATGACAATACTG
AAATAAGCTATGAATTCACACTTAAGCATTGGAAGGAAGTCTCTCTTATA
AAATTCAAGAAATGTTTTGATGCAGACGCAGGTGAGGAACTCAGTATATT
TATGAAAGATAAGGCAATTAGTGCCCCAAAACAAGACTGGATGAGTGTGT
TTAGAAGAAGCCTAATCAAACAGCGCCATCAGCATCATCAGGTCCCCCTA
CCAAATCCATTCAATCGACGGCTGTTGCTAAACTTTCTCGGAGATGACAA
ATTCGACCCGAATGTGGAGCTACAGTATGTAACATCAGGTGAGTATCTAC
ATGATGACACGTTTTGTGCATCATATTCACTAAAAGAGAAGGAAATTAAA
CCTGATGGTCGAATTTTTGCAAAGTTGACTAAGAGAATGAGATCATGTCA
AGTTATAGCAGAATCTCTTTTAGCGAACCATGCTGGGAAGTTAATGAAAG
AGAATGGTGTTGTGATGAATCAGCTATCATTAACAAAATCACTATTAACA
ATGAGTCAGATTGGAATAATATCCGAGAAAGCTAGAAAGTCAACTCGAGA
TAACATAAATCAACCTGGTTTCCAGAATATCCAGAGAAATAAATCACATC
ACTCCAAGCAAGTCAATCAGCGAGATCCAAGTGATGACTTTGAATTGGCA
GCATCTTTTTTAACTACTGATCTCAAAAAATATTGTTTACAATGGAGGTAC
CAGACAATTATCCCATTTGCTCAATCATTAAACAGAATGTATGGTTATCCT
CATCTCTTTGAGTGGATTCACTTACGGCTAATGCGTAGTACACTTTACGTG
GGGGATCCCTTCAACCCACCAGCAGATACCAGTCAATTTGATCTAGATAA
AGTAATTAATGGAGATATCTTCATTGTATCACCCAGAGGTGGAATTGAAG
GGCTGTGTCAAAAGGCTTGGACAATGATATCTATCGCTGTGATAATTCTAT
CTGCCACAGAGTCTGGCACACGAGTAATGAGTATGGTGCAGGGAGATAAT
CAAGCAATTGCTGTCACCACACGAGTACCAAGGAGCCTGCCGACTCTTGA
GAAAAAGACTATTGCTTTTAGATCTTGTAATCTATTCTTTGAGAGGTTAAA
ATGTAATAATTTTGGATTAGGTCACCATTTGAAAGAACAAGAGACTATCA
TTAGTTCTCACTTCTTTGTTTATAGCAAGAGAATATTCTATCAGGGGAGGA
TTCTAACGCAAGCCTTAAAAAATGCTAGTAAGCTCTGCTTGACAGCTGAT
GTCCTAGGAGAATGCACCCAATCATCATGTTCTAATCTTGCAACTACTGTC
ATGAGGTTAACTGAGAATGGTGTTGAAAAAGATATCTGTTTCTACTTGAAT
ATCTATATGACCATCAAACAGCTCTCCTATGATATCATCTTCCCTCAAGTG
TCAATTCCTGGAGATCAGATCACATTAGAATACATAAATAATCCACACCT
GGTATCACGATTGGCTCTTTTGCCATCCCAGTTAGGAGGTCTAAACTACCT
GTCATGCAGTAGGCTGTTCAATCGAAACATAGGCGACCCGGTGGTTTCCG
CAGTTGCAGATCTTAAGAGATTAATTAAATCAGGATGTATGGATTACTGG
ATCCTTTATAACTTATTAGGGAGAAAACCGGGAAACGGCTCATGGGCTAC
TTTAGCAGCTGACCCGTACTCAATCAATATAGAGTATCAATACCCTCCAAC
TACAGCTCTTAAGAGGCACACCCAACAAGCTCTGATGGAACTCAGTACGA
ATCCAATGTTACGTGGCATATTCTCTGACAATGCACAGGCAGAAGAAAAT
AACCTTGCTAGGTTTCTCCTGGATAGGGAGGTGATCTTTCCGCGTGTAGCT
CACATCATCATTGAGCAAACCAGTGTCGGGAGGAGAAAACAGATTCAAG
GATATTTGGATTCAACTAGATCGATAATGAGGAAATCACTAGAAATTAAG
CCCTTATCCAATAGGAAGCTTAATGAAATACTGGATTACAACATCAATTA
CCTAGCTTACAATTTGGCATTACTCAAGAATGCTATTGAACCTCCGACTTA
TTTGAAGGCAATGACACTTGAAACATGTAGCATCGACATTGCAAGGAACC
TCCGGAAGCTCTCCTGGGCCCCACTCTTGGGTGGGAGAAATCTTGAAGGA
TTAGAGACGCCAGATCCCATTGAAATTACTGCAGGAGCATTAATTGTTGG
ATCGGGCTACTGTGAACAGTGTGCTGCAGGAGACAATCGATTCACATGGT
TTTTCTTGCCATCTGGTATCGAGATAGGAGGGGATCCCCGTGATAATCCTC
CTATCCGTGTACCGTACATTGGCTCCAGGACTGATGAGAGGAGGGTAGCC
TCAATGGCATACATCAGGGGTGCCTCGAGTAGCTTAAAAGCAGTTCTTAG
ACTGGCGGGAGTGTACATCTGGGCATTCGGAGATACTCTGGAGAATTGGA
TAGATGCACTGGATTTGTCTCACACTAGAGTTAACATCACACTTGAACAGC
TGCAATCCCTCACCCCACTTCCAACCTCTGCCAATCTAACCCATCGGTTGG
ATGATGGCACAACTACCCTAAAGTTTACTCCTGCGAGCTCTTACACCTTTT
CAAGTTTCACTCATATATCAAATGATGAGCAATACCTGACAATTAATGAC
AAAACTGCAGATTCAAATATAATCTACCAACAGTTAATGATCACTGGACT
CGGAATCTTAGAAACATGGAATAATCCCCCAATCAATAGAACATTCGAAG
AATCTACCCTACATTTGCACACTGGTGCATCATGTTGTGTCCGACCTGTGG
ACTCCTGCATTCTCTCAGAAGCATTAACAGTCAAGCCACATATTACAGTAC
CGTACAGCAATAAATTTGTATTTGATGAGGACCCGCTATCTGAATATGAA
ACTGCAAAACTGGAATCGTTATCATTCCAAGCCCAATTAGGCAACATTGA
TGCTGTAGATATGACAGGTAAATTAACATTATTGTCCCAATTCACTGCAAG
GCAGATTATCAATGCAATCACTGGACTCGATGAGTCTGTCTCTCTTACTAA
TGATGCCATTGTTGCATCAGACTATGTCTCCAATTGGATTAGTGAATGCAT
GTATACCAAATTAGATGAATTATTTATGTATTGTGGGTGGGAACTACTATT
GGAACTATCCTATCAAATGTATTATCTGAGGGTAGTTGGGTGGAGTAATA
TAGTGGATTATTCTTACATGATCTTGAGAAGAATCCCGGGTGCAGCATTAA
ACAATCTGGCATCTACATTAAGTCATCCAAAACTTTTCCGACGAGCTATCA
ACCTAGATATAGTTGCCCCCTTAAATGCTCCTCATTTTGCATCTCTGGACT
ACATCAAGATGAGTGTGGATGCAATACTCTGGGGCTGTAAAAGAGTCATC
AATGTGCTCTCCAATGGAGGGGACTTAGAATTAGTTGTGACATCTGAAGA
TAGCCTTATTCTCAGTGACCGATCCATGAATCTCATTGCAAGGAAATTAAC
TTTATTATCACTGATTCACCATAATGGTTTGGAACTACCAAAGATTAAGGG
GTTCTCTCCTGATGAGAAGTGTTTCGCTTTGACAGAATTTTTGAGGAAAGT
GGTGAACTCAGGGTTGAGTTCAATAGAGAACCTATCAAATTTTATGTACA
ATGTGGAGAACCCACGGCTTGCAGCATTCGCCAGCAACAATTACTACCTG
ACCAGAAAATTATTGAATTCAATACGAGATACTGAGTCGGGTCAAGTAGC
AGTCACCTCATATTATGAATCATTAGAATATATTGATAGTCTTAAGCTAAC
CCCACATGTGCCTGGCACCTCATGCATTGAGGATGATAGTCTATGTACAA
ATGATTACATAATCTGGATCATAGAGTCTAATGCAAACTTGGAGAAGTAT
CCAATTCCAAATAGCCCTGAGGATGATTCCAATTTCCATAACTTTAAGTTG
AATGCTCCATCGCACCATACCTTACGCCCATTAGGGTTGTCATCAACTGCT
TGGTATAAGGGTATAAGCTGCTGCAGGTACCTTGAGCGATTAAAGCTACC
ACAAGGTGATCATTTATATATTGCAGAAGGTAGTGGTGCCAGTATGACAA
TCATAGAATACCTATTCCCAGGAAGAAAGATATATTACAATTCTTTATTTA
GTAGTGGTGACAATCCCCCACAAAGAAATTATGCACCAATGCCTACTCAG
TTCATTGAGAGTGTCCCATACAAGCTCTGGCAAGCACACACAGATCAATA
TCCCGAGATTTTTGAGGACTTCATCCCTCTATGGAACGGAAACGCCGCCAT
GACTGACATAGGAATGACAGCTTGTGTAGAATTCATCATCAATCGAGTCG
GCCCAAGGACTTGCAGTTTAGTACATGTAGATTTGGAATCAAGTGCAAGC
TTAAATCAACAATGCCTGTCAAAGCCGATAATTAATGCTATCATCACTGCT
ACAACTGTTTTGTGCCCTCATGGGGTGCTTATTCTGAAATATAGTTGGTTG
CCATTTACTAGATTTAGTACTTTGATCACTTTCTTATGGTGCTACTTTGAGA
GAATCACTGTTCTTAGGAGCACATATTCTGATCCAGCTAATCATGAGGTTT
ATTTAATTTGTATCCTTGCCAACAACTTTGCATTCCAGACTGTCTCGCAGG
CAACAGGAATGGCGATGACTTTAACTGATCAAGGGTTTACTTTGATATCA
CCTGAAAGAATAAATCAGTATTGGGATGGTCACTTGAAGCAAGAACGTAT
CGTAGCAGAAGCAATTGATAAGGTGGTTCTAGGAGAAAATGCTCTATTTA
ATTCGAGTGATAATGAATTAATTCTCAAATGTGGAGGGACACCAAATGCA
CGGAATCTCATCGATATCGAGCCAGTCGCAACTTTCATAGAATTTGAACA
ATTGATCTGCACAATGTTGACAACCCACTTGAAGGAAATAATTGATATAA
CAAGGTCTGGAACCCAGGATTATGAAAGTTTATTACTCACTCCTTACAATT
TAGGTCTTCTTGGTAAAATCAGTACGATAGTGAGATTATTAACAGAAAGG
ATTCTAAATCATACTATCAGGAATTGGTTGATCCTCCCACCTTCGCTCCGG
ATGATCGTGAAGCAGGACTTGGAATTCGGCATATTCAGGATTACTTCCATC
CTCAATTCTGATCGGTTCCTGAAGCTTTCTCCAAATAGGAAATACTTGATT
GCACAATTAACTGCAGGCTACATTAGGAAATTGATTGAGGGGGATTGCAA
TATCGATCTAACCAGACCTATCCAAAAGCAAATCTGGAAAGCATTAGGTT
GTGTAGTCTATTGTCACGATCCAATGGATCAAAGGGAGTCAACAGAGTTT
ATTGATATAAATATTAATGAAGAAATAGACCGCGGGATCGATGGCGAGGA
AATCTAAACATATCAAGAATCAGAATTAGTTTAAGAAAAAAGAAGAGGA
TTAATCTTGGTTTTCCCCTTGGTGGGTCGGCATGGCATCTCCACCTCCTCGC
GGTCCGACCTGGGCATCCGAAGGAGGACGCACGTCCACTCGGATGGCTAA
GGGAGCGGCCGGGGATCCGGCTGCTAACAAAGCCCGAAAGGAAGCTGAG
TTGGCTGCTGCCACCGCTGAGCAATAACTAGCATAACCCCTTGGGGCCTCT
AAACGGGTCTTGAGGGGTTTTTTGCTGAAAGGAGGAACTATATCCGGATC
ATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATC
GACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAA
GCTGGGCTGTGTGCACGAACCCCCCGTTCAGCCCGACCGCTGCGCCTTATC
CGGTAACTATCGTCTTGAGTCCAACCCGGTAAGACACGACTTATCGCCACT
GGCAGCAGCCACTGGTAACAGGATTAGCAGAGCGAGGTATGTAGGCGGT
GCTACAGAGTTCTTGAAGTGGTGGCCTAACTACGGCTACACTAGAAGAAC
AGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTT
TGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTT
AAGGGATTTTGGTCATGAGATTATCAAAAAGGATCTTCACCTAGATCCTTT
TAAATTAAAAATGAAGTTTTAAATCAATCTAAAGTATATATGAGTAAACT
TGGTCTGACAGTTACCAATGCTTAATCAGTGAGGCACCTATCTCAGCGATC
TGTCTATTTCGTTCATCCATAGTTGCCTGACTCCCCGTCGTGTAGATAACT
ACGATACGGGAGGGCTTACCATCTGGCCCCAGTGCTGCAATGATACCGCG
AGACCCACGCTCACCGGCTCCAGATTTATCAGCAATAAACCAGCCAGCCG
GAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGT
TTGCGCAACGTTGTTGCCATTGCTACAGGCATCGTGGTGTCACGCTCGTCG
TTTGGTATGGCTTCATTCAGCTCCGGTTCCCAACGATCAAGGCGAGTTACA
TGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGC
ACTGCATAATTCTCTTACTGTCATGCCATCCGTAAGATGCTTTTCTGTGACT
GGTGAGTACTCAACCAAGTCATTCTGAGAATAGTGTATGCGGCGACCGAG
TTGCTCTTGCCCGGCGTCAATACGGGATAATACCGCGCCACATAGCAGAA
CTTTAAAAGTGCTCATCATTGGAAAACGTTCTTCGGGGCGAAAACTCTCA
AGGATCTTACCGCTGTTGAGATCCAGTTCGATGTAACCCACTCGTGCACCC
AACTGATCTTCAGCATCTTTTACTTTCACCAGCGTTTCTGGGTGAGCAAAA
ACAGGAAGGCAAAATGCCGCAAAAAAGGGAATAAGGGCGACACGGAAAT
GTTGAATACTCATACTCTTCCTTTTTCAATATTATTGAAGCATTTATCAGGG
TTATTGTCTCATGAGCGGATACATATTTGAATGTATTTAGAAAAATAAACA
AATAGGGGTTCCGCGCACATTTCCCCGAAAAGT.

vi. CVL110

The nucleic acid sequence for CVL110 is:

(SEQ ID NO: 8)
GCCACCTGACGTCTAAGAAACCATTATTATCATGACATTAACCTA
TAAAAATAGGCGTATCACGAGGCCCTTTCGTCTCGCGCGTTTCGG
TGATGACGGTGAAAACCTCTGACACATGCAGCTCCCGGAGACGGT
CACAGCTTGTCTGTAAGCGGATGCCGGGAGCAGACAAGCCCGTCA
GGGCGCGTCAGCGGGTGTTGGCGGGTGTCGGGGCTGGCTTAACTA
TGCGGCATCAGAGCAGATTGTACTGAGAGTGCACCATATATGCGG
TGTGAAATACCGCACAGATGCGTAAGGAGAAAATACCGCATCAGG
CTAATACGACTCACTATAGGGACCAAGGGGAAAATGAAGTGGTGA
CTCAAATCATCGAAGACCCTCGAGATTACATAGGTCCGGAACCTA
TGGCCTTCGTGACCGACCTCGAGTCAGAGTAGTTCAATAAGGACC
TATCAAGTTTGGGCAATTTTTCGTCCCCGACACAAAAATGTCATC
CGTGCTTAAAGCATATGAGCGATTCACGCTCACTCAAGAACTGCA
AGATCAGAGTGAGGAAGGTACAATCCCACCTACAACACTAAAACC
GGTAATCAGGGTATTTATACTAACCTCTAATAACCCAGAGCTAAG
ATCCCGGCTTCTTCTATTCTGCCTACGGATTGTTCTCAGTAATGG
TGCAAGGGATTCCCATCGCTTTGGAGCATTACTCACAATGTTTTC
GCTACCATCAGCCACAATGCTCAATCATGTCAAATTAGCTGACCA
GTCACCAGAAGCTGATATCGAAAGGGTAGAGATCGATGGCTTTGA
GGAGGGATCATTCCGCTTAATCCCCAATGCACGTTCAGGTATGAG
CCGTGGAGAGATCAATGCCTATGCTGCACTTGCAGAAGATCTACC
TGACACACTAAACCATGCAACACCTTTCGTTGATTCCGAAGTCGA
GGGAACTGCATGGGATGAGATTGAGACTTTCTTAGATATGTGTTA
CAGTGTCCTAATGCAGGCATGGATAGTGACTTGCAAGTGCATGAC
TGCGCCAGACCAACCTGCTGCTTCTATTGAGAAACGCCTGCAAAA
ATATCGTCAGCAAGGCAGGATCAACCCGAGATATCTCCTGCAACC
GGAGGCTCGACGAATAATCCAGAATGTAATCCGGAAGGGAATGGT
GGTCAGACATTTCCTCACCTTTGAACTGCAGCTTGCCCGAGCACA
AAGCCTTGTATCAAATAGGTATTATGCTATGGTAGGGGATGTTGG
AAAGTATATAGAGAATTGTGGAATGGGAGGCTTCTTTTTGACACT
AAAATATGCATTAGGAACTAGATGGCCCACACTTGCTTTAGCTGC
ATTTTCAGGAGAGCTAACAAAGCTAAAGTCCCTCATGGCATTATA
CCAGACCCTTGGTGAGCAGGCCCGATATTTGGCCCTATTGGAGTC
ACCACATTTGATGGATTTTGCTGCAGCAAACTACCCACTGCTATA
TAGCTATGCTATGGGAATAGGCTATGTGTTAGATGTCAACATGAG
GAACTACGCTTTCTCCAGATCATACATGAACAAGACATATTTCCA
ATTGGGAATGGAAACTGCAAGAAAACAACAGGGTGCAGTTGACAT
GAGGATGGCAGAAGATCTCGGTCTAACTCAAGCCGAACGCACCGA
GATGGCAAATACACTTGCCAAATTGACCACAGCAAATCGAGGGGC
AGACACCAGGGGAGGAGTCAACCCGTTCTCATCTGTCACTGGGAC
AACTCAGGTGCCCGCTGCAGCAACAGGTGACACACTCGAGAGTTA
CATGGCAGCGGATCGACTGAGGCAGAGATATGCTGATGCAGGCAC
CCATGATGATGAGATGCCACCATTGGAAGAGGAGGAAGAGGACGA
CACATCTGCAGGTCCACGCACTGGACCAACTCTTGAACAAGTGGC
CTTGGACATCCAGAACGCAGCAGTTGGAGCTCCCATCCATACAGA
TGACCTGAATGCCGCACTGGGTGATCTTGACATCTAGACAATTCA
GATCCCAATCTAAAATTGACATACCTAATTGATTAGTTAGATGGA
ACTACAGTGGATTCCATAAGGTTCCTGCCTACCATCGGCTTTAAA
GAAAAAAATAGGCCCGGACGGGTTAGCAACAAGCGACTGCCGGTG
CCAACAGCGCAATCCACAATCTACAATGGATCCCACTGATCTGAG
CTTCTCCCCAGATGAGATCAATAAGCTCATAGAGACAGGCCTGAA
TACTGTAGAGTATTTTACTTCCCAACAAGTCACAGGAACATCCTC
TCTTGGAAAGAATACAATACCACCAGGGGTCACAGGACTACTAAC
CAATGCTGCAGAGGCAAAGATCCAAGAGTCAACTAACCATCAGAA
GGGCTCAGTTGGTGGGGGTGCAAAACCAAAGAAACCGCGACCAAA
AATTGCCATTGTGCCAGCAGATGACAAAACAGTGCCCGGAAAGCC
GATCCCAAACCCTCTATTAGGTCTGGACTCCACCCCGAGCACCCA
AACTGTGCTTGATCTAAGTGGGAAAACATTACCATCAGGATCCTA
TAAGGGGGTTAAGCTTGCGAAATTTGGAAAAGAAAATCTGATGAC
ACGGTTCATCGAGGAACCCAGAGAGAATCCTATCGCAACCAGTTt
CCCCATCGATTTTAAGAGGGGCAGGGATACCGGCGGGTTCCATAG
AAGGGAGTACTCAATCGGATGGGTGGGAGATGAAGTCAAGGTCAC
TGAGTGGTGCAATCCATCCTGTTCTCCAATCACCGCTGCAGCAAG
GCGATTTGAATGCACTTGTCACCAGTGTCCAGTCACTTGCTCTGA
ATGTGAACGAGATACTTAATACAGTGAGAAATTTGGACTCTCGGA
TGAATCAACTGGAGACAAAAGTAGATCGCATTCTCTCATCTCAGT
CTCTAATCCAGACCATCAAGAATGACATAGTTGGACTTAAAGCAG
GGATGGCTACTTTAGAAGGAATGATTACAACTGTGAAAATCATGG
ACCCGGGAGTTCCCAGTAATGTTACTGTGGAAGATGTACGCAAGA
CACTAAGTAACCATGCTGTTGTTGTGCCAGAATCATTCAATGATA
GTTTCTTGACTCAATCTGAAGATGTAATTTCACTTGATGAGTTGG
CTCGACCAACTGCAACAAGTGTTAAGAAGATTGTCAGGAAGGTTC
CTCCTCAGAAGGATCTGACTGGATTGAAGATTACACTAGAGCAAT
TGGCAAAGGATTGCATCAGCAAACCGAAGATGAGGGAAGAGTATC
TCCTCAAAATCAACCAGGCTTCCAGTGAGGCTCAGCTAATTGACC
TCAAGAAAGCAATCATCCGCAGTGCAATTTGATCAAGAAACACCC
AATTACACTACACTGGTATGACACTGTACTAACCCTGAGGGTTTT
AGAAAAAACGATTAACGATAAATAAGCCCGAACACTACACACTAC
CTGAGGCAGCCATGCCATCCATCAGCATTCCCGCAGACCCCACCA
ATCCACGTCAATCAATAAAAGCGTTCCCAATTGTGATCAACAGTG
ATGGGGGTGAGAAAGGCCGCTTGGTTAAACAACTACGCACAACCT
ACTTGAATGACCTAGATACTCATGAGCCACTGGTGACATTCATAA
ATACCTATGGATTCATCTACGAACAGGATCGGGGGAATACCATTG
TCGGAGAGGATCAACTTGGGAAGAAAAGAGAGGCTGTGACCGCTG
CAATGGTTACCCTTGGATGTGGGCCTAATCTACCATCATTAGGGA
ATGTCCTGGGACAACTGAGGGAATTCCAGGTCACTGTTAGGAAGA
CATCCAGCAAAGCGGAAGAGATGGTCTTTGAAATTGTTAAGTATC
CGAGAATATTTCGGGGTCATACATTAATCCAGAAAGGACTAGTCT
GTGTCTCCGCAGAAAAATTTGTTAAGTCACCAGGGAAAATACAAT
CTGGAATGGACTATCTCTTCATTCCGACATTTCTGTCAGTGACTT
ACTGTCCAGCTGCAATCAAATTTCAGGTACCTGGCCCCATGTTGA
AAATGAGATCAAGATACACTCAGAGCTTACAACTTGAACTAATGA
TAAGAATCCTGTGTAAGCCCGATTCGCCACTTATGAAGGTCCATA
CCCCTGACAAGGAGGGAAGAGGATGTCTTGTATCAGTATGGCTGC
ATGTATGCAACATCTTCAAATCAGGAAACAAGAATGGCAGTGAGT
GGCAGGAATACTGGATGAGAAAGTGTGCTAACATGCAACTTGAAG
TGTCGATTGCAGATATGTGGGGACCAACTATCATAATTCATGCCA
GAGGTCACATTCCCAAAAGTGCTAAGTTGTTTTTTGGAAAGGGTG
GATGGAGCTGCCATCCACTTCACGAAGTTGTTCCAAGTGTCACTA
AAACACTATGGTCCGTGGGCTGTGAGATTACAAAGGCGAAGGCAA
TAATACAAGAGAGTAGCATCTCTCTTCTCGTGGAGACTACTGACA
TCATAAGTCCAAAAGTCAAAATTTCATCTAAGCATCGCCGCTTTG
TGAAATCAAATTGGGGTCTGTTCAAGAAAACTAAATCACTGCCTA
ACCTGACGGAGCTGGAATGACTGACCTCTAATCGAGACTACACCG
CCGCAAACTATAGGTGGGTGGTACCTCAGTGATTAATCTTGTAAG
CACTGACCGTAGGCTACAACACACTAATATTATCCAGATTAGAGA
GCTTAATTAGCTCTGTATTAATAATAACACTACTATTCCAATAAC
TGGAATCACCAGCTTGATTTATCTCCAAAATGATTCAAAGAAAAC
AAATCATATTAAGACTATCCTAAGCACGAACCCATATCGTCCTTC
AAATCATGGGTACTATAATTCAATTTCTGGTGGTCTCCTGTCTAT
TGGCAGGAGCAGGCAGCCTTGATCCAGCAGCCCTCATGCAAATCG
GTGTCATTCCAACAAATGTCCGGCAACTTATGTATTATACTGAAG
CCTCATCAGCATTCATTGTTGTGAAGTTAATGCCTACAATTGACT
CGCCGATTAGTGGATGTAATATAACATCAATTTCAAGCTATAATG
CAACAGTGACAAAACTCCTACAGCCGATCGGTGAGAATTTGGAGA
CGATTAGGAACCAGTTGATTCCAACTCGGAGGAGACGCCGGTTTG
CAGGGGTGGTGATTGGATTAGCTGCATTAGGAGTAGCTACTGCCG
CACAGGTCACTGCCGCAGTGGCACTAGTAAAGGCAAATGAAAATG
CTGCGGCTATACTCAATCTCAAAAATGCAATCCAAAAAACAAATG
CAGCAGTTGCAGATGTGGTCCAGGCCACACAATCACTAGGAACGG
CAGTTCAAGCAGTTCAAGATCACATAAACAGTGTGGTAAGTCCAG
CAATTACAGCAGCCAATTGTAAGGCCCAAGATGCTATCATTGGCT
CAATCCTCAATCTCTATTTGACCGAGTTGACAACCATCTTCCACA
ATCAAATTACAAACCCTGCATTGAGTCCCATTACAATTCAAGCTT
TAAGGATCCTACTGGGGAGTACCTTGCCGACTGTGGTCGAAAAAT
CTTTCAATACCCAGATAAGTGCAGCTGAGCTTCTCTCATCAGGGT
TATTGACAGGCCAGATTGTGGGATTAGATTTGACCTATATGCAGA
TGGTCATAAAAATTGAGCTGCCAACTTTAACTGTACAACCTGCAA
CCCAGATCATAGATCTGGCCACCATTTCTGCATTCATTAACAATC
AAGAAGTCATGGCCCAATTACCAACACGTGTTATGGTGACTGGCA
GCTTGATCCAAGCCTATCCCGCATCGCAATGCACCATTACACCCA
ACACTGTGTACTGTAGGTATAATGATGCCCAAGTACTCTCAGATG
ATACTATGGCTTGCCTCCAAGGTAACTTGACAAGATGCACCTTCT
CTCCAGTGGTTGGGAGCTTTCTCACTCGATTCGTGCTGTTCGATG
GAATAGTTTATGCAAATTGCAGGTCGATGTTGTGCAAGTGCATGC
AACCTGCTGCTGTGATCCTACAGCCGAGTTCATCCCCTGTAACTG
TCATTGACATGTACAAATGTGTGAGTCTGCAGCTTGACAATCTCA
GATTCACCATCACTCAATTGGCCAATGTAACCTACAATAGCACCA
TCAAGCTTGAATCATCCCAGATCTTGTCTATTGATCCGTTGGATA
TATCCCAAAATCTAGCTGCGGTGAATAAGAGTCTAAGTGATGCAC
TACAACACTTAGCACAAAGTGACACATATCTTTCTGCAATCACAT
CAGCTACGACTACAAGTGTATTATCCATAATAGCAATCTGTCTTG
GATCGTTAGGTTTAATATTAATAATCTTGCTCAGTGTAGTTGTGT
GGAAGTTATTGACCATTGTCGTTGCTAATCGAAATAGAATGGAGA
ATTTTGTTTATCATAAATAAGCATTCCACCACTCACGATCTGATC
TCAGTGAGAAAAATCAACCTGCAACTCTTGGAACAAGATAAGACA
GTCATCCATTAGTAATTTTTAAGAAAAAAACGATAGGACCGAACC
TAGTATTGAAAGAACCGTCTCGGTCAATCTAGGTAATCGAGCTGA
TACCGTCTCGGAAAGCTCAAATCAGCTAGCCGGCAGTGCCACTGC
ACACACAACACTACACATACAATACACTACAATGGTTGCAGAAGA
TGCCCCTGTTAGGGCCACTTGCCGAGTATTATTTCGAACAACAAC
TTTAATCTTTCTATGCACACTACTAGCATTAAGCATCTCTATCCT
TTATGAGAGTTTAATAACCCAAAAGCAAATCATGAGCCAAGCAGG
CTCAACTGGATCTAATTCTGGATTAGGAAGTATCACTGATCTTCT
TAATAATATTCTCTCTGTCGCAAATCAGATTATATATAACTCTGC
AGTCGCTCTACCTCTACAATTGGACACTCTTGAATCAACACTCCT
TACAGCCATTAAGTCTCTTCAAACCAGTGACAAGCTAGAACAGAA
CTGCTCGTGGAGTGCTGCACTGATTAATGATAATAGATACATTAA
TGGCATCAATCAGTTCTATTTTTCAATTGCTGAGGGTCGCAATCT
GACACTTGGCCCACTTCTTAATATGCCTAGTTTCATTCCAACTGC
CACGACACCAGAGGGCTGCACCAGGATCCCATCATTCTCGCTCAC
TAAGACACACTGGTGTTATACACACAATGTTATCCTGAATGGATG
CCAGGATCATGTATCCTCAAATCAATTTGTTTCTATGGGAATCAT
TGAACCCACTTCTGCCGGGTTTCCATTCTTTCGAACCCTAAAGAC
TCTATATCTCAGCGATGGGGTCAATCGTAAGAGCTGCTCTATCAG
TACAGTTCCGGGGGGTTGTATGATGTACTGTTTTGTTTCTACTCA
ACCAGAGAGGGATGACTACTTTTCTGCCGCTCCTCCAGAACAACG
AATTATTATAATGTACTATAATGATACAATCGTGGAGCGCATAAT
TAATCCACCCGGGGTACTAGATGTATGGGCAACATTGAACCCAGG
AACAGGAAGCGGGGTATATTATTTAGGTTGGGTGCTCTTTCCAAT
ATATGGCGGCGTGATTAAAGGTACGAGTTTATGGAATAATCAAGC
AAATAAATACTTTATCCCCCAGATGGTTGCTGCTCTCTGCTCACA
AAACCAGGCAACTCAAGTCCAAAATGCTAAGTCATCATACTATAG
CAGCTGGTTTGGCAATCGAATGATTCAGTCTGGGATCCTGGCATG
TCCTCTTCGACAGGATCTAACCAATGAGTGTTTAGTTCTGCCCTT
TTCTAATGATCAGGTGCTTATGGGTGCTGAAGGGAGATTATACAT
GTATGGTGACTCGGTGTATTACTATCAAAGAAGCAATAGTTGGTG
GCCTATGACCATGCTGTATAAGGTAACCATAACATTCACTAATGG
TCAGCCATCTGCTATATCAGCTCAGAATGTGCCCACACAGCAGGT
CCCTAGACCTGGGACAGGAGACTGCTCTGCAACCAATAGATGTCC
CGGTTTTTGCTTGACAGGAGTGTATGCCGATGCCTGGTTACTGAC
CAACCCTTCGTCTACCAGTACATTTGGATCAGAAGCAACCTTCAC
TGGTTCTTATCTCAACACAGCAACTCAGCGTATCAATCCGACGAT
GTATATCGCGAACAACACACAGATCATAAGCTCACAGCAATTTGG
ATCAAGCGGTCAAGAAGCAGCATATGGCCACACAACHTGTTTTAG
GGACACAGGCTCTGTTATGGTATACTGTATCTATATTATTGAATT
GTCCTCATCTCTCTTAGGACAATTTCAGATTGTCCCATTTATCCG
TCAGGTGACACTATCCTAAAGGCAGAAGCCTTCAGGTCTGACCCA
GCCAATCAAAGCATTATACCAGACCATGGCCTACCATCGGCTTTA
AAGAAAAAAATAGGCCCGGACGGGTTAGCAACAAGCGcGCGGCCG
CCACCatgttcgtcttcctggtcctgctgcctctggtctcctcac
agtgcgtcaatctgatcactcggactcagtcctatactaatagct
tcaccagaggcgtgtactatcctgacaaggtgtttagaagctccg
tgctgcactctacacaggatctgtttctgccattctttagcaacg
tgacctggttccacgccatcagcggcaccaatggcacaaagcggt
tcgacaatcccgtgctgccttttaacgatggcgtgtacttcgcct
ctaccgagaagagcaacatcatcagaggctggatctttggcacca
cactggactccaagacacagtctctgctgatcgtgaacaatgcca
ccaacgtggtcatcaaggtgtgcgagttccagttttgtaatgatc
ccttcctggacgtgtactatcacaagaacaataagagctggatgg
agtccgagtttagagtgtattctagcgccaacaactgcacatttg
agtacgtgagccagcctttcctgatggacctggagggcaagcagg
gcaatttcaagaacctgagggagttcgtgtttaagaatatcgacg
gctacttcaaaatctactctaagcacacccccatcaacctgggcc
gcgacctgcctcagggcttcagcgccctggagcccctggtggatc
tgcctatcggcatcaacatcacccggtttcagacactgctggccc
tgcacagaagctacctgacacccggcgactcctctagcggatgga
ccgccggcgctgccgcctactatgtgggctacctccagccccgga
ccttcctgctgaagtacaacgagaatggcaccatcacagacgcag
tggattgcgccctggaccccctgagcgagacaaagtgtacactga
agtcctttaccgtggagaagggcatctatcagacatccaatttca
gggtgcagccaaccgagtctatcgtgcgctttcctaatatcacaa
acctgtgcccatttgacgaggtgttcaacgcaacccgcttcgcca
gcgtgtacgcctggaataggaagcggatcagcaactgcgtggccg
actatagcgtgctgtacaacctggcccccttcttcgcctttaagt
gctatggcgtgtcccccacaaagctgaatgacctgtgctttacca
acgtctacgccgattctttcgtgatcaggggcaacgaggtgtccc
agatcgcccccggccagacaggcaatatcgcagactacaattata
agctgccagacgatttcaccggctgcgtgatcgcctggaacagca
acaagctggattccaaagtgggcggcaactacaattatagatacc
ggctgtttagaaagagcaatctgaagcccttcgagagggacatct
ctacagaaatctaccaggccggcaataagccttgcaatggcgtgg
ccggcgtgaactgttatttcccactccagtcctacggcttccgcc
ccacatatggcgtgggccaccagccttaccgcgtggtggtgctga
gctttgagctgctgcacgccccagcaacagtgtgcggccccaaga
agtccaccaatctggtgaagaacaagtgcgtgaacttcaacttca
acggcctgaccggcacaggcgtgctgaccgagtccaacaagaagt
tcctgccatttcagcagttcggcagggacatcgcagataccacag
acgccgtgcgcgacccacagaccctggagatcctggacatcacac
cctgctctttcggcggcgtgagcgtgatcacacccggcaccaata
caagcaaccaggtggccgtgctgtatcagggcgtgaattgtaccg
aggtgcccgtggctatccacgccgatcagctgaccccaacatggc
gggtgtacagcaccggctccaacgtcttccagacaagagccggat
gcctgatcggagcagagtacgtgaacaattcctatgagtgcgaca
tcccaatcggcgccggcatctgtgcctcttaccagacccagacaa
agtctcacagaagagcccggagcgtggcctcccagtctatcatcg
cctataccatgtccctgggcgccgagaacagcgtggcctactcta
acaatagcatcgccatcccaaccaacttcacaatctctgtgacca
cagagatcctgcccgtgtccatgaccaagacatctgtggactgca
caatgtatatctgtggcgattctaccgagtgcagcaacctgctgc
tccagtacggcagcttttgtacccagctgaagagagccctgacag
gcatcgccgtggagcaggataagaacacacaggaggtgttcgccc
aggtgaagcaaatctacaagaccccccctatcaagtactttggcg
gcttcaatttttcccagatcctgcctgatccatccaagccttcta
agcggagctttatcgaggacctgctgttcaacaaggtgaccctgg
ccgatgccggcttcatcaagcagtatggcgattgcctgggcgaca
tcgcagccagggacctgatctgcgcccagaagtttaatggcctga
ccgtgctgccacccctgctgacagatgagatgatcgcacagtaca
caagcgccctgctggccggcaccatcacatccggatggaccttcg
gcgcaggagccgccctccagatcccctttgccatgcagatggcct
ataggttcaacggcatcggcgtgacccagaatgtgctgtacgaga
accagaagctgatcgccaatcagtttaactccgccatcggcaaga
tccaggacagcctgtcctctacagccagcgccctgggcaagctcc
aggatgtggtgaatcacaacgcccaggccctgaataccctggtga
agcagctgagcagcaagttcggcgccatctctagcgtgctgaatg
acatcctgagccggctggacaaggtggaggcagaggtgcagatcg
accggctgatcaccggccggctccagagcctccagacctatgtga
cacagcagctgatcagggccgccgagatcagggccagcgccaatc
tggcagcaaccaagatgtccgagtgcgtgctgggccagtctaaga
gagtggacttttgtggcaagggctatcacctgatgtccttccctc
agtctgccccacacggcgtggtgtttctgcacgtgacctacgtgc
ccgcccaggagaagaacttcaccacagcccctgccatctgccacg
atggcaaggcccactttccaagggagggcgtgttcgtgtccaacg
gcacccactggtttgtgacacagcgcaatttctacgagccccaga
tcatcaccacagacaacaccttcgtgagcggcaactgtgacgtgg
tcatcggcatcgtgaacaataccgtgtatgatccactccagcccg
agctggacagctttaaggaggagctggataagtatttcaagaatc
acacctcccctgacgtggatctgggcgacatcagcggcatcaatg
cctccgtggtgaacatccagaaggagatcgaccgcctgaacgagg
tggctaagaatctgaacgagagcctgatcgacctccaggagctgg
gcaagtatgagcagtacatcaagtggccctggtacatctggctgg
gcttcatcgccggcctgatcgccatcgtgatggtgaccatcatgc
tgtgctgtatgacatcctgctgttcttgcctgaagggctgctgta
gctgtggctcctgctgtaagttattgaccattgtcgttgctaatc
gaaatagaatggagaattttgtttatcataaatgaACGCGTGTCG
ACTCATGGAATGCATACCAAACATTATTGACACTAATGACACACA
AAATTGGTTTTAAGAAAAACCAAGAGAACAATAGGCCAGAATGGC
TGGGTCTCGGGAGATATTACTCCCTGAAGTCCATCTCAATTCACC
AATTGTAAAGCATAAGCTATACTATTACATTCTACTTGGAAACCT
CCCAAATGAGATCGACCTTGACGATTTAGGTCCATTACATAATCA
AAATTGGAATCAGATAGCACATGAAGAGTCTAACTTAGCTCAACG
CTTGGTAAATGTAAGAAATTTTCTAATTACCCACATCCCTGATCT
TAGAAAGGGCCATTGGCAAGAGTATGTCAATGTAATACTGTGGCC
GCGAATTCTTCCCTTGATCCCGGATTTTAAAATCAATGACCAATT
GCCTCTGCTCAAAAATTGGGACAAGTTAGTTAAAGAATCATGTTC
AGTAATCAATGCAGGTACTTCCCAGTGCATTCAGAATCTCAGCTA
TGGACTGACAGGTCGTGGGAACCTCTTTACACGATCACGTGAACT
CTCTGGTGACCGCAGGGATATTGATCTTAAGACAGTTGTGGCAGC
ATGGCATGACTCAGACTGGAAAAGAATAAGTGATTTTTGGATTAT
GATCAAATTCCAGATGAGACAATTAATTGTTAGGCAAACAGATCA
TAATGATTCTGATTTAATCACGTATATCGAAAATAGAGAAGGCAT
AATCATCATAACCCCTGAACTGGTAGCATTATTTAACACTGAGAA
TCATACACTAACATACATGACCTTTGAAATTGTACTGATGGTTTC
AGATATGTACGAAGGTCGTCACAACATTTTATCACTATGCACAGT
TAGCACTTACCTGAATCCTCTGAAGAAAAGAATAACATATTTATT
GAGCCTTGTAGATAACTTAGCTTTTCAGATAGGTGATGCTGTATA
TAACATAATTGCTTTGCTAGAATCCTTTGTATATGCACAGTTGCA
AATGTCAGATCCCATCCCAGAACTCAGAGGACAATTCCATGCATT
CGTATGTTCTGAGATTCTTGATGCACTAAGAGGAACTAATAGTTT
CACCCAGGATGAATTAAGAACTGTGACAACTAATTTGATATCCCC
ATTCCAAGATCTGACCCCAGATCTTACGGCTGAATTGCTCTGTAT
AATGAGGCTTTGGGGACACCCCATGCTCACTGCCAGTCAAGCTGC
AGGAAAGGTACGCGAGTCTATGTGTGCTGGAAAAGTATTAGACTT
TCCCACCATTATGAAAACACTAGCCTTTTTCCATACTATTCTGAT
CAATGGATACAGGAGGAAGCATCATGGAGTATGGCCACCCTTAAA
CTTACCGGGTAATGCTTCAAAGGGTCTCACGGAACTTATGAATGA
CAATACTGAAATAAGCTATGAATTCACACTTAAGCATTGGAAGGA
AGTCTCTCTTATAAAATTCAAGAAATGTTTTGATGCAGACGCAGG
TGAGGAACTCAGTATATTTATGAAAGATAAGGCAATTAGTGCCCC
AAAACAAGACTGGATGAGTGTGTTTAGAAGAAGCCTAATCAAACA
GCGCCATCAGCATCATCAGGTCCCCCTACCAAATCCATTCAATCG
ACGGCTGTTGCTAAACTTTCTCGGAGATGACAAATTCGACCCGAA
TGTGGAGCTACAGTATGTAACATCAGGTGAGTATCTACATGATGA
CACGTTTTGTGCATCATATTCACTAAAAGAGAAGGAAATTAAACC
TGATGGTCGAATTTTTGCAAAGTTGACTAAGAGAATGAGATCATG
TCAAGTTATAGCAGAATCTCTTTTAGCGAACCATGCTGGGAAGTT
AATGAAAGAGAATGGTGTTGTGATGAATCAGCTATCATTAACAAA
ATCACTATTAACAATGAGTCAGATTGGAATAATATCCGAGAAAGC
TAGAAAGTCAACTCGAGATAACATAAATCAACCTGGTTTCCAGAA
TATCCAGAGAAATAAATCACATCACTCCAAGCAAGTCAATCAGCG
AGATCCAAGTGATGACTTTGAATTGGCAGCATCTTTTTTAACTAC
TGATCTCAAAAAATATTGTTTACAATGGAGGTACCAGACAATTAT
CCCATTTGCTCAATCATTAAACAGAATGTATGGTTATCCTCATCT
CTTTGAGTGGATTCACTTACGGCTAATGCGTAGTACACTTTACGT
GGGGGATCCCTTCAACCCACCAGCAGATACCAGTCAATTTGATCT
AGATAAAGTAATTAATGGAGATATCTTCATTGTATCACCCAGAGG
TGGAATTGAAGGGCTGTGTCAAAAGGCTTGGACAATGATATCTAT
CGCTGTGATAATTCTATCTGCCACAGAGTCTGGCACACGAGTAAT
GAGTATGGTGCAGGGAGATAATCAAGCAATTGCTGTCACCACACG
AGTACCAAGGAGCCTGCCGACTCTTGAGAAAAAGACTATTGCTTT
TAGATCTTGTAATCTATTCTTTGAGAGGTTAAAATGTAATAATTT
TGGATTAGGTCACCATTTGAAAGAACAAGAGACTATCATTAGTTC
TCACTTCTTTGTTTATAGCAAGAGAATATTCTATCAGGGGAGGAT
TCTAACGCAAGCCTTAAAAAATGCTAGTAAGCTCTGCTTGACAGC
TGATGTCCTAGGAGAATGCACCCAATCATCATGTTCTAATCTTGC
AACTACTGTCATGAGGTTAACTGAGAATGGTGTTGAAAAAGATAT
CTGTTTCTACTTGAATATCTATATGACCATCAAACAGCTCTCCTA
TGATATCATCTTCCCTCAAGTGTCAATTCCTGGAGATCAGATCAC
ATTAGAATACATAAATAATCCACACCTGGTATCACGATTGGCTCT
TTTGCCATCCCAGTTAGGAGGTCTAAACTACCTGTCATGCAGTAG
GCTGTTCAATCGAAACATAGGCGACCCGGTGGTTTCCGCAGTTGC
AGATCTTAAGAGATTAATTAAATCAGGATGTATGGATTACTGGAT
CCTTTATAACTTATTAGGGAGAAAACCGGGAAACGGCTCATGGGC
TACTTTAGCAGCTGACCCGTACTCAATCAATATAGAGTATCAATA
CCCTCCAACTACAGCTCTTAAGAGGCACACCCAACAAGCTCTGAT
GGAACTCAGTACGAATCCAATGTTACGTGGCATATTCTCTGACAA
TGCACAGGCAGAAGAAAATAACCTTGCTAGGTTTCTCCTGGATAG
GGAGGTGATCTTTCCGCGTGTAGCTCACATCATCATTGAGCAAAC
CAGTGTCGGGAGGAGAAAACAGATTCAAGGATATTTGGATTCAAC
TAGATCGATAATGAGGAAATCACTAGAAATTAAGCCCTTATCCAA
TAGGAAGCTTAATGAAATACTGGATTACAACATCAATTACCTAGC
TTACAATTTGGCATTACTCAAGAATGCTATTGAACCTCCGACTTA
TTTGAAGGCAATGACACTTGAAACATGTAGCATCGACATTGCAAG
GAACCTCCGGAAGCTCTCCTGGGCCCCACTCTTGGGTGGGAGAAA
TCTTGAAGGATTAGAGACGCCAGATCCCATTGAAATTACTGCAGG
AGCATTAATTGTTGGATCGGGCTACTGTGAACAGTGTGCTGCAGG
AGACAATCGATTCACATGGTTTTTCTTGCCATCTGGTATCGAGAT
AGGAGGGGATCCCCGTGATAATCCTCCTATCCGTGTACCGTACAT
TGGCTCCAGGACTGATGAGAGGAGGGTAGCCTCAATGGCATACAT
CAGGGGTGCCTCGAGTAGCTTAAAAGCAGTTCTTAGACTGGCGGG
AGTGTACATCTGGGCATTCGGAGATACTCTGGAGAATTGGATAGA
TGCACTGGATTTGTCTCACACTAGAGTTAACATCACACTTGAACA
GCTGCAATCCCTCACCCCACTTCCAACCTCTGCCAATCTAACCCA
TCGGTTGGATGATGGCACAACTACCCTAAAGTTTACTCCTGCGAG
CTCTTACACCTTTTCAAGTTTCACTCATATATCAAATGATGAGCA
ATACCTGACAATTAATGACAAAACTGCAGATTCAAATATAATCTA
CCAACAGTTAATGATCACTGGACTCGGAATCTTAGAAACATGGAA
TAATCCCCCAATCAATAGAACATTCGAAGAATCTACCCTACATTT
GCACACTGGTGCATCATGTTGTGTCCGACCTGTGGACTCCTGCAT
TCTCTCAGAAGCATTAACAGTCAAGCCACATATTACAGTACCGTA
CAGCAATAAATTTGTATTTGATGAGGACCCGCTATCTGAATATGA
AACTGCAAAACTGGAATCGTTATCATTCCAAGCCCAATTAGGCAA
CATTGATGCTGTAGATATGACAGGTAAATTAACATTATTGTCCCA
ATTCACTGCAAGGCAGATTATCAATGCAATCACTGGACTCGATGA
GTCTGTCTCTCTTACTAATGATGCCATTGTTGCATCAGACTATGT
CTCCAATTGGATTAGTGAATGCATGTATACCAAATTAGATGAATT
ATTTATGTATTGTGGGTGGGAACTACTATTGGAACTATCCTATCA
AATGTATTATCTGAGGGTAGTTGGGTGGAGTAATATAGTGGATTA
TTCTTACATGATCTTGAGAAGAATCCCGGGTGCAGCATTAAACAA
TCTGGCATCTACATTAAGTCATCCAAAACTTTTCCGACGAGCTAT
CAACCTAGATATAGTTGCCCCCTTAAATGCTCCTCATTTTGCATC
TCTGGACTACATCAAGATGAGTGTGGATGCAATACTCTGGGGCTG
TAAAAGAGTCATCAATGTGCTCTCCAATGGAGGGGACTTAGAATT
AGTTGTGACATCTGAAGATAGCCTTATTCTCAGTGACCGATCCAT
GAATCTCATTGCAAGGAAATTAACTTTATTATCACTGATTCACCA
TAATGGTTTGGAACTACCAAAGATTAAGGGGTTCTCTCCTGATGA
GAAGTGTTTCGCTTTGACAGAATTTTTGAGGAAAGTGGTGAACTC
AGGGTTGAGTTCAATAGAGAACCTATCAAATTTTATGTACAATGT
GGAGAACCCACGGCTTGCAGCATTCGCCAGCAACAATTACTACCT
GACCAGAAAATTATTGAATTCAATACGAGATACTGAGTCGGGTCA
AGTAGCAGTCACCTCATATTATGAATCATTAGAATATATTGATAG
TCTTAAGCTAACCCCACATGTGCCTGGCACCTCATGCATTGAGGA
TGATAGTCTATGTACAAATGATTACATAATCTGGATCATAGAGTC
TAATGCAAACTTGGAGAAGTATCCAATTCCAAATAGCCCTGAGGA
TGATTCCAATTTCCATAACTTTAAGTTGAATGCTCCATCGCACCA
TACCTTACGCCCATTAGGGTTGTCATCAACTGCTTGGTATAAGGG
TATAAGCTGCTGCAGGTACCTTGAGCGATTAAAGCTACCACAAGG
TGATCATTTATATATTGCAGAAGGTAGTGGTGCCAGTATGACAAT
CATAGAATACCTATTCCCAGGAAGAAAGATATATTACAATTCTTT
ATTTAGTAGTGGTGACAATCCCCCACAAAGAAATTATGCACCAAT
GCCTACTCAGTTCATTGAGAGTGTCCCATACAAGCTCTGGCAAGC
ACACACAGATCAATATCCCGAGATTTTTGAGGACTTCATCCCTCT
ATGGAACGGAAACGCCGCCATGACTGACATAGGAATGACAGCTTG
TGTAGAATTCATCATCAATCGAGTCGGCCCAAGGACTTGCAGTTT
AGTACATGTAGATTTGGAATCAAGTGCAAGCTTAAATCAACAATG
CCTGTCAAAGCCGATAATTAATGCTATCATCACTGCTACAACTGT
TTTGTGCCCTCATGGGGTGCTTATTCTGAAATATAGTTGGTTGCC
ATTTACTAGATTTAGTACTTTGATCACTTTCTTATGGTGCTACTT
TGAGAGAATCACTGTTCTTAGGAGCACATATTCTGATCCAGCTAA
TCATGAGGTTTATTTAATTTGTATCCTTGCCAACAACTTTGCATT
CCAGACTGTCTCGCAGGCAACAGGAATGGCGATGACTTTAACTGA
TCAAGGGTTTACTTTGATATCACCTGAAAGAATAAATCAGTATTG
GGATGGTCACTTGAAGCAAGAACGTATCGTAGCAGAAGCAATTGA
TAAGGTGGTTCTAGGAGAAAATGCTCTATTTAATTCGAGTGATAA
TGAATTAATTCTCAAATGTGGAGGGACACCAAATGCACGGAATCT
CATCGATATCGAGCCAGTCGCAACTTTCATAGAATTTGAACAATT
GATCTGCACAATGTTGACAACCCACTTGAAGGAAATAATTGATAT
AACAAGGTCTGGAACCCAGGATTATGAAAGTTTATTACTCACTCC
TTACAATTTAGGTCTTCTTGGTAAAATCAGTACGATAGTGAGATT
ATTAACAGAAAGGATTCTAAATCATACTATCAGGAATTGGTTGAT
CCTCCCACCTTCGCTCCGGATGATCGTGAAGCAGGACTTGGAATT
CGGCATATTCAGGATTACTTCCATCCTCAATTCTGATCGGTTCCT
GAAGCTTTCTCCAAATAGGAAATACTTGATTGCACAATTAACTGC
AGGCTACATTAGGAAATTGATTGAGGGGGATTGCAATATCGATCT
AACCAGACCTATCCAAAAGCAAATCTGGAAAGCATTAGGTTGTGT
AGTCTATTGTCACGATCCAATGGATCAAAGGGAGTCAACAGAGTT
TATTGATATAAATATTAATGAAGAAATAGACCGCGGGATCGATGG
CGAGGAAATCTAAACATATCAAGAATCAGAATTAGTTTAAGAAAA
AAGAAGAGGATTAATCTTGGTTTTCCCCTTGGTGGGTCGGCATGG
CATCTCCACCTCCTCGCGGTCCGACCTGGGCATCCGAAGGAGGAC
GCACGTCCACTCGGATGGCTAAGGGAGCGGCCGGGGATCCGGCTG
CTAACAAAGCCCGAAAGGAAGCTGAGTTGGCTGCTGCCACCGCTG
AGCAATAACTAGCATAACCCCTTGGGGCCTCTAAACGGGTCTTGA
GGGGTTTTTTGCTGAAAGGAGGAACTATATCCGGATCATGTGAGC
AAAAGGCCAGCAAAAGGCCAGGAACCGTAAAAAGGCCGCGTTGCT
GGCGTTTTTCCATAGGCTCCGCCCCCCTGACGAGCATCACAAAAA
TCGACGCTCAAGTCAGAGGTGGCGAAACCCGACAGGACTATAAAG
ATACCAGGCGTTTCCCCCTGGAAGCTCCCTCGTGCGCTCTCCTGT
TCCGACCCTGCCGCTTACCGGATACCTGTCCGCCTTTCTCCCTTC
GGGAAGCGTGGCGCTTTCTCATAGCTCACGCTGTAGGTATCTCAG
TTCGGTGTAGGTCGTTCGCTCCAAGCTGGGCTGTGTGCACGAACC
CCCCGTTCAGCCCGACCGCTGCGCCTTATCCGGTAACTATCGTCT
TGAGTCCAACCCGGTAAGACACGACTTATCGCCACTGGCAGCAGC
CACTGGTAACAGGATTAGCAGAGCGAGGTATGTAGGCGGTGCTAC
AGAGTTCTTGAAGTGGTGGCCTAACTACGGCTACACTAGAAGGAC
AGTATTTGGTATCTGCGCTCTGCTGAAGCCAGTTACCTTCGGAAA
AAGAGTTGGTAGCTCTTGATCCGGCAAACAAACCACCGCTGGTAG
CGGTGGTTTTTTTGTTTGCAAGCAGCAGATTACGCGCAGAAAAAA
AGGATCTCAAGAAGATCCTTTGATCTTTTCTACGGGGTCTGACGC
TCAGTGGAACGAAAACTCACGTTAAGGGATTTTGGTCATGAGATT
ATCAAAAAGGATCTTCACCTAGATCCTTTTAAATTAAAAATGAAG
TTTTAAATCAATCTAAAGTATATATGAGTAAACTTGGTCTGACAG
TTACCAATGCTTAATCAGTGAGGCACCTATCTCAGCGATCTGTCT
ATTTCGTTCATCCATAGTTGCCTGACTCCCCGTCGTGTAGATAAC
TACGATACGGGAGGGCTTACCATCTGGCCCCAGTGCTGCAATGAT
ACCGCGAGACCCACGCTCACCGGCTCCAGATTTATCAGCAATAAA
CCAGCCAGCCGGAAGGGCCGAGCGCAGAAGTGGTCCTGCAACTTT
ATCCGCCTCCATCCAGTCTATTAATTGTTGCCGGGAAGCTAGAGT
AAGTAGTTCGCCAGTTAATAGTTTGCGCAACGTTGTTGCCATTGC
TACAGGCATCGTGGTGTCACGCTCGTCGTTTGGTATGGCTTCATT
CAGCTCCGGTTCCCAACGATCAAGGCGAGTTACATGATCCCCCAT
GTTGTGCAAAAAAGCGGTTAGCTCCTTCGGTCCTCCGATCGTTGT
CAGAAGTAAGTTGGCCGCAGTGTTATCACTCATGGTTATGGCAGC
ACTGCATAATTCTCTTACTGTCATGCCATCCGTAAGATGCTTTTC
TGTGACTGGTGAGTACTCAACCAAGTCATTCTGAGAATAGTGTAT
GCGGCGACCGAGTTGCTCTTGCCCGGCGTCAATACGGGATAATAC
CGCGCCACATAGCAGAACTTTAAAAGTGCTCATCATTGGAAAACG
TTCTTCGGGGCGAAAACTCTCAAGGATCTTACCGCTGTTGAGATC
CAGTTCGATGTAACCCACTCGTGCACCCAACTGATCTTCAGCATC
TTTTACTTTCACCAGCGTTTCTGGGTGAGCAAAAACAGGAAGGCA
AAATGCCGCAAAAAAGGGAATAAGGGCGACACGGAAATGTTGAAT
ACTCATACTCTTCCTTTTTCAATATTATTGAAGCATTTATCAGGG
TTATTGTCTCATGAGCGGATACATATTTGAATGTATTTAGAAAAA
TAAACAAATAGGGGTTCCGCGCACATTTCCCCGAAAAGT.

vii. CVL112

The nucleic acid sequence for CVL112 is:

(SEQ ID NO: 9)
GCCACCTGACGTCTAAGAAACCATTATTATCATGACATTAACCTA
TAAAAATAGGCGTATCACGAGGCCCTTTCGTCTCGCGCGTTTCGG
TGATGACGGTGAAAACCTCTGACACATGCAGCTCCCGGAGACGGT
CACAGCTTGTCTGTAAGCGGATGCCGGGAGCAGACAAGCCCGTCA
GGGCGCGTCAGCGGGTGTTGGCGGGTGTCGGGGCTGGCTTAACTA
TGCGGCATCAGAGCAGATTGTACTGAGAGTGCACCATATATGCGG
TGTGAAATACCGCACAGATGCGTAAGGAGAAAATACCGCATCAGG
CTAATACGACTCACTATAGGGACCAAGGGGAAAATGAAGTGGTGA
CTCAAATCATCGAAGACCCTCGAGATTACATAGGTCCGGAACCTA
TGGCCTTCGTGACCGACCTCGAGTCAGAGTAGTTCAATAAGGACC
TATCAAGTTTGGGCAATTTTTCGTCCCCGACACAAAAATGTCATC
CGTGCTTAAAGCATATGAGCGATTCACGCTCACTCAAGAACTGCA
AGATCAGAGTGAGGAAGGTACAATCCCACCTACAACACTAAAACC
GGTAATCAGGGTATTTATACTAACCTCTAATAACCCAGAGCTAAG
ATCCCGGCTTCTTCTATTCTGCCTACGGATTGTTCTCAGTAATGG
TGCAAGGGATTCCCATCGCTTTGGAGCATTACTCACAATGTTTTC
GCTACCATCAGCCACAATGCTCAATCATGTCAAATTAGCTGACCA
GTCACCAGAAGCTGATATCGAAAGGGTAGAGATCGATGGCTTTGA
GGAGGGATCATTCCGCTTAATCCCCAATGCTCGTTCAGGTATGAG
CCGTGGAGAGATCAATGCCTATGCTGCACTTGCAGAAGATCTACC
TGACACACTAAACCATGCAACACCTTTCGTTGATTCCGAAGTCGA
GGGAACTGCATGGGATGAGATTGAGACTTTCTTAGATATGTGTTA
CAGTGTCCTAATGCAGGCATGGATAGTGACTTGCAAGTGCATGAC
TGCGCCAGACCAACCTGCTGCTTCTATTGAGAAACGCCTGCAAAA
ATATCGTCAGCAAGGCAGGATCAACCCGAGATATCTCCTGCAACC
GGAGGCTCGACGAATAATCCAGAATGTAATCCGGAAGGGAATGGT
GGTCAGACATTTCCTCACCTTTGAACTGCAGCTTGCCCGAGCACA
AAGCCTTGTATCAAATAGGTATTATGCTATGGTAGGGGATGTTGG
AAAGTATATAGAGAATTGTGGAATGGGAGGCTTCTTTTTGACACT
AAAATATGCATTAGGAACTAGATGGCCCACACTTGCTTTAGCTGC
ATTTTCAGGAGAGCTAACAAAGCTAAAGTCCCTCATGGCATTATA
CCAGACCCTTGGTGAGCAGGCCCGATATTTGGCCCTATTGGAGTC
ACCACATTTGATGGATTTTGCTGCAGCAAACTACCCACTGCTATA
TAGCTATGCTATGGGAATAGGCTATGTGTTAGATGTCAACATGAG
GAACTACGCTTTCTCCAGATCATACATGAACAAGACATATTTCCA
ATTGGGAATGGAAACTGCAAGAAAACAACAGGGTGCAGTTGACAT
GAGGATGGCAGAAGATCTCGGTCTAACTCAAGCCGAACGCACCGA
GATGGCAAATACACTTGCCAAATTGACCACAGCAAATCGAGGGGC
AGACACCAGGGGAGGAGTCAACCCGTTCTCATCTGTCACTGGGAC
AACTCAGGTGCCCGCTGCAGCAACAGGTGACACACTCGAGAGTTA
CATGGCAGCGGATCGACTGAGGCAGAGATATGCTGATGCAGGCAC
CCATGATGATGAGATGCCACCATTGGAAGAGGAGGAAGAGGACGA
CACATCTGCAGGTCCACGCACTGGACCAACTCTTGAACAAGTGGC
CTTGGACATCCAGAACGCAGCAGTTGGAGCTCCCATCCATACAGA
TGACCTGAATGCCGCACTGGGTGATCTTGACATCTAGACAATTCA
GATCCCAATCTAAAATTGACATACCTAATTGATTAGTTAGATGGA
ACTACAGTGGATTCCATAAGGTTCCTGCCTACCATCGGCTTTAAA
GAAAAAAATAGGCCCGGACGGGTTAGCAACAAGCGACTGCCGGTG
CCAACAGCGCAATCCACAATCTACAATGGATCCCACTGATCTGAG
CTTCTCCCCAGATGAGATCAATAAGCTCATAGAGACAGGCCTGAA
TACTGTAGAGTATTTTACTTCCCAACAAGTCACAGGAACATCCTC
TCTTGGAAAGAATACAATACCACCAGGGGTCACAGGACTACTAAC
CAATGCTGCAGAGGCAAAGATCCAAGAGTCAACTAACCATCAGAA
GGGCTCAGTTGGTGGGGGTGCAAAACCAAAGAAACCGCGACCAAA
AATTGCCATTGTGCCAGCAGATGACAAAACAGTGCCCGGAAAGCC
GATCCCAAACCCTCTATTAGGTCTGGACTCCACCCCGAGCACCCA
AACTGTGCTTGATCTAAGTGGGAAAACATTACCATCAGGATCCTA
TAAGGGGGTTAAGCTTGCGAAATTTGGAAAAGAAAATCTGATGAC
ACGGTTCATCGAGGAACCCAGAGAGAATCCTATCGCAACCAGTTt
CCCCATCGATTTTAAGAGGGGCAGGGATACCGGCGGGTTCCATAG
AAGGGAGTACTCAATCGGATGGGTGGGAGATGAAGTCAAGGTCAC
TGAGTGGTGCAATCCATCCTGTTCTCCAATCACCGCTGCAGCAAG
GCGATTTGAATGCACTTGTCACCAGTGTCCAGTCACTTGCTCTGA
ATGTGAACGAGATACTTAATACAGTGAGAAATTTGGACTCTCGGA
TGAATCAACTGGAGACAAAAGTAGATCGCATTCTCTCATCTCAGT
CTCTAATCCAGACCATCAAGAATGACATAGTTGGACTTAAAGCAG
GGATGGCTACTTTAGAAGGAATGATTACAACTGTGAAAATCATGG
ACCCGGGAGTTCCCAGTAATGTTACTGTGGAAGATGTACGCAAGA
CACTAAGTAACCATGCTGTTGTTGTGCCAGAATCATTCAATGATA
GTTTCTTGACTCAATCTGAAGATGTAATTTCACTTGATGAGTTGG
CTCGACCAACTGCAACAAGTGTTAAGAAGATTGTCAGGAAGGTTC
CTCCTCAGAAGGATCTGACTGGATTGAAGATTACACTAGAGCAAT
TGGCAAAGGATTGCATCAGCAAACCGAAGATGAGGGAAGAGTATC
TCCTCAAAATCAACCAGGCTTCCAGTGAGGCTCAGCTAATTGACC
TCAAGAAAGCAATCATCCGCAGTGCAATTTGATCAAGAAACACCC
AATTACACTACACTGGTATGACACTGTACTAACCCTGAGGGTTTT
AGAAAAAACGATTAACGATAAATAAGCCCGAACACTACACACTAC
CTGAGGCAGCCATGCCATCCATCAGCATTCCCGCAGACCCCACCA
ATCCACGTCAATCAATAAAAGCGTTCCCAATTGTGATCAACAGTG
ATGGGGGTGAGAAAGGCCGCTTGGTTAAACAACTACGCACAACCT
ACTTGAATGACCTAGATACTCATGAGCCACTGGTGACATTCATAA
ATACCTATGGATTCATCTACGAACAGGATCGGGGGAATACCATTG
TCGGAGAGGATCAACTTGGGAAGAAAAGAGAGGCTGTGACCGCTG
CAATGGTTACCCTTGGATGTGGGCCTAATCTACCATCATTAGGGA
ATGTCCTGGGACAACTGAGGGAATTCCAGGTCACTGTTAGGAAGA
CATCCAGCAAAGCGGAAGAGATGGTCTTTGAAATTGTTAAGTATC
CGAGAATATTTCGGGGTCATACATTAATCCAGAAAGGACTAGTCT
GTGTCTCCGCAGAAAAATTTGTTAAGTCACCAGGGAAAATACAAT
CTGGAATGGACTATCTCTTCATTCCGACATTTCTGTCAGTGACTT
ACTGTCCAGCTGCAATCAAATTTCAGGTACCTGGCCCCATGTTGA
AAATGAGATCAAGATACACTCAGAGCTTACAACTTGAACTAATGA
TAAGAATCCTGTGTAAGCCCGATTCGCCACTTATGAAGGTCCATA
CCCCTGACAAGGAGGGAAGAGGATGTCTTGTATCAGTATGGCTGC
ATGTATGCAACATCTTCAAATCAGGAAACAAGAATGGCAGTGAGT
GGCAGGAATACTGGATGAGAAAGTGTGCTAACATGCAACTTGAAG
TGTCGATTGCAGATATGTGGGGACCAACTATCATAATTCATGCCA
GAGGTCACATTCCCAAAAGTGCTAAGTTGTTTTTTGGAAAGGGTG
GATGGAGCTGCCATCCACTTCACGAAGTTGTTCCAAGTGTCACTA
AAACACTATGGTCCGTGGGCTGTGAGATTACAAAGGCGAAGGCAA
TAATACAAGAGAGTAGCATCTCTCTTCTCGTGGAGACTACTGACA
TCATAAGTCCAAAAGTCAAAATTTCATCTAAGCATCGCCGCTTTG
GGAAATCAAATTGGGGTCTGTTCAAGAAAACTAAATCACTGCCTA
ACCTGACGGAGCTGGAATGAgctagcGGCAGAAGCCTTCAGGTCT
GACCCAGCCAATCAAAGCATTATACCAGACCATGGCCTACCATCG
GCTTTAAAGAAAAAAATAGGCCCGGACGGGTTAGCAACAAGCGGC
GGCCGCCatgttcgtcttcctggtcctgctgcctctggtctcctc
acagtgcgtcaatctgacaactcggactcagctgccacctgctta
tactaatagcttcaccagaggcgtgtactatcctgacaaggtgtt
tagaagctccgtgctgcactctacacaggatctgtttctgccatt
ctttagcaacgtgacctggttccacgccatccacgtgagcggcac
caatggcacaaagcggttcgacaatcccgtgctgccttttaacga
tggcgtgtacttcgcctctaccgagaagagcaacatcatcagagg
ctggatctttggcaccacactggactccaagacacagtctctgct
gatcgtgaacaatgccaccaacgtggtcatcaaggtgtgcgagtt
ccagttttgtaatgatcccttcctgggcgtgtactatcacaagaa
caataagagctggatggagtccgagtttagagtgtattctagcgc
caacaactgcacatttgagtacgtgagccagcctttcctgatgga
cctggagggcaagcagggcaatttcaagaacctgagggagttcgt
gtttaagaatatcgacggctacttcaaaatctactctaagcacac
ccccatcaacctggtgcgcgacctgcctcagggcttcagcgccct
ggagcccctggtggatctgcctatcggcatcaacatcacccggtt
tcagacactgctggccctgcacagaagctacctgacacccggcga
ctcctctagcggatggaccgccggcgctgccgcctactatgtggg
ctacctccagccccggaccttcctgctgaagtacaacgagaatgg
caccatcacagacgcagtggattgcgccctggaccccctgagcga
gacaaagtgtacactgaagtcctttaccgtggagaagggcatcta
tcagacatccaatttcagggtgcagccaaccgagtctatcgtgcg
ctttcctaatatcacaaacctgtgcccatttggcgaggtgttcaa
cgcaacccgcttcgccagcgtgtacgcctggaataggaagcggat
cagcaactgcgtggccgactatagcgtgctgtacaactccgcctc
tttcagcacctttaagtgctatggcgtgtcccccacaaagctgaa
tgacctgtgctttaccaacgtctacgccgattctttcgtgatcag
gggcgacgaggtgcgccagatcgcccccggccagacaggcaagat
cgcagactacaattataagctgccagacgatttcaccggctgcgt
gatcgcctggaacagcaacaatctggattccaaagtgggcggcaa
ctacaattatctgtaccggctgtttagaaagagcaatctgaagcc
cttcgagagggacatctctacagaaatctaccaggccggcagcac
cccttgcaatggcgtggagggctttaactgttatttcccactcca
gtcctacggcttccagcccacaaacggcgtgggctatcagcctta
ccgcgtggtggtgctgagctttgagctgctgcacgccccagcaac
agtgtgcggccccaagaagtccaccaatctggtgaagaacaagtg
cgtgaacttcaacttcaacggcctgaccggcacaggcgtgctgac
cgagtccaacaagaagttcctgccatttcagcagttcggcaggga
catcgcagataccacagacgccgtgcgcgacccacagaccctgga
gatcctggacatcacaccctgctctttcggcggcgtgagcgtgat
cacacccggcaccaatacaagcaaccaggtggccgtgctgtatca
ggacgtgaattgtaccgaggtgcccgtggctatccacgccgatca
gctgaccccaacatggcgggtgtacagcaccggctccaacgtctt
ccagacaagagccggatgcctgatcggagcagagcacgtgaacaa
ttcctatgagtgcgacatcccaatcggcgccggcatctgtgcctc
ttaccagacccagacaaactctcccagaagagcccggagcgtggc
ctcccagtctatcatcgcctataccatgtccctgggcgccgagaa
cagcgtggcctactctaacaatagcatcgccatcccaaccaactt
cacaatctctgtgaccacagagatcctgcccgtgtccatgaccaa
gacatctgtggactgcacaatgtatatctgtggcgattctaccga
gtgcagcaacctgctgctccagtacggcagcttttgtacccagct
gaatagagccctgacaggcatcgccgtggagcaggataagaacac
acaggaggtgttcgcccaggtgaagcaaatctacaagaccccccc
tatcaaggactttggcggcttcaatttttcccagatcctgcctga
tccatccaagccttctaagcggagctttatcgaggacctgctgtt
caacaaggtgaccctggccgatgccggcttcatcaagcagtatgg
cgattgcctgggcgacatcgcagccagggacctgatctgcgccca
gaagtttaatggcctgaccgtgctgccacccctgctgacagatga
gatgatcgcacagtacacaagcgccctgctggccggcaccatcac
atccggatggaccttcggcgcaggagccgccctccagatcccctt
tgccatgcagatggcctataggttcaacggcatcggcgtgaccca
gaatgtgctgtacgagaaccagaagctgatcgccaatcagtttaa
ctccgccatcggcaagatccaggacagcctgtcctctacagccag
cgccctgggcaagctccaggatgtggtgaatcagaacgcccaggc
cctgaataccctggtgaagcagctgagcagcaacttcggcgccat
ctctagcgtgctgaatgacatcctgagccggctggacaaggtgga
ggcagaggtgcagatcgaccggctgatcaccggccggctccagag
cctccagacctatgtgacacagcagctgatcagggccgccgagat
cagggccagcgccaatctggcagcaaccaagatgtccgagtgcgt
gctgggccagtctaagagagtggacttttgtggcaagggctatca
cctgatgtccttccctcagtctgccccacacggcgtggtgtttct
gcacgtgacctacgtgcccgcccaggagaagaacttcaccacagc
ccctgccatctgccacgatggcaaggcccactttccaagggaggg
cgtgttcgtgtccaacggcacccactggtttgtgacacagcgcaa
tttctacgagccccagatcatcaccacagacaacaccttcgtgag
cggcaactgtgacgtggtcatcggcatcgtgaacaataccgtgta
tgatccactccagcccgagctggacagctttaaggaggagctgga
taagtatttcaagaatcacacctcccctgacgtggatctgggcga
catcagcggcatcaatgcctccgtggtgaacatccagaaggagat
cgaccgcctgaacgaggtggctaagaatctgaacgagagcctgat
cgacctccaggagctgggcaagtatgagcagtacatcaagtggcc
ctggtacatctggctgggcttcatcgccggcctgatcgccatcgt
gatggtgaccatcatgctgtgctgtatgacatcctgctgttcttg
cctgaagggctgctgtagctgtggctcctgctgtaagttattgac
cattgtcgttgctaatcgaaatagaatggagaattttgtttatca
taaatgaAGTCGACTCATGGAATGCATACCAAACATTATTGACAC
TAATGACACACAAAATTGGTTTTAAGAAAAACCAAGAGAACAATA
GGCCAGAATGGCTGGGTCTCGGGAGATATTACTCCCTGAAGTCCA
TCTCAATTCACCAATTGTAAAGCATAAGCTATACTATTACATTCT
ACTTGGAAACCTCCCAAATGAGATCGACCTTGACGATTTAGGTCC
ATTACATAATCAAAATTGGAATCAGATAGCACATGAAGAGTCTAA
CTTAGCTCAACGCTTGGTAAATGTAAGAAATTTTCTAATTACCCA
CATCCCTGATCTTAGAAAGGGCCATTGGCAAGAGTATGTCAATGT
AATACTGTGGCCGCGAATTCTTCCCTTGATCCCGGATTTTAAAAT
CAATGACCAATTGCCTCTGCTCAAAAATTGGGACAAGTTAGTTAA
AGAATCATGTTCAGTAATCAATGCAGGTACTTCCCAGTGCATTCA
GAATCTCAGCTATGGACTGACAGGTCGTGGGAACCTCTTTACACG
ATCACGTGAACTCTCTGGTGACCGCAGGGATATTGATCTTAAGAC
AGTTGTGGCAGCATGGCATGACTCAGACTGGAAAAGAATAAGTGA
TTTTTGGATTATGATCAAATTCCAGATGAGACAATTAATTGTTAG
GCAAACAGATCATAATGATTCTGATTTAATCACGTATATCGAAAA
TAGAGAAGGCATAATCATCATAACCCCTGAACTGGTAGCATTATT
TAACACTGAGAATCATACACTAACATACATGACCTTTGAAATTGT
ACTGATGGTTTCAGATATGTACGAAGGTCGTCACAACATTTTATC
ACTATGCACAGTTAGCACTTACCTGAATCCTCTGAAGAAAAGAAT
AACATATTTATTGAGCCTTGTAGATAACTTAGCTTTTCAGATAGG
TGATGCTGTATATAACATAATTGCTTTGCTAGAATCCTTTGTATA
TGCACAGTTGCAAATGTCAGATCCCATCCCAGAACTCAGAGGACA
ATTCCATGCATTCGTATGTTCTGAGATTCTTGATGCACTAAGAGG
AACTAATAGTTTCACCCAGGATGAATTAAGAACTGTGACAACTAA
TTTGATATCCCCATTCCAAGATCTGACCCCAGATCTTACGGCTGA
ATTGCTCTGTATAATGAGGCTTTGGGGACACCCCATGCTCACTGC
CAGTCAAGCTGCAGGAAAGGTACGCGAGTCTATGTGTGCTGGAAA
AGTATTAGACTTTCCCACCATTATGAAAACACTAGCCTTTTTCCA
TACTATTCTGATCAATGGATACAGGAGGAAGCATCATGGAGTATG
GCCACCCTTAAACTTACCGGGTAATGCTTCAAAGGGTCTCACGGA
ACTTATGAATGACAATACTGAAATAAGCTATGAATTCACACTTAA
GCATTGGAAGGAAGTCTCTCTTATAAAATTCAAGAAATGTTTTGA
TGCAGACGCAGGTGAGGAACTCAGTATATTTATGAAAGATAAGGC
AATTAGTGCCCCAAAACAAGACTGGATGAGTGTGTTTAGAAGAAG
CCTAATCAAACAGCGCCATCAGCATCATCAGGTCCCCCTACCAAA
TCCATTCAATCGACGGCTGTTGCTAAACTTTCTCGGAGATGACAA
ATTCGACCCGAATGTGGAGCTACAGTATGTAACATCAGGTGAGTA
TCTACATGATGACACGTTTTGTGCATCATATTCACTAAAAGAGAA
GGAAATTAAACCTGATGGTCGAATTTTTGCAAAGTTGACTAAGAG
AATGAGATCATGTCAAGTTATAGCAGAATCTCTTTTAGCGAACCA
TGCTGGGAAGTTAATGAAAGAGAATGGTGTTGTGATGAATCAGCT
ATCATTAACAAAATCACTATTAACAATGAGTCAGATTGGAATAAT
ATCCGAGAAAGCTAGAAAGTCAACTCGAGATAACATAAATCAACC
TGGTTTCCAGAATATCCAGAGAAATAAATCACATCACTCCAAGCA
AGTCAATCAGCGAGATCCAAGTGATGACTTTGAATTGGCAGCATC
TTTTTTAACTACTGATCTCAAAAAATATTGTTTACAATGGAGGTA
CCAGACAATTATCCCATTTGCTCAATCATTAAACAGAATGTATGG
TTATCCTCATCTCTTTGAGTGGATTCACTTACGGCTAATGCGTAG
TACACTTTACGTGGGGGATCCCTTCAACCCACCAGCAGATACCAG
TCAATTTGATCTAGATAAAGTAATTAATGGAGATATCTTCATTGT
ATCACCCAGAGGTGGAATTGAAGGGCTGTGTCAAAAGGCTTGGAC
AATGATATCTATCGCTGTGATAATTCTATCTGCCACAGAGTCTGG
CACACGAGTAATGAGTATGGTGCAGGGAGATAATCAAGCAATTGC
TGTCACCACACGAGTACCAAGGAGCCTGCCGACTCTTGAGAAAAA
GACTATTGCTTTTAGATCTTGTAATCTATTCTTTGAGAGGTTAAA
ATGTAATAATTTTGGATTAGGTCACCATTTGAAAGAACAAGAGAC
TATCATTAGTTCTCACTTCTTTGTTTATAGCAAGAGAATATTCTA
TCAGGGGAGGATTCTAACGCAAGCCTTAAAAAATGCTAGTAAGCT
CTGCTTGACAGCTGATGTCCTAGGAGAATGCACCCAATCATCATG
TTCTAATCTTGCAACTACTGTCATGAGGTTAACTGAGAATGGTGT
TGAAAAAGATATCTGTTTCTACTTGAATATCTATATGACCATCAA
ACAGCTCTCCTATGATATCATCTTCCCTCAAGTGTCAATTCCTGG
AGATCAGATCACATTAGAATACATAAATAATCCACACCTGGTATC
ACGATTGGCTCTTTTGCCATCCCAGTTAGGAGGTCTAAACTACCT
GTCATGCAGTAGGCTGTTCAATCGAAACATAGGCGACCCGGTGGT
TTCCGCAGTTGCAGATCTTAAGAGATTAATTAAATCAGGATGTAT
GGATTACTGGATCCTTTATAACTTATTAGGGAGAAAACCGGGAAA
CGGCTCATGGGCTACTTTAGCAGCTGACCCGTACTCAATCAATAT
AGAGTATCAATACCCTCCAACTACAGCTCTTAAGAGGCACACCCA
ACAAGCTCTGATGGAACTCAGTACGAATCCAATGTTACGTGGCAT
ATTCTCTGACAATGCACAGGCAGAAGAAAATAACCTTGCTAGGTT
TCTCCTGGATAGGGAGGTGATCTTTCCGCGTGTAGCTCACATCAT
CATTGAGCAAACCAGTGTCGGGAGGAGAAAACAGATTCAAGGATA
TTTGGATTCAACTAGATCGATAATGAGGAAATCACTAGAAATTAA
GCCCTTATCCAATAGGAAGCTTAATGAAATACTGGATTACAACAT
CAATTACCTAGCTTACAATTTGGCATTACTCAAGAATGCTATTGA
ACCTCCGACTTATTTGAAGGCAATGACACTTGAAACATGTAGCAT
CGACATTGCAAGGAACCTCCGGAAGCTCTCCTGGGCCCCACTCTT
GGGTGGGAGAAATCTTGAAGGATTAGAGACGCCAGATCCCATTGA
AATTACTGCAGGAGCATTAATTGTTGGATCGGGCTACTGTGAACA
GTGTGCTGCAGGAGACAATCGATTCACATGGTTTTTCTTGCCATC
TGGTATCGAGATAGGAGGGGATCCCCGTGATAATCCTCCTATCCG
TGTACCGTACATTGGCTCCAGGACTGATGAGAGGAGGGTAGCCTC
AATGGCATACATCAGGGGTGCCTCGAGTAGCTTAAAAGCAGTTCT
TAGACTGGCGGGAGTGTACATCTGGGCATTCGGAGATACTCTGGA
GAATTGGATAGATGCACTGGATTTGTCTCACACTAGAGTTAACAT
CACACTTGAACAGCTGCAATCCCTCACCCCACTTCCAACCTCTGC
CAATCTAACCCATCGGTTGGATGATGGCACAACTACCCTAAAGTT
TACTCCTGCGAGCTCTTACACCTTTTCAAGTTTCACTCATATATC
AAATGATGAGCAATACCTGACAATTAATGACAAAACTGCAGATTC
AAATATAATCTACCAACAGTTAATGATCACTGGACTCGGAATCTT
AGAAACATGGAATAATCCCCCAATCAATAGAACATTCGAAGAATC
TACCCTACATTTGCACACTGGTGCATCATGTTGTGTCCGACCTGT
GGACTCCTGCATTCTCTCAGAAGCATTAACAGTCAAGCCACATAT
TACAGTACCGTACAGCAATAAATTTGTATTTGATGAGGACCCGCT
ATCTGAATATGAAACTGCAAAACTGGAATCGTTATCATTCCAAGC
CCAATTAGGCAACATTGATGCTGTAGATATGACAGGTAAATTAAC
ATTATTGTCCCAATTCACTGCAAGGCAGATTATCAATGCAATCAC
TGGACTCGATGAGTCTGTCTCTCTTACTAATGATGCCATTGTTGC
ATCAGACTATGTCTCCAATTGGATTAGTGAATGCATGTATACCAA
ATTAGATGAATTATTTATGTATTGTGGGTGGGAACTACTATTGGA
ACTATCCTATCAAATGTATTATCTGAGGGTAGTTGGGTGGAGTAA
TATAGTGGATTATTCTTACATGATCTTGAGAAGAATCCCGGGTGC
AGCATTAAACAATCTGGCATCTACATTAAGTCATCCAAAACTTTT
CCGACGAGCTATCAACCTAGATATAGTTGCCCCCTTAAATGCTCC
TCATTTTGCATCTCTGGACTACATCAAGATGAGTGTGGATGCAAT
ACTCTGGGGCTGTAAAAGAGTCATCAATGTGCTCTCCAATGGAGG
GGACTTAGAATTAGTTGTGACATCTGAAGATAGCCTTATTCTCAG
TGACCGATCCATGAATCTCATTGCAAGGAAATTAACTTTATTATC
ACTGATTCACCATAATGGTTTGGAACTACCAAAGATTAAGGGGTT
CTCTCCTGATGAGAAGTGTTTCGCTTTGACAGAATTTTTGAGGAA
AGTGGTGAACTCAGGGTTGAGTTCAATAGAGAACCTATCAAATTT
TATGTACAATGTGGAGAACCCACGGCTTGCAGCATTCGCCAGCAA
CAATTACTACCTGACCAGAAAATTATTGAATTCAATACGAGATAC
TGAGTCGGGTCAAGTAGCAGTCACCTCATATTATGAATCATTAGA
ATATATTGATAGTCTTAAGCTAACCCCACATGTGCCTGGCACCTC
ATGCATTGAGGATGATAGTCTATGTACAAATGATTACATAATCTG
GATCATAGAGTCTAATGCAAACTTGGAGAAGTATCCAATTCCAAA
TAGCCCTGAGGATGATTCCAATTTCCATAACTTTAAGTTGAATGC
TCCATCGCACCATACCTTACGCCCATTAGGGTTGTCATCAACTGC
TTGGTATAAGGGTATAAGCTGCTGCAGGTACCTTGAGCGATTAAA
GCTACCACAAGGTGATCATTTATATATTGCAGAAGGTAGTGGTGC
CAGTATGACAATCATAGAATACCTATTCCCAGGAAGAAAGATATA
TTACAATTCTTTATTTAGTAGTGGTGACAATCCCCCACAAAGAAA
TTATGCACCAATGCCTACTCAGTTCATTGAGAGTGTCCCATACAA
GCTCTGGCAAGCACACACAGATCAATATCCCGAGATTTTTGAGGA
CTTCATCCCTCTATGGAACGGAAACGCCGCCATGACTGACATAGG
AATGACAGCTTGTGTAGAATTCATCATCAATCGAGTCGGCCCAAG
GACTTGCAGTTTAGTACATGTAGATTTGGAATCAAGTGCAAGCTT
AAATCAACAATGCCTGTCAAAGCCGATAATTAATGCTATCATCAC
TGCTACAACTGTTTTGTGCCCTCATGGGGTGCTTATTCTGAAATA
TAGTTGGTTGCCATTTACTAGATTTAGTACTTTGATCACTTTCTT
ATGGTGCTACTTTGAGAGAATCACTGTTCTTAGGAGCACATATTC
TGATCCAGCTAATCATGAGGTTTATTTAATTTGTATCCTTGCCAA
CAACTTTGCATTCCAGACTGTCTCGCAGGCAACAGGAATGGCGAT
GACTTTAACTGATCAAGGGTTTACTTTGATATCACCTGAAAGAAT
AAATCAGTATTGGGATGGTCACTTGAAGCAAGAACGTATCGTAGC
AGAAGCAATTGATAAGGTGGTTCTAGGAGAAAATGCTCTATTTAA
TTCGAGTGATAATGAATTAATTCTCAAATGTGGAGGGACACCAAA
TGCACGGAATCTCATCGATATCGAGCCAGTCGCAACTTTCATAGA
ATTTGAACAATTGATCTGCACAATGTTGACAACCCACTTGAAGGA
AATAATTGATATAACAAGGTCTGGAACCCAGGATTATGAAAGTTT
ATTACTCACTCCTTACAATTTAGGTCTTCTTGGTAAAATCAGTAC
GATAGTGAGATTATTAACAGAAAGGATTCTAAATCATACTATCAG
GAATTGGTTGATCCTCCCACCTTCGCTCCGGATGATCGTGAAGCA
GGACTTGGAATTCGGCATATTCAGGATTACTTCCATCCTCAATTC
TGATCGGTTCCTGAAGCTTTCTCCAAATAGGAAATACTTGATTGC
ACAATTAACTGCAGGCTACATTAGGAAATTGATTGAGGGGGATTG
CAATATCGATCTAACCAGACCTATCCAAAAGCAAATCTGGAAAGC
ATTAGGTTGTGTAGTCTATTGTCACGATCCAATGGATCAAAGGGA
GTCAACAGAGTTTATTGATATAAATATTAATGAAGAAATAGACCG
CGGGATCGATGGCGAGGAAATCTAAACATATCAAGAATCAGAATT
AGTTTAAGAAAAAAGAAGAGGATTAATCTTGGTTTTCCCCTTGGT
GGGTCGGCATGGCATCTCCACCTCCTCGCGGTCCGACCTGGGCAT
CCGAAGGAGGACGCACGTCCACTCGGATGGCTAAGGGAGCGGCCG
GGGATCCGGCTGCTAACAAAGCCCGAAAGGAAGCTGAGTTGGCTG
CTGCCACCGCTGAGCAATAACTAGCATAACCCCTTGGGGCCTCTA
AACGGGTCTTGAGGGGTTTTTTGCTGAAAGGAGGAACTATATCCG
GATCATGTGAGCAAAAGGCCAGCAAAAGGCCAGGAACCGTAAAAA
GGCCGCGTTGCTGGCGTTTTTCCATAGGCTCCGCCCCCCTGACGA
GCATCACAAAAATCGACGCTCAAGTCAGAGGTGGCGAAACCCGAC
AGGACTATAAAGATACCAGGCGTTTCCCCCTGGAAGCTCCCTCGT
GCGCTCTCCTGTTCCGACCCTGCCGCTTACCGGATACCTGTCCGC
CTTTCTCCCTTCGGGAAGCGTGGCGCTTTCTCATAGCTCACGCTG
TAGGTATCTCAGTTCGGTGTAGGTCGTTCGCTCCAAGCTGGGCTG
TGTGCACGAACCCCCCGTTCAGCCCGACCGCTGCGCCTTATCCGG
TAACTATCGTCTTGAGTCCAACCCGGTAAGACACGACTTATCGCC
ACTGGCAGCAGCCACTGGTAACAGGATTAGCAGAGCGAGGTATGT
AGGCGGTGCTACAGAGTTCTTGAAGTGGTGGCCTAACTACGGCTA
CACTAGAAGAACAGTATTTGGTATCTGCGCTCTGCTGAAGCCAGT
TACCTTCGGAAAAAGAGTTGGTAGCTCTTGATCCGGCAAACAAAC
CACCGCTGGTAGCGGTGGTTTTTTTGTTTGCAAGCAGCAGATTAC
GCGCAGAAAAAAAGGATCTCAAGAAGATCCTTTGATCTTTTCTAC
GGGGTCTGACGCTCAGTGGAACGAAAACTCACGTTAAGGGATTTT
GGTCATGAGATTATCAAAAAGGATCTTCACCTAGATCCTTTTAAA
TTAAAAATGAAGTTTTAAATCAATCTAAAGTATATATGAGTAAAC
TTGGTCTGACAGTTACCAATGCTTAATCAGTGAGGCACCTATCTC
AGCGATCTGTCTATTTCGTTCATCCATAGTTGCCTGACTCCCCGT
CGTGTAGATAACTACGATACGGGAGGGCTTACCATCTGGCCCCAG
TGCTGCAATGATACCGCGAGACCCACGCTCACCGGCTCCAGATTT
ATCAGCAATAAACCAGCCAGCCGGAAGGGCCGAGCGCAGAAGTGG
TCCTGCAACTTTATCCGCCTCCATCCAGTCTATTAATTGTTGCCG
GGAAGCTAGAGTAAGTAGTTCGCCAGTTAATAGTTTGCGCAACGT
TGTTGCCATTGCTACAGGCATCGTGGTGTCACGCTCGTCGTTTGG
TATGGCTTCATTCAGCTCCGGTTCCCAACGATCAAGGCGAGTTAC
ATGATCCCCCATGTTGTGCAAAAAAGCGGTTAGCTCCTTCGGTCC
TCCGATCGTTGTCAGAAGTAAGTTGGCCGCAGTGTTATCACTCAT
GGTTATGGCAGCACTGCATAATTCTCTTACTGTCATGCCATCCGT
AAGATGCTTTTCTGTGACTGGTGAGTACTCAACCAAGTCATTCTG
AGAATAGTGTATGCGGCGACCGAGTTGCTCTTGCCCGGCGTCAAT
ACGGGATAATACCGCGCCACATAGCAGAACTTTAAAAGTGCTCAT
CATTGGAAAACGTTCTTCGGGGCGAAAACTCTCAAGGATCTTACC
GCTGTTGAGATCCAGTTCGATGTAACCCACTCGTGCACCCAACTG
ATCTTCAGCATCTTTTACTTTCACCAGCGTTTCTGGGTGAGCAAA
AACAGGAAGGCAAAATGCCGCAAAAAAGGGAATAAGGGCGACACG
GAAATGTTGAATACTCATACTCTTCCTTTTTCAATATTATTGAAG
CATTTATCAGGGTTATTGTCTCATGAGCGGATACATATTTGAATG
TATTTAGAAAAATAAACAAATAGGGGTTCCGCGCACATTTCCCCG
AAAAGT.

viii. CVL119

The nucleic acid sequence for CVL119 is:

(SEQ ID NO: 10)
GCCACCTGACGTCTAAGAAACCATTATTATCATGACATTAACCTA
TAAAAATAGGCGTATCACGAGGCCCTTTCGTCTCGCGCGTTTCGG
TGATGACGGTGAAAACCTCTGACACATGCAGCTCCCGGAGACGGT
CACAGCTTGTCTGTAAGCGGATGCCGGGAGCAGACAAGCCCGTCA
GGGCGCGTCAGCGGGTGTTGGCGGGTGTCGGGGCTGGCTTAACTA
TGCGGCATCAGAGCAGATTGTACTGAGAGTGCACCATATATGCGG
TGTGAAATACCGCACAGATGCGTAAGGAGAAAATACCGCATCAGG
CTAATACGACTCACTATAGGGACCAAGGGGAAAATGAAGTGGTGA
CTCAAATCATCGAAGACCCTCGAGATTACATAGGTCCGGAACCTA
TGGCCTTCGTGACCGACCTCGAGTCAGAGTAGTTCAATAAGGACC
TATCAAGTTTGGGCAATTTTTCGTCCCCGACACAAAAATGTCATC
CGTGCTTAAAGCATATGAGCGATTCACGCTCACTCAAGAACTGCA
AGATCAGAGTGAGGAAGGTACAATCCCACCTACAACACTAAAACC
GGTAATCAGGGTATTTATACTAACCTCTAATAACCCAGAGCTAAG
ATCCCGGCTTCTTCTATTCTGCCTACGGATTGTTCTCAGTAATGG
TGCAAGGGATTCCCATCGCTTTGGAGCATTACTCACAATGTTTTC
GCTACCATCAGCCACAATGCTCAATCATGTCAAATTAGCTGACCA
GTCACCAGAAGCTGATATCGAAAGGGTAGAGATCGATGGCTTTGA
GGAGGGATCATTCCGCTTAATCCCCAATGCTCGTTCAGGTATGAG
CCGTGGAGAGATCAATGCCTATGCTGCACTTGCAGAAGATCTACC
TGACACACTAAACCATGCAACACCTTTCGTTGATTCCGAAGTCGA
GGGAACTGCATGGGATGAGATTGAGACTTTCTTAGATATGTGTTA
CAGTGTCCTAATGCAGGCATGGATAGTGACTTGCAAGTGCATGAC
TGCGCCAGACCAACCTGCTGCTTCTATTGAGAAACGCCTGCAAAA
ATATCGTCAGCAAGGCAGGATCAACCCGAGATATCTCCTGCAACC
GGAGGCTCGACGAATAATCCAGAATGTAATCCGGAAGGGAATGGT
GGTCAGACATTTCCTCACCTTTGAACTGCAGCTTGCCCGAGCACA
AAGCCTTGTATCAAATAGGTATTATGCTATGGTAGGGGATGTTGG
AAAGTATATAGAGAATTGTGGAATGGGAGGCTTCTTTTTGACACT
AAAATATGCATTAGGAACTAGATGGCCCACACTTGCTTTAGCTGC
ATTTTCAGGAGAGCTAACAAAGCTAAAGTCCCTCATGGCATTATA
CCAGACCCTTGGTGAGCAGGCCCGATATTTGGCCCTATTGGAGTC
ACCACATTTGATGGATTTTGCTGCAGCAAACTACCCACTGCTATA
TAGCTATGCTATGGGAATAGGCTATGTGTTAGATGTCAACATGAG
GAACTACGCTTTCTCCAGATCATACATGAACAAGACATATTTCCA
ATTGGGAATGGAAACTGCAAGAAAACAACAGGGTGCAGTTGACAT
GAGGATGGCAGAAGATCTCGGTCTAACTCAAGCCGAACGCACCGA
GATGGCAAATACACTTGCCAAATTGACCACAGCAAATCGAGGGGC
AGACACCAGGGGAGGAGTCAACCCGTTCTCATCTGTCACTGGGAC
AACTCAGGTGCCCGCTGCAGCAACAGGTGACACACTCGAGAGTTA
CATGGCAGCGGATCGACTGAGGCAGAGATATGCTGATGCAGGCAC
CCATGATGATGAGATGCCACCATTGGAAGAGGAGGAAGAGGACGA
CACATCTGCAGGTCCACGCACTGGACCAACTCTTGAACAAGTGGC
CTTGGACATCCAGAACGCAGCAGTTGGAGCTCCCATCCATACAGA
TGACCTGAATGCCGCACTGGGTGATCTTGACATCTAGACAATTCA
GATCCCAATCTAAAATTGACATACCTAATTGATTAGTTAGATGGA
ACTACAGTGGATTCCATAAGGTTCCTGCCTACCATCGGCTTTAAA
GAAAAAAATAGGCCCGGACGGGTTAGCAACAAGCGACTGCCGGTG
CCAACAGCGCAATCCACAATCTACAATGGATCCCACTGATCTGAG
CTTCTCCCCAGATGAGATCAATAAGCTCATAGAGACAGGCCTGAA
TACTGTAGAGTATTTTACTTCCCAACAAGTCACAGGAACATCCTC
TCTTGGAAAGAATACAATACCACCAGGGGTCACAGGACTACTAAC
CAATGCTGCAGAGGCAAAGATCCAAGAGTCAACTAACCATCAGAA
GGGCTCAGTTGGTGGGGGTGCAAAACCAAAGAAACCGCGACCAAA
AATTGCCATTGTGCCAGCAGATGACAAAACAGTGCCCGGAAAGCC
GATCCCAAACCCTCTATTAGGTCTGGACTCCACCCCGAGCACCCA
AACTGTGCTTGATCTAAGTGGGAAAACATTACCATCAGGATCCTA
TAAGGGGGTTAAGCTTGCGAAATTTGGAAAAGAAAATCTGATGAC
ACGGTTCATCGAGGAACCCAGAGAGAATCCTATCGCAACCAGTTt
CCCCATCGATTTTAAGAGGGGCAGGGATACCGGCGGGTTCCATAG
AAGGGAGTACTCAATCGGATGGGTGGGAGATGAAGTCAAGGTCAC
TGAGTGGTGCAATCCATCCTGTTCTCCAATCACCGCTGCAGCAAG
GCGATTTGAATGCACTTGTCACCAGTGTCCAGTCACTTGCTCTGA
ATGTGAACGAGATACTTAATACAGTGAGAAATTTGGACTCTCGGA
TGAATCAACTGGAGACAAAAGTAGATCGCATTCTCTCATCTCAGT
CTCTAATCCAGACCATCAAGAATGACATAGTTGGACTTAAAGCAG
GGATGGCTACTTTAGAAGGAATGATTACAACTGTGAAAATCATGG
ACCCGGGAGTTCCCAGTAATGTTACTGTGGAAGATGTACGCAAGA
CACTAAGTAACCATGCTGTTGTTGTGCCAGAATCATTCAATGATA
GTTTCTTGACTCAATCTGAAGATGTAATTTCACTTGATGAGTTGG
CTCGACCAACTGCAACAAGTGTTAAGAAGATTGTCAGGAAGGTTC
CTCCTCAGAAGGATCTGACTGGATTGAAGATTACACTAGAGCAAT
TGGCAAAGGATTGCATCAGCAAACCGAAGATGAGGGAAGAGTATC
TCCTCAAAATCAACCAGGCTTCCAGTGAGGCTCAGCTAATTGACC
TCAAGAAAGCAATCATCCGCAGTGCAATTTGATCAAGAAACACCC
AATTACACTACACTGGTATGACACTGTACTAACCCTGAGGGTTTT
AGAAAAAACGATTAACGATAAATAAGCCCGAACACTACACACTAC
CTGAGGCAGCCATGCCATCCATCAGCATTCCCGCAGACCCCACCA
ATCCACGTCAATCAATAAAAGCGTTCCCAATTGTGATCAACAGTG
ATGGGGGTGAGAAAGGCCGCTTGGTTAAACAACTACGCACAACCT
ACTTGAATGACCTAGATACTCATGAGCCACTGGTGACATTCATAA
ATACCTATGGATTCATCTACGAACAGGATCGGGGGAATACCATTG
TCGGAGAGGATCAACTTGGGAAGAAAAGAGAGGCTGTGACCGCTG
CAATGGTTACCCTTGGATGTGGGCCTAATCTACCATCATTAGGGA
ATGTCCTGGGACAACTGAGGGAATTCCAGGTCACTGTTAGGAAGA
CATCCAGCAAAGCGGAAGAGATGGTCTTTGAAATTGTTAAGTATC
CGAGAATATTTCGGGGTCATACATTAATCCAGAAAGGACTAGTCT
GTGTCTCCGCAGAAAAATTTGTTAAGTCACCAGGGAAAATACAAT
CTGGAATGGACTATCTCTTCATTCCGACATTTCTGTCAGTGACTT
ACTGTCCAGCTGCAATCAAATTTCAGGTACCTGGCCCCATGTTGA
AAATGAGATCAAGATACACTCAGAGCTTACAACTTGAACTAATGA
TAAGAATCCTGTGTAAGCCCGATTCGCCACTTATGAAGGTCCATA
CCCCTGACAAGGAGGGAAGAGGATGTCTTGTATCAGTATGGCTGC
ATGTATGCAACATCTTCAAATCAGGAAACAAGAATGGCAGTGAGT
GGCAGGAATACTGGATGAGAAAGTGTGCTAACATGCAACTTGAAG
TGTCGATTGCAGATATGTGGGGACCAACTATCATAATTCATGCCA
GAGGTCACATTCCCAAAAGTGCTAAGTTGTTTTTTGGAAAGGGTG
GATGGAGCTGCCATCCACTTCACGAAGTTGTTCCAAGTGTCACTA
AAACACTATGGTCCGTGGGCTGTGAGATTACAAAGGCGAAGGCAA
TAATACAAGAGAGTAGCATCTCTCTTCTCGTGGAGACTACTGACA
TCATAAGTCCAAAAGTCAAAATTTCATCTAAGCATCGCCGCTTTG
GGAAATCAAATTGGGGTCTGTTCAAGAAAACTAAATCACTGCCTA
ACCTGACGGAGCTGGAATGACTGACCTCTAATCGAGACTACACCG
CCGCAAACTATAGGTGGGTGGTACCTCAGTGATTAATCTTGTAAG
CACTGATCGTAGGCTACAACACACTAATATTATCCAGATTAGAGA
GCTTAATTAGCTCTGTATTAATAATAACACTACTATTCCAATAAC
TGGAATCACCAGCTTGATTTATCTCCAAAATGATTCAAAGAAAAC
AAATCATATTAAGACTATCCTAAGCACGAACCCATATCGTCCTTC
AAATCATGGGTACTATAATTCAATTTCTGGTGGTCTCCTGTCTAT
TGGCAGGAGCAGGCAGCCTTGATCCAGCAGCCCTCATGCAAATCG
GTGTCATTCCAACAAATGTCCGGCAACTTATGTATTATACTGAAG
CTTCATCAGCATTCATTGTTGTGAAGTTAATGCCTACAATTGACT
CGCCGATTAGTGGATGTAATATAACATCAATTTCAAGCTATAATG
CAACAGTGACAAAACTCCTACAGCCGATCGGTGAGAATTTGGAGA
CGATTAGGAACCAGTTGATTCCAACTCGGAGGAGACGCCGGTTTG
CAGGGGTGGTGATTGGATTAGCTGCATTAGGAGTAGCTACTGCCG
CACAGGTCACTGCCGCAGTGGCACTAGTAAAGGCAAATGAAAATG
CTGCGGCTATACTCAATCTCAAAAATGCAATCCAAAAAACAAATG
CAGCAGTTGCAGATGTGGTCCAGGCCACACAATCACTAGGAACGG
CAGTTCAAGCAGTTCAAGATCACATAAACAGTGTGGTAAGTCCAG
CAATTACAGCAGCCAATTGTAAGGCCCAAGATGCTATCATTGGCT
CAATCCTCAATCTCTATTTGACCGAGTTGACAACCATCTTCCACA
ATCAAATTACAAACCCTGCATTGAGTCCCATTACAATTCAAGCTT
TAAGGATCCTACTGGGGAGTACCTTGCCGACTGTGGTCGAAAAAT
CTTTCAATACCCAGATAAGTGCAGCTGAGCTTCTCTCATCAGGGT
TATTGACAGGCCAGATTGTGGGATTAGATTTGACCTATATGCAGA
TGGTCATAAAAATTGAGCTGCCAACTTTAACTGTACAACCTGCAA
CCCAGATCATAGATCTGGCCACCATTTCTGCATTCATTAACAATC
AAGAAGTCATGGCCCAATTACCAACACGTGTTATGGTGACTGGCA
GCTTGATCCAAGCCTATCCCGCATCGCAATGCACCATTACACCCA
ACACTGTGTACTGTAGGTATAATGATGCCCAAGTACTCTCAGATG
ATACTATGGCTTGCCTCCAAGGTAACTTGACAAGATGCACCTTCT
CTCCAGTGGTTGGGAGCTTTCTCACTCGATTCGTGCTGTTCGATG
GAATAGTTTATGCAAATTGCAGGTCGATGTTGTGCAAGTGCATGC
AACCTGCTGCTGTGATCCTACAGCCGAGTTCATCCCCTGTAACTG
TCATTGACATGTACAAATGTGTGAGTCTGCAGCTTGACAATCTCA
GATTCACCATCACTCAATTGGCCAATGTAACCTACAATAGCACCA
TCAAGCTTGAATCATCCCAGATCTTGTCTATTGATCCGTTGGATA
TATCCCAGAATCTAGCTGCGGTGAATAAGAGTCTAAGTGATGCAC
TACAACACTTAGCACAAAGTGACACATATCTTTCTGCAATCACAT
CAGCTACGACTACAAGTGTATTATCCATAATAGCAATCTGTCTTG
GATCGTTAGGTTTAATATTAATAATCTTGCTCAGTGTAGTTGTGT
GGAAGTTATTGACCATTGTCGTTGCTAATCGAAATAGAATGGAGA
ATTTTGTTTATCATAAATAAGCATTCCACCACTCACGATCTGATC
TCAGTGAGAAAAATCAACCTGCAACTCTTGGAACAAGATAAGACA
GTCATCCATTAGTAATTTTTAAGAAAAAAACGATAGGACCGAACC
TAGTATTGAAAGAACCGTCTCGGTCAATCTAGGTAATCGAGCTGA
TACCGTCTCGGAAAGCTCAAATCGCGCGCCACCatgtcagataac
ggaccacagaaccagaggaacgcacccaggattactttcggagga
ccaagcgatagcaccgggagcaaccagaatggagagcggagcgga
gcaagatccaagcagagacggccccagggcctgccaaacaatacc
gcatcctggttcaccgccctgacacagcacggcaaggaggacctg
aagtttccaaggggacagggagtgcctatcaacaccaatagctcc
cctgacgatcagatcggctactataggagggcaacaaggagaatc
aggggaggcgacggcaagatgaaggatctgagcccacgctggtac
ttctactatctgggaaccggacctgaggcaggcctgccatatggc
gccaacaaggacggaatcatctgggtggcaaccgagggcgccctg
aacacaccaaaggatcacatcggcacaagaaatcccgccaacaat
gcagcaatcgtgctgcagctgccacagggaaccacactgcccaag
ggcttttacgcagagggctctcggggaggcagccaggcatctagc
agatcctctagccggagcagaaactcctctaggaattccacccca
ggaagctccaggggcacatcccctgcccgcatggcaggaaacgga
ggcgacgccgccctggccctgctgctgctggatcgcctgaatcag
ctggagtccaagatgtctggcaagggacagcagcagcagggacag
accgtgacaaagaagtccgccgccgaggcctctaagaagccaagg
cagaagcgcaccgccacaaaggcctacaacgtgacccaggccttc
ggcaggcgcggaccagagcagacacagggcaattttggcgaccag
gagctgatcaggcagggaaccgattataagcactggcctcagatc
gcccagttcgccccatctgccagcgccttctttggcatgtctaga
atcggcatggaggtgacccccagcggcacatggctgacctacaca
ggcgccatcaagctggacgataaggaccctaacttcaaggatcag
gtcatcctgctgaacaagcacatcgacgcctataagacctttccc
cctacagagcccaagaaggacaagaagaagaaggccgatgagaca
caggccctgcctcagaggcagaagaagcagcagaccgtgacactg
ctgccagccgccgatctggacgatttctccaaacagctgcagcag
agcatgtccagtgccgactccacccaggcttgaCGTACGACCTGC
TATAGGCTATCCACTGCATCATCTCTCCTGCCATACTTCCTACTC
ACATCATATCTATTTTAAAGAAAAAATAGGCCCGAACACTAATCG
TGCCGGCAGTGCCACTGCACACACAACACTACACATACAATACAC
TACAATGGTTGCAGAAGATGCCCCTGTTAGGGCCACTTGCCGAGT
ATTATTTCGAACAACAACTTTAATCTTTCTATGCACACTACTAGC
ATTAAGCATCTCTATCCTTTATGAGAGTTTAATAACCCAAAAGCA
AATCATGAGCCAAGCAGGCTCAACTGGATCTAATTCTGGATTAGG
AAGTATCACTGATCTTCTTAATAATATTCTCTCTGTCGCAAATCA
GATTATATATAACTCTGCAGTCGCTCTACCTCTACAATTGGACAC
TCTTGAATCAACACTCCTTACAGCCATTAAGTCTCTTCAAACCAG
TGACAAGCTAGAACAGAACTGCTCGTGGAGTGCTGCACTGATTAA
TGATAATAGATACATTAATGGCATCAATCAGTTCTATTTTTCAAT
TGCTGAGGGTCGCAATCTGACACTTGGCCCACTTCTTAATATGCC
TAGTTTCATTCCAACTGCCACGACACCAGAGGGCTGCACCAGGAT
CCCATCATTCTCGCTCACTAAGACACACTGGTGTTATACACACAA
TGTTATCCTGAATGGATGCCAGGATCATGTATCCTCAAATCAATT
TGTTTCTATGGGAATCATTGAACCCACTTCTGCCGGGTTTCCATT
CTTTCGAACCCTAAAGACTCTATATCTCAGCGATGGGGTCAATCG
TAAGAGCTGCTCTATCAGTACAGTTCCGGGGGGTTGTATGATGTA
CTGTTTTGTTTCTACTCAACCAGAGAGGGATGACTACTTTTCTGC
CGCTCCTCCAGAACAACGAATTATTATAATGTACTATAATGATAC
AATCGTGGAGCGCATAATTAATCCACCCGGGGTACTAGATGTATG
GGCAACATTGAACCCAGGAACAGGAAGCGGGGTATATTATTTAGG
TTGGGTGCTCTTTCCAATATATGGCGGCGTGATTAAAGGTACGAG
TTTATGGAATAATCAAGCAAATAAATACTTTATCCCCCAGATGGT
TGCTGCTCTCTGCTCACAAAACCAGGCAACTCAAGTCCAAAATGC
TAAGTCATCATACTATAGCAGCTGGTTTGGCAATCGAATGATTCA
GTCTGGGATCCTGGCATGTCCTCTTCGACAGGATCTAACCAATGA
GTGTTTAGTTCTGCCCTTTTCTAATGATCAGGTGCTTATGGGTGC
TGAAGGGAGATTATACATGTATGGTGACTCGGTGTATTACTATCA
AAGAAGCAATAGTTGGTGGCCTATGACCATGCTGTATAAGGTAAC
CATAACATTCACTAATGGTCAGCCATCTGCTATATCAGCTCAGAA
TGTGCCCACACAGCAGGTCCCTAGACCTGGGACAGGAGACTGCTC
TGCAACCAATAGATGTCCCGGTTTTTGCTTGACAGGAGTGTATGC
CGATGCCTGGTTACTGACCAACCCTTCGTCTACCAGTACATTTGG
ATCAGAAGCAACCTTCACTGGTTCTTATCTCAACACAGCAACTCA
GCGTATCAATCCGACGATGTATATCGCGAACAACACACAGATCAT
AAGCTCACAGCAATTTGGATCAAGCGGTCAAGAAGCAGCATATGG
CCACACAACTTGTTTTAGGGACACAGGCTCTGTTATGGTATACTG
TATCTATATTATTGAATTGTCCTCATCTCTCTTAGGACAATTTCA
GATTGTCCCATTTATCCGTCAGGTGACACTATCCTAAAGGCAGAA
GCCTTCAGGTCTGACCCAGCCAATCAAAGCATTATACCAGACCAT
GGCCTACCATCGGCTTTAAAGAAAAAAATAGGCCCGGACGGGTTA
GCAACAAGCGGCGGCCGCCGCCACCatgttcgtcttcctggtcct
gctgcctctggtctcctcacagtgcgtcaatctgacaactcggac
tcagctgccacctgcttatactaatagcttcaccagaggcgtgta
ctatcctgacaaggtgtttagaagctccgtgctgcactctacaca
ggatctgtttctgccattctttagcaacgtgacctggttccacgc
catccacgtgagcggcaccaatggcacaaagcggttcgacaatcc
cgtgctgccttttaacgatggcgtgtacttcgcctctaccgagaa
gagcaacatcatcagaggctggatctttggcaccacactggactc
caagacacagtctctgctgatcgtgaacaatgccaccaacgtggt
catcaaggtgtgcgagttccagttttgtaatgatcccttcctggg
cgtgtactatcacaagaacaataagagctggatggagtccgagtt
tagagtgtattctagcgccaacaactgcacatttgagtacgtgag
ccagcctttcctgatggacctggagggcaagcagggcaatttcaa
gaacctgagggagttcgtgtttaagaatatcgacggctacttcaa
aatctactctaagcacacccccatcaacctggtgcgcgacctgcc
tcagggcttcagcgccctggagcccctggtggatctgcctatcgg
catcaacatcacccggtttcagacactgctggccctgcacagaag
ctacctgacacccggcgactcctctagcggatggaccgccggcgc
tgccgcctactatgtgggctacctccagccccggaccttcctgct
gaagtacaacgagaatggcaccatcacagacgcagtggattgcgc
cctggaccccctgagcgagacaaagtgtacactgaagtcctttac
cgtggagaagggcatctatcagacatccaatttcagggtgcagcc
aaccgagtctatcgtgcgctttcctaatatcacaaacctgtgccc
atttggcgaggtgttcaacgcaacccgcttcgccagcgtgtacgc
ctggaataggaagcggatcagcaactgcgtggccgactatagcgt
gctgtacaactccgcctctttcagcacctttaagtgctatggcgt
gtcccccacaaagctgaatgacctgtgctttaccaacgtctacgc
cgattctttcgtgatcaggggcgacgaggtgcgccagatcgcccc
cggccagacaggcaagatcgcagactacaattataagctgccaga
cgatttcaccggctgcgtgatcgcctggaacagcaacaatctgga
ttccaaagtgggcggcaactacaattatctgtaccggctgtttag
aaagagcaatctgaagcccttcgagagggacatctctacagaaat
ctaccaggccggcagcaccccttgcaatggcgtggagggctttaa
ctgttatttcccactccagtcctacggcttccagcccacaaacgg
cgtgggctatcagccttaccgcgtggtggtgctgagctttgagct
gctgcacgccccagcaacagtgtgcggccccaagaagtccaccaa
tctggtgaagaacaagtgcgtgaacttcaacttcaacggcctgac
cggcacaggcgtgctgaccgagtccaacaagaagttcctgccatt
tcagcagttcggcagggacatcgcagataccacagacgccgtgcg
cgacccacagaccctggagatcctggacatcacaccctgctcttt
cggcggcgtgagcgtgatcacacccggcaccaatacaagcaacca
ggtggccgtgctgtatcaggacgtgaattgtaccgaggtgcccgt
ggctatccacgccgatcagctgaccccaacatggcgggtgtacag
caccggctccaacgtcttccagacaagagccggatgcctgatcgg
agcagagcacgtgaacaattcctatgagtgcgacatcccaatcgg
cgccggcatctgtgcctcttaccagacccagacaaactctcccag
aagagcccggagcgtggcctcccagtctatcatcgcctataccat
gtccctgggcgccgagaacagcgtggcctactctaacaatagcat
cgccatcccaaccaacttcacaatctctgtgaccacagagatcct
gcccgtgtccatgaccaagacatctgtggactgcacaatgtatat
ctgtggcgattctaccgagtgcagcaacctgctgctccagtacgg
cagcttttgtacccagctgaatagagccctgacaggcatcgccgt
ggagcaggataagaacacacaggaggtgttcgcccaggtgaagca
aatctacaagaccccccctatcaaggactttggcggcttcaattt
ttcccagatcctgcctgatccatccaagccttctaagcggagctt
tatcgaggacctgctgttcaacaaggtgaccctggccgatgccgg
cttcatcaagcagtatggcgattgcctgggcgacatcgcagccag
ggacctgatctgcgcccagaagtttaatggcctgaccgtgctgcc
acccctgctgacagatgagatgatcgcacagtacacaagcgccct
gctggccggcaccatcacatccggatggaccttcggcgcaggagc
cgccctccagatcccctttgccatgcagatggcctataggttcaa
cggcatcggcgtgacccagaatgtgctgtacgagaaccagaagct
gatcgccaatcagtttaactccgccatcggcaagatccaggacag
cctgtcctctacagccagcgccctgggcaagctccaggatgtggt
gaatcagaacgcccaggccctgaataccctggtgaagcagctgag
cagcaacttcggcgccatctctagcgtgctgaatgacatcctgag
ccggctggacaaggtggaggcagaggtgcagatcgaccggctgat
caccggccggctccagagcctccagacctatgtgacacagcagct
gatcagggccgccgagatcagggccagcgccaatctggcagcaac
caagatgtccgagtgcgtgctgggccagtctaagagagtggactt
ttgtggcaagggctatcacctgatgtccttccctcagtctgcccc
acacggcgtggtgtttctgcacgtgacctacgtgcccgcccagga
gaagaacttcaccacagcccctgccatctgccacgatggcaaggc
ccactttccaagggagggcgtgttcgtgtccaacggcacccactg
gtttgtgacacagcgcaatttctacgagccccagatcatcaccac
agacaacaccttcgtgagcggcaactgtgacgtggtcatcggcat
cgtgaacaataccgtgtatgatccactccagcccgagctggacag
ctttaaggaggagctggataagtatttcaagaatcacacctcccc
tgacgtggatctgggcgacatcagcggcatcaatgcctccgtggt
gaacatccagaaggagatcgaccgcctgaacgaggtggctaagaa
tctgaacgagagcctgatcgacctccaggagctgggcaagtatga
gcagtacatcaagtggccctggtacatctggctgggcttcatcgc
cggcctgatcgccatcgtgatggtgaccatcatgctgtgctgtat
gacatcctgctgttcttgcctgaagggctgctgtagctgtggctc
ctgctgtaagtttgacgaggatgactctgaacctgtgctgaaggg
cgtgaagctgcattacacctaaTAAGGTCGACTCATGGAATGCAT
ACCAAACATTATTGACACTAATGACACACAAAATTGGTTTTAAGA
AAAACCAAGAGAACAATAGGCCAGAATGGCTGGGTCTCGGGAGAT
ATTACTCCCTGAAGTCCATCTCAATTCACCAATTGTAAAGCATAA
GCTATACTATTACATTCTACTTGGAAACCTCCCAAATGAGATCGA
CCTTGACGATTTAGGTCCATTACATAATCAAAATTGGAATCAGAT
AGCACATGAAGAGTCTAACTTAGCTCAACGCTTGGTAAATGTAAG
AAATTTTCTAATTACCCACATCCCTGATCTTAGAAAGGGCCATTG
GCAAGAGTATGTCAATGTAATACTGTGGCCGCGAATTCTTCCCTT
GATCCCGGATTTTAAAATCAATGACCAATTGCCTCTGCTCAAAAA
TTGGGACAAGTTAGTTAAAGAATCATGTTCAGTAATCAATGCAGG
TACTTCCCAGTGCATTCAGAATCTCAGCTATGGACTGACAGGTCG
TGGGAACCTCTTTACACGATCACGTGAACTCTCTGGTGACCGCAG
GGATATTGATCTTAAGACAGTTGTGGCAGCATGGCATGACTCAGA
CTGGAAAAGAATAAGTGATTTTTGGATTATGATCAAATTCCAGAT
GAGACAATTAATTGTTAGGCAAACAGATCATAATGATTCTGATTT
AATCACGTATATCGAAAATAGAGAAGGCATAATCATCATAACCCC
TGAACTGGTAGCATTATTTAACACTGAGAATCATACACTAACATA
CATGACCTTTGAAATTGTACTGATGGTTTCAGATATGTACGAAGG
TCGTCACAACATTTTATCACTATGCACAGTTAGCACTTACCTGAA
TCCTCTGAAGAAAAGAATAACATATTTATTGAGCCTTGTAGATAA
CTTAGCTTTTCAGATAGGTGATGCTGTATATAACATAATTGCTTT
GCTAGAATCCTTTGTATATGCACAGTTGCAAATGTCAGATCCCAT
CCCAGAACTCAGAGGACAATTCCATGCATTCGTATGTTCTGAGAT
TCTTGATGCACTAAGAGGAACTAATAGTTTCACCCAGGATGAATT
AAGAACTGTGACAACTAATTTGATATCCCCATTCCAAGATCTGAC
CCCAGATCTTACGGCTGAATTGCTCTGTATAATGAGGCTTTGGGG
ACACCCCATGCTCACTGCCAGTCAAGCTGCAGGAAAGGTACGCGA
GTCTATGTGTGCTGGAAAAGTATTAGACTTTCCCACCATTATGAA
AACACTAGCCTTTTTCCATACTATTCTGATCAATGGATACAGGAG
GAAGCATCATGGAGTATGGCCACCCTTAAACTTACCGGGTAATGC
TTCAAAGGGTCTCACGGAACTTATGAATGACAATACTGAAATAAG
CTATGAATTCACACTTAAGCATTGGAAGGAAGTCTCTCTTATAAA
ATTCAAGAAATGTTTTGATGCAGACGCAGGTGAGGAACTCAGTAT
ATTTATGAAAGATAAGGCAATTAGTGCCCCAAAACAAGACTGGAT
GAGTGTGTTTAGAAGAAGCCTAATCAAACAGCGCCATCAGCATCA
TCAGGTCCCCCTACCAAATCCATTCAATCGACGGCTGTTGCTAAA
CTTTCTCGGAGATGACAAATTCGACCCGAATGTGGAGCTACAGTA
TGTAACATCAGGTGAGTATCTACATGATGACACGTTTTGTGCATC
ATATTCACTAAAAGAGAAGGAAATTAAACCTGATGGTCGAATTTT
TGCAAAGTTGACTAAGAGAATGAGATCATGTCAAGTTATAGCAGA
ATCTCTTTTAGCGAACCATGCTGGGAAGTTAATGAAAGAGAATGG
TGTTGTGATGAATCAGCTATCATTAACAAAATCACTATTAACAAT
GAGTCAGATTGGAATAATATCCGAGAAAGCTAGAAAGTCAACTCG
AGATAACATAAATCAACCTGGTTTCCAGAATATCCAGAGAAATAA
ATCACATCACTCCAAGCAAGTCAATCAGCGAGATCCAAGTGATGA
CTTTGAATTGGCAGCATCTTTTTTAACTACTGATCTCAAAAAATA
TTGTTTACAATGGAGGTACCAGACAATTATCCCATTTGCTCAATC
ATTAAACAGAATGTATGGTTATCCTCATCTCTTTGAGTGGATTCA
CTTACGGCTAATGCGTAGTACACTTTACGTGGGGGATCCCTTCAA
CCCACCAGCAGATACCAGTCAATTTGATCTAGATAAAGTAATTAA
TGGAGATATCTTCATTGTATCACCCAGAGGTGGAATTGAAGGGCT
GTGTCAAAAGGCTTGGACAATGATATCTATCGCTGTGATAATTCT
ATCTGCCACAGAGTCTGGCACACGAGTAATGAGTATGGTGCAGGG
AGATAATCAAGCAATTGCTGTCACCACACGAGTACCAAGGAGCCT
GCCGACTCTTGAGAAAAAGACTATTGCTTTTAGATCTTGTAATCT
ATTCTTTGAGAGGTTAAAATGTAATAATTTTGGATTAGGTCACCA
TTTGAAAGAACAAGAGACTATCATTAGTTCTCACTTCTTTGTTTA
TAGCAAGAGAATATTCTATCAGGGGAGGATTCTAACGCAAGCCTT
AAAAAATGCTAGTAAGCTCTGCTTGACAGCTGATGTCCTAGGAGA
ATGCACCCAATCATCATGTTCTAATCTTGCAACTACTGTCATGAG
GTTAACTGAGAATGGTGTTGAAAAAGATATCTGTTTCTACTTGAA
TATCTATATGACCATCAAACAGCTCTCCTATGATATCATCTTCCC
TCAAGTGTCAATTCCTGGAGATCAGATCACATTAGAATACATAAA
TAATCCACACCTGGTATCACGATTGGCTCTTTTGCCATCCCAGTT
AGGAGGTCTAAACTACCTGTCATGCAGTAGGCTGTTCAATCGAAA
CATAGGCGACCCGGTGGTTTCCGCAGTTGCAGATCTTAAGAGATT
AATTAAATCAGGATGTATGGATTACTGGATCCTTTATAACTTATT
AGGGAGAAAACCGGGAAACGGCTCATGGGCTACTTTAGCAGCTGA
CCCGTACTCAATCAATATAGAGTATCAATACCCTCCAACTACAGC
TCTTAAGAGGCACACCCAACAAGCTCTGATGGAACTCAGTACGAA
TCCAATGTTACGTGGCATATTCTCTGACAATGCACAGGCAGAAGA
AAATAACCTTGCTAGGTTTCTCCTGGATAGGGAGGTGATCTTTCC
GCGTGTAGCTCACATCATCATTGAGCAAACCAGTGTCGGGAGGAG
AAAACAGATTCAAGGATATTTGGATTCAACTAGATCGATAATGAG
GAAATCACTAGAAATTAAGCCCTTATCCAATAGGAAGCTTAATGA
AATACTGGATTACAACATCAATTACCTAGCTTACAATTTGGCATT
ACTCAAGAATGCTATTGAACCTCCGACTTATTTGAAGGCAATGAC
ACTTGAAACATGTAGCATCGACATTGCAAGGAACCTCCGGAAGCT
CTCCTGGGCCCCACTCTTGGGTGGGAGAAATCTTGAAGGATTAGA
GACGCCAGATCCCATTGAAATTACTGCAGGAGCATTAATTGTTGG
ATCGGGCTACTGTGAACAGTGTGCTGCAGGAGACAATCGATTCAC
ATGGTTTTTCTTGCCATCTGGTATCGAGATAGGAGGGGATCCCCG
TGATAATCCTCCTATCCGTGTACCGTACATTGGCTCCAGGACTGA
TGAGAGGAGGGTAGCCTCAATGGCATACATCAGGGGTGCCTCGAG
TAGCTTAAAAGCAGTTCTTAGACTGGCGGGAGTGTACATCTGGGC
ATTCGGAGATACTCTGGAGAATTGGATAGATGCACTGGATTTGTC
TCACACTAGAGTTAACATCACACTTGAACAGCTGCAATCCCTCAC
CCCACTTCCAACCTCTGCCAATCTAACCCATCGGTTGGATGATGG
CACAACTACCCTAAAGTTTACTCCTGCGAGCTCTTACACCTTTTC
AAGTTTCACTCATATATCAAATGATGAGCAATACCTGACAATTAA
TGACAAAACTGCAGATTCAAATATAATCTACCAACAGTTAATGAT
CACTGGACTCGGAATCTTAGAAACATGGAATAATCCCCCAATCAA
TAGAACATTCGAAGAATCTACCCTACATTTGCACACTGGTGCATC
ATGTTGTGTCCGACCTGTGGACTCCTGCATTCTCTCAGAAGCATT
AACAGTCAAGCCACATATTACAGTACCGTACAGCAATAAATTTGT
ATTTGATGAGGACCCGCTATCTGAATATGAAACTGCAAAACTGGA
ATCGTTATCATTCCAAGCCCAATTAGGCAACATTGATGCTGTAGA
TATGACAGGTAAATTAACATTATTGTCCCAATTCACTGCAAGGCA
GATTATCAATGCAATCACTGGACTCGATGAGTCTGTCTCTCTTAC
TAATGATGCCATTGTTGCATCAGACTATGTCTCCAATTGGATTAG
TGAATGCATGTATACCAAATTAGATGAATTATTTATGTATTGTGG
GTGGGAACTACTATTGGAACTATCCTATCAAATGTATTATCTGAG
GGTAGTTGGGTGGAGTAATATAGTGGATTATTCTTACATGATCTT
GAGAAGAATCCCGGGTGCAGCATTAAACAATCTGGCATCTACATT
AAGTCATCCAAAACTTTTCCGACGAGCTATCAACCTAGATATAGT
TGCCCCCTTAAATGCTCCTCATTTTGCATCTCTGGACTACATCAA
GATGAGTGTGGATGCAATACTCTGGGGCTGTAAAAGAGTCATCAA
TGTGCTCTCCAATGGAGGGGACTTAGAATTAGTTGTGACATCTGA
AGATAGCCTTATTCTCAGTGACCGATCCATGAATCTCATTGCAAG
GAAATTAACTTTATTATCACTGATTCACCATAATGGTTTGGAACT
ACCAAAGATTAAGGGGTTCTCTCCTGATGAGAAGTGTTTCGCTTT
GACAGAATTTTTGAGGAAAGTGGTGAACTCAGGGTTGAGTTCAAT
AGAGAACCTATCAAATTTTATGTACAATGTGGAGAACCCACGGCT
TGCAGCATTCGCCAGCAACAATTACTACCTGACCAGAAAATTATT
GAATTCAATACGAGATACTGAGTCGGGTCAAGTAGCAGTCACCTC
ATATTATGAATCATTAGAATATATTGATAGTCTTAAGCTAACCCC
ACATGTGCCTGGCACCTCATGCATTGAGGATGATAGTCTATGTAC
AAATGATTACATAATCTGGATCATAGAGTCTAATGCAAACTTGGA
GAAGTATCCAATTCCAAATAGCCCTGAGGATGATTCCAATTTCCA
TAACTTTAAGTTGAATGCTCCATCGCACCATACCTTACGCCCATT
AGGGTTGTCATCAACTGCTTGGTATAAGGGTATAAGCTGCTGCAG
GTACCTTGAGCGATTAAAGCTACCACAAGGTGATCATTTATATAT
TGCAGAAGGTAGTGGTGCCAGTATGACAATCATAGAATACCTATT
CCCAGGAAGAAAGATATATTACAATTCTTTATTTAGTAGTGGTGA
CAATCCCCCACAAAGAAATTATGCACCAATGCCTACTCAGTTCAT
TGAGAGTGTCCCATACAAGCTCTGGCAAGCACACACAGATCAATA
TCCCGAGATTTTTGAGGACTTCATCCCTCTATGGAACGGAAACGC
CGCCATGACTGACATAGGAATGACAGCTTGTGTAGAATTCATCAT
CAATCGAGTCGGCCCAAGGACTTGCAGTTTAGTACATGTAGATTT
GGAATCAAGTGCAAGCTTAAATCAACAATGCCTGTCAAAGCCGAT
AATTAATGCTATCATCACTGCTACAACTGTTTTGTGCCCTCATGG
GGTGCTTATTCTGAAATATAGTTGGTTGCCATTTACTAGATTTAG
TACTTTGATCACTTTCTTATGGTGCTACTTTGAGAGAATCACTGT
TCTTAGGAGCACATATTCTGATCCAGCTAATCATGAGGTTTATTT
AATTTGTATCCTTGCCAACAACTTTGCATTCCAGACTGTCTCGCA
GGCAACAGGAATGGCGATGACTTTAACTGATCAAGGGTTTACTTT
GATATCACCTGAAAGAATAAATCAGTATTGGGATGGTCACTTGAA
GCAAGAACGTATCGTAGCAGAAGCAATTGATAAGGTGGTTCTAGG
AGAAAATGCTCTATTTAATTCGAGTGATAATGAATTAATTCTCAA
ATGTGGAGGGACACCAAATGCACGGAATCTCATCGATATCGAGCC
AGTCGCAACTTTCATAGAATTTGAACAATTGATCTGCACAATGTT
GACAACCCACTTGAAGGAAATAATTGATATAACAAGGTCTGGAAC
CCAGGATTATGAAAGTTTATTACTCACTCCTTACAATTTAGGTCT
TCTTGGTAAAATCAGTACGATAGTGAGATTATTAACAGAAAGGAT
TCTAAATCATACTATCAGGAATTGGTTGATCCTCCCACCTTCGCT
CCGGATGATCGTGAAGCAGGACTTGGAATTCGGCATATTCAGGAT
TACTTCCATCCTCAATTCTGATCGGTTCCTGAAGCTTTCTCCAAA
TAGGAAATACTTGATTGCACAATTAACTGCAGGCTACATTAGGAA
ATTGATTGAGGGGGATTGCAATATCGATCTAACCAGACCTATCCA
AAAGCAAATCTGGAAAGCATTAGGTTGTGTAGTCTATTGTCACGA
TCCAATGGATCAAAGGGAGTCAACAGAGTTTATTGATATAAATAT
TAATGAAGAAATAGACCGCGGGATCGATGGCGAGGAAATCTAAAC
ATATCAAGAATCAGAATTAGTTTAAGAAAAAAGAAGAGGATTAAT
CTTGGTTTTCCCCTTGGTGGGTCGGCATGGCATCTCCACCTCCTC
GCGGTCCGACCTGGGCATCCGAAGGAGGACGCACGTCCACTCGGA
TGGCTAAGGGAGCGGCCGGGGATCCGGCTGCTAACAAAGCCCGAA
AGGAAGCTGAGTTGGCTGCTGCCACCGCTGAGCAATAACTAGCAT
AACCCCTTGGGGCCTCTAAACGGGTCTTGAGGGGTTTTTTGCTGA
AAGGAGGAACTATATCCGGATCATGTGAGCAAAAGGCCAGCAAAA
GGCCAGGAACCGTAAAAAGGCCGCGTTGCTGGCGTTTTTCCATAG
GCTCCGCCCCCCTGACGAGCATCACAAAAATCGACGCTCAAGTCA
GAGGTGGCGAAACCCGACAGGACTATAAAGATACCAGGCGTTTCC
CCCTGGAAGCTCCCTCGTGCGCTCTCCTGTTCCGACCCTGCCGCT
TACCGGATACCTGTCCGCCTTTCTCCCTTCGGGAAGCGTGGCGCT
TTCTCATAGCTCACGCTGTAGGTATCTCAGTTCGGTGTAGGTCGT
TCGCTCCAAGCTGGGCTGTGTGCACGAACCCCCCGTTCAGCCCGA
CCGCTGCGCCTTATCCGGTAACTATCGTCTTGAGTCCAACCCGGT
AAGACACGACTTATCGCCACTGGCAGCAGCCACTGGTAACAGGAT
TAGCAGAGCGAGGTATGTAGGCGGTGCTACAGAGTTCTTGAAGTG
GTGGCCTAACTACGGCTACACTAGAAGAACAGTATTTGGTATCTG
CGCTCTGCTGAAGCCAGTTACCTTCGGAAAAAGAGTTGGTAGCTC
TTGATCCGGCAAACAAACCACCGCTGGTAGCGGTGGTTTTTTTGT
TTGCAAGCAGCAGATTACGCGCAGAAAAAAAGGATCTCAAGAAGA
TCCTTTGATCTTTTCTACGGGGTCTGACGCTCAGTGGAACGAAAA
CTCACGTTAAGGGATTTTGGTCATGAGATTATCAAAAAGGATCTT
CACCTAGATCCTTTTAAATTAAAAATGAAGTTTTAAATCAATCTA
AAGTATATATGAGTAAACTTGGTCTGACAGTTACCAATGCTTAAT
CAGTGAGGCACCTATCTCAGCGATCTGTCTATTTCGTTCATCCAT
AGTTGCCTGACTCCCCGTCGTGTAGATAACTACGATACGGGAGGG
CTTACCATCTGGCCCCAGTGCTGCAATGATACCGCGAGACCCACG
CTCACCGGCTCCAGATTTATCAGCAATAAACCAGCCAGCCGGAAG
GGCCGAGCGCAGAAGTGGTCCTGCAACTTTATCCGCCTCCATCCA
GTCTATTAATTGTTGCCGGGAAGCTAGAGTAAGTAGTTCGCCAGT
TAATAGTTTGCGCAACGTTGTTGCCATTGCTACAGGCATCGTGGT
GTCACGCTCGTCGTTTGGTATGGCTTCATTCAGCTCCGGTTCCCA
ACGATCAAGGCGAGTTACATGATCCCCCATGTTGTGCAAAAAAGC
GGTTAGCTCCTTCGGTCCTCCGATCGTTGTCAGAAGTAAGTTGGC
CGCAGTGTTATCACTCATGGTTATGGCAGCACTGCATAATTCTCT
TACTGTCATGCCATCCGTAAGATGCTTTTCTGTGACTGGTGAGTA
CTCAACCAAGTCATTCTGAGAATAGTGTATGCGGCGACCGAGTTG
CTCTTGCCCGGCGTCAATACGGGATAATACCGCGCCACATAGCAG
AACTTTAAAAGTGCTCATCATTGGAAAACGTTCTTCGGGGCGAAA
ACTCTCAAGGATCTTACCGCTGTTGAGATCCAGTTCGATGTAACC
CACTCGTGCACCCAACTGATCTTCAGCATCTTTTACTTTCACCAG
CGTTTCTGGGTGAGCAAAAACAGGAAGGCAAAATGCCGCAAAAAA
GGGAATAAGGGCGACACGGAAATGTTGAATACTCATACTCTTCCT
TTTTCAATATTATTGAAGCATTTATCAGGGTTATTGTCTCATGAG
CGGATACATATTTGAATGTATTTAGAAAAATAAACAAATAGGGGT
TCCGCGCACATTTCCCCGAAAAGT.

ix. CVL120

The nucleic acid sequence for CVL120 is

(SEQ ID NO: 11)
GCCACCTGACGTCTAAGAAACCATTATTATCATGACATTAACCTA
TAAAAATAGGCGTATCACGAGGCCCTTTCGTCTCGCGCGTTTCGG
TGATGACGGTGAAAACCTCTGACACATGCAGCTCCCGGAGACGGT
CACAGCTTGTCTGTAAGCGGATGCCGGGAGCAGACAAGCCCGTCA
GGGCGCGTCAGCGGGTGTTGGCGGGTGTCGGGGCTGGCTTAACTA
TGCGGCATCAGAGCAGATTGTACTGAGAGTGCACCATATATGCGG
TGTGAAATACCGCACAGATGCGTAAGGAGAAAATACCGCATCAGG
CTAATACGACTCACTATAGGGACCAAGGGGAAAATGAAGTGGTGA
CTCAAATCATCGAAGACCCTCGAGATTACATAGGTCCGGAACCTA
TGGCCTTCGTGACCGACCTCGAGTCAGAGTAGTTCAATAAGGACC
TATCAAGTTTGGGCAATTTTTCGTCCCCGACACAAAAATGTCATC
CGTGCTTAAAGCATATGAGCGATTCACGCTCACTCAAGAACTGCA
AGATCAGAGTGAGGAAGGTACAATCCCACCTACAACACTAAAACC
GGTAATCAGGGTATTTATACTAACCTCTAATAACCCAGAGCTAAG
ATCCCGGCTTCTTCTATTCTGCCTACGGATTGTTCTCAGTAATGG
TGCAAGGGATTCCCATCGCTTTGGAGCATTACTCACAATGTTTTC
GCTACCATCAGCCACAATGCTCAATCATGTCAAATTAGCTGACCA
GTCACCAGAAGCTGATATCGAAAGGGTAGAGATCGATGGCTTTGA
GGAGGGATCATTCCGCTTAATCCCCAATGCTCGTTCAGGTATGAG
CCGTGGAGAGATCAATGCCTATGCTGCACTTGCAGAAGATCTACC
TGACACACTAAACCATGCAACACCTTTCGTTGATTCCGAAGTCGA
GGGAACTGCATGGGATGAGATTGAGACTTTCTTAGATATGTGTTA
CAGTGTCCTAATGCAGGCATGGATAGTGACTTGCAAGTGCATGAC
TGCGCCAGACCAACCTGCTGCTTCTATTGAGAAACGCCTGCAAAA
ATATCGTCAGCAAGGCAGGATCAACCCGAGATATCTCCTGCAACC
GGAGGCTCGACGAATAATCCAGAATGTAATCCGGAAGGGAATGGT
GGTCAGACATTTCCTCACCTTTGAACTGCAGCTTGCCCGAGCACA
AAGCCTTGTATCAAATAGGTATTATGCTATGGTAGGGGATGTTGG
AAAGTATATAGAGAATTGTGGAATGGGAGGCTTCTTTTTGACACT
AAAATATGCATTAGGAACTAGATGGCCCACACTTGCTTTAGCTGC
ATTTTCAGGAGAGCTAACAAAGCTAAAGTCCCTCATGGCATTATA
CCAGACCCTTGGTGAGCAGGCCCGATATTTGGCCCTATTGGAGTC
ACCACATTTGATGGATTTTGCTGCAGCAAACTACCCACTGCTATA
TAGCTATGCTATGGGAATAGGCTATGTGTTAGATGTCAACATGAG
GAACTACGCTTTCTCCAGATCATACATGAACAAGACATATTTCCA
ATTGGGAATGGAAACTGCAAGAAAACAACAGGGTGCAGTTGACAT
GAGGATGGCAGAAGATCTCGGTCTAACTCAAGCCGAACGCACCGA
GATGGCAAATACACTTGCCAAATTGACCACAGCAAATCGAGGGGC
AGACACCAGGGGAGGAGTCAACCCGTTCTCATCTGTCACTGGGAC
AACTCAGGTGCCCGCTGCAGCAACAGGTGACACACTCGAGAGTTA
CATGGCAGCGGATCGACTGAGGCAGAGATATGCTGATGCAGGCAC
CCATGATGATGAGATGCCACCATTGGAAGAGGAGGAAGAGGACGA
CACATCTGCAGGTCCACGCACTGGACCAACTCTTGAACAAGTGGC
CTTGGACATCCAGAACGCAGCAGTTGGAGCTCCCATCCATACAGA
TGACCTGAATGCCGCACTGGGTGATCTTGACATCTAGACAATTCA
GATCCCAATCTAAAATTGACATACCTAATTGATTAGTTAGATGGA
ACTACAGTGGATTCCATAAGGTTCCTGCCTACCATCGGCTTTAAA
GAAAAAAATAGGCCCGGACGGGTTAGCAACAAGCGACTGCCGGTG
CCAACAGCGCAATCCACAATCTACAATGGATCCCACTGATCTGAG
CTTCTCCCCAGATGAGATCAATAAGCTCATAGAGACAGGCCTGAA
TACTGTAGAGTATTTTACTTCCCAACAAGTCACAGGAACATCCTC
TCTTGGAAAGAATACAATACCACCAGGGGTCACAGGACTACTAAC
CAATGCTGCAGAGGCAAAGATCCAAGAGTCAACTAACCATCAGAA
GGGCTCAGTTGGTGGGGGTGCAAAACCAAAGAAACCGCGACCAAA
AATTGCCATTGTGCCAGCAGATGACAAAACAGTGCCCGGAAAGCC
GATCCCAAACCCTCTATTAGGTCTGGACTCCACCCCGAGCACCCA
AACTGTGCTTGATCTAAGTGGGAAAACATTACCATCAGGATCCTA
TAAGGGGGTTAAGCTTGCGAAATTTGGAAAAGAAAATCTGATGAC
ACGGTTCATCGAGGAACCCAGAGAGAATCCTATCGCAACCAGTTt
CCCCATCGATTTTAAGAGGGGCAGGGATACCGGCGGGTTCCATAG
AAGGGAGTACTCAATCGGATGGGTGGGAGATGAAGTCAAGGTCAC
TGAGTGGTGCAATCCATCCTGTTCTCCAATCACCGCTGCAGCAAG
GCGATTTGAATGCACTTGTCACCAGTGTCCAGTCACTTGCTCTGA
ATGTGAACGAGATACTTAATACAGTGAGAAATTTGGACTCTCGGA
TGAATCAACTGGAGACAAAAGTAGATCGCATTCTCTCATCTCAGT
CTCTAATCCAGACCATCAAGAATGACATAGTTGGACTTAAAGCAG
GGATGGCTACTTTAGAAGGAATGATTACAACTGTGAAAATCATGG
ACCCGGGAGTTCCCAGTAATGTTACTGTGGAAGATGTACGCAAGA
CACTAAGTAACCATGCTGTTGTTGTGCCAGAATCATTCAATGATA
GTTTCTTGACTCAATCTGAAGATGTAATTTCACTTGATGAGTTGG
CTCGACCAACTGCAACAAGTGTTAAGAAGATTGTCAGGAAGGTTC
CTCCTCAGAAGGATCTGACTGGATTGAAGATTACACTAGAGCAAT
TGGCAAAGGATTGCATCAGCAAACCGAAGATGAGGGAAGAGTATC
TCCTCAAAATCAACCAGGCTTCCAGTGAGGCTCAGCTAATTGACC
TCAAGAAAGCAATCATCCGCAGTGCAATTTGATCAAGAAACACCC
AATTACACTACACTGGTATGACACTGTACTAACCCTGAGGGTTTT
AGAAAAAACGATTAACGATAAATAAGCCCGAACACTACACACTAC
CTGAGGCAGCCATGCCATCCATCAGCATTCCCGCAGACCCCACCA
ATCCACGTCAATCAATAAAAGCGTTCCCAATTGTGATCAACAGTG
ATGGGGGTGAGAAAGGCCGCTTGGTTAAACAACTACGCACAACCT
ACTTGAATGACCTAGATACTCATGAGCCACTGGTGACATTCATAA
ATACCTATGGATTCATCTACGAACAGGATCGGGGGAATACCATTG
TCGGAGAGGATCAACTTGGGAAGAAAAGAGAGGCTGTGACCGCTG
CAATGGTTACCCTTGGATGTGGGCCTAATCTACCATCATTAGGGA
ATGTCCTGGGACAACTGAGGGAATTCCAGGTCACTGTTAGGAAGA
CATCCAGCAAAGCGGAAGAGATGGTCTTTGAAATTGTTAAGTATC
CGAGAATATTTCGGGGTCATACATTAATCCAGAAAGGACTAGTCT
GTGTCTCCGCAGAAAAATTTGTTAAGTCACCAGGGAAAATACAAT
CTGGAATGGACTATCTCTTCATTCCGACATTTCTGTCAGTGACTT
ACTGTCCAGCTGCAATCAAATTTCAGGTACCTGGCCCCATGTTGA
AAATGAGATCAAGATACACTCAGAGCTTACAACTTGAACTAATGA
TAAGAATCCTGTGTAAGCCCGATTCGCCACTTATGAAGGTCCATA
CCCCTGACAAGGAGGGAAGAGGATGTCTTGTATCAGTATGGCTGC
ATGTATGCAACATCTTCAAATCAGGAAACAAGAATGGCAGTGAGT
GGCAGGAATACTGGATGAGAAAGTGTGCTAACATGCAACTTGAAG
TGTCGATTGCAGATATGTGGGGACCAACTATCATAATTCATGCCA
GAGGTCACATTCCCAAAAGTGCTAAGTTGTTTTTTGGAAAGGGTG
GATGGAGCTGCCATCCACTTCACGAAGTTGTTCCAAGTGTCACTA
AAACACTATGGTCCGTGGGCTGTGAGATTACAAAGGCGAAGGCAA
TAATACAAGAGAGTAGCATCTCTCTTCTCGTGGAGACTACTGACA
TCATAAGTCCAAAAGTCAAAATTTCATCTAAGCATCGCCGCTTTG
GGAAATCAAATTGGGGTCTGTTCAAGAAAACTAAATCACTGCCTA
ACCTGACGGAGCTGGAATGACTGACCTCTAATCGAGACTACACCG
CCGCAAACTATAGGTGGGTGGTACCTCAGTGATTAATCTTGTAAG
CACTGATCGTAGGCTACAACACACTAATATTATCCAGATTAGAGA
GCTTAATTAGCTCTGTATTAATAATAACACTACTATTCCAATAAC
TGGAATCACCAGCTTGATTTATCTCCAAAATGATTCAAAGAAAAC
AAATCATATTAAGACTATCCTAAGCACGAACCCATATCGTCCTTC
AAATCATGGGTACTATAATTCAATTTCTGGTGGTCTCCTGTCTAT
TGGCAGGAGCAGGCAGCCTTGATCCAGCAGCCCTCATGCAAATCG
GTGTCATTCCAACAAATGTCCGGCAACTTATGTATTATACTGAAG
CTTCATCAGCATTCATTGTTGTGAAGTTAATGCCTACAATTGACT
CGCCGATTAGTGGATGTAATATAACATCAATTTCAAGCTATAATG
CAACAGTGACAAAACTCCTACAGCCGATCGGTGAGAATTTGGAGA
CGATTAGGAACCAGTTGATTCCAACTCGGAGGAGACGCCGGTTTG
CAGGGGTGGTGATTGGATTAGCTGCATTAGGAGTAGCTACTGCCG
CACAGGTCACTGCCGCAGTGGCACTAGTAAAGGCAAATGAAAATG
CTGCGGCTATACTCAATCTCAAAAATGCAATCCAAAAAACAAATG
CAGCAGTTGCAGATGTGGTCCAGGCCACACAATCACTAGGAACGG
CAGTTCAAGCAGTTCAAGATCACATAAACAGTGTGGTAAGTCCAG
CAATTACAGCAGCCAATTGTAAGGCCCAAGATGCTATCATTGGCT
CAATCCTCAATCTCTATTTGACCGAGTTGACAACCATCTTCCACA
ATCAAATTACAAACCCTGCATTGAGTCCCATTACAATTCAAGCTT
TAAGGATCCTACTGGGGAGTACCTTGCCGACTGTGGTCGAAAAAT
CTTTCAATACCCAGATAAGTGCAGCTGAGCTTCTCTCATCAGGGT
TATTGACAGGCCAGATTGTGGGATTAGATTTGACCTATATGCAGA
TGGTCATAAAAATTGAGCTGCCAACTTTAACTGTACAACCTGCAA
CCCAGATCATAGATCTGGCCACCATTTCTGCATTCATTAACAATC
AAGAAGTCATGGCCCAATTACCAACACGTGTTATGGTGACTGGCA
GCTTGATCCAAGCCTATCCCGCATCGCAATGCACCATTACACCCA
ACACTGTGTACTGTAGGTATAATGATGCCCAAGTACTCTCAGATG
ATACTATGGCTTGCCTCCAAGGTAACTTGACAAGATGCACCTTCT
CTCCAGTGGTTGGGAGCTTTCTCACTCGATTCGTGCTGTTCGATG
GAATAGTTTATGCAAATTGCAGGTCGATGTTGTGCAAGTGCATGC
AACCTGCTGCTGTGATCCTACAGCCGAGTTCATCCCCTGTAACTG
TCATTGACATGTACAAATGTGTGAGTCTGCAGCTTGACAATCTCA
GATTCACCATCACTCAATTGGCCAATGTAACCTACAATAGCACCA
TCAAGCTTGAATCATCCCAGATCTTGTCTATTGATCCGTTGGATA
TATCCCAGAATCTAGCTGCGGTGAATAAGAGTCTAAGTGATGCAC
TACAACACTTAGCACAAAGTGACACATATCTTTCTGCAATCACAT
CAGCTACGACTACAAGTGTATTATCCATAATAGCAATCTGTCTTG
GATCGTTAGGTTTAATATTAATAATCTTGCTCAGTGTAGTTGTGT
GGAAGTTATTGACCATTGTCGTTGCTAATCGAAATAGAATGGAGA
ATTTTGTTTATCATAAATAAGCATTCCACCACTCACGATCTGATC
TCAGTGAGAAAAATCAACCTGCAACTCTTGGAACAAGATAAGACA
GTCATCCATTAGTAATTTTTAAGAAAAAAACGATAGGACCGAACC
TAGTATTGAAAGAACCGTCTCGGTCAATCTAGGTAATCGAGCTGA
TACCGTCTCGGAAAGCTCAAATCGCGCGCCACCatggccgatagc
aacggcaccatcaccgtggaagagcttaaaaagctgctcgagcag
tggaacctggttatcggctttctgttcctgacctggatctgcctg
ctccagttcgcttatgccaaccggaacaggttcctgtacatcatc
aagctgatcttcctgtggctgctgtggcctgtgacactggcctgc
ttcgtgctggccgctgtgtaccggattaactggatcaccggcgga
atcgccatcgccatggcctgtctggtcggcctgatgtggctgtct
tacttcatcgccagcttcagactgtttgccagaaccagaagcatg
tggtccttcaaccccgagacaaacatcctgctgaatgtgccactg
cacggcaccatcctgacaagacctctgctggaaagcgagctggtg
attggagccgtgatcctgagaggccatctgcggatcgctggccac
cacctgggaagatgcgacatcaaggacctgcctaaggaaatcaca
gtggctacatctcggaccctgagctactacaaactgggcgcctct
caaagagtggccggcgacagcggcttcgccgcctacagcagatac
agaatcggcaactacaagctgaataccgatcacagcagctccagc
gacaacatcgccctgctggtgcagtgaTGACGTACGACCTGCTAT
AGGCTATCCACTGCATCATCTCTCCTGCCATACTTCCTACTCACA
TCATATCTATTTTAAAGAAAAAATAGGCCCGAACACTAATCGTGC
CGGCAGTGCCACTGCACACACAACACTACACATACAATACACTAC
AATGGTTGCAGAAGATGCCCCTGTTAGGGCCACTTGCCGAGTATT
ATTTCGAACAACAACTTTAATCTTTCTATGCACACTACTAGCATT
AAGCATCTCTATCCTTTATGAGAGTTTAATAACCCAAAAGCAAAT
CATGAGCCAAGCAGGCTCAACTGGATCTAATTCTGGATTAGGAAG
TATCACTGATCTTCTTAATAATATTCTCTCTGTCGCAAATCAGAT
TATATATAACTCTGCAGTCGCTCTACCTCTACAATTGGACACTCT
TGAATCAACACTCCTTACAGCCATTAAGTCTCTTCAAACCAGTGA
CAAGCTAGAACAGAACTGCTCGTGGAGTGCTGCACTGATTAATGA
TAATAGATACATTAATGGCATCAATCAGTTCTATTTTTCAATTGC
TGAGGGTCGCAATCTGACACTTGGCCCACTTCTTAATATGCCTAG
TTTCATTCCAACTGCCACGACACCAGAGGGCTGCACCAGGATCCC
ATCATTCTCGCTCACTAAGACACACTGGTGTTATACACACAATGT
TATCCTGAATGGATGCCAGGATCATGTATCCTCAAATCAATTTGT
TTCTATGGGAATCATTGAACCCACTTCTGCCGGGTTTCCATTCTT
TCGAACCCTAAAGACTCTATATCTCAGCGATGGGGTCAATCGTAA
GAGCTGCTCTATCAGTACAGTTCCGGGGGGTTGTATGATGTACTG
TTTTGTTTCTACTCAACCAGAGAGGGATGACTACTTTTCTGCCGC
TCCTCCAGAACAACGAATTATTATAATGTACTATAATGATACAAT
CGTGGAGCGCATAATTAATCCACCCGGGGTACTAGATGTATGGGC
AACATTGAACCCAGGAACAGGAAGCGGGGTATATTATTTAGGTTG
GGTGCTCTTTCCAATATATGGCGGCGTGATTAAAGGTACGAGTTT
ATGGAATAATCAAGCAAATAAATACTTTATCCCCCAGATGGTTGC
TGCTCTCTGCTCACAAAACCAGGCAACTCAAGTCCAAAATGCTAA
GTCATCATACTATAGCAGCTGGTTTGGCAATCGAATGATTCAGTC
TGGGATCCTGGCATGTCCTCTTCGACAGGATCTAACCAATGAGTG
TTTAGTTCTGCCCTTTTCTAATGATCAGGTGCTTATGGGTGCTGA
AGGGAGATTATACATGTATGGTGACTCGGTGTATTACTATCAAAG
AAGCAATAGTTGGTGGCCTATGACCATGCTGTATAAGGTAACCAT
AACATTCACTAATGGTCAGCCATCTGCTATATCAGCTCAGAATGT
GCCCACACAGCAGGTCCCTAGACCTGGGACAGGAGACTGCTCTGC
AACCAATAGATGTCCCGGTTTTTGCTTGACAGGAGTGTATGCCGA
TGCCTGGTTACTGACCAACCCTTCGTCTACCAGTACATTTGGATC
AGAAGCAACCTTCACTGGTTCTTATCTCAACACAGCAACTCAGCG
TATCAATCCGACGATGTATATCGCGAACAACACACAGATCATAAG
CTCACAGCAATTTGGATCAAGCGGTCAAGAAGCAGCATATGGCCA
CACAACTTGTTTTAGGGACACAGGCTCTGTTATGGTATACTGTAT
CTATATTATTGAATTGTCCTCATCTCTCTTAGGACAATTTCAGAT
TGTCCCATTTATCCGTCAGGTGACACTATCCTAAAGGCAGAAGCC
TTCAGGTCTGACCCAGCCAATCAAAGCATTATACCAGACCATGGC
CTACCATCGGCTTTAAAGAAAAAAATAGGCCCGGACGGGTTAGCA
ACAAGCGGCGGCCGCCGCCACCatgttcgtcttcctggtcctgct
gcctctggtctcctcacagtgcgtcaatctgacaactcggactca
gctgccacctgcttatactaatagcttcaccagaggcgtgtacta
tcctgacaaggtgtttagaagctccgtgctgcactctacacagga
tctgtttctgccattctttagcaacgtgacctggttccacgccat
ccacgtgagcggcaccaatggcacaaagcggttcgacaatcccgt
gctgccttttaacgatggcgtgtacttcgcctctaccgagaagag
caacatcatcagaggctggatctttggcaccacactggactccaa
gacacagtctctgctgatcgtgaacaatgccaccaacgtggtcat
caaggtgtgcgagttccagttttgtaatgatcccttcctgggcgt
gtactatcacaagaacaataagagctggatggagtccgagtttag
agtgtattctagcgccaacaactgcacatttgagtacgtgagcca
gcctttcctgatggacctggagggcaagcagggcaatttcaagaa
cctgagggagttcgtgtttaagaatatcgacggctacttcaaaat
ctactctaagcacacccccatcaacctggtgcgcgacctgcctca
gggcttcagegccctggagcccctggtggatctgcctatcggcat
caacatcacccggtttcagacactgctggccctgcacagaagcta
cctgacacccggcgactcctctagcggatggaccgccggcgctgc
cgcctactatgtgggctacctccagccccggaccttcctgctgaa
gtacaacgagaatggcaccatcacagacgcagtggattgcgccct
ggaccccctgagcgagacaaagtgtacactgaagtcctttaccgt
ggagaagggcatctatcagacatccaatttcagggtgcagccaac
cgagtctatcgtgcgctttcctaatatcacaaacctgtgcccatt
tggcgaggtgttcaacgcaacccgcttcgccagcgtgtacgcctg
gaataggaagcggatcagcaactgcgtggccgactatagcgtgct
gtacaactccgcctctttcagcacctttaagtgctatggcgtgtc
ccccacaaagctgaatgacctgtgctttaccaacgtctacgccga
ttctttcgtgatcaggggcgacgaggtgcgccagategcccccgg
ccagacaggcaagatcgcagactacaattataagctgccagacga
tttcaccggctgcgtgatcgcctggaacagcaacaatctggattc
caaagtgggcggcaactacaattatctgtaccggctgtttagaaa
gagcaatctgaagcccttcgagagggacatctctacagaaatcta
ccaggccggcagcaccccttgcaatggcgtggagggctttaactg
ttatttcccactccagtcctacggcttccagcccacaaacggcgt
gggctatcagccttaccgcgtggtggtgctgagctttgagctgct
gcacgccccagcaacagtgtgcggccccaagaagtccaccaatct
ggtgaagaacaagtgcgtgaacttcaacttcaacggcctgaccgg
cacaggcgtgctgaccgagtccaacaagaagttcctgccatttca
gcagttcggcagggacatcgcagataccacagacgccgtgcgcga
cccacagaccctggagatcctggacatcacaccctgctctttcgg
cggcgtgagcgtgatcacacccggcaccaatacaagcaaccaggt
ggccgtgctgtatcaggacgtgaattgtaccgaggtgcccgtggc
tatccacgccgatcagctgaccccaacatggcgggtgtacagcac
cggctccaacgtcttccagacaagagccggatgcctgatcggagc
agagcacgtgaacaattcctatgagtgcgacatcccaatcggcgc
cggcatctgtgcctcttaccagacccagacaaactctcccagaag
agcccggagcgtggcctcccagtctatcatcgcctataccatgtc
cctgggcgccgagaacagegtggcctactctaacaatagcatcgc
catcccaaccaacttcacaatctctgtgaccacagagatcctgcc
cgtgtccatgaccaagacatctgtggactgcacaatgtatatctg
tggcgattctaccgagtgcagcaacctgctgctccagtacggcag
cttttgtacccagctgaatagagccctgacaggcatcgccgtgga
gcaggataagaacacacaggaggtgttcgcccaggtgaagcaaat
ctacaagaccccccctatcaaggactttggcggcttcaatttttc
ccagatcctgcctgatccatccaagccttctaagcggagctttat
cgaggacctgctgttcaacaaggtgaccctggccgatgccggctt
catcaagcagtatggcgattgcctgggcgacatcgcagccaggga
cctgatctgcgcccagaagtttaatggcctgaccgtgctgccacc
cctgctgacagatgagatgatcgcacagtacacaagcgccctgct
ggccggcaccatcacatccggatggaccttcggcgcaggagccgc
cctccagatcccctttgccatgcagatggcctataggttcaacgg
catcggcgtgacccagaatgtgctgtacgagaaccagaagctgat
cgccaatcagtttaactccgccatcggcaagatccaggacagcct
gtcctctacagccagcgccctgggcaagctccaggatgtggtgaa
tcagaacgcccaggccctgaataccctggtgaagcagctgagcag
caacttcggcgccatctctagcgtgctgaatgacatcctgagccg
gctggacaaggtggaggcagaggtgcagatcgaccggctgatcac
cggccggctccagagcctccagacctatgtgacacagcagctgat
cagggccgccgagatcagggccagcgccaatctggcagcaaccaa
gatgtccgagtgcgtgctgggccagtctaagagagtggacttttg
tggcaagggctatcacctgatgtccttccctcagtctgccccaca
cggcgtggtgtttctgcacgtgacctacgtgcccgcccaggagaa
gaacttcaccacagcccctgccatctgccacgatggcaaggccca
ctttccaagggagggcgtgttcgtgtccaacggcacccactggtt
tgtgacacagcgcaatttctacgagccccagatcatcaccacaga
caacaccttcgtgagcggcaactgtgacgtggtcatcggcatcgt
gaacaataccgtgtatgatccactccagcccgagctggacagctt
taaggaggagctggataagtatttcaagaatcacacctcccctga
cgtggatctgggcgacatcagcggcatcaatgcctccgtggtgaa
catccagaaggagatcgaccgcctgaacgaggtggctaagaatct
gaacgagagcctgatcgacctccaggagctgggcaagtatgagca
gtacatcaagtggccctggtacatctggctgggcttcategccgg
cctgatcgccatcgtgatggtgaccatcatgctgtgctgtatgac
atcctgctgttcttgcctgaagggctgctgtagctgtggctcctg
ctgtaagtttgacgaggatgactctgaacctgtgctgaagggcgt
gaagctgcattacacctaaTAAGGTCGACTCATGGAATGCATACC
AAACATTATTGACACTAATGACACACAAAATTGGTTTTAAGAAAA
ACCAAGAGAACAATAGGCCAGAATGGCTGGGTCTCGGGAGATATT
ACTCCCTGAAGTCCATCTCAATTCACCAATTGTAAAGCATAAGCT
ATACTATTACATTCTACTTGGAAACCTCCCAAATGAGATCGACCT
TGACGATTTAGGTCCATTACATAATCAAAATTGGAATCAGATAGC
ACATGAAGAGTCTAACTTAGCTCAACGCTTGGTAAATGTAAGAAA
TTTTCTAATTACCCACATCCCTGATCTTAGAAAGGGCCATTGGCA
AGAGTATGTCAATGTAATACTGTGGCCGCGAATTCTTCCCTTGAT
CCCGGATTTTAAAATCAATGACCAATTGCCTCTGCTCAAAAATTG
GGACAAGTTAGTTAAAGAATCATGTTCAGTAATCAATGCAGGTAC
TTCCCAGTGCATTCAGAATCTCAGCTATGGACTGACAGGTCGTGG
GAACCTCTTTACACGATCACGTGAACTCTCTGGTGACCGCAGGGA
TATTGATCTTAAGACAGTTGTGGCAGCATGGCATGACTCAGACTG
GAAAAGAATAAGTGATTTTTGGATTATGATCAAATTCCAGATGAG
ACAATTAATTGTTAGGCAAACAGATCATAATGATTCTGATTTAAT
CACGTATATCGAAAATAGAGAAGGCATAATCATCATAACCCCTGA
ACTGGTAGCATTATTTAACACTGAGAATCATACACTAACATACAT
GACCTTTGAAATTGTACTGATGGTTTCAGATATGTACGAAGGTCG
TCACAACATTTTATCACTATGCACAGTTAGCACTTACCTGAATCC
TCTGAAGAAAAGAATAACATATTTATTGAGCCTTGTAGATAACTT
AGCTTTTCAGATAGGTGATGCTGTATATAACATAATTGCTTTGCT
AGAATCCTTTGTATATGCACAGTTGCAAATGTCAGATCCCATCCC
AGAACTCAGAGGACAATTCCATGCATTCGTATGTTCTGAGATTCT
TGATGCACTAAGAGGAACTAATAGTTTCACCCAGGATGAATTAAG
AACTGTGACAACTAATTTGATATCCCCATTCCAAGATCTGACCCC
AGATCTTACGGCTGAATTGCTCTGTATAATGAGGCTTTGGGGACA
CCCCATGCTCACTGCCAGTCAAGCTGCAGGAAAGGTACGCGAGTC
TATGTGTGCTGGAAAAGTATTAGACTTTCCCACCATTATGAAAAC
ACTAGCCTTTTTCCATACTATTCTGATCAATGGATACAGGAGGAA
GCATCATGGAGTATGGCCACCCTTAAACTTACCGGGTAATGCTTC
AAAGGGTCTCACGGAACTTATGAATGACAATACTGAAATAAGCTA
TGAATTCACACTTAAGCATTGGAAGGAAGTCTCTCTTATAAAATT
CAAGAAATGTTTTGATGCAGACGCAGGTGAGGAACTCAGTATATT
TATGAAAGATAAGGCAATTAGTGCCCCAAAACAAGACTGGATGAG
TGTGTTTAGAAGAAGCCTAATCAAACAGCGCCATCAGCATCATCA
GGTCCCCCTACCAAATCCATTCAATCGACGGCTGTTGCTAAACTT
TCTCGGAGATGACAAATTCGACCCGAATGTGGAGCTACAGTATGT
AACATCAGGTGAGTATCTACATGATGACACGTTTTGTGCATCATA
TTCACTAAAAGAGAAGGAAATTAAACCTGATGGTCGAATTTTTGC
AAAGTTGACTAAGAGAATGAGATCATGTCAAGTTATAGCAGAATC
TCTTTTAGCGAACCATGCTGGGAAGTTAATGAAAGAGAATGGTGT
TGTGATGAATCAGCTATCATTAACAAAATCACTATTAACAATGAG
TCAGATTGGAATAATATCCGAGAAAGCTAGAAAGTCAACTCGAGA
TAACATAAATCAACCTGGTTTCCAGAATATCCAGAGAAATAAATC
ACATCACTCCAAGCAAGTCAATCAGCGAGATCCAAGTGATGACTT
TGAATTGGCAGCATCTTTTTTAACTACTGATCTCAAAAAATATTG
TTTACAATGGAGGTACCAGACAATTATCCCATTTGCTCAATCATT
AAACAGAATGTATGGTTATCCTCATCTCTTTGAGTGGATTCACTT
ACGGCTAATGCGTAGTACACTTTACGTGGGGGATCCCTTCAACCC
ACCAGCAGATACCAGTCAATTTGATCTAGATAAAGTAATTAATGG
AGATATCTTCATTGTATCACCCAGAGGTGGAATTGAAGGGCTGTG
TCAAAAGGCTTGGACAATGATATCTATCGCTGTGATAATTCTATC
TGCCACAGAGTCTGGCACACGAGTAATGAGTATGGTGCAGGGAGA
TAATCAAGCAATTGCTGTCACCACACGAGTACCAAGGAGCCTGCC
GACTCTTGAGAAAAAGACTATTGCTTTTAGATCTTGTAATCTATT
CTTTGAGAGGTTAAAATGTAATAATTTTGGATTAGGTCACCATTT
GAAAGAACAAGAGACTATCATTAGTTCTCACTTCTTTGTTTATAG
CAAGAGAATATTCTATCAGGGGAGGATTCTAACGCAAGCCTTAAA
AAATGCTAGTAAGCTCTGCTTGACAGCTGATGTCCTAGGAGAATG
CACCCAATCATCATGTTCTAATCTTGCAACTACTGTCATGAGGTT
AACTGAGAATGGTGTTGAAAAAGATATCTGTTTCTACTTGAATAT
CTATATGACCATCAAACAGCTCTCCTATGATATCATCTTCCCTCA
AGTGTCAATTCCTGGAGATCAGATCACATTAGAATACATAAATAA
TCCACACCTGGTATCACGATTGGCTCTTTTGCCATCCCAGTTAGG
AGGTCTAAACTACCTGTCATGCAGTAGGCTGTTCAATCGAAACAT
AGGCGACCCGGTGGTTTCCGCAGTTGCAGATCTTAAGAGATTAAT
TAAATCAGGATGTATGGATTACTGGATCCTTTATAACTTATTAGG
GAGAAAACCGGGAAACGGCTCATGGGCTACTTTAGCAGCTGACCC
GTACTCAATCAATATAGAGTATCAATACCCTCCAACTACAGCTCT
TAAGAGGCACACCCAACAAGCTCTGATGGAACTCAGTACGAATCC
AATGTTACGTGGCATATTCTCTGACAATGCACAGGCAGAAGAAAA
TAACCTTGCTAGGTTTCTCCTGGATAGGGAGGTGATCTTTCCGCG
TGTAGCTCACATCATCATTGAGCAAACCAGTGTCGGGAGGAGAAA
ACAGATTCAAGGATATTTGGATTCAACTAGATCGATAATGAGGAA
ATCACTAGAAATTAAGCCCTTATCCAATAGGAAGCTTAATGAAAT
ACTGGATTACAACATCAATTACCTAGCTTACAATTTGGCATTACT
CAAGAATGCTATTGAACCTCCGACTTATTTGAAGGCAATGACACT
TGAAACATGTAGCATCGACATTGCAAGGAACCTCCGGAAGCTCTC
CTGGGCCCCACTCTTGGGTGGGAGAAATCTTGAAGGATTAGAGAC
GCCAGATCCCATTGAAATTACTGCAGGAGCATTAATTGTTGGATC
GGGCTACTGTGAACAGTGTGCTGCAGGAGACAATCGATTCACATG
GTTTTTCTTGCCATCTGGTATCGAGATAGGAGGGGATCCCCGTGA
TAATCCTCCTATCCGTGTACCGTACATTGGCTCCAGGACTGATGA
GAGGAGGGTAGCCTCAATGGCATACATCAGGGGTGCCTCGAGTAG
CTTAAAAGCAGTTCTTAGACTGGCGGGAGTGTACATCTGGGCATT
CGGAGATACTCTGGAGAATTGGATAGATGCACTGGATTTGTCTCA
CACTAGAGTTAACATCACACTTGAACAGCTGCAATCCCTCACCCC
ACTTCCAACCTCTGCCAATCTAACCCATCGGTTGGATGATGGCAC
AACTACCCTAAAGTTTACTCCTGCGAGCTCTTACACCTTTTCAAG
TTTCACTCATATATCAAATGATGAGCAATACCTGACAATTAATGA
CAAAACTGCAGATTCAAATATAATCTACCAACAGTTAATGATCAC
TGGACTCGGAATCTTAGAAACATGGAATAATCCCCCAATCAATAG
AACATTCGAAGAATCTACCCTACATTTGCACACTGGTGCATCATG
TTGTGTCCGACCTGTGGACTCCTGCATTCTCTCAGAAGCATTAAC
AGTCAAGCCACATATTACAGTACCGTACAGCAATAAATTTGTATT
TGATGAGGACCCGCTATCTGAATATGAAACTGCAAAACTGGAATC
GTTATCATTCCAAGCCCAATTAGGCAACATTGATGCTGTAGATAT
GACAGGTAAATTAACATTATTGTCCCAATTCACTGCAAGGCAGAT
TATCAATGCAATCACTGGACTCGATGAGTCTGTCTCTCTTACTAA
TGATGCCATTGTTGCATCAGACTATGTCTCCAATTGGATTAGTGA
ATGCATGTATACCAAATTAGATGAATTATTTATGTATTGTGGGTG
GGAACTACTATTGGAACTATCCTATCAAATGTATTATCTGAGGGT
AGTTGGGTGGAGTAATATAGTGGATTATTCTTACATGATCTTGAG
AAGAATCCCGGGTGCAGCATTAAACAATCTGGCATCTACATTAAG
TCATCCAAAACTTTTCCGACGAGCTATCAACCTAGATATAGTTGC
CCCCTTAAATGCTCCTCATTTTGCATCTCTGGACTACATCAAGAT
GAGTGTGGATGCAATACTCTGGGGCTGTAAAAGAGTCATCAATGT
GCTCTCCAATGGAGGGGACTTAGAATTAGTTGTGACATCTGAAGA
TAGCCTTATTCTCAGTGACCGATCCATGAATCTCATTGCAAGGAA
ATTAACTTTATTATCACTGATTCACCATAATGGTTTGGAACTACC
AAAGATTAAGGGGTTCTCTCCTGATGAGAAGTGTTTCGCTTTGAC
AGAATTTTTGAGGAAAGTGGTGAACTCAGGGTTGAGTTCAATAGA
GAACCTATCAAATTTTATGTACAATGTGGAGAACCCACGGCTTGC
AGCATTCGCCAGCAACAATTACTACCTGACCAGAAAATTATTGAA
TTCAATACGAGATACTGAGTCGGGTCAAGTAGCAGTCACCTCATA
TTATGAATCATTAGAATATATTGATAGTCTTAAGCTAACCCCACA
TGTGCCTGGCACCTCATGCATTGAGGATGATAGTCTATGTACAAA
TGATTACATAATCTGGATCATAGAGTCTAATGCAAACTTGGAGAA
GTATCCAATTCCAAATAGCCCTGAGGATGATTCCAATTTCCATAA
CTTTAAGTTGAATGCTCCATCGCACCATACCTTACGCCCATTAGG
GTTGTCATCAACTGCTTGGTATAAGGGTATAAGCTGCTGCAGGTA
CCTTGAGCGATTAAAGCTACCACAAGGTGATCATTTATATATTGC
AGAAGGTAGTGGTGCCAGTATGACAATCATAGAATACCTATTCCC
AGGAAGAAAGATATATTACAATTCTTTATTTAGTAGTGGTGACAA
TCCCCCACAAAGAAATTATGCACCAATGCCTACTCAGTTCATTGA
GAGTGTCCCATACAAGCTCTGGCAAGCACACACAGATCAATATCC
CGAGATTTTTGAGGACTTCATCCCTCTATGGAACGGAAACGCCGC
CATGACTGACATAGGAATGACAGCTTGTGTAGAATTCATCATCAA
TCGAGTCGGCCCAAGGACTTGCAGTTTAGTACATGTAGATTTGGA
ATCAAGTGCAAGCTTAAATCAACAATGCCTGTCAAAGCCGATAAT
TAATGCTATCATCACTGCTACAACTGTTTTGTGCCCTCATGGGGT
GCTTATTCTGAAATATAGTTGGTTGCCATTTACTAGATTTAGTAC
TTTGATCACTTTCTTATGGTGCTACTTTGAGAGAATCACTGTTCT
TAGGAGCACATATTCTGATCCAGCTAATCATGAGGTTTATTTAAT
TTGTATCCTTGCCAACAACTTTGCATTCCAGACTGTCTCGCAGGC
AACAGGAATGGCGATGACTTTAACTGATCAAGGGTTTACTTTGAT
ATCACCTGAAAGAATAAATCAGTATTGGGATGGTCACTTGAAGCA
AGAACGTATCGTAGCAGAAGCAATTGATAAGGTGGTTCTAGGAGA
AAATGCTCTATTTAATTCGAGTGATAATGAATTAATTCTCAAATG
TGGAGGGACACCAAATGCACGGAATCTCATCGATATCGAGCCAGT
CGCAACTTTCATAGAATTTGAACAATTGATCTGCACAATGTTGAC
AACCCACTTGAAGGAAATAATTGATATAACAAGGTCTGGAACCCA
GGATTATGAAAGTTTATTACTCACTCCTTACAATTTAGGTCTTCT
TGGTAAAATCAGTACGATAGTGAGATTATTAACAGAAAGGATTCT
AAATCATACTATCAGGAATTGGTTGATCCTCCCACCTTCGCTCCG
GATGATCGTGAAGCAGGACTTGGAATTCGGCATATTCAGGATTAC
TTCCATCCTCAATTCTGATCGGTTCCTGAAGCTTTCTCCAAATAG
GAAATACTTGATTGCACAATTAACTGCAGGCTACATTAGGAAATT
GATTGAGGGGGATTGCAATATCGATCTAACCAGACCTATCCAAAA
GCAAATCTGGAAAGCATTAGGTTGTGTAGTCTATTGTCACGATCC
AATGGATCAAAGGGAGTCAACAGAGTTTATTGATATAAATATTAA
TGAAGAAATAGACCGCGGGATCGATGGCGAGGAAATCTAAACATA
TCAAGAATCAGAATTAGTTTAAGAAAAAAGAAGAGGATTAATCTT
GGTTTTCCCCTTGGTGGGTCGGCATGGCATCTCCACCTCCTCGCG
GTCCGACCTGGGCATCCGAAGGAGGACGCACGTCCACTCGGATGG
CTAAGGGAGCGGCCGGGGATCCGGCTGCTAACAAAGCCCGAAAGG
AAGCTGAGTTGGCTGCTGCCACCGCTGAGCAATAACTAGCATAAC
CCCTTGGGGCCTCTAAACGGGTCTTGAGGGGTTTTTTGCTGAAAG
GAGGAACTATATCCGGATCATGTGAGCAAAAGGCCAGCAAAAGGC
CAGGAACCGTAAAAAGGCCGCGTTGCTGGCGTTTTTCCATAGGCT
CCGCCCCCCTGACGAGCATCACAAAAATCGACGCTCAAGTCAGAG
GTGGCGAAACCCGACAGGACTATAAAGATACCAGGCGTTTCCCCC
TGGAAGCTCCCTCGTGCGCTCTCCTGTTCCGACCCTGCCGCTTAC
CGGATACCTGTCCGCCTTTCTCCCTTCGGGAAGCGTGGCGCTTTC
TCATAGCTCACGCTGTAGGTATCTCAGTTCGGTGTAGGTCGTTCG
CTCCAAGCTGGGCTGTGTGCACGAACCCCCCGTTCAGCCCGACCG
CTGCGCCTTATCCGGTAACTATCGTCTTGAGTCCAACCCGGTAAG
ACACGACTTATCGCCACTGGCAGCAGCCACTGGTAACAGGATTAG
CAGAGCGAGGTATGTAGGCGGTGCTACAGAGTTCTTGAAGTGGTG
GCCTAACTACGGCTACACTAGAAGAACAGTATTTGGTATCTGCGC
TCTGCTGAAGCCAGTTACCTTCGGAAAAAGAGTTGGTAGCTCTTG
ATCCGGCAAACAAACCACCGCTGGTAGCGGTGGTTTTTTTGTTTG
CAAGCAGCAGATTACGCGCAGAAAAAAAGGATCTCAAGAAGATCC
TTTGATCTTTTCTACGGGGTCTGACGCTCAGTGGAACGAAAACTC
ACGTTAAGGGATTTTGGTCATGAGATTATCAAAAAGGATCTTCAC
CTAGATCCTTTTAAATTAAAAATGAAGTTTTAAATCAATCTAAAG
TATATATGAGTAAACTTGGTCTGACAGTTACCAATGCTTAATCAG
TGAGGCACCTATCTCAGCGATCTGTCTATTTCGTTCATCCATAGT
TGCCTGACTCCCCGTCGTGTAGATAACTACGATACGGGAGGGCTT
ACCATCTGGCCCCAGTGCTGCAATGATACCGCGAGACCCACGCTC
ACCGGCTCCAGATTTATCAGCAATAAACCAGCCAGCCGGAAGGGC
CGAGCGCAGAAGTGGTCCTGCAACTTTATCCGCCTCCATCCAGTC
TATTAATTGTTGCCGGGAAGCTAGAGTAAGTAGTTCGCCAGTTAA
TAGTTTGCGCAACGTTGTTGCCATTGCTACAGGCATCGTGGTGTC
ACGCTCGTCGTTTGGTATGGCTTCATTCAGCTCCGGTTCCCAACG
ATCAAGGCGAGTTACATGATCCCCCATGTTGTGCAAAAAAGCGGT
TAGCTCCTTCGGTCCTCCGATCGTTGTCAGAAGTAAGTTGGCCGC
AGTGTTATCACTCATGGTTATGGCAGCACTGCATAATTCTCTTAC
TGTCATGCCATCCGTAAGATGCTTTTCTGTGACTGGTGAGTACTC
AACCAAGTCATTCTGAGAATAGTGTATGCGGCGACCGAGTTGCTC
TTGCCCGGCGTCAATACGGGATAATACCGCGCCACATAGCAGAAC
TTTAAAAGTGCTCATCATTGGAAAACGTTCTTCGGGGCGAAAACT
CTCAAGGATCTTACCGCTGTTGAGATCCAGTTCGATGTAACCCAC
TCGTGCACCCAACTGATCTTCAGCATCTTTTACTTTCACCAGCGT
TTCTGGGTGAGCAAAAACAGGAAGGCAAAATGCCGCAAAAAAGGG
AATAAGGGCGACACGGAAATGTTGAATACTCATACTCTTCCTTTT
TCAATATTATTGAAGCATTTATCAGGGTTATTGTCTCATGAGCGG
ATACATATTTGAATGTATTTAGAAAAATAAACAAATAGGGGTTCC
GCGCACATTTCCCCGAAAAGT.

x. CVL121

The nucleic acid sequence for CVL121 is:

(SEQ ID NO: 12)
GCCACCTGACGTCTAAGAAACCATTATTATCATGACATTAACCTA
TAAAAATAGGCGTATCACGAGGCCCTTTCGTCTCGCGCGTTTCGG
TGATGACGGTGAAAACCTCTGACACATGCAGCTCCCGGAGACGGT
CACAGCTTGTCTGTAAGCGGATGCCGGGAGCAGACAAGCCCGTCA
GGGCGCGTCAGCGGGTGTTGGCGGGTGTCGGGGCTGGCTTAACTA
TGCGGCATCAGAGCAGATTGTACTGAGAGTGCACCATATATGCGG
TGTGAAATACCGCACAGATGCGTAAGGAGAAAATACCGCATCAGG
CTAATACGACTCACTATAGGGACCAAGGGGAAAATGAAGTGGTGA
CTCAAATCATCGAAGACCCTCGAGATTACATAGGTCCGGAACCTA
TGGCCTTCGTGACCGACCTCGAGTCAGAGTAGTTCAATAAGGACC
TATCAAGTTTGGGCAATTTTTCGTCCCCGACACAAAAATGTCATC
CGTGCTTAAAGCATATGAGCGATTCACGCTCACTCAAGAACTGCA
AGATCAGAGTGAGGAAGGTACAATCCCACCTACAACACTAAAACC
GGTAATCAGGGTATTTATACTAACCTCTAATAACCCAGAGCTAAG
ATCCCGGCTTCTTCTATTCTGCCTACGGATTGTTCTCAGTAATGG
TGCAAGGGATTCCCATCGCTTTGGAGCATTACTCACAATGTTTTC
GCTACCATCAGCCACAATGCTCAATCATGTCAAATTAGCTGACCA
GTCACCAGAAGCTGATATCGAAAGGGTAGAGATCGATGGCTTTGA
GGAGGGATCATTCCGCTTAATCCCCAATGCTCGTTCAGGTATGAG
CCGTGGAGAGATCAATGCCTATGCTGCACTTGCAGAAGATCTACC
TGACACACTAAACCATGCAACACCTTTCGTTGATTCCGAAGTCGA
GGGAACTGCATGGGATGAGATTGAGACTTTCTTAGATATGTGTTA
CAGTGTCCTAATGCAGGCATGGATAGTGACTTGCAAGTGCATGAC
TGCGCCAGACCAACCTGCTGCTTCTATTGAGAAACGCCTGCAAAA
ATATCGTCAGCAAGGCAGGATCAACCCGAGATATCTCCTGCAACC
GGAGGCTCGACGAATAATCCAGAATGTAATCCGGAAGGGAATGGT
GGTCAGACATTTCCTCACCTTTGAACTGCAGCTTGCCCGAGCACA
AAGCCTTGTATCAAATAGGTATTATGCTATGGTAGGGGATGTTGG
AAAGTATATAGAGAATTGTGGAATGGGAGGCTTCTTTTTGACACT
AAAATATGCATTAGGAACTAGATGGCCCACACTTGCTTTAGCTGC
ATTTTCAGGAGAGCTAACAAAGCTAAAGTCCCTCATGGCATTATA
CCAGACCCTTGGTGAGCAGGCCCGATATTTGGCCCTATTGGAGTC
ACCACATTTGATGGATTTTGCTGCAGCAAACTACCCACTGCTATA
TAGCTATGCTATGGGAATAGGCTATGTGTTAGATGTCAACATGAG
GAACTACGCTTTCTCCAGATCATACATGAACAAGACATATTTCCA
ATTGGGAATGGAAACTGCAAGAAAACAACAGGGTGCAGTTGACAT
GAGGATGGCAGAAGATCTCGGTCTAACTCAAGCCGAACGCACCGA
GATGGCAAATACACTTGCCAAATTGACCACAGCAAATCGAGGGGC
AGACACCAGGGGAGGAGTCAACCCGTTCTCATCTGTCACTGGGAC
AACTCAGGTGCCCGCTGCAGCAACAGGTGACACACTCGAGAGTTA
CATGGCAGCGGATCGACTGAGGCAGAGATATGCTGATGCAGGCAC
CCATGATGATGAGATGCCACCATTGGAAGAGGAGGAAGAGGACGA
CACATCTGCAGGTCCACGCACTGGACCAACTCTTGAACAAGTGGC
CTTGGACATCCAGAACGCAGCAGTTGGAGCTCCCATCCATACAGA
TGACCTGAATGCCGCACTGGGTGATCTTGACATCTAGACAATTCA
GATCCCAATCTAAAATTGACATACCTAATTGATTAGTTAGATGGA
ACTACAGTGGATTCCATAAGGTTCCTGCCTACCATCGGCTTTAAA
GAAAAAAATAGGCCCGGACGGGTTAGCAACAAGCGACTGCCGGTG
CCAACAGCGCAATCCACAATCTACAATGGATCCCACTGATCTGAG
CTTCTCCCCAGATGAGATCAATAAGCTCATAGAGACAGGCCTGAA
TACTGTAGAGTATTTTACTTCCCAACAAGTCACAGGAACATCCTC
TCTTGGAAAGAATACAATACCACCAGGGGTCACAGGACTACTAAC
CAATGCTGCAGAGGCAAAGATCCAAGAGTCAACTAACCATCAGAA
GGGCTCAGTTGGTGGGGGTGCAAAACCAAAGAAACCGCGACCAAA
AATTGCCATTGTGCCAGCAGATGACAAAACAGTGCCCGGAAAGCC
GATCCCAAACCCTCTATTAGGTCTGGACTCCACCCCGAGCACCCA
AACTGTGCTTGATCTAAGTGGGAAAACATTACCATCAGGATCCTA
TAAGGGGGTTAAGCTTGCGAAATTTGGAAAAGAAAATCTGATGAC
ACGGTTCATCGAGGAACCCAGAGAGAATCCTATCGCAACCAGTTt
CCCCATCGATTTTAAGAGGGGCAGGGATACCGGCGGGTTCCATAG
AAGGGAGTACTCAATCGGATGGGTGGGAGATGAAGTCAAGGTCAC
TGAGTGGTGCAATCCATCCTGTTCTCCAATCACCGCTGCAGCAAG
GCGATTTGAATGCACTTGTCACCAGTGTCCAGTCACTTGCTCTGA
ATGTGAACGAGATACTTAATACAGTGAGAAATTTGGACTCTCGGA
TGAATCAACTGGAGACAAAAGTAGATCGCATTCTCTCATCTCAGT
CTCTAATCCAGACCATCAAGAATGACATAGTTGGACTTAAAGCAG
GGATGGCTACTTTAGAAGGAATGATTACAACTGTGAAAATCATGG
ACCCGGGAGTTCCCAGTAATGTTACTGTGGAAGATGTACGCAAGA
CACTAAGTAACCATGCTGTTGTTGTGCCAGAATCATTCAATGATA
GTTTCTTGACTCAATCTGAAGATGTAATTTCACTTGATGAGTTGG
CTCGACCAACTGCAACAAGTGTTAAGAAGATTGTCAGGAAGGTTC
CTCCTCAGAAGGATCTGACTGGATTGAAGATTACACTAGAGCAAT
TGGCAAAGGATTGCATCAGCAAACCGAAGATGAGGGAAGAGTATC
TCCTCAAAATCAACCAGGCTTCCAGTGAGGCTCAGCTAATTGACC
TCAAGAAAGCAATCATCCGCAGTGCAATTTGATCAAGAAACACCC
AATTACACTACACTGGTATGACACTGTACTAACCCTGAGGGTTTT
AGAAAAAACGATTAACGATAAATAAGCCCGAACACTACACACTAC
CTGAGGCAGCCATGCCATCCATCAGCATTCCCGCAGACCCCACCA
ATCCACGTCAATCAATAAAAGCGTTCCCAATTGTGATCAACAGTG
ATGGGGGTGAGAAAGGCCGCTTGGTTAAACAACTACGCACAACCT
ACTTGAATGACCTAGATACTCATGAGCCACTGGTGACATTCATAA
ATACCTATGGATTCATCTACGAACAGGATCGGGGGAATACCATTG
TCGGAGAGGATCAACTTGGGAAGAAAAGAGAGGCTGTGACCGCTG
CAATGGTTACCCTTGGATGTGGGCCTAATCTACCATCATTAGGGA
ATGTCCTGGGACAACTGAGGGAATTCCAGGTCACTGTTAGGAAGA
CATCCAGCAAAGCGGAAGAGATGGTCTTTGAAATTGTTAAGTATC
CGAGAATATTTCGGGGTCATACATTAATCCAGAAAGGACTAGTCT
GTGTCTCCGCAGAAAAATTTGTTAAGTCACCAGGGAAAATACAAT
CTGGAATGGACTATCTCTTCATTCCGACATTTCTGTCAGTGACTT
ACTGTCCAGCTGCAATCAAATTTCAGGTACCTGGCCCCATGTTGA
AAATGAGATCAAGATACACTCAGAGCTTACAACTTGAACTAATGA
TAAGAATCCTGTGTAAGCCCGATTCGCCACTTATGAAGGTCCATA
CCCCTGACAAGGAGGGAAGAGGATGTCTTGTATCAGTATGGCTGC
ATGTATGCAACATCTTCAAATCAGGAAACAAGAATGGCAGTGAGT
GGCAGGAATACTGGATGAGAAAGTGTGCTAACATGCAACTTGAAG
TGTCGATTGCAGATATGTGGGGACCAACTATCATAATTCATGCCA
GAGGTCACATTCCCAAAAGTGCTAAGTTGTTTTTTGGAAAGGGTG
GATGGAGCTGCCATCCACTTCACGAAGTTGTTCCAAGTGTCACTA
AAACACTATGGTCCGTGGGCTGTGAGATTACAAAGGCGAAGGCAA
TAATACAAGAGAGTAGCATCTCTCTTCTCGTGGAGACTACTGACA
TCATAAGTCCAAAAGTCAAAATTTCATCTAAGCATCGCCGCTTTG
GGAAATCAAATTGGGGTCTGTTCAAGAAAACTAAATCACTGCCTA
ACCTGACGGAGCTGGAATGACTGACCTCTAATCGAGACTACACCG
CCGCAAACTATAGGTGGGTGGTACCTCAGTGATTAATCTTGTAAG
CACTGATCGTAGGCTACAACACACTAATATTATCCAGATTAGAGA
GCTTAATTAGCTCTGTATTAATAATAACACTACTATTCCAATAAC
TGGAATCACCAGCTTGATTTATCTCCAAAATGATTCAAAGAAAAC
AAATCATATTAAGACTATCCTAAGCACGAACCCATATCGTCCTTC
AAATCATGGGTACTATAATTCAATTTCTGGTGGTCTCCTGTCTAT
TGGCAGGAGCAGGCAGCCTTGATCCAGCAGCCCTCATGCAAATCG
GTGTCATTCCAACAAATGTCCGGCAACTTATGTATTATACTGAAG
CTTCATCAGCATTCATTGTTGTGAAGTTAATGCCTACAATTGACT
CGCCGATTAGTGGATGTAATATAACATCAATTTCAAGCTATAATG
CAACAGTGACAAAACTCCTACAGCCGATCGGTGAGAATTTGGAGA
CGATTAGGAACCAGTTGATTCCAACTCGGAGGAGACGCCGGTTTG
CAGGGGTGGTGATTGGATTAGCTGCATTAGGAGTAGCTACTGCCG
CACAGGTCACTGCCGCAGTGGCACTAGTAAAGGCAAATGAAAATG
CTGCGGCTATACTCAATCTCAAAAATGCAATCCAAAAAACAAATG
CAGCAGTTGCAGATGTGGTCCAGGCCACACAATCACTAGGAACGG
CAGTTCAAGCAGTTCAAGATCACATAAACAGTGTGGTAAGTCCAG
CAATTACAGCAGCCAATTGTAAGGCCCAAGATGCTATCATTGGCT
CAATCCTCAATCTCTATTTGACCGAGTTGACAACCATCTTCCACA
ATCAAATTACAAACCCTGCATTGAGTCCCATTACAATTCAAGCTT
TAAGGATCCTACTGGGGAGTACCTTGCCGACTGTGGTCGAAAAAT
CTTTCAATACCCAGATAAGTGCAGCTGAGCTTCTCTCATCAGGGT
TATTGACAGGCCAGATTGTGGGATTAGATTTGACCTATATGCAGA
TGGTCATAAAAATTGAGCTGCCAACTTTAACTGTACAACCTGCAA
CCCAGATCATAGATCTGGCCACCATTTCTGCATTCATTAACAATC
AAGAAGTCATGGCCCAATTACCAACACGTGTTATGGTGACTGGCA
GCTTGATCCAAGCCTATCCCGCATCGCAATGCACCATTACACCCA
ACACTGTGTACTGTAGGTATAATGATGCCCAAGTACTCTCAGATG
ATACTATGGCTTGCCTCCAAGGTAACTTGACAAGATGCACCTTCT
CTCCAGTGGTTGGGAGCTTTCTCACTCGATTCGTGCTGTTCGATG
GAATAGTTTATGCAAATTGCAGGTCGATGTTGTGCAAGTGCATGC
AACCTGCTGCTGTGATCCTACAGCCGAGTTCATCCCCTGTAACTG
TCATTGACATGTACAAATGTGTGAGTCTGCAGCTTGACAATCTCA
GATTCACCATCACTCAATTGGCCAATGTAACCTACAATAGCACCA
TCAAGCTTGAATCATCCCAGATCTTGTCTATTGATCCGTTGGATA
TATCCCAGAATCTAGCTGCGGTGAATAAGAGTCTAAGTGATGCAC
TACAACACTTAGCACAAAGTGACACATATCTTTCTGCAATCACAT
CAGCTACGACTACAAGTGTATTATCCATAATAGCAATCTGTCTTG
GATCGTTAGGTTTAATATTAATAATCTTGCTCAGTGTAGTTGTGT
GGAAGTTATTGACCATTGTCGTTGCTAATCGAAATAGAATGGAGA
ATTTTGTTTATCATAAATAAGCATTCCACCACTCACGATCTGATC
TCAGTGAGAAAAATCAACCTGCAACTCTTGGAACAAGATAAGACA
GTCATCCATTAGTAATTTTTAAGAAAAAAACGATAGGACCGAACC
TAGTATTGAAAGAACCGTCTCGGTCAATCTAGGTAATCGAGCTGA
TACCGTCTCGGAAAGCTCAAATCGCGCGCCACCatggccgatagc
aacggcaccatcaccgtggaagagcttaaaaagctgctcgagcag
tggaacctggttatcggctttctgttcctgacctggatctgcctg
ctccagttcgcttatgccaaccggaacaggttcctgtacatcatc
aagctgatcttcctgtggctgctgtggcctgtgacactggcctgc
ttcgtgctggccgctgtgtaccggattaactggatcaccggcgga
atcgccatcgccatggcctgtctggtcggcctgatgtggctgtct
tacttcatcgccagcttcagactgtttgccagaaccagaagcatg
tggtccttcaaccccgagacaaacatcctgctgaatgtgccactg
cacggcaccatcctgacaagacctctgctggaaagcgagctggtg
attggagccgtgatcctgagaggccatctgcggatcgctggccac
cacctgggaagatgcgacatcaaggacctgcctaaggaaatcaca
gtggctacatctcggaccctgagctactacaaactgggcgcctct
caaagagtggccggcgacageggcttcgccgcctacagcagatac
agaatcggcaactacaagctgaataccgatcacagcagctccagc
gacaacatcgccctgctggtgcagtgaTGACGTACGGATCCCAAT
CTTAAATCGACACACCTAATTGACCAGTTAGATGGAACTACAGTG
GATTCCATAAGGTTCCTGCCTACCATCGGCTTTTAAGAAAAAAAT
AGGCCCGGACGGGTTAGCAACAAGCGACTGCCGATGCCAATAACA
CAATCCACGCGTGCCACCatgtattctttcgtgtctgaagagaca
ggcaccctgatcgttaatagcgtgctgctgtttctggccttcgtg
gtgttcctgctggtgaccctggctatcctgaccgccctgagactg
tgtgcctactgctgcaacatcgtgaacgtgtccctggtcaagcct
agcttctacgtgtacagccgggtgaagaacctgaacagcagcaga
gtgcccgacctgctcgtgtaaGCTAGCACCTGCTATAGGCTATCC
ACTGCATCATCTCTCCTGCCATACTTCCTACTCACATCATATCTA
TTTTAAAGAAAAAATAGGCCCGAACACTAATCGTGCCGGCAGTGC
CACTGCACACACAACACTACACATACAATACACTACAATGGTTGC
AGAAGATGCCCCTGTTAGGGCCACTTGCCGAGTATTATTTCGAAC
AACAACTTTAATCTTTCTATGCACACTACTAGCATTAAGCATCTC
TATCCTTTATGAGAGTTTAATAACCCAAAAGCAAATCATGAGCCA
AGCAGGCTCAACTGGATCTAATTCTGGATTAGGAAGTATCACTGA
TCTTCTTAATAATATTCTCTCTGTCGCAAATCAGATTATATATAA
CTCTGCAGTCGCTCTACCTCTACAATTGGACACTCTTGAATCAAC
ACTCCTTACAGCCATTAAGTCTCTTCAAACCAGTGACAAGCTAGA
ACAGAACTGCTCGTGGAGTGCTGCACTGATTAATGATAATAGATA
CATTAATGGCATCAATCAGTTCTATTTTTCAATTGCTGAGGGTCG
CAATCTGACACTTGGCCCACTTCTTAATATGCCTAGTTTCATTCC
AACTGCCACGACACCAGAGGGCTGCACCAGGATCCCATCATTCTC
GCTCACTAAGACACACTGGTGTTATACACACAATGTTATCCTGAA
TGGATGCCAGGATCATGTATCCTCAAATCAATTTGTTTCTATGGG
AATCATTGAACCCACTTCTGCCGGGTTTCCATTCTTTCGAACCCT
AAAGACTCTATATCTCAGCGATGGGGTCAATCGTAAGAGCTGCTC
TATCAGTACAGTTCCGGGGGGTTGTATGATGTACTGTTTTGTTTC
TACTCAACCAGAGAGGGATGACTACTTTTCTGCCGCTCCTCCAGA
ACAACGAATTATTATAATGTACTATAATGATACAATCGTGGAGCG
CATAATTAATCCACCCGGGGTACTAGATGTATGGGCAACATTGAA
CCCAGGAACAGGAAGCGGGGTATATTATTTAGGTTGGGTGCTCTT
TCCAATATATGGCGGCGTGATTAAAGGTACGAGTTTATGGAATAA
TCAAGCAAATAAATACTTTATCCCCCAGATGGTTGCTGCTCTCTG
CTCACAAAACCAGGCAACTCAAGTCCAAAATGCTAAGTCATCATA
CTATAGCAGCTGGTTTGGCAATCGAATGATTCAGTCTGGGATCCT
GGCATGTCCTCTTCGACAGGATCTAACCAATGAGTGTTTAGTTCT
GCCCTTTTCTAATGATCAGGTGCTTATGGGTGCTGAAGGGAGATT
ATACATGTATGGTGACTCGGTGTATTACTATCAAAGAAGCAATAG
TTGGTGGCCTATGACCATGCTGTATAAGGTAACCATAACATTCAC
TAATGGTCAGCCATCTGCTATATCAGCTCAGAATGTGCCCACACA
GCAGGTCCCTAGACCTGGGACAGGAGACTGCTCTGCAACCAATAG
ATGTCCCGGTTTTTGCTTGACAGGAGTGTATGCCGATGCCTGGTT
ACTGACCAACCCTTCGTCTACCAGTACATTTGGATCAGAAGCAAC
CTTCACTGGTTCTTATCTCAACACAGCAACTCAGCGTATCAATCC
GACGATGTATATCGCGAACAACACACAGATCATAAGCTCACAGCA
ATTTGGATCAAGCGGTCAAGAAGCAGCATATGGCCACACAACTTG
TTTTAGGGACACAGGCTCTGTTATGGTATACTGTATCTATATTAT
TGAATTGTCCTCATCTCTCTTAGGACAATTTCAGATTGTCCCATT
TATCCGTCAGGTGACACTATCCTAAAGGCAGAAGCCTTCAGGTCT
GACCCAGCCAATCAAAGCATTATACCAGACCATGGCCTACCATCG
GCTTTAAAGAAAAAAATAGGCCCGGACGGGTTAGCAACAAGCGGC
GGCCGCCGCCACCatgttcgtcttcctggtcctgctgcctctggt
ctcctcacagtgcgtcaatctgacaacteggactcagctgccacc
tgcttatactaatagcttcaccagaggcgtgtactatcctgacaa
ggtgtttagaagctccgtgctgcactctacacaggatctgtttct
gccattctttagcaacgtgacctggttccacgccatccacgtgag
cggcaccaatggcacaaagcggttcgacaatcccgtgctgccttt
taacgatggcgtgtacttcgcctctaccgagaagagcaacatcat
cagaggctggatctttggcaccacactggactccaagacacagtc
tctgctgatcgtgaacaatgccaccaacgtggtcatcaaggtgtg
cgagttccagttttgtaatgatcccttcctgggcgtgtactatca
caagaacaataagagctggatggagtccgagtttagagtgtattc
tagcgccaacaactgcacatttgagtacgtgagccagcctttcct
gatggacctggagggcaagcagggcaatttcaagaacctgaggga
gttcgtgtttaagaatatcgacggctacttcaaaatctactctaa
gcacacccccatcaacctggtgcgcgacctgcctcagggcttcag
cgccctggagcccctggtggatctgcctatcggcatcaacatcac
ccggtttcagacactgctggccctgcacagaagctacctgacacc
cggcgactcctctagcggatggaccgccggcgctgccgcctacta
tgtgggctacctccagccccggaccttcctgctgaagtacaacga
gaatggcaccatcacagacgcagtggattgcgccctggaccccct
gagcgagacaaagtgtacactgaagtcctttaccgtggagaaggg
catctatcagacatccaatttcagggtgcagccaaccgagtctat
cgtgcgctttcctaatatcacaaacctgtgcccatttggcgaggt
gttcaacgcaacccgcttcgccagcgtgtacgcctggaataggaa
gcggatcagcaactgcgtggccgactatagcgtgctgtacaactc
cgcctctttcagcacctttaagtgctatggcgtgtcccccacaaa
gctgaatgacctgtgctttaccaacgtctacgccgattctttcgt
gatcaggggcgacgaggtgcgccagatcgcccccggccagacagg
caagatcgcagactacaattataagctgccagacgatttcaccgg
ctgcgtgatcgcctggaacagcaacaatctggattccaaagtggg
cggcaactacaattatctgtaccggctgtttagaaagagcaatct
gaagcccttcgagagggacatctctacagaaatctaccaggccgg
cagcaccccttgcaatggcgtggagggctttaactgttatttccc
actccagtcctacggcttccagcccacaaacggcgtgggctatca
gccttaccgcgtggtggtgctgagctttgagctgctgcacgcccc
agcaacagtgtgcggccccaagaagtccaccaatctggtgaagaa
caagtgcgtgaacttcaacttcaacggcctgaccggcacaggcgt
gctgaccgagtccaacaagaagttcctgccatttcagcagttcgg
cagggacatcgcagataccacagacgccgtgcgcgacccacagac
cctggagatcctggacatcacaccctgctctttcggcggcgtgag
cgtgatcacacccggcaccaatacaagcaaccaggtggccgtgct
gtatcaggacgtgaattgtaccgaggtgcccgtggctatccacgc
cgatcagctgaccccaacatggcgggtgtacagcaccggctccaa
cgtcttccagacaagagccggatgcctgatcggagcagagcacgt
gaacaattcctatgagtgcgacatcccaatcggcgccggcatctg
tgcctcttaccagacccagacaaactctcccagaagagcccggag
cgtggcctcccagtctatcatcgcctataccatgtccctgggcgc
cgagaacagcgtggcctactctaacaatagcategccatcccaac
caacttcacaatctctgtgaccacagagatcctgcccgtgtccat
gaccaagacatctgtggactgcacaatgtatatctgtggcgattc
taccgagtgcagcaacctgctgctccagtacggcagcttttgtac
ccagctgaatagagccctgacaggcatcgccgtggagcaggataa
gaacacacaggaggtgttcgcccaggtgaagcaaatctacaagac
cccccctatcaaggactttggcggcttcaatttttcccagatcct
gcctgatccatccaagccttctaagcggagctttatcgaggacct
gctgttcaacaaggtgaccctggccgatgccggcttcatcaagca
gtatggcgattgcctgggcgacatcgcagccagggacctgatctg
cgcccagaagtttaatggcctgaccgtgctgccacccctgctgac
agatgagatgatcgcacagtacacaagcgccctgctggccggcac
catcacatccggatggaccttcggcgcaggagccgccctccagat
cccctttgccatgcagatggcctataggttcaacggcatcggcgt
gacccagaatgtgctgtacgagaaccagaagctgatcgccaatca
gtttaactccgccatoggcaagatccaggacagcctgtcctctac
agccagcgccctgggcaagctccaggatgtggtgaatcagaacgc
ccaggccctgaataccctggtgaagcagctgagcagcaacttcgg
cgccatctctagcgtgctgaatgacatcctgagccggctggacaa
ggtggaggcagaggtgcagatcgaccggctgatcaccggccggct
ccagagcctccagacctatgtgacacagcagctgatcagggccgc
cgagatcagggccagcgccaatctggcagcaaccaagatgtccga
gtgcgtgctgggccagtctaagagagtggacttttgtggcaaggg
ctatcacctgatgtccttccctcagtctgccccacacggcgtggt
gtttctgcacgtgacctacgtgcccgcccaggagaagaacttcac
cacagcccctgccatctgccacgatggcaaggcccactttccaag
ggagggcgtgttcgtgtccaacggcacccactggtttgtgacaca
gcgcaatttctacgagccccagatcatcaccacagacaacacctt
cgtgagcggcaactgtgacgtggtcatcggcatcgtgaacaatac
cgtgtatgatccactccagcccgagctggacagctttaaggagga
gctggataagtatttcaagaatcacacctcccctgacgtggatct
gggcgacatcagcggcatcaatgcctccgtggtgaacatccagaa
ggagatcgaccgcctgaacgaggtggctaagaatctgaacgagag
cctgatcgacctccaggagctgggcaagtatgagcagtacatcaa
gtggccctggtacatctggctgggcttcatcgccggcctgatcgc
catcgtgatggtgaccatcatgctgtgctgtatgacatcctgctg
ttcttgcctgaagggctgctgtagctgtggctcctgctgtaagtt
tgacgaggatgactctgaacctgtgctgaagggcgtgaagctgca
ttacacctaaTAAGGTCGACTCATGGAATGCATACCAAACATTAT
TGACACTAATGACACACAAAATTGGTTTTAAGAAAAACCAAGAGA
ACAATAGGCCAGAATGGCTGGGTCTCGGGAGATATTACTCCCTGA
AGTCCATCTCAATTCACCAATTGTAAAGCATAAGCTATACTATTA
CATTCTACTTGGAAACCTCCCAAATGAGATCGACCTTGACGATTT
AGGTCCATTACATAATCAAAATTGGAATCAGATAGCACATGAAGA
GTCTAACTTAGCTCAACGCTTGGTAAATGTAAGAAATTTTCTAAT
TACCCACATCCCTGATCTTAGAAAGGGCCATTGGCAAGAGTATGT
CAATGTAATACTGTGGCCGCGAATTCTTCCCTTGATCCCGGATTT
TAAAATCAATGACCAATTGCCTCTGCTCAAAAATTGGGACAAGTT
AGTTAAAGAATCATGTTCAGTAATCAATGCAGGTACTTCCCAGTG
CATTCAGAATCTCAGCTATGGACTGACAGGTCGTGGGAACCTCTT
TACACGATCACGTGAACTCTCTGGTGACCGCAGGGATATTGATCT
TAAGACAGTTGTGGCAGCATGGCATGACTCAGACTGGAAAAGAAT
AAGTGATTTTTGGATTATGATCAAATTCCAGATGAGACAATTAAT
TGTTAGGCAAACAGATCATAATGATTCTGATTTAATCACGTATAT
CGAAAATAGAGAAGGCATAATCATCATAACCCCTGAACTGGTAGC
ATTATTTAACACTGAGAATCATACACTAACATACATGACCTTTGA
AATTGTACTGATGGTTTCAGATATGTACGAAGGTCGTCACAACAT
TTTATCACTATGCACAGTTAGCACTTACCTGAATCCTCTGAAGAA
AAGAATAACATATTTATTGAGCCTTGTAGATAACTTAGCTTTTCA
GATAGGTGATGCTGTATATAACATAATTGCTTTGCTAGAATCCTT
TGTATATGCACAGTTGCAAATGTCAGATCCCATCCCAGAACTCAG
AGGACAATTCCATGCATTCGTATGTTCTGAGATTCTTGATGCACT
AAGAGGAACTAATAGTTTCACCCAGGATGAATTAAGAACTGTGAC
AACTAATTTGATATCCCCATTCCAAGATCTGACCCCAGATCTTAC
GGCTGAATTGCTCTGTATAATGAGGCTTTGGGGACACCCCATGCT
CACTGCCAGTCAAGCTGCAGGAAAGGTACGCGAGTCTATGTGTGC
TGGAAAAGTATTAGACTTTCCCACCATTATGAAAACACTAGCCTT
TTTCCATACTATTCTGATCAATGGATACAGGAGGAAGCATCATGG
AGTATGGCCACCCTTAAACTTACCGGGTAATGCTTCAAAGGGTCT
CACGGAACTTATGAATGACAATACTGAAATAAGCTATGAATTCAC
ACTTAAGCATTGGAAGGAAGTCTCTCTTATAAAATTCAAGAAATG
TTTTGATGCAGACGCAGGTGAGGAACTCAGTATATTTATGAAAGA
TAAGGCAATTAGTGCCCCAAAACAAGACTGGATGAGTGTGTTTAG
AAGAAGCCTAATCAAACAGCGCCATCAGCATCATCAGGTCCCCCT
ACCAAATCCATTCAATCGACGGCTGTTGCTAAACTTTCTCGGAGA
TGACAAATTCGACCCGAATGTGGAGCTACAGTATGTAACATCAGG
TGAGTATCTACATGATGACACGTTTTGTGCATCATATTCACTAAA
AGAGAAGGAAATTAAACCTGATGGTCGAATTTTTGCAAAGTTGAC
TAAGAGAATGAGATCATGTCAAGTTATAGCAGAATCTCTTTTAGC
GAACCATGCTGGGAAGTTAATGAAAGAGAATGGTGTTGTGATGAA
TCAGCTATCATTAACAAAATCACTATTAACAATGAGTCAGATTGG
AATAATATCCGAGAAAGCTAGAAAGTCAACTCGAGATAACATAAA
TCAACCTGGTTTCCAGAATATCCAGAGAAATAAATCACATCACTC
CAAGCAAGTCAATCAGCGAGATCCAAGTGATGACTTTGAATTGGC
AGCATCTTTTTTAACTACTGATCTCAAAAAATATTGTTTACAATG
GAGGTACCAGACAATTATCCCATTTGCTCAATCATTAAACAGAAT
GTATGGTTATCCTCATCTCTTTGAGTGGATTCACTTACGGCTAAT
GCGTAGTACACTTTACGTGGGGGATCCCTTCAACCCACCAGCAGA
TACCAGTCAATTTGATCTAGATAAAGTAATTAATGGAGATATCTT
CATTGTATCACCCAGAGGTGGAATTGAAGGGCTGTGTCAAAAGGC
TTGGACAATGATATCTATCGCTGTGATAATTCTATCTGCCACAGA
GTCTGGCACACGAGTAATGAGTATGGTGCAGGGAGATAATCAAGC
AATTGCTGTCACCACACGAGTACCAAGGAGCCTGCCGACTCTTGA
GAAAAAGACTATTGCTTTTAGATCTTGTAATCTATTCTTTGAGAG
GTTAAAATGTAATAATTTTGGATTAGGTCACCATTTGAAAGAACA
AGAGACTATCATTAGTTCTCACTTCTTTGTTTATAGCAAGAGAAT
ATTCTATCAGGGGAGGATTCTAACGCAAGCCTTAAAAAATGCTAG
TAAGCTCTGCTTGACAGCTGATGTCCTAGGAGAATGCACCCAATC
ATCATGTTCTAATCTTGCAACTACTGTCATGAGGTTAACTGAGAA
TGGTGTTGAAAAAGATATCTGTTTCTACTTGAATATCTATATGAC
CATCAAACAGCTCTCCTATGATATCATCTTCCCTCAAGTGTCAAT
TCCTGGAGATCAGATCACATTAGAATACATAAATAATCCACACCT
GGTATCACGATTGGCTCTTTTGCCATCCCAGTTAGGAGGTCTAAA
CTACCTGTCATGCAGTAGGCTGTTCAATCGAAACATAGGCGACCC
GGTGGTTTCCGCAGTTGCAGATCTTAAGAGATTAATTAAATCAGG
ATGTATGGATTACTGGATCCTTTATAACTTATTAGGGAGAAAACC
GGGAAACGGCTCATGGGCTACTTTAGCAGCTGACCCGTACTCAAT
CAATATAGAGTATCAATACCCTCCAACTACAGCTCTTAAGAGGCA
CACCCAACAAGCTCTGATGGAACTCAGTACGAATCCAATGTTACG
TGGCATATTCTCTGACAATGCACAGGCAGAAGAAAATAACCTTGC
TAGGTTTCTCCTGGATAGGGAGGTGATCTTTCCGCGTGTAGCTCA
CATCATCATTGAGCAAACCAGTGTCGGGAGGAGAAAACAGATTCA
AGGATATTTGGATTCAACTAGATCGATAATGAGGAAATCACTAGA
AATTAAGCCCTTATCCAATAGGAAGCTTAATGAAATACTGGATTA
CAACATCAATTACCTAGCTTACAATTTGGCATTACTCAAGAATGC
TATTGAACCTCCGACTTATTTGAAGGCAATGACACTTGAAACATG
TAGCATCGACATTGCAAGGAACCTCCGGAAGCTCTCCTGGGCCCC
ACTCTTGGGTGGGAGAAATCTTGAAGGATTAGAGACGCCAGATCC
CATTGAAATTACTGCAGGAGCATTAATTGTTGGATCGGGCTACTG
TGAACAGTGTGCTGCAGGAGACAATCGATTCACATGGTTTTTCTT
GCCATCTGGTATCGAGATAGGAGGGGATCCCCGTGATAATCCTCC
TATCCGTGTACCGTACATTGGCTCCAGGACTGATGAGAGGAGGGT
AGCCTCAATGGCATACATCAGGGGTGCCTCGAGTAGCTTAAAAGC
AGTTCTTAGACTGGCGGGAGTGTACATCTGGGCATTCGGAGATAC
TCTGGAGAATTGGATAGATGCACTGGATTTGTCTCACACTAGAGT
TAACATCACACTTGAACAGCTGCAATCCCTCACCCCACTTCCAAC
CTCTGCCAATCTAACCCATCGGTTGGATGATGGCACAACTACCCT
AAAGTTTACTCCTGCGAGCTCTTACACCTTTTCAAGTTTCACTCA
TATATCAAATGATGAGCAATACCTGACAATTAATGACAAAACTGC
AGATTCAAATATAATCTACCAACAGTTAATGATCACTGGACTCGG
AATCTTAGAAACATGGAATAATCCCCCAATCAATAGAACATTCGA
AGAATCTACCCTACATTTGCACACTGGTGCATCATGTTGTGTCCG
ACCTGTGGACTCCTGCATTCTCTCAGAAGCATTAACAGTCAAGCC
ACATATTACAGTACCGTACAGCAATAAATTTGTATTTGATGAGGA
CCCGCTATCTGAATATGAAACTGCAAAACTGGAATCGTTATCATT
CCAAGCCCAATTAGGCAACATTGATGCTGTAGATATGACAGGTAA
ATTAACATTATTGTCCCAATTCACTGCAAGGCAGATTATCAATGC
AATCACTGGACTCGATGAGTCTGTCTCTCTTACTAATGATGCCAT
TGTTGCATCAGACTATGTCTCCAATTGGATTAGTGAATGCATGTA
TACCAAATTAGATGAATTATTTATGTATTGTGGGTGGGAACTACT
ATTGGAACTATCCTATCAAATGTATTATCTGAGGGTAGTTGGGTG
GAGTAATATAGTGGATTATTCTTACATGATCTTGAGAAGAATCCC
GGGTGCAGCATTAAACAATCTGGCATCTACATTAAGTCATCCAAA
ACTTTTCCGACGAGCTATCAACCTAGATATAGTTGCCCCCTTAAA
TGCTCCTCATTTTGCATCTCTGGACTACATCAAGATGAGTGTGGA
TGCAATACTCTGGGGCTGTAAAAGAGTCATCAATGTGCTCTCCAA
TGGAGGGGACTTAGAATTAGTTGTGACATCTGAAGATAGCCTTAT
TCTCAGTGACCGATCCATGAATCTCATTGCAAGGAAATTAACTTT
ATTATCACTGATTCACCATAATGGTTTGGAACTACCAAAGATTAA
GGGGTTCTCTCCTGATGAGAAGTGTTTCGCTTTGACAGAATTTTT
GAGGAAAGTGGTGAACTCAGGGTTGAGTTCAATAGAGAACCTATC
AAATTTTATGTACAATGTGGAGAACCCACGGCTTGCAGCATTCGC
CAGCAACAATTACTACCTGACCAGAAAATTATTGAATTCAATACG
AGATACTGAGTCGGGTCAAGTAGCAGTCACCTCATATTATGAATC
ATTAGAATATATTGATAGTCTTAAGCTAACCCCACATGTGCCTGG
CACCTCATGCATTGAGGATGATAGTCTATGTACAAATGATTACAT
AATCTGGATCATAGAGTCTAATGCAAACTTGGAGAAGTATCCAAT
TCCAAATAGCCCTGAGGATGATTCCAATTTCCATAACTTTAAGTT
GAATGCTCCATCGCACCATACCTTACGCCCATTAGGGTTGTCATC
AACTGCTTGGTATAAGGGTATAAGCTGCTGCAGGTACCTTGAGCG
ATTAAAGCTACCACAAGGTGATCATTTATATATTGCAGAAGGTAG
TGGTGCCAGTATGACAATCATAGAATACCTATTCCCAGGAAGAAA
GATATATTACAATTCTTTATTTAGTAGTGGTGACAATCCCCCACA
AAGAAATTATGCACCAATGCCTACTCAGTTCATTGAGAGTGTCCC
ATACAAGCTCTGGCAAGCACACACAGATCAATATCCCGAGATTTT
TGAGGACTTCATCCCTCTATGGAACGGAAACGCCGCCATGACTGA
CATAGGAATGACAGCTTGTGTAGAATTCATCATCAATCGAGTCGG
CCCAAGGACTTGCAGTTTAGTACATGTAGATTTGGAATCAAGTGC
AAGCTTAAATCAACAATGCCTGTCAAAGCCGATAATTAATGCTAT
CATCACTGCTACAACTGTTTTGTGCCCTCATGGGGTGCTTATTCT
GAAATATAGTTGGTTGCCATTTACTAGATTTAGTACTTTGATCAC
TTTCTTATGGTGCTACTTTGAGAGAATCACTGTTCTTAGGAGCAC
ATATTCTGATCCAGCTAATCATGAGGTTTATTTAATTTGTATCCT
TGCCAACAACTTTGCATTCCAGACTGTCTCGCAGGCAACAGGAAT
GGCGATGACTTTAACTGATCAAGGGTTTACTTTGATATCACCTGA
AAGAATAAATCAGTATTGGGATGGTCACTTGAAGCAAGAACGTAT
CGTAGCAGAAGCAATTGATAAGGTGGTTCTAGGAGAAAATGCTCT
ATTTAATTCGAGTGATAATGAATTAATTCTCAAATGTGGAGGGAC
ACCAAATGCACGGAATCTCATCGATATCGAGCCAGTCGCAACTTT
CATAGAATTTGAACAATTGATCTGCACAATGTTGACAACCCACTT
GAAGGAAATAATTGATATAACAAGGTCTGGAACCCAGGATTATGA
AAGTTTATTACTCACTCCTTACAATTTAGGTCTTCTTGGTAAAAT
CAGTACGATAGTGAGATTATTAACAGAAAGGATTCTAAATCATAC
TATCAGGAATTGGTTGATCCTCCCACCTTCGCTCCGGATGATCGT
GAAGCAGGACTTGGAATTCGGCATATTCAGGATTACTTCCATCCT
CAATTCTGATCGGTTCCTGAAGCTTTCTCCAAATAGGAAATACTT
GATTGCACAATTAACTGCAGGCTACATTAGGAAATTGATTGAGGG
GGATTGCAATATCGATCTAACCAGACCTATCCAAAAGCAAATCTG
GAAAGCATTAGGTTGTGTAGTCTATTGTCACGATCCAATGGATCA
AAGGGAGTCAACAGAGTTTATTGATATAAATATTAATGAAGAAAT
AGACCGCGGGATCGATGGCGAGGAAATCTAAACATATCAAGAATC
AGAATTAGTTTAAGAAAAAAGAAGAGGATTAATCTTGGTTTTCCC
CTTGGTGGGTCGGCATGGCATCTCCACCTCCTCGCGGTCCGACCT
GGGCATCCGAAGGAGGACGCACGTCCACTCGGATGGCTAAGGGAG
CGGCCGGGGATCCGGCTGCTAACAAAGCCCGAAAGGAAGCTGAGT
TGGCTGCTGCCACCGCTGAGCAATAACTAGCATAACCCCTTGGGG
CCTCTAAACGGGTCTTGAGGGGTTTTTTGCTGAAAGGAGGAACTA
TATCCGGATCATGTGAGCAAAAGGCCAGCAAAAGGCCAGGAACCG
TAAAAAGGCCGCGTTGCTGGCGTTTTTCCATAGGCTCCGCCCCCC
TGACGAGCATCACAAAAATCGACGCTCAAGTCAGAGGTGGCGAAA
CCCGACAGGACTATAAAGATACCAGGCGTTTCCCCCTGGAAGCTC
CCTCGTGCGCTCTCCTGTTCCGACCCTGCCGCTTACCGGATACCT
GTCCGCCTTTCTCCCTTCGGGAAGCGTGGCGCTTTCTCATAGCTC
ACGCTGTAGGTATCTCAGTTCGGTGTAGGTCGTTCGCTCCAAGCT
GGGCTGTGTGCACGAACCCCCCGTTCAGCCCGACCGCTGCGCCTT
ATCCGGTAACTATCGTCTTGAGTCCAACCCGGTAAGACACGACTT
ATCGCCACTGGCAGCAGCCACTGGTAACAGGATTAGCAGAGCGAG
GTATGTAGGCGGTGCTACAGAGTTCTTGAAGTGGTGGCCTAACTA
CGGCTACACTAGAAGAACAGTATTTGGTATCTGCGCTCTGCTGAA
GCCAGTTACCTTCGGAAAAAGAGTTGGTAGCTCTTGATCCGGCAA
ACAAACCACCGCTGGTAGCGGTGGTTTTTTTGTTTGCAAGCAGCA
GATTACGCGCAGAAAAAAAGGATCTCAAGAAGATCCTTTGATCTT
TTCTACGGGGTCTGACGCTCAGTGGAACGAAAACTCACGTTAAGG
GATTTTGGTCATGAGATTATCAAAAAGGATCTTCACCTAGATCCT
TTTAAATTAAAAATGAAGTTTTAAATCAATCTAAAGTATATATGA
GTAAACTTGGTCTGACAGTTACCAATGCTTAATCAGTGAGGCACC
TATCTCAGCGATCTGTCTATTTCGTTCATCCATAGTTGCCTGACT
CCCCGTCGTGTAGATAACTACGATACGGGAGGGCTTACCATCTGG
CCCCAGTGCTGCAATGATACCGCGAGACCCACGCTCACCGGCTCC
AGATTTATCAGCAATAAACCAGCCAGCCGGAAGGGCCGAGCGCAG
AAGTGGTCCTGCAACTTTATCCGCCTCCATCCAGTCTATTAATTG
TTGCCGGGAAGCTAGAGTAAGTAGTTCGCCAGTTAATAGTTTGCG
CAACGTTGTTGCCATTGCTACAGGCATCGTGGTGTCACGCTCGTC
GTTTGGTATGGCTTCATTCAGCTCCGGTTCCCAACGATCAAGGCG
AGTTACATGATCCCCCATGTTGTGCAAAAAAGCGGTTAGCTCCTT
CGGTCCTCCGATCGTTGTCAGAAGTAAGTTGGCCGCAGTGTTATC
ACTCATGGTTATGGCAGCACTGCATAATTCTCTTACTGTCATGCC
ATCCGTAAGATGCTTTTCTGTGACTGGTGAGTACTCAACCAAGTC
ATTCTGAGAATAGTGTATGCGGCGACCGAGTTGCTCTTGCCCGGC
GTCAATACGGGATAATACCGCGCCACATAGCAGAACTTTAAAAGT
GCTCATCATTGGAAAACGTTCTTCGGGGCGAAAACTCTCAAGGAT
CTTACCGCTGTTGAGATCCAGTTCGATGTAACCCACTCGTGCACC
CAACTGATCTTCAGCATCTTTTACTTTCACCAGCGTTTCTGGGTG
AGCAAAAACAGGAAGGCAAAATGCCGCAAAAAAGGGAATAAGGGC
GACACGGAAATGTTGAATACTCATACTCTTCCTTTTTCAATATTA
TTGAAGCATTTATCAGGGTTATTGTCTCATGAGCGGATACATATT
TGAATGTATTTAGAAAAATAAACAAATAGGGGTTCCGCGCACATT
TCCCCGAAAAGT.

xi. CVL128

The nucleic acid sequence for CVL128 is:

(SEQ ID NO: 13)
GCCACCTGACGTCTAAGAAACCATTATTATCATGACATTAACCTATA
AAAATAGGCGTATCACGAGGCCCTTTCGTCTCGCGCGTTTCGGTGAT
GACGGTGAAAACCTCTGACACATGCAGCTCCCGGAGACGGTCACAGC
TTGTCTGTAAGCGGATGCCGGGAGCAGACAAGCCCGTCAGGGCGCGT
CAGCGGGTGTTGGCGGGTGTCGGGGCTGGCTTAACTATGCGGCATCA
GAGCAGATTGTACTGAGAGTGCACCATATATGCGGTGTGAAATACCG
CACAGATGCGTAAGGAGAAAATACCGCATCAGGCTAATACGACTCAC
TATAGGGACCAAGGGGAAAATGAAGTGGTGACTCAAATCATCGAAGA
CCCTCGAGATTACATAGGTCCGGAACCTATGGCCTTCGTGACCGACC
TCGAGTCAGAGTAGTTCAATAAGGACCTATCAAGTTTGGGCAATTTT
TCGTCCCCGACACAAAAATGTCATCCGTGCTTAAAGCATATGAGCGA
TTCACGCTCACTCAAGAACTGCAAGATCAGAGTGAGGAAGGTACAAT
CCCACCTACAACACTAAAACCGGTAATCAGGGTATTTATACTAACCT
CTAATAACCCAGAGCTAAGATCCCGGCTTCTTCTATTCTGCCTACGG
ATTGTTCTCAGTAATGGTGCAAGGGATTCCCATCGCTTTGGAGCATT
ACTCACAATGTTTTCGCTACCATCAGCCACAATGCTCAATCATGTCA
AATTAGCTGACCAGTCACCAGAAGCTGATATCGAAAGGGTAGAGATC
GATGGCTTTGAGGAGGGATCATTCCGCTTAATCCCCAATGCTCGTTC
AGGTATGAGCCGTGGAGAGATCAATGCCTATGCTGCACTTGCAGAAG
ATCTACCTGACACACTAAACCATGCAACACCTTTCGTTGATTCCGAA
GTCGAGGGAACTGCATGGGATGAGATTGAGACTTTCTTAGATATGTG
TTACAGTGTCCTAATGCAGGCATGGATAGTGACTTGCAAGTGCATGA
CTGCGCCAGACCAACCTGCTGCTTCTATTGAGAAACGCCTGCAAAAA
TATCGTCAGCAAGGCAGGATCAACCCGAGATATCTCCTGCAACCGGA
GGCTCGACGAATAATCCAGAATGTAATCCGGAAGGGAATGGTGGTCA
GACATTTCCTCACCTTTGAACTGCAGCTTGCCCGAGCACAAAGCCTT
GTATCAAATAGGTATTATGCTATGGTAGGGGATGTTGGAAAGTATAT
AGAGAATTGTGGAATGGGAGGCTTCTTTTTGACACTAAAATATGCAT
TAGGAACTAGATGGCCCACACTTGCTTTAGCTGCATTTTCAGGAGAG
CTAACAAAGCTAAAGTCCCTCATGGCATTATACCAGACCCTTGGTGA
GCAGGCCCGATATTTGGCCCTATTGGAGTCACCACATTTGATGGATT
TTGCTGCAGCAAACTACCCACTGCTATATAGCTATGCTATGGGAATA
GGCTATGTGTTAGATGTCAACATGAGGAACTACGCTTTCTCCAGATC
ATACATGAACAAGACATATTTCCAATTGGGAATGGAAACTGCAAGAA
AACAACAGGGTGCAGTTGACATGAGGATGGCAGAAGATCTCGGTCTA
ACTCAAGCCGAACGCACCGAGATGGCAAATACACTTGCCAAATTGAC
CACAGCAAATCGAGGGGCAGACACCAGGGGAGGAGTCAACCCGTTCT
CATCTGTCACTGGGACAACTCAGGTGCCCGCTGCAGCAACAGGTGAC
ACACTCGAGAGTTACATGGCAGCGGATCGACTGAGGCAGAGATATGC
TGATGCAGGCACCCATGATGATGAGATGCCACCATTGGAAGAGGAGG
AAGAGGACGACACATCTGCAGGTCCACGCACTGGACCAACTCTTGAA
CAAGTGGCCTTGGACATCCAGAACGCAGCAGTTGGAGCTCCCATCCA
TACAGATGACCTGAATGCCGCACTGGGTGATCTTGACATCTAGACAA
TTCAGATCCCAATCTAAAATTGACATACCTAATTGATTAGTTAGATG
GAACTACAGTGGATTCCATAAGGTTCCTGCCTACCATCGGCTTTAAA
GAAAAAAATAGGCCCGGACGGGTTAGCAACAAGCGACTGCCGGTGCC
AACAGCGCAATCCACAATCTACAATGGATCCCACTGATCTGAGCTTC
TCCCCAGATGAGATCAATAAGCTCATAGAGACAGGCCTGAATACTGT
AGAGTATTTTACTTCCCAACAAGTCACAGGAACATCCTCTCTTGGAA
AGAATACAATACCACCAGGGGTCACAGGACTACTAACCAATGCTGCA
GAGGCAAAGATCCAAGAGTCAACTAACCATCAGAAGGGCTCAGTTGG
TGGGGGTGCAAAACCAAAGAAACCGCGACCAAAAATTGCCATTGTGC
CAGCAGATGACAAAACAGTGCCCGGAAAGCCGATCCCAAACCCTCTA
TTAGGTCTGGACTCCACCCCGAGCACCCAAACTGTGCTTGATCTAAG
TGGGAAAACATTACCATCAGGATCCTATAAGGGGGTTAAGCTTGCGA
AATTTGGAAAAGAAAATCTGATGACACGGTTCATCGAGGAACCCAGA
GAGAATCCTATCGCAACCAGTTtCCCCATCGATTTTAAGAGGGGCAG
GGATACCGGCGGGTTCCATAGAAGGGAGTACTCAATCGGATGGGTGG
GAGATGAAGTCAAGGTCACTGAGTGGTGCAATCCATCCTGTTCTCCA
ATCACCGCTGCAGCAAGGCGATTTGAATGCACTTGTCACCAGTGTCC
AGTCACTTGCTCTGAATGTGAACGAGATACTTAATACAGTGAGAAAT
TTGGACTCTCGGATGAATCAACTGGAGACAAAAGTAGATCGCATTCT
CTCATCTCAGTCTCTAATCCAGACCATCAAGAATGACATAGTTGGAC
TTAAAGCAGGGATGGCTACTTTAGAAGGAATGATTACAACTGTGAAA
ATCATGGACCCGGGAGTTCCCAGTAATGTTACTGTGGAAGATGTACG
CAAGACACTAAGTAACCATGCTGTTGTTGTGCCAGAATCATTCAATG
ATAGTTTCTTGACTCAATCTGAAGATGTAATTTCACTTGATGAGTTG
GCTCGACCAACTGCAACAAGTGTTAAGAAGATTGTCAGGAAGGTTCC
TCCTCAGAAGGATCTGACTGGATTGAAGATTACACTAGAGCAATTGG
CAAAGGATTGCATCAGCAAACCGAAGATGAGGGAAGAGTATCTCCTC
AAAATCAACCAGGCTTCCAGTGAGGCTCAGCTAATTGACCTCAAGAA
AGCAATCATCCGCAGTGCAATTTGATCAAGAAACACCCAATTACACT
ACACTGGTATGACACTGTACTAACCCTGAGGGTTTTAGAAAAAACGA
TTAACGATAAATAAGCCCGAACACTACACACTACCTGAGGCAGCCAT
GCCATCCATCAGCATTCCCGCAGACCCCACCAATCCACGTCAATCAA
TAAAAGCGTTCCCAATTGTGATCAACAGTGATGGGGGTGAGAAAGGC
CGCTTGGTTAAACAACTACGCACAACCTACTTGAATGACCTAGATAC
TCATGAGCCACTGGTGACATTCATAAATACCTATGGATTCATCTACG
AACAGGATCGGGGGAATACCATTGTCGGAGAGGATCAACTTGGGAAG
AAAAGAGAGGCTGTGACCGCTGCAATGGTTACCCTTGGATGTGGGCC
TAATCTACCATCATTAGGGAATGTCCTGGGACAACTGAGGGAATTCC
AGGTCACTGTTAGGAAGACATCCAGCAAAGCGGAAGAGATGGTCTTT
GAAATTGTTAAGTATCCGAGAATATTTCGGGGTCATACATTAATCCA
GAAAGGACTAGTCTGTGTCTCCGCAGAAAAATTTGTTAAGTCACCAG
GGAAAATACAATCTGGAATGGACTATCTCTTCATTCCGACATTTCTG
TCAGTGACTTACTGTCCAGCTGCAATCAAATTTCAGGTACCTGGCCC
CATGTTGAAAATGAGATCAAGATACACTCAGAGCTTACAACTTGAAC
TAATGATAAGAATCCTGTGTAAGCCCGATTCGCCACTTATGAAGGTC
CATACCCCTGACAAGGAGGGAAGAGGATGTCTTGTATCAGTATGGCT
GCATGTATGCAACATCTTCAAATCAGGAAACAAGAATGGCAGTGAGT
GGCAGGAATACTGGATGAGAAAGTGTGCTAACATGCAACTTGAAGTG
TCGATTGCAGATATGTGGGGACCAACTATCATAATTCATGCCAGAGG
TCACATTCCCAAAAGTGCTAAGTTGTTTTTTGGAAAGGGTGGATGGA
GCTGCCATCCACTTCACGAAGTTGTTCCAAGTGTCACTAAAACACTA
TGGTCCGTGGGCTGTGAGATTACAAAGGCGAAGGCAATAATACAAGA
GAGTAGCATCTCTCTTCTCGTGGAGACTACTGACATCATAAGTCCAA
AAGTCAAAATTTCATCTAAGCATCGCCGCTTTGGGAAATCAAATTGG
GGTCTGTTCAAGAAAACTAAATCACTGCCTAACCTGACGGAGCTGGA
ATGACTGACCTCTAATCGAGACTACACCGCCGCAAACTATAGGTGGG
TGGTACCTCAGTGATTAATCTTGTAAGCACTGATCGTAGGCTACAAC
ACACTAATATTATCCAGATTAGAGAGCTTAATTAGCTCTGTATTAAT
AATAACACTACTATTCCAATAACTGGAATCACCAGCTTGATTTATCT
CCAAAATGATTCAAAGAAAACAAATCATATTAAGACTATCCTAAGCA
CGAACCCATATCGTCCTTCAAATCATGGGTACTATAATTCAATTTCT
GGTGGTCTCCTGTCTATTGGCAGGAGCAGGCAGCCTTGATCCAGCAG
CCCTCATGCAAATCGGTGTCATTCCAACAAATGTCCGGCAACTTATG
TATTATACTGAAGCTTCATCAGCATTCATTGTTGTGAAGTTAATGCC
TACAATTGACTCGCCGATTAGTGGATGTAATATAACATCAATTTCAA
GCTATAATGCAACAGTGACAAAACTCCTACAGCCGATCGGTGAGAAT
TTGGAGACGATTAGGAACCAGTTGATTCCAACTCGGAGGAGACGCCG
GTTTGCAGGGGTGGTGATTGGATTAGCTGCATTAGGAGTAGCTACTG
CCGCACAGGTCACTGCCGCAGTGGCACTAGTAAAGGCAAATGAAAAT
GCTGCGGCTATACTCAATCTCAAAAATGCAATCCAAAAAACAAATGC
AGCAGTTGCAGATGTGGTCCAGGCCACACAATCACTAGGAACGGCAG
TTCAAGCAGTTCAAGATCACATAAACAGTGTGGTAAGTCCAGCAATT
ACAGCAGCCAATTGTAAGGCCCAAGATGCTATCATTGGCTCAATCCT
CAATCTCTATTTGACCGAGTTGACAACCATCTTCCACAATCAAATTA
CAAACCCTGCATTGAGTCCCATTACAATTCAAGCTTTAAGGATCCTA
CTGGGGAGTACCTTGCCGACTGTGGTCGAAAAATCTTTCAATACCCA
GATAAGTGCAGCTGAGCTTCTCTCATCAGGGTTATTGACAGGCCAGA
TTGTGGGATTAGATTTGACCTATATGCAGATGGTCATAAAAATTGAG
CTGCCAACTTTAACTGTACAACCTGCAACCCAGATCATAGATCTGGC
CACCATTTCTGCATTCATTAACAATCAAGAAGTCATGGCCCAATTAC
CAACACGTGTTATGGTGACTGGCAGCTTGATCCAAGCCTATCCCGCA
TCGCAATGCACCATTACACCCAACACTGTGTACTGTAGGTATAATGA
TGCCCAAGTACTCTCAGATGATACTATGGCTTGCCTCCAAGGTAACT
TGACAAGATGCACCTTCTCTCCAGTGGTTGGGAGCTTTCTCACTCGA
TTCGTGCTGTTCGATGGAATAGTTTATGCAAATTGCAGGTCGATGTT
GTGCAAGTGCATGCAACCTGCTGCTGTGATCCTACAGCCGAGTTCAT
CCCCTGTAACTGTCATTGACATGTACAAATGTGTGAGTCTGCAGCTT
GACAATCTCAGATTCACCATCACTCAATTGGCCAATGTAACCTACAA
TAGCACCATCAAGCTTGAATCATCCCAGATCTTGTCTATTGATCCGT
TGGATATATCCCAGAATCTAGCTGCGGTGAATAAGAGTCTAAGTGAT
GCACTACAACACTTAGCACAAAGTGACACATATCTTTCTGCAATCAC
ATCAGCTACGACTACAAGTGTATTATCCATAATAGCAATCTGTCTTG
GATCGTTAGGTTTAATATTAATAATCTTGCTCAGTGTAGTTGTGTGG
AAGTTATTGACCATTGTCGTTGCTAATCGAAATAGAATGGAGAATTT
TGTTTATCATAAATAAGCATTCCACCACTCACGATCTGATCTCAGTG
AGAAAAATCAACCTGCAACTCTTGGAACAAGATAAGACAGTCATCCA
TTAGTAATTTTTAAGAAAAAAACGATAGGACCGAACCTAGTATTGAA
AGAACCGTCTCGGTCAATCTAGGTAATCGAGCTGATACCGTCTCGGA
AAGCTCAAATCGCGCGCCACCatggccgatagcaacggcaccatcac
cgtggaagagcttaaaaagctgctcgagcagtggaacctggttatcg
gctttctgttcctgacctggatctgcctgctccagttcgcttatgcc
aaccggaacaggttcctgtacatcatcaagctgatcttcctgtggct
gctgtggcctgtgacactggcctgcttcgtgctggccgctgtgtacc
ggattaactggatcaccggcggaatcgccatcgccatggcctgtctg
gtcggcctgatgtggctgtcttacttcatcgccagcttcagactgtt
tgccagaaccagaagcatgtggtccttcaaccccgagacaaacatcc
tgctgaatgtgccactgcacggcaccatcctgacaagacctctgctg
gaaagcgagctggtgattggagccgtgatcctgagaggccatctgcg
gatcgctggccaccacctgggaagatgcgacatcaaggacctgccta
aggaaatcacagtggctacatctcggaccctgagctactacaaactg
ggcgcctctcaaagagtggccggcgacagcggcttcgccgcctacag
cagatacagaatcggcaactacaagctgaataccgatcacagcagct
ccagcgacaacatcgccctgctggtgcagtgaTGACGTACGAGAAAC
ACCCAATTACACTACACTGGTATGACACTGTACTAACCCTGAGGGTT
TTAGAAAAAACGATTAACGATAAATAAGCCCGAACACTACACACTAC
CTGAGGCAGCCACCatgtcagataacggaccacagaaccagaggaac
gcacccaggattactttcggaggaccaagcgatagcaccgggagcaa
ccagaatggagagcggagcggagcaagatccaagcagagacggcccc
agggcctgccaaacaataccgcatcctggttcaccgccctgacacag
cacggcaaggaggacctgaagtttccaaggggacagggagtgcctat
caacaccaatagctcccctgacgatcagatcggctactataggaggg
caacaaggagaatcaggggaggcgacggcaagatgaaggatctgagc
ccacgctggtacttctactatctgggaaccggacctgaggcaggcct
gccatatggcgccaacaaggacggaatcatctgggtggcaaccgagg
ggccctgaacacaccaaaggatcacatcggcacaagaaatcccgcca
acaatgcagcaatcgtgctgcagctgccacagggaaccacactgccc
aagggcttttacgcagagggctctcggggaggcagccaggcatctag
cagatcctctagccggagcagaaactcctctaggaattccaccccag
gaagctccaggggcacatcccctgcccgcatggcaggaaacggaggc
gacgccgccctggccctgctgctgctggatcgcctgaatcagctgga
gtccaagatgtctggcaagggacagcagcagcagggacagaccgtga
caaagaagtccgccgccgaggcctctaagaagccaaggcagaagcgc
accgccacaaaggcctacaacgtgacccaggccttcggcaggcgcgg
accagagcagacacagggcaattttggcgaccaggagctgatcaggc
agggaaccgattataagcactggcctcagatcgcccagttcgcccca
tctgccagcgccttctttggcatgtctagaatcggcatggaggtgac
ccccagcggcacatggctgacctacacaggcgccatcaagctggacg
ataaggaccctaacttcaaggatcaggtcatcctgctgaacaagcac
atcgacgcctataagacctttccccctacagagcccaagaaggacaa
gaagaagaaggccgatgagacacaggccctgcctcagaggcagaaga
agcagcagaccgtgacactgctgccagccgccgatctggacgatttc
tccaaacagctgcagcagagcatgtccagtgccgactccacccaggc
ttgaCGTACGGATCCCAATCTTAAATCGACACACCTAATTGACCAGT
TAGATGGAACTACAGTGGATTCCATAAGGTTCCTGCCTACCATCGGC
TTTTAAGAAAAAAATAGGCCCGGACGGGTTAGCAACAAGCGACTGCC
GATGCCAATAACACAATCCACGCGTGCCACCatgtattctttcgtgt
ctgaagagacaggcaccctgatcgttaatagcgtgctgctgtttctg
gccttcgtggtgttcctgctggtgaccctggctatcctgaccgccct
gagactgtgtgcctactgctgcaacatcgtgaacgtgtccctggtca
agcctagcttctacgtgtacagccgggtgaagaacctgaacagcagc
agagtgcccgacctgctcgtgtaaGCTAGCACCTGCTATAGGCTATC
CACTGCATCATCTCTCCTGCCATACTTCCTACTCACATCATATCTAT
TTTAAAGAAAAAATAGGCCCGAACACTAATCGTGCCGGCAGTGCCAC
TGCACACACAACACTACACATACAATACACTACAATGGTTGCAGAAG
ATGCCCCTGTTAGGGCCACTTGCCGAGTATTATTTCGAACAACAACT
TTAATCTTTCTATGCACACTACTAGCATTAAGCATCTCTATCCTTTA
TGAGAGTTTAATAACCCAAAAGCAAATCATGAGCCAAGCAGGCTCAA
CTGGATCTAATTCTGGATTAGGAAGTATCACTGATCTTCTTAATAAT
ATTCTCTCTGTCGCAAATCAGATTATATATAACTCTGCAGTCGCTCT
ACCTCTACAATTGGACACTCTTGAATCAACACTCCTTACAGCCATTA
AGTCTCTTCAAACCAGTGACAAGCTAGAACAGAACTGCTCGTGGAGT
GCTGCACTGATTAATGATAATAGATACATTAATGGCATCAATCAGTT
CTATTTTTCAATTGCTGAGGGTCGCAATCTGACACTTGGCCCACTTC
TTAATATGCCTAGTTTCATTCCAACTGCCACGACACCAGAGGGCTGC
ACCAGGATCCCATCATTCTCGCTCACTAAGACACACTGGTGTTATAC
ACACAATGTTATCCTGAATGGATGCCAGGATCATGTATCCTCAAATC
AATTTGTTTCTATGGGAATCATTGAACCCACTTCTGCCGGGTTTCCA
TTCTTTCGAACCCTAAAGACTCTATATCTCAGCGATGGGGTCAATCG
TAAGAGCTGCTCTATCAGTACAGTTCCGGGGGGTTGTATGATGTACT
GTTTTGTTTCTACTCAACCAGAGAGGGATGACTACTTTTCTGCCGCT
CCTCCAGAACAACGAATTATTATAATGTACTATAATGATACAATCGT
GGAGCGCATAATTAATCCACCCGGGGTACTAGATGTATGGGCAACAT
TGAACCCAGGAACAGGAAGCGGGGTATATTATTTAGGTTGGGTGCTC
TTTCCAATATATGGCGGCGTGATTAAAGGTACGAGTTTATGGAATAA
TCAAGCAAATAAATACTTTATCCCCCAGATGGTTGCTGCTCTCTGCT
CACAAAACCAGGCAACTCAAGTCCAAAATGCTAAGTCATCATACTAT
AGCAGCTGGTTTGGCAATCGAATGATTCAGTCTGGGATCCTGGCATG
TCCTCTTCGACAGGATCTAACCAATGAGTGTTTAGTTCTGCCCTTTT
CTAATGATCAGGTGCTTATGGGTGCTGAAGGGAGATTATACATGTAT
GGTGACTCGGTGTATTACTATCAAAGAAGCAATAGTTGGTGGCCTAT
GACCATGCTGTATAAGGTAACCATAACATTCACTAATGGTCAGCCAT
CTGCTATATCAGCTCAGAATGTGCCCACACAGCAGGTCCCTAGACCT
GGGACAGGAGACTGCTCTGCAACCAATAGATGTCCCGGTTTTTGCTT
GACAGGAGTGTATGCCGATGCCTGGTTACTGACCAACCCTTCGTCTA
CCAGTACATTTGGATCAGAAGCAACCTTCACTGGTTCTTATCTCAAC
ACAGCAACTCAGCGTATCAATCCGACGATGTATATCGCGAACAACAC
ACAGATCATAAGCTCACAGCAATTTGGATCAAGCGGTCAAGAAGCAG
CATATGGCCACACAACTTGTTTTAGGGACACAGGCTCTGTTATGGTA
TACTGTATCTATATTATTGAATTGTCCTCATCTCTCTTAGGACAATT
TCAGATTGTCCCATTTATCCGTCAGGTGACACTATCCTAAAGGCAGA
AGCCTTCAGGTCTGACCCAGCCAATCAAAGCATTATACCAGACCATG
GCCTACCATCGGCTTTAAAGAAAAAAATAGGCCCGGACGGGTTAGCA
ACAAGCGGCGGCCGCCGCCACCatgttcgtcttcctggtcctgctgc
ctctggtctcctcacagtgcgtcaatctgacaactcggactcagctg
ccacctgcttatactaatagcttcaccagaggcgtgtactatcctga
caaggtgtttagaagctccgtgctgcactctacacaggatctgtttc
tgccattctttagcaacgtgacctggttccacgccatccacgtgagc
ggcaccaatggcacaaagcggttcgacaatcccgtgctgccttttaa
cgatggcgtgtacttcgcctctaccgagaagagcaacatcatcagag
gctggatctttggcaccacactggactccaagacacagtctctgctg
atcgtgaacaatgccaccaacgtggtcatcaaggtgtgcgagttcca
gttttgtaatgatcccttcctgggcgtgtactatcacaagaacaata
agagctggatggagtccgagtttagagtgtattctagcgccaacaac
tgcacatttgagtacgtgagccagcctttcctgatggacctggaggg
caagcagggcaatttcaagaacctgagggagttcgtgtttaagaata
tcgacggctacttcaaaatctactctaagcacacccccatcaacctg
gtgcgcgacctgcctcagggcttcagcgccctggagcccctggtgga
tctgcctatcggcatcaacatcacccggtttcagacactgctggccc
tgcacagaagctacctgacacccggcgactcctctagcggatggacc
gccggcgctgccgcctactatgtgggctacctccagccccggacctt
cctgctgaagtacaacgagaatggcaccatcacagacgcagtggatt
gcgccctggaccccctgagcgagacaaagtgtacactgaagtccttt
accgtggagaagggcatctatcagacatccaatttcagggtgcagcc
aaccgagtctatcgtgcgctttcctaatatcacaaacctgtgcccat
ttggcgaggtgttcaacgcaacccgcttcgccagcgtgtacgcctgg
aataggaagcggatcagcaactgcgtggccgactatagcgtgctgta
caactccgcctctttcagcacctttaagtgctatggcgtgtccccca
caaagctgaatgacctgtgctttaccaacgtctacgccgattctttc
gtgatcaggggcgacgaggtgcgccagatcgcccccggccagacagg
caagatcgcagactacaattataagctgccagacgatttcaccggct
gcgtgatcgcctggaacagcaacaatctggattccaaagtgggcggc
aactacaattatctgtaccggctgtttagaaagagcaatctgaagcc
cttcgagagggacatctctacagaaatctaccaggccggcagcaccc
cttgcaatggcgtggagggctttaactgttatttcccactccagtcc
tacggcttccagcccacaaacggcgtgggctatcagccttaccgcgt
ggtggtgctgagctttgagctgctgcacgccccagcaacagtgtgcg
gccccaagaagtccaccaatctggtgaagaacaagtgcgtgaacttc
aacttcaacggcctgaccggcacaggcgtgctgaccgagtccaacaa
gaagttcctgccatttcagcagttcggcagggacatcgcagatacca
cagacgccgtgcgcgacccacagaccctggagatcctggacatcaca
ccctgctctttcggcggcgtgagcgtgatcacacccggcaccaatac
aagcaaccaggtggccgtgctgtatcaggacgtgaattgtaccgagg
tgcccgtggctatccacgccgatcagctgaccccaacatggcgggtg
tacagcaccggctccaacgtcttccagacaagagccggatgcctgat
cggagcagagcacgtgaacaattcctatgagtgcgacatcccaatcg
gcgccggcatctgtgcctcttaccagacccagacaaactctcccaga
agagcccggagcgtggcctcccagtctatcatcgcctataccatgtc
cctgggcgccgagaacagcgtggcctactctaacaatagcatcgcca
tcccaaccaacttcacaatctctgtgaccacagagatcctgcccgtg
tccatgaccaagacatctgtggactgcacaatgtatatctgtggcga
ttctaccgagtgcagcaacctgctgctccagtacggcagcttttgta
cccagctgaatagagccctgacaggcatcgccgtggagcaggataag
aacacacaggaggtgttcgcccaggtgaagcaaatctacaagacccc
ccctatcaaggactttggcggcttcaatttttcccagatcctgcctg
atccatccaagccttctaagcggagctttatcgaggacctgctgttc
aacaaggtgaccctggccgatgccggcttcatcaagcagtatggcga
ttgcctgggcgacatcgcagccagggacctgatctgcgcccagaagt
ttaatggcctgaccgtgctgccacccctgctgacagatgagatgatc
gcacagtacacaagcgccctgctggccggcaccatcacatccggatg
gaccttcggcgcaggagccgccctccagatcccctttgccatgcaga
tggcctataggttcaacggcatcggcgtgacccagaatgtgctgtac
gagaaccagaagctgatcgccaatcagtttaactccgccatcggcaa
gatccaggacagcctgtcctctacagccagcgccctgggcaagctcc
aggatgtggtgaatcagaacgcccaggccctgaataccctggtgaag
cagctgagcagcaacttcggcgccatctctagcgtgctgaatgacat
cctgagccggctggacaaggtggaggcagaggtgcagatcgaccggc
tgatcaccggccggctccagagcctccagacctatgtgacacagcag
ctgatcagggccgccgagatcagggccagcgccaatctggcagcaac
caagatgtccgagtgcgtgctgggccagtctaagagagtggactttt
gtggcaagggctatcacctgatgtccttccctcagtctgccccacac
ggcgtggtgtttctgcacgtgacctacgtgcccgcccaggagaagaa
cttcaccacagcccctgccatctgccacgatggcaaggcccactttc
caagggagggcgtgttcgtgtccaacggcacccactggtttgtgaca
cagcgcaatttctacgagccccagatcatcaccacagacaacacctt
cgtgagcggcaactgtgacgtggtcatcggcatcgtgaacaataccg
tgtatgatccactccagcccgagctggacagctttaaggaggagctg
gataagtatttcaagaatcacacctcccctgacgtggatctgggcga
catcagcggcatcaatgcctccgtggtgaacatccagaaggagatcg
accgcctgaacgaggtggctaagaatctgaacgagagcctgatcgac
ctccaggagctgggcaagtatgagcagtacatcaagtggccctggta
catctggctgggcttcatcgccggcctgatcgccatcgtgatggtga
ccatcatgctgtgctgtatgacatcctgctgttcttgcctgaagggc
tgctgtagctgtggctcctgctgtaagtttgacgaggatgactctga
acctgtgctgaagggcgtgaagctgcattacacctaaTAAGGTCGAC
TCATGGAATGCATACCAAACATTATTGACACTAATGACACACAAAAT
TGGTTTTAAGAAAAACCAAGAGAACAATAGGCCAGAATGGCTGGGTC
TCGGGAGATATTACTCCCTGAAGTCCATCTCAATTCACCAATTGTAA
AGCATAAGCTATACTATTACATTCTACTTGGAAACCTCCCAAATGAG
ATCGACCTTGACGATTTAGGTCCATTACATAATCAAAATTGGAATCA
GATAGCACATGAAGAGTCTAACTTAGCTCAACGCTTGGTAAATGTAA
GAAATTTTCTAATTACCCACATCCCTGATCTTAGAAAGGGCCATTGG
CAAGAGTATGTCAATGTAATACTGTGGCCGCGAATTCTTCCCTTGAT
CCCGGATTTTAAAATCAATGACCAATTGCCTCTGCTCAAAAATTGGG
ACAAGTTAGTTAAAGAATCATGTTCAGTAATCAATGCAGGTACTTCC
CAGTGCATTCAGAATCTCAGCTATGGACTGACAGGTCGTGGGAACCT
CTTTACACGATCACGTGAACTCTCTGGTGACCGCAGGGATATTGATC
TTAAGACAGTTGTGGCAGCATGGCATGACTCAGACTGGAAAAGAATA
AGTGATTTTTGGATTATGATCAAATTCCAGATGAGACAATTAATTGT
TAGGCAAACAGATCATAATGATTCTGATTTAATCACGTATATCGAAA
ATAGAGAAGGCATAATCATCATAACCCCTGAACTGGTAGCATTATTT
AACACTGAGAATCATACACTAACATACATGACCTTTGAAATTGTACT
GATGGTTTCAGATATGTACGAAGGTCGTCACAACATTTTATCACTAT
GCACAGTTAGCACTTACCTGAATCCTCTGAAGAAAAGAATAACATAT
TTATTGAGCCTTGTAGATAACTTAGCTTTTCAGATAGGTGATGCTGT
ATATAACATAATTGCTTTGCTAGAATCCTTTGTATATGCACAGTTGC
AAATGTCAGATCCCATCCCAGAACTCAGAGGACAATTCCATGCATTC
GTATGTTCTGAGATTCTTGATGCACTAAGAGGAACTAATAGTTTCAC
CCAGGATGAATTAAGAACTGTGACAACTAATTTGATATCCCCATTCC
AAGATCTGACCCCAGATCTTACGGCTGAATTGCTCTGTATAATGAGG
CTTTGGGGACACCCCATGCTCACTGCCAGTCAAGCTGCAGGAAAGGT
ACGCGAGTCTATGTGTGCTGGAAAAGTATTAGACTTTCCCACCATTA
TGAAAACACTAGCCTTTTTCCATACTATTCTGATCAATGGATACAGG
AGGAAGCATCATGGAGTATGGCCACCCTTAAACTTACCGGGTAATGC
TTCAAAGGGTCTCACGGAACTTATGAATGACAATACTGAAATAAGCT
ATGAATTCACACTTAAGCATTGGAAGGAAGTCTCTCTTATAAAATTC
AAGAAATGTTTTGATGCAGACGCAGGTGAGGAACTCAGTATATTTAT
GAAAGATAAGGCAATTAGTGCCCCAAAACAAGACTGGATGAGTGTGT
TTAGAAGAAGCCTAATCAAACAGCGCCATCAGCATCATCAGGTCCCC
CTACCAAATCCATTCAATCGACGGCTGTTGCTAAACTTTCTCGGAGA
TGACAAATTCGACCCGAATGTGGAGCTACAGTATGTAACATCAGGTG
AGTATCTACATGATGACACGTTTTGTGCATCATATTCACTAAAAGAG
AAGGAAATTAAACCTGATGGTCGAATTTTTGCAAAGTTGACTAAGAG
AATGAGATCATGTCAAGTTATAGCAGAATCTCTTTTAGCGAACCATG
CTGGGAAGTTAATGAAAGAGAATGGTGTTGTGATGAATCAGCTATCA
TTAACAAAATCACTATTAACAATGAGTCAGATTGGAATAATATCCGA
GAAAGCTAGAAAGTCAACTCGAGATAACATAAATCAACCTGGTTTCC
AGAATATCCAGAGAAATAAATCACATCACTCCAAGCAAGTCAATCAG
CGAGATCCAAGTGATGACTTTGAATTGGCAGCATCTTTTTTAACTAC
TGATCTCAAAAAATATTGTTTACAATGGAGGTACCAGACAATTATCC
CATTTGCTCAATCATTAAACAGAATGTATGGTTATCCTCATCTCTTT
GAGTGGATTCACTTACGGCTAATGCGTAGTACACTTTACGTGGGGGA
TCCCTTCAACCCACCAGCAGATACCAGTCAATTTGATCTAGATAAAG
TAATTAATGGAGATATCTTCATTGTATCACCCAGAGGTGGAATTGAA
GGGCTGTGTCAAAAGGCTTGGACAATGATATCTATCGCTGTGATAAT
TCTATCTGCCACAGAGTCTGGCACACGAGTAATGAGTATGGTGCAGG
GAGATAATCAAGCAATTGCTGTCACCACACGAGTACCAAGGAGCCTG
CCGACTCTTGAGAAAAAGACTATTGCTTTTAGATCTTGTAATCTATT
CTTTGAGAGGTTAAAATGTAATAATTTTGGATTAGGTCACCATTTGA
AAGAACAAGAGACTATCATTAGTTCTCACTTCTTTGTTTATAGCAAG
AGAATATTCTATCAGGGGAGGATTCTAACGCAAGCCTTAAAAAATGC
TAGTAAGCTCTGCTTGACAGCTGATGTCCTAGGAGAATGCACCCAAT
CATCATGTTCTAATCTTGCAACTACTGTCATGAGGTTAACTGAGAAT
GGTGTTGAAAAAGATATCTGTTTCTACTTGAATATCTATATGACCAT
CAAACAGCTCTCCTATGATATCATCTTCCCTCAAGTGTCAATTCCTG
GAGATCAGATCACATTAGAATACATAAATAATCCACACCTGGTATCA
CGATTGGCTCTTTTGCCATCCCAGTTAGGAGGTCTAAACTACCTGTC
ATGCAGTAGGCTGTTCAATCGAAACATAGGCGACCCGGTGGTTTCCG
CAGTTGCAGATCTTAAGAGATTAATTAAATCAGGATGTATGGATTAC
TGGATCCTTTATAACTTATTAGGGAGAAAACCGGGAAACGGCTCATG
GGCTACTTTAGCAGCTGACCCGTACTCAATCAATATAGAGTATCAAT
ACCCTCCAACTACAGCTCTTAAGAGGCACACCCAACAAGCTCTGATG
GAACTCAGTACGAATCCAATGTTACGTGGCATATTCTCTGACAATGC
ACAGGCAGAAGAAAATAACCTTGCTAGGTTTCTCCTGGATAGGGAGG
TGATCTTTCCGCGTGTAGCTCACATCATCATTGAGCAAACCAGTGTC
GGGAGGAGAAAACAGATTCAAGGATATTTGGATTCAACTAGATCGAT
AATGAGGAAATCACTAGAAATTAAGCCCTTATCCAATAGGAAGCTTA
ATGAAATACTGGATTACAACATCAATTACCTAGCTTACAATTTGGCA
TTACTCAAGAATGCTATTGAACCTCCGACTTATTTGAAGGCAATGAC
ACTTGAAACATGTAGCATCGACATTGCAAGGAACCTCCGGAAGCTCT
CCTGGGCCCCACTCTTGGGTGGGAGAAATCTTGAAGGATTAGAGACG
CCAGATCCCATTGAAATTACTGCAGGAGCATTAATTGTTGGATCGGG
CTACTGTGAACAGTGTGCTGCAGGAGACAATCGATTCACATGGTTTT
TCTTGCCATCTGGTATCGAGATAGGAGGGGATCCCCGTGATAATCCT
CCTATCCGTGTACCGTACATTGGCTCCAGGACTGATGAGAGGAGGGT
AGCCTCAATGGCATACATCAGGGGTGCCTCGAGTAGCTTAAAAGCAG
TTCTTAGACTGGCGGGAGTGTACATCTGGGCATTCGGAGATACTCTG
GAGAATTGGATAGATGCACTGGATTTGTCTCACACTAGAGTTAACAT
CACACTTGAACAGCTGCAATCCCTCACCCCACTTCCAACCTCTGCCA
ATCTAACCCATCGGTTGGATGATGGCACAACTACCCTAAAGTTTACT
CCTGCGAGCTCTTACACCTTTTCAAGTTTCACTCATATATCAAATGA
TGAGCAATACCTGACAATTAATGACAAAACTGCAGATTCAAATATAA
TCTACCAACAGTTAATGATCACTGGACTCGGAATCTTAGAAACATGG
AATAATCCCCCAATCAATAGAACATTCGAAGAATCTACCCTACATTT
GCACACTGGTGCATCATGTTGTGTCCGACCTGTGGACTCCTGCATTC
TCTCAGAAGCATTAACAGTCAAGCCACATATTACAGTACCGTACAGC
AATAAATTTGTATTTGATGAGGACCCGCTATCTGAATATGAAACTGC
AAAACTGGAATCGTTATCATTCCAAGCCCAATTAGGCAACATTGATG
CTGTAGATATGACAGGTAAATTAACATTATTGTCCCAATTCACTGCA
AGGCAGATTATCAATGCAATCACTGGACTCGATGAGTCTGTCTCTCT
TACTAATGATGCCATTGTTGCATCAGACTATGTCTCCAATTGGATTA
GTGAATGCATGTATACCAAATTAGATGAATTATTTATGTATTGTGGG
TGGGAACTACTATTGGAACTATCCTATCAAATGTATTATCTGAGGGT
AGTTGGGTGGAGTAATATAGTGGATTATTCTTACATGATCTTGAGAA
GAATCCCGGGTGCAGCATTAAACAATCTGGCATCTACATTAAGTCAT
CCAAAACTTTTCCGACGAGCTATCAACCTAGATATAGTTGCCCCCTT
AAATGCTCCTCATTTTGCATCTCTGGACTACATCAAGATGAGTGTGG
ATGCAATACTCTGGGGCTGTAAAAGAGTCATCAATGTGCTCTCCAAT
GGAGGGGACTTAGAATTAGTTGTGACATCTGAAGATAGCCTTATTCT
CAGTGACCGATCCATGAATCTCATTGCAAGGAAATTAACTTTATTAT
CACTGATTCACCATAATGGTTTGGAACTACCAAAGATTAAGGGGTTC
TCTCCTGATGAGAAGTGTTTCGCTTTGACAGAATTTTTGAGGAAAGT
GGTGAACTCAGGGTTGAGTTCAATAGAGAACCTATCAAATTTTATGT
ACAATGTGGAGAACCCACGGCTTGCAGCATTCGCCAGCAACAATTAC
TACCTGACCAGAAAATTATTGAATTCAATACGAGATACTGAGTCGGG
TCAAGTAGCAGTCACCTCATATTATGAATCATTAGAATATATTGATA
GTCTTAAGCTAACCCCACATGTGCCTGGCACCTCATGCATTGAGGAT
GATAGTCTATGTACAAATGATTACATAATCTGGATCATAGAGTCTAA
TGCAAACTTGGAGAAGTATCCAATTCCAAATAGCCCTGAGGATGATT
CCAATTTCCATAACTTTAAGTTGAATGCTCCATCGCACCATACCTTA
CGCCCATTAGGGTTGTCATCAACTGCTTGGTATAAGGGTATAAGCTG
CTGCAGGTACCTTGAGCGATTAAAGCTACCACAAGGTGATCATTTAT
ATATTGCAGAAGGTAGTGGTGCCAGTATGACAATCATAGAATACCTA
TTCCCAGGAAGAAAGATATATTACAATTCTTTATTTAGTAGTGGTGA
CAATCCCCCACAAAGAAATTATGCACCAATGCCTACTCAGTTCATTG
AGAGTGTCCCATACAAGCTCTGGCAAGCACACACAGATCAATATCCC
GAGATTTTTGAGGACTTCATCCCTCTATGGAACGGAAACGCCGCCAT
GACTGACATAGGAATGACAGCTTGTGTAGAATTCATCATCAATCGAG
TCGGCCCAAGGACTTGCAGTTTAGTACATGTAGATTTGGAATCAAGT
GCAAGCTTAAATCAACAATGCCTGTCAAAGCCGATAATTAATGCTAT
CATCACTGCTACAACTGTTTTGTGCCCTCATGGGGTGCTTATTCTGA
AATATAGTTGGTTGCCATTTACTAGATTTAGTACTTTGATCACTTTC
TTATGGTGCTACTTTGAGAGAATCACTGTTCTTAGGAGCACATATTC
TGATCCAGCTAATCATGAGGTTTATTTAATTTGTATCCTTGCCAACA
ACTTTGCATTCCAGACTGTCTCGCAGGCAACAGGAATGGCGATGACT
TTAACTGATCAAGGGTTTACTTTGATATCACCTGAAAGAATAAATCA
GTATTGGGATGGTCACTTGAAGCAAGAACGTATCGTAGCAGAAGCAA
TTGATAAGGTGGTTCTAGGAGAAAATGCTCTATTTAATTCGAGTGAT
AATGAATTAATTCTCAAATGTGGAGGGACACCAAATGCACGGAATCT
CATCGATATCGAGCCAGTCGCAACTTTCATAGAATTTGAACAATTGA
TCTGCACAATGTTGACAACCCACTTGAAGGAAATAATTGATATAACA
AGGTCTGGAACCCAGGATTATGAAAGTTTATTACTCACTCCTTACAA
TTTAGGTCTTCTTGGTAAAATCAGTACGATAGTGAGATTATTAACAG
AAAGGATTCTAAATCATACTATCAGGAATTGGTTGATCCTCCCACCT
TCGCTCCGGATGATCGTGAAGCAGGACTTGGAATTCGGCATATTCAG
GATTACTTCCATCCTCAATTCTGATCGGTTCCTGAAGCTTTCTCCAA
ATAGGAAATACTTGATTGCACAATTAACTGCAGGCTACATTAGGAAA
TTGATTGAGGGGGATTGCAATATCGATCTAACCAGACCTATCCAAAA
GCAAATCTGGAAAGCATTAGGTTGTGTAGTCTATTGTCACGATCCAA
TGGATCAAAGGGAGTCAACAGAGTTTATTGATATAAATATTAATGAA
GAAATAGACCGCGGGATCGATGGCGAGGAAATCTAAACATATCAAGA
ATCAGAATTAGTTTAAGAAAAAAGAAGAGGATTAATCTTGGTTTTCC
CCTTGGTGGGTCGGCATGGCATCTCCACCTCCTCGCGGTCCGACCTG
GGCATCCGAAGGAGGACGCACGTCCACTCGGATGGCTAAGGGAGCGG
CCGGGGATCCGGCTGCTAACAAAGCCCGAAAGGAAGCTGAGTTGGCT
GCTGCCACCGCTGAGCAATAACTAGCATAACCCCTTGGGGCCTCTAA
ACGGGTCTTGAGGGGTTTTTTGCTGAAAGGAGGAACTATATCCGGAT
CATGTGAGCAAAAGGCCAGCAAAAGGCCAGGAACCGTAAAAAGGCCG
CGTTGCTGGCGTTTTTCCATAGGCTCCGCCCCCCTGACGAGCATCAC
AAAAATCGACGCTCAAGTCAGAGGTGGCGAAACCCGACAGGACTATA
AAGATACCAGGCGTTTCCCCCTGGAAGCTCCCTCGTGCGCTCTCCTG
TTCCGACCCTGCCGCTTACCGGATACCTGTCCGCCTTTCTCCCTTCG
GGAAGCGTGGCGCTTTCTCATAGCTCACGCTGTAGGTATCTCAGTTC
GGTGTAGGTCGTTCGCTCCAAGCTGGGCTGTGTGCACGAACCCCCCG
TTCAGCCCGACCGCTGCGCCTTATCCGGTAACTATCGTCTTGAGTCC
AACCCGGTAAGACACGACTTATCGCCACTGGCAGCAGCCACTGGTAA
CAGGATTAGCAGAGCGAGGTATGTAGGCGGTGCTACAGAGTTCTTGA
AGTGGTGGCCTAACTACGGCTACACTAGAAGAACAGTATTTGGTATC
TGCGCTCTGCTGAAGCCAGTTACCTTCGGAAAAAGAGTTGGTAGCTC
TTGATCCGGCAAACAAACCACCGCTGGTAGCGGTGGTTTTTTTGTTT
GCAAGCAGCAGATTACGCGCAGAAAAAAAGGATCTCAAGAAGATCCT
TTGATCTTTTCTACGGGGTCTGACGCTCAGTGGAACGAAAACTCACG
TTAAGGGATTTTGGTCATGAGATTATCAAAAAGGATCTTCACCTAGA
TCCTTTTAAATTAAAAATGAAGTTTTAAATCAATCTAAAGTATATAT
GAGTAAACTTGGTCTGACAGTTACCAATGCTTAATCAGTGAGGCACC
TATCTCAGCGATCTGTCTATTTCGTTCATCCATAGTTGCCTGACTCC
CCGTCGTGTAGATAACTACGATACGGGAGGGCTTACCATCTGGCCCC
AGTGCTGCAATGATACCGCGAGACCCACGCTCACCGGCTCCAGATTT
ATCAGCAATAAACCAGCCAGCCGGAAGGGCCGAGCGCAGAAGTGGTC
CTGCAACTTTATCCGCCTCCATCCAGTCTATTAATTGTTGCCGGGAA
GCTAGAGTAAGTAGTTCGCCAGTTAATAGTTTGCGCAACGTTGTTGC
CATTGCTACAGGCATCGTGGTGTCACGCTCGTCGTTTGGTATGGCTT
CATTCAGCTCCGGTTCCCAACGATCAAGGCGAGTTACATGATCCCCC
ATGTTGTGCAAAAAAGCGGTTAGCTCCTTCGGTCCTCCGATCGTTGT
CAGAAGTAAGTTGGCCGCAGTGTTATCACTCATGGTTATGGCAGCAC
TGCATAATTCTCTTACTGTCATGCCATCCGTAAGATGCTTTTCTGTG
ACTGGTGAGTACTCAACCAAGTCATTCTGAGAATAGTGTATGCGGCG
ACCGAGTTGCTCTTGCCCGGCGTCAATACGGGATAATACCGCGCCAC
ATAGCAGAACTTTAAAAGTGCTCATCATTGGAAAACGTTCTTCGGGG
CGAAAACTCTCAAGGATCTTACCGCTGTTGAGATCCAGTTCGATGTA
ACCCACTCGTGCACCCAACTGATCTTCAGCATCTTTTACTTTCACCA
GCGTTTCTGGGTGAGCAAAAACAGGAAGGCAAAATGCCGCAAAAAAG
GGAATAAGGGCGACACGGAAATGTTGAATACTCATACTCTTCCTTTT
TCAATATTATTGAAGCATTTATCAGGGTTATTGTCTCATGAGCGGAT
ACATATTTGAATGTATTTAGAAAAATAAACAAATAGGGGTTCCGCGC
ACATTTCCCCGAAAAGT.

### V. Methods of Making

Also included in the present invention are methods of making and using CVB viral expression vectors, including, but not limited to any of those described herein.

For example, the present invention includes methods of expressing a coronavirus S protein, including but not limited to the S protein of SARS-CoV-2, in a cell by infecting the cell with a CVB viral expression vector, viral particle, or composition as described herein.

The present invention includes methods of inducing an immune response in a subject to a coronavirus S protein, including but not limited to the S protein of SARS-CoV-2, by administering a viral expression vector, viral particle, or composition as described herein to the subject. The immune response may include a humoral immune response and/or a cellular immune response. The immune response may enhance an innate and/or adaptive immune response.

The present invention includes methods expressing a heterologous coronavirus S protein, including but not limited to the S protein of SARS-CoV-2, in a subject by administering a viral expression vector, viral particle, or composition as described herein to the subject.

The present invention includes methods expressing a heterologous coronavirus S protein, including but not limited to the S protein of SARS-CoV-2 alpha, gamma, delta, and omicron strains, in a subject by administering a viral expression vector, viral particle, or composition as described herein to the subject.

### VI. Methods of Treatment

The disclosure can be used in gene therapy and/or therapeutic approaches for the treatment of disease which involve the increase or decrease of a nucleotide sequence of interest in a host-cell. In these embodiments, the expressible heterologous nucleotide sequence may be derived from a mammalian genome. It may be particularly useful in some embodiments to have the expressible heterologous nucleotide sequence derived from a human genome, wherein expression of the wild-type RNA and/or protein can produce therapeutic effects in a patient. For example, the expressible heterologous nucleotide sequence can encode CFTR, NeuroD1, Cas9 and Guide RNAs, or any other such sequence. In other embodiments, the heterologous nucleotide sequence encodes a secreted protein.

In other embodiments, the expressible heterologous nucleotide sequence responds to positive selection stimuli. In other embodiments, the expressible heterologous nucleotide sequence also responds to negative selection stimuli. In further embodiments, it may be useful for the polynucleotide sequences to further comprise a reporter gene. For example, the report gene can be a luciferase or green fluorescent protein.

In some embodiments, CVB expresses one or more nucleotide sequences (e.g., siRNAs) that modify the translation and/or transcription of a host-cell nucleotide sequence of interest within a host cell. In some embodiments, transcription and/or translation of the expressible heterologous nucleotide sequence is modified so that its nucleotide sequence is codon degenerated with respect to the endogenous gene in a cell. Additionally, the expressible heterologous nucleotide sequence can be modified so that it co-expresses inhibitory or silencing sequences capable of inhibiting or silencing a host-cell nucleotide sequence of interest within a host cell.

In certain embodiments of the disclosure, CVB compositions can be utilized to prepare antigenic preparations that be used as vaccines. Any suitable antigen(s) can be prepared in accordance with the disclosure, including antigens obtained from prions, viruses, mycobacterium, protozoa (e.g., Plasmodium falciparum (malaria)), trypanosomes, bacteria (e.g., Streptococcus, Neisseria, etc.), etc.

Host cells can be transfected with single CVB particles containing one or more heterologous polynucleotide sequences, or with a plurality of CVB particles, where each comprises the same or different heterologous polynucleotide sequence(s). For example, a multi-subunit antigen (including intracellular and cell-surface multi-subunit components) can be prepared by expressing the individual subunits on separate vectors, but infecting the same host cell with all the vectors, such that assembly occurs within the host cell.

Vaccines often contain a plurality of antigen components, e.g., derived from different proteins, and/or from different epitopic regions of the same protein. For example, a vaccine against a viral disease can comprise one or more polypeptide sequences obtained from the virus which, when administered to a host, elicit an immunogenic or protective response to viral challenge.

As mentioned, the disclosure can also be utilized to prepare polypeptide multimers, e.g., where an antigenic preparation is produced which is comprised of more than one polypeptide. For instance, virus capsids can be made up of more than one polypeptide subunit. By transducing a host cell with vectors carrying different viral envelope sequences, the proteins, when expressed in the cell, can self-assemble into three-dimensional structures containing more than one protein subunit (e.g., in their native configuration).

In further embodiments, the expressible heterologous nucleotide sequence is derived from another virus, other than PIV5. For example, the heterologous nucleotide sequence may encode (from any strain) influenza HA, RSV F, HIV Gag and/or Env, etc. Such embodiments can be useful for developing vaccines and/or methods of vaccination. The examples given here are non-limiting, as it will be understood by those in the art that nucleotide sequences from a variety of pathogenic agents (including also bacteria, parasites, etc.) may be desirable to use for an CVB vaccine composition and/or method of vaccination.

Examples of viruses to which vaccines can be produced in accordance with the disclosure include, e.g., coronaviruses, orthomyxoviruses, influenza virus A (including all strains varying in their HA and NA proteins, such as (non-limiting examples) H1N1, H1N2, H2N2, H3N2, H5N1, H6N1, H7N7, H7N9, and H3N8 etc); influenza B, influenza C, thogoto virus (including Dhori, Batken virus, SiAR 126 virus), and isavirus (e.g., infectious salmon anemia virus) and the like. These include coronaviruses isolated or transmitted from all species types, including isolates from invertebrates, vertebrates, mammals, humans, non-human primates, monkeys, pigs, cows, and other livestock, birds, domestic poultry such as turkeys, chickens, quail, and ducks, wild birds (including aquatic and terrestrial birds), reptiles, etc. These also include existing strains which have changed, e.g., through mutation, antigenic drift, antigenic shift, recombination, etc., especially strains which have increased virulence and/or interspecies transmission (e.g., human-to-human).

### VII. Methods of Administration

### A. Administration by Vaccination

The present invention includes methods of vaccinating a subject by administering a viral expression vector, viral particle, or composition as described herein to the subject.

The disclosure provides vaccines against all coronaviruses, including existing subtypes, derivatives thereof, and recombinants thereof, such as subtypes and recombinants which have the ability to spread from human-to-human. Various isolates have been characterized, especially for SARS-COV-2.

The disclosure also provides methods for producing CVB compositions. Examples of host cells which can be utilized to produce CVB compositions, include, any mammalian or human cell line or primary cell. Non-limiting examples include, e.g., 293, HT1080, Jurkat, and SupTI cells. Other examples are CHO, 293, Hela, Vero, L929, BHK, NIH 3T3, MRC-5, BAE-1, HEP-G2, NSO, U937, Namalwa, HL60, WEHI 231, YAC 1, U 266B1, SH-SY5Y, CHO, e.g., CHO-K1 (CCL-61), 293 (e.g., CRL-1573). Cells are cultured under conditions effective to produce transfection and expression. Such conditions include, e.g., the particular milieu needed to achieve protein production. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including cell media, substrates, oxygen, carbon dioxide, glucose and other sugar substrates, serum, growth factors, etc.).

The disclosure also provides various treatment methods involving delivering CVB to host cells in vivo. In some embodiments, CVB is delivered into a subject for treating or preventing coronaviruses. In other embodiments, CVB is delivered into a subject for treating or preventing SARS-COV-2 alpha, delta, omicron strains, or variants thereof or eliciting an immune response to SARS-COV-2 in a subject.

It is contemplated that when used to treat various diseases, the compositions and methods of the disclosure can be combined with other therapeutic agents suitable for the same or similar diseases. Also, two or more embodiments of the disclosure may be also co-administered to generate additive or synergistic effects. When co-administered with a second therapeutic agent, the embodiment of the disclosure and the second therapeutic agent may be simultaneously or sequentially (in any order). Suitable therapeutically effective dosages for each agent may be lowered due to the additive action or synergy.

As a non-limiting example, the disclosure can be combined with other therapies that block inflammation through (e.g., via inhibition, reduction and/or blockage of IL1, INFα/β, IL6, TNF, L13, IL23, etc.). In some embodiments, CVB compositions and methods disclosed herein are useful to enhance the efficacy of vaccines directed to SARS-COV-2 infections. The compositions and methods of the disclosure can be administered to a subject either simultaneously with or before (e.g., 1-30 days before) a reagent (including but not limited to small molecules, antibodies, or cellular reagents) that acts to elicit an immune response (e.g., to treat cancer or an infection). The compositions and methods of the disclosure can be also administered in combination with an anti-tumor antibody or an antibody directed at a pathogenic antigen or allergen.

The pharmaceutical compositions of the invention can be readily employed in a variety of therapeutic or prophylactic applications, e.g., for treating SARS-COV-2 infection or eliciting an immune response to SARS-COV-2 in a subject. In various embodiments, the vaccine compositions can be used for treating or preventing infections caused by a pathogen from which the displayed immunogen polypeptide in the PIV5-based vaccine is derived. Thus, the vaccine compositions of the invention can be used in diverse clinical settings for treating or preventing infections caused by various viruses. As exemplification, a SARS-COV-2 CVB-based vaccine composition can be administered to a subject to induce an immune response to SARS-COV-2, e.g., to induce production of broadly neutralizing antibodies to the virus. For subjects at risk of developing an SARS-COV-2 infection, a vaccine composition of the invention can be administered to provide prophylactic protection against viral infection. Therapeutic and prophylactic applications of vaccines derived from the other immunogens described herein can be similarly performed. Depending on the specific subject and conditions, pharmaceutical compositions of the invention can be administered to subjects by a variety of administration modes known to the person of ordinary skill in the art, for example, topical, oral, intranasal, intramuscular, subcutaneous, intravenous, intra-arterial, intra-articular, intraperitoneal, or parenteral routes. In some aspects, administration is to a mucosal surface. A vaccine may be administered by mass administration techniques such as by placing the vaccine in drinking water or by spraying the animals' environment. When administered by injection, the immunogenic composition or vaccine may be administered parenterally. Parenteral administration includes, for example, administration by intravenous, subcutaneous, intramuscular, or intraperitoneal injection.

B. i. Administration by Inhalation

In one embodiment, the disclosed CVB vaccine compositions are formulated to allow intranasal administration. Intranasal compositions may comprise an inhalable dry powder pharmaceutical formulation comprising a therapeutic agent, wherein the therapeutic agent is present as a freebase or as a mixture of a salt and a freebase. Pharmaceutical formulations disclosed herein can be formulated as suitable for airway administration, for example, nasal, intranasal, sinusoidal, peroral, and/or pulmonary administration. Typically, formulations are produced such that they have an appropriate particle size for the route, or target, of airway administration. As such, the formulations disclosed herein can be produced so as to be of defined particle size distribution.

For example, the particle size distribution for a salt form of a therapeutic agent for intranasal administration can be between about 5 μm and about 350 μm. More particularly, the salt form of the therapeutic agent can have a particle size distribution for intranasal administration between about 5 to about 250 μm, about 10 μm to about 200 μm, about 15 μm to about 150 μm, about 20 μm to about 100 μm, about 38 μm to about 100 μm, about 53 μm to about 100, about 53 μm to about 150 μm, or about 20 μm to about 53 μm. The salt form of the therapeutic agent in the pharmaceutical compositions of the invention can a particle size distribution range for intranasal administration that is less than about 200 μm. In other embodiments, the salt form of the therapeutic agent in the pharmaceutical compositions has a particle size distribution that is less than about 150 μm, less than about 100 μm, less than about 53 μm, less than about 38 μm, less than about 20 μm, less than about 10 μm, or less than about 5 μm. The salt form of the therapeutic agent in the pharmaceutical compositions of the invention can have a particle size distribution range for intranasal administration that is greater than about 5 μm, greater than about 10 μm, greater than about 15 μm, greater than about 20 μm, greater than about 38 μm, less than about 53 μm, less than about 70 μm, greater than about 100 μm, or greater than about 150 μm.

Additionally, the salt form of the therapeutic agent in the pharmaceutical compositions of the invention can have a particle size distribution range for pulmonary administration between about 1 μm and about 10 μm. In other embodiments for pulmonary administration, particle size distribution range is between about 1 μm and about 5 μm, or about 2 μm and about 5 μm. In other embodiments, the salt form of the therapeutic agent has a mean particle size of at least 1 μm, at least 2 μm, at least 3 μm, at least 4 μm, at least 5 μm, at least 10 μm, at least 20 μm, at least 25 μm, at least 30 μm, at least 40 μm, at least 50 μm, at least 60 μm, at least 70 μm, at least 80 μm, at least 90 μm, or at least 100 μm.

In some embodiments the disclosed cannabinoid compositions include one or more cannabinoids or pharmaceutically acceptable derivatives or salts thereof, a propellant, an alcohol, and a glycol and/or glycol ether. The alcohol may be a monohydric alcohol or a polyhydric alcohol, and is preferably a monohydric alcohol. Monohydric alcohol has a lower viscosity than a glycol or glycol ether. Accordingly, the composition is able to form droplets of a smaller diameter in comparison to compositions in which the monohydric alcohol is not present. The present inventors have surprisingly found that a specific ratio of monohydric alcohol to glycol or glycol ether results in a composition with a desired combination of both long term stability (for example the composition remains as a single phase for at least a week at a temperature of 2-40° C.) and small droplet size.

ii. Pulmonary Compositions

One embodiment provides a formulation and method for treating SARS-COV-2 in the pulmonary system by inhalation or pulmonary administration. The diffusion characteristics of the particular drug formulation through the pulmonary tissues are chosen to obtain an efficacious concentration and an efficacious residence time in the tissue to be treated. Doses may be escalated or reduced or given more or less frequently to achieve selected blood levels. Additionally, the timing of administration of administration and amount of the formulation is preferably controlled to optimize the therapeutic effects of the administered formulation on the tissue to be treated and/or titrate to a specific blood level.

Diffusion through the pulmonary tissues can additionally be modified by various excipients that can be added to the formulation to slow or accelerate the absorption of drugs into the pulmonary tissues. For example, the drug may be combined with surfactants such as the phospholipids, dimyristoylphosphatidyl choline, and of administration dimyristoylphosphatidyl glycerol. The drugs may also be used in conjunction with bronchodilators that can relax the bronchial airways and allow easier entry of the antineoplastic drug to the lung. Albuterol is an example of the latter with many others known in the art. Further, the drug may be complexed with biocompatible polymers, micelle forming structures or cyclodextrins.

Particle size for the aerosolized drug used in the present examples was measured at about 1.0-5.0 μm with a GSD less than about 2.0 for deposition within the central and peripheral compartments of the lung. As noted elsewhere herein particle sizes are selected depending on the site of desired deposition of the drug particles within the respiratory tract.

Aerosols useful in the invention include aqueous vehicles such as water or saline with or without ethanol and may contain preservatives or antimicrobial agents such as benzalkonium chloride, paraben, and the like, and/or stabilizing agents such as polyethyleneglycol.

Powders useful in the invention include formulations of the neat drug or formulations of the drug combined with excipients or carriers such as mannitol, lactose, or other sugars. The powders used herein are effectively suspended in a carrier gas for administration. Alternatively, the powder may be dispersed in a chamber containing a gas or gas mixture which is then inhaled by the patient.

An agent of the present disclosure may be administered at once or may be divided into a number of multiple doses to be administered at intervals of time. For example, agents of the invention may be administered repeatedly, e.g., at least 2, 3, 4, 5, 6, 7, 8, or more times, or may be administered by continuous infusion. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that any concentration ranges set forth herein are exemplary only and are not intended to limit the scope or practice of the claimed compositions and methods.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

In some aspects, any of the PIV5-based constructs and methods described in WO 2013/112690 and WO 2013/112720 (which is hereby incorporated by reference herein in its entirety) may be used in the present invention.

As used herein, the term “subject” represents an organism, including, for example, a mammal. A mammal includes, but is not limited to, a human, a non-human primate, and other non-human vertebrates. A subject may be an “individual,” “patient,” or “host.” Non-human vertebrates include livestock animals (such as, but not limited to, a cow, a horse, a goat, and a pig), a domestic pet or companion animal, such as, but not limited to, a dog or a cat, and laboratory animals. Non-human subjects also include non-human primates as well as rodents, such as, but not limited to, a rat or a mouse. Non-human subjects also include, without limitation, poultry, horses, cows, pigs, goats, dogs, cats, guinea pigs, hamsters, mink, and rabbits.

As used herein “in vitro” is in cell culture and “in vivo” is within the body of a subject. As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

### B. Booster Vaccines

The present disclosure provides for the administration of a booster CVB vaccine for use in such a method for inducing in a human subject an immune response, wherein said subject has previously received a primary vaccination against SARS-COV-2.

The method of booster vaccination according to the disclosure comprises the step of administering the vaccine composition to the subject.

The immune response induced by the vaccine composition of the disclosure or by the method of the disclosure is preferably a humoral response, especially a response comprising the production of neutralizing antibodies against the COVID-19 virus, i.e. a neutralizing antibody response.

### EXAMPLES

### Example 1: Immunogenicity of CPI-RSV-F in African Green Monkeys (AGM) (Non-GLP Study)

Objective: To compare immunogenicity of CPI-RSV-F and W3AΔSH-RSV-F administered intranasally as a single dose in the African green monkey model.

### Methods

Four monkeys in each dose group were immunized at Day 0 with 10⁶ PFU intranasally of either CPI-RSV-F or W3AΔSH-RSV-F.

### Results

The antibody responses to RSV F-protein and PIV5 vector at Day 28 were determined by ELISA assays (plates coated with recombinant RSV F protein, source Sino Biologicals: 11049-V088 LC120C 2910, or PIV5 wild type virus) as shown in Table 4. In summary, all animals developed antibodies against the F protein expressed by the CPI or W3AΔSH with comparable RSV F ELISA titers between the two groups. Yet, W3AΔSH-RSV-F elicited higher PIV5 antibodies than CPI-RSV-F.

TABLE 4
Serum antibody response to RSV F and PIV5 in the AGM study.

| | Animal | RSV F ELISA titer | | PIV5 ELISA titer | |
|---|---|---|---|---|---|
| Group | ID | Day 0 | Day 28 | Day 0 | Day 28 |
| W3AΔSH- | #1_09818 | 120 | 3240 | <40 | 3240 |
| RSV-F | #2_09836 | 120 | 3240 | 40 | 3240 |
| | #3_90071 | 40 | 1080 | 40 | 9720 |
| | #4_58181 | 40 | 1080 | 120 | 9720 |
| CPI-RSV-F | #1_09669 | 360 | 1080 | <40 | 1080 |
| | #2_09839 | 360 | 1080 | 40 | 1080 |
| | #3_09854 | 40 | 3240 | <40 | 360 |
| | #4_09861 | 120 | 3240 | <40 | 9720 |

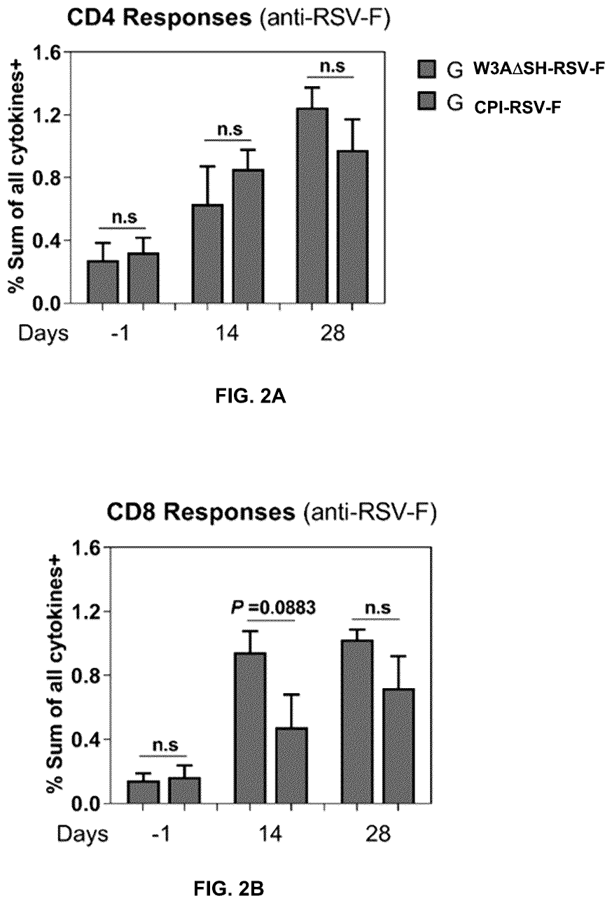

Cell Mediated Responses: RSV F protein-specific cellular responses were evaluated by intracellular cytokine staining (ICS) assay. Following immunization, blood was collected at days −1, 14, and 28 to quantify the RSV F protein-specific CD4 and CD8 cellular responses. INFγ, TNFα, MIP-1b, IL-13, and CD107a positive cells were quantitated (see FIG. 2). PBMCs collected at day −1 prior to immunization failed to respond to RSV F peptide stimulation as expected. Following immunization, AGMs immunized with W3AΔSH-RSV-F and CPI-RSV-F generated CD4 and CD8 cellular responses that are RSV F protein-specific as assessed on Day 14 and Day 28. No F-specific cellular response was detected in the control animals (data not shown), and the cellular responses from animals immunized with W3AΔSH-RSV-F and CPI-RSV-F increased from day 14 to day 28 post-immunization (FIGS. 2A-2B). W3AΔSH-RSV-F elicited lower CD4 responses than CPI-RSV-F at day 14, but the difference was not statistically significant. At day 28 post-immunization, W3AΔSH-RSV-F induced higher CD4 responses than CPI-RSV-F, but again this difference was not statistically significant. The CD8 specific responses elicited by W3AΔSH-RSV-F were higher than CPI-RSV-F, however, the difference was not statistically significant on day 14 (P=0.0883) or day 28.

Both CPI-RSV-F and W3AΔSH-RSV-F elicited immune responses against RSV F protein. The antibody responses against RSV F protein elicited by either CPI-RSV-F or W3AΔSH-RSV-F were comparable following a single intranasal dose at 10⁶ PFU. The CD4 and CD8 cellular responses elicited by CPI-RSV-F were slightly lower than W3AΔSH-RSV-F, but the difference was not statistically significant.

### Example 2: Safety and Immunogenicity of CPI-RSV-F in Cotton Rat Challenge Study (Non-GLP)(NIH Sponsored Study: Study XV-238)

Objective: To evaluate the efficacy and safety of RSV F protein vaccine candidates based on the CPI strain or the W3A strain of PIV5 (CPI-RSV-F and W3AΔSH-RSV-F, respectively) in the cotton rat Sigmodon hispidus model of RSV A/A2 challenge.

### Methods

Animals were first immunized with 100 μl of the PIV5-based vaccines containing 10⁴, 10⁵, or 10⁶ PFU of virus intranasally and then challenged with 10⁵ PFU RSV A/A2 four weeks later. The primary infection control group was mock-immunized with PBS and then infected with RSV A/A2. The secondary infection control group was infected with RSV A/A2 and re-infected seven weeks later. The vaccine-enhanced disease control group was immunized with FI-RSV (formalin inactivated RSV) twice by the i.m. route with an interval of four weeks and infected with RSV three weeks after the second immunization. See Table 5 for study groups.

TABLE 5
Study Groups in study XV-238.

| | | | | | | RSV | Day of harvest |
|---|---|---|---|---|---|---|---|
| | | | TA Dose | Immunization | | Challenge | (post-RSV |
| Group | N | Treatment | (per rat) | days | Route | (PFU/rat) | challenge) |
| A | 4 | PBS (Mock) | n/a | 0, 28 | IM | PBS | 5 |
| B | 5 | PBS (Mock) | n/a | 0, 28 | IN | 10⁵ PFU | 5 |
| C | 5 | FI-RSV | 1:100 in | 0, 28 | IM | 10⁵ PFU | 5 |
| | | Lot#100 | PBS | | | | |
| D | 5 | RSV/A2 Live | 5.0 | 0 only | IN | 10⁵ PFU | 5 |
| | | | Log10 | | | | |
| E | 5 | CPI-RSV-F | 10⁴ PFU | 0 only | IN | 10⁵ PFU | 5 |
| F | 5 | CPI-RSV-F | 10⁵ PFU | 0 only | IN | 10⁵ PFU | 5 |
| G | 5 | CPI-RSV-F | 10⁶ PFU | 0 only | IN | 10⁵ PFU | 5 |
| H | 5 | W3AΔSH- | 10⁴ PFU | 0 only | IN | 10⁵ PFU | 5 |
| | | RSV-F | | | | | |
| I | 5 | W3AΔSH- | 10⁵ PFU | 0 only | IN | 10⁵ PFU | 5 |
| | | RSV-F | | | | | |
| J | 5 | W3AΔSH- | 10⁶ PFU | 0 only | IN | 10⁵ PFU | 5 |
| | | RSV-F | | | | | |
| K | 3 | CPI-RSV-F | 10⁶ PFU | 0 only | IN | Harvest on | |
| L | 3 | W3AΔSH- | 10⁶ PFU | 0 only | IN | day 4 post | |
| | | RSV-F | | | | dosing | |

Animals were sacrificed five days after RSV challenge for sample collection. RSV replication in the lung and nose, pulmonary histopathology, pulmonary cytokine and RSV NS1 mRNA expression, and the RSV serum neutralizing antibody (NA) and the anti-F protein binding antibody titers were measured. To confirm replication of the PIV5 vaccines in the respiratory tract of cotton rats, groups of 3 animals were inoculated with 10⁶ PFU of CPI-RSV-F or control intranasally and sacrificed four days later.

### Results

Both candidate vaccine viruses replicated efficiently in the upper and lower respiratory track of cotton rats (Group K and L) (Table 6). W3AΔSH-RSV-F vaccine virus replicated to higher levels in the upper respiratory tract compared to CPI-RSV-F, but it replicated to lower levels in the lower respiratory tract compared to CPI-RSV-F.

TABLE 6
PIV5 vaccine virus titers in upper and lower respiratory tract

| | | | | Titer | Average titer |
|---|---|---|---|---|---|
| Group | Animal ID | Tissue | Day | (Log₁₀ PFU/mL) | (Log₁₀ PFU/mL) |
| Group K | 124880 | Nose | 4 | 3.7 | 3.9 |
| CPI-RSV-F | 124881 | Nose | 4 | 3.9 | |
| Day 4 | 124882 | Nose | 4 | 4.0 | |
| | 124880 | Lung | 4 | 3.8 | 3.5 |
| | 124881 | Lung | 4 | 3.3 | |
| | 124882 | Lung | 4 | 2.9 | |
| Group L | 124883 | Nose | 4 | 4.5 | 5.0 |
| W3AΔSH- | 124884 | Nose | 4 | 4.9 | |
| RSV-F | 124885 | Nose | 4 | 5.2 | |
| Day 4 | 124883 | Lung | 4 | 2.3 | 2.2 |
| | 124884 | Lung | 4 | 2.2 | |
| | 124885 | Lung | 4 | 1.7 | |

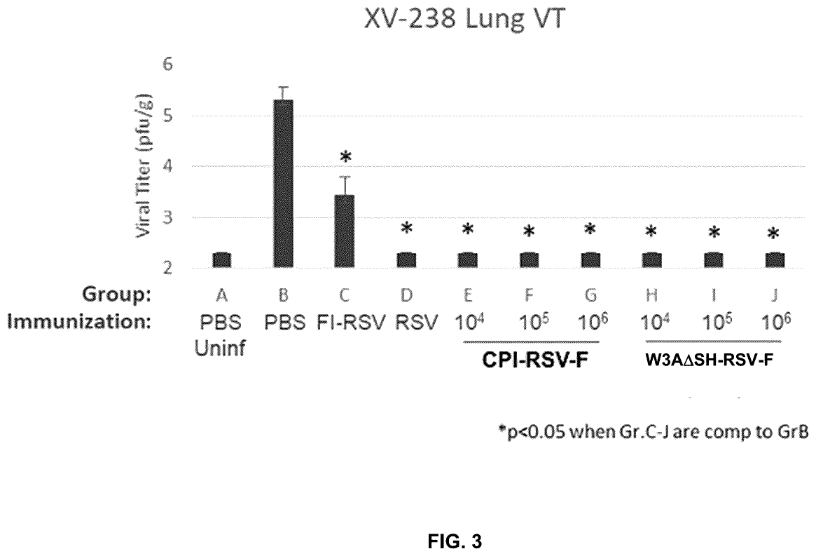

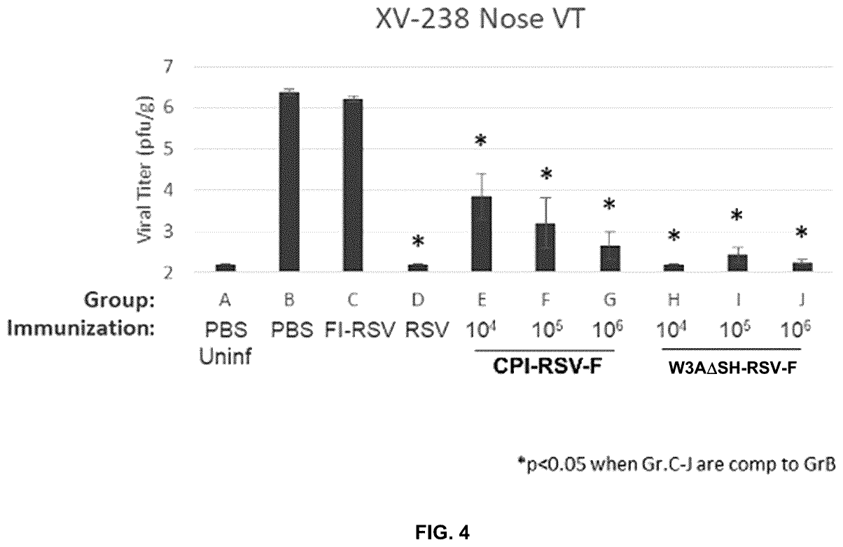

Both CPI-RSV-F and W3AΔSH-RSV-F vaccines were highly efficacious at protecting RSV challenge virus replication in the lungs of cotton rats as indicated by the very low levels of viral titer in the lung tissues (FIG. 3). All doses of both vaccines also induced statistically significant protection when evaluating viral load in nasal washes with higher reduction observed for W3AΔSH-RSV-F compared to CPI-RSV-F immunization (FIG. 4). W3AΔSH-RSV-F reduced viral load to almost undetectable levels at all three vaccine doses tested. For CPI-RSV-F, the 10⁶ PFU dose induced the strongest protection in the upper respiratory tract, followed by the 10⁵ PFU and 10⁴ PFU doses.

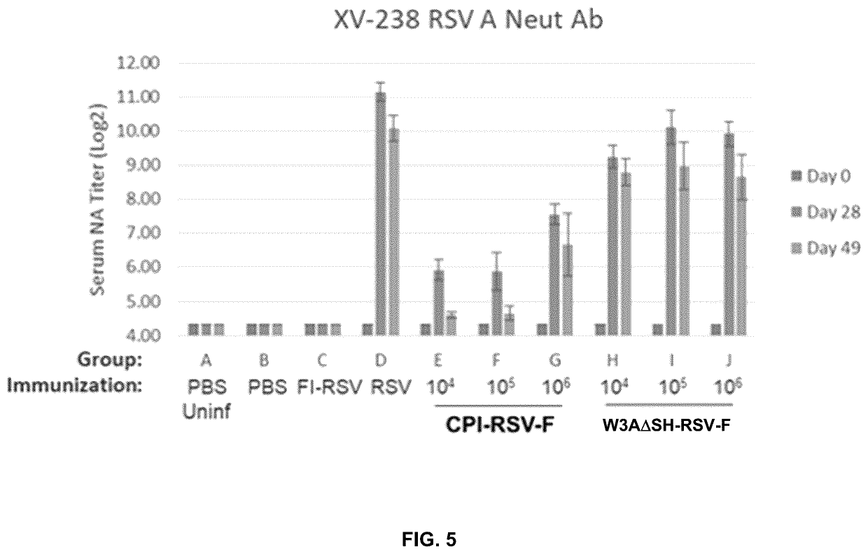

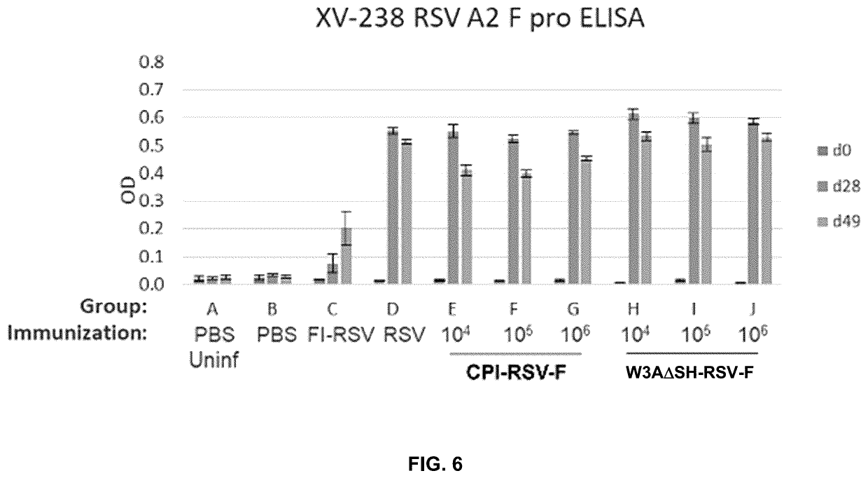

Both CPI-RSV-F and W3AΔSH-RSV-F vaccines induced strong neutralizing antibody (nAb) responses, with W3AΔSH-RSV-F at all three doses tested inducing higher titers of nAb compared to CPI-RSV-F administered at the highest dose (10⁶ PFU). Lower doses (10⁴ and 10⁵ PFU) of CPI-RSV-F induced weaker nAb responses (FIG. 5). Both CPI-RSV-F and W3AΔSH-RSV-F immunizations induced high levels of anti-RSV IgG antibodies that were only slightly higher for W3AΔSH-RSV-F vaccination (FIG. 6).

None of the vaccines induced the level of pulmonary histopathology or IL-4 mRNA expression that was seen in FI-RSV-immunized animals (positive control animals for enhanced disease), with the exception of one animal immunized with the intermediate dose (10⁵ PFU) of W3AΔSH-RSV-F showing elevated interstitial inflammation and alveolitis (which coincided with elevated IL-4 levels in this animal). Levels of IFN-γ, Il-2 and IL-4 were low in both vaccine groups (Data not shown). No deaths were observed in the PIV5-vectored vaccine groups. A single animal in the FI-RSV (positive control) group died at Day 24.

Both PIV5 CPI and W3A based RSV vaccine constructs were effective in eliciting neutralizing RSV antibody responses and protecting lungs following RSV challenge infection following a single vaccination. Overall, the efficacy of W3AΔSH-RSV-F vaccine appeared to be higher than that of CPI-RSV-F in regard to nasal protection and neutralizing antibody response at all dose levels tested. There was no indication of enhanced lung pathology when compared to positive control group (RSV-FI group) except for one animal in the 10⁵ PFU W3AΔSH-RSV-F group.

### Example 3: Manufacturing Process and Process Controls

### Step 1: Vaccine Vector Rescue

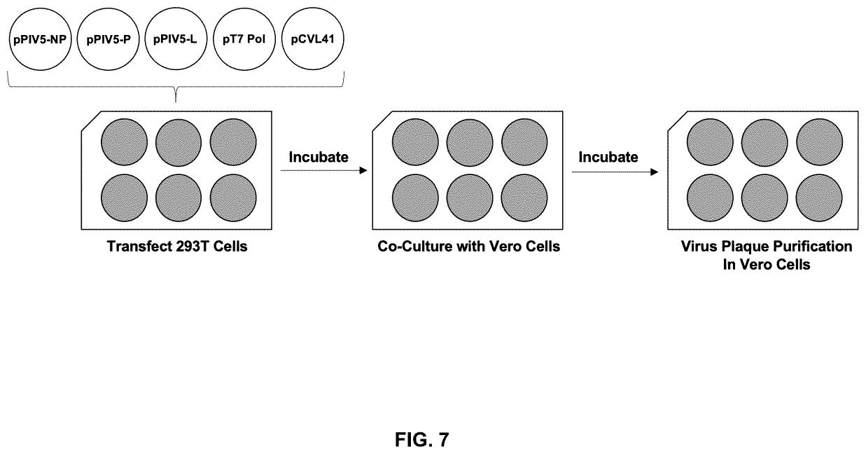

Rescue of the recombinant vector virus used in the production of CPI−, W3AΔSH-, CVB-based vaccines was performed. For the rescue of the recombinant virus, the antigenomic cDNA plasmid was transfected into serum-free 293T suspension cells (obtained from GenHunter Corporation) together with plasmids encoding the PIV5 NP, P, L proteins and T7 RNA Polymerase allowing for the rescue of recombinant virus from the transfected cell culture (FIG. 7).

The supporting PIV5 plasmid clones encoding the nucleoprotein (N), phosphoprotein (P) or large polymerase protein (L) were described previously (1,2) each gene is under the T7 promoter in pCAGGS vector. The following 5 plasmids were used in the generation of research virus seed (RVS).
1. Antigenomic cDNA plasmid of CPI−, W3AΔSH-, CVB-based PIV5: This plasmid is a high-copy plasmid and it contains an ampicillin resistance gene.
2. pCAGGS-NP Plasmid: This plasmid contains the PIV5 NP gene under the T7 promoter with anampicillin resistance gene.
3. pCAGGS-P Plasmid: This plasmid contains the PIV5 P gene under the T7 promoter with anampicillin resistance gene.
4. pCAGGS-L Plasmid: This plasmid contains the PIV5 L gene under the T7 promoter with anampicillin resistance gene.
5. pCAGGS-T7 Plasmid (same as pBH437-T7): This plasmid encodes the T7RNA polymerase gene under the SV40 promoter.

The medium used for virus rescue was CDM4HEK293 medium (Hyclone) with 4 mM GlutaMAX (Gibco).

Following two days of incubation, the 293T cells were co-cultured with serum-free Vero cells (P159) passaged from Vero MCB cells (obtained from CRL: African Green Monkey Kidney (WHO Vero 10-87) CyanVac MCB DOM:19aug.2020-P148). After 4 days of incubation, 2 mL of supernatant containing the rescued virus was obtained and mixed with 10×SPG and stored at −80° C.

### Step 2: Plaque Purification and Expansion of Virus Rescue Seed

Aliquots of the frozen stock with rescued virus were serially diluted to perform a plaque assay on serum-free Vero cells in 6-well plates, with the objective of obtaining several isolated single plaques. A 1000 μL pipette tip was used to poke a single plaque and re-suspend in VP-SFM media containing 4 mM GlutaMAX. The re-suspended plaque was then used to infect fresh serum-free Vero cells in a 6-well plate. After 6 days, the supernatant (2 mL) from the 6-well plate infected with single plaque was mixed with 10% 10×SPG and stored at −80° C. (to result in 1×SPG). Part of the supernatant (140 μL was used to do RNA extraction and RT-PCR to verify viral genomic sequence. The RT-PCR was done with the primers described in Table 7.

TABLE 7
Primers used for amplifying viral
cDNAs for sequencing of
CPI-RSV-F genomic cDNA

| | | | SEQ |
|---|---|---|---|
| | | | ID |
| Number | Name | Sequence (5' to 3') | NO: |
| 1 | CVL1 | ACCAAGGGGAAAATGAAGTGGTGAC | 14 |
| 2 | CVL6 | GGAGAAGCTCAGATCAGTGGGATC | 15 |
| 3 | CVL5 | ACAGTGGATTCCATAAGGTTCCTGC | 16 |
| 4 | CVL11 | CACTGACAGAAATGTCGGAATGAAGAG | 17 |
| 5 | CVL10 | TCCAGGTCACTGTTAGGAAGACATC | 18 |
| 6 | CVL20 | GCTCGATTACCTAGATTGACCGAGAC | 19 |
| 7 | CVL17 | CAAGATGCACCTTCTCTCCAGTG | 20 |
| 8 | CVL154 | ATCACACTCGAGCTGACATCTGTC | 21 |
| 9 | CVL152 | CGTGAGTACCTACATGCTGACA | 22 |
| 10 | CVL25 | CGGATTGATACGCTGAGTTGCTG | 23 |
| 11 | CVL19 | TAACTCTGCAGTCGCTCTACCTC | 24 |
| 12 | CVL27 | CAGTCTGAGTCATGCCATGCTG | 25 |
| 13 | CVL26 | GCATTCAGAATCTCAGCTATGGACTG | 26 |
| 14 | CVL33 | TCAATGATGATGTGAGCTACACGC | 27 |
| 15 | CVL32 | GCTCTGATGGAACTCAGTACGAATC | 28 |
| 16 | CVL37 | GGTTCTCTATTGAACTCAACCCTGAG | 29 |
| 17 | CVL36 | GTGACCGATCCATGAATCTCATTGC | 30 |
| 18 | CVL42 | ACCAAGGGGAAAACCAAGATTAATCC | 31 |

Step 3: Manufacture of pre-MVS on Serum Free Vero Cells

An aliquot from step 2 (plaque purified virus) was used to infect serum-free Vero cells in T75 flask, incubated at 37° C. for 5 days and the cell culture supernatants were centrifuged at 1,500 rpm for 10 minutes at 4° C. to remove cell debris. The clarified supernatants (20 mL) were mixed with 10% 1×SPG with 10% Arginine (Sigma Aldrich), fast frozen in 1 mL aliquots and stored at −80° C. as pre-MVS stock. The pre-MVS was sequenced from the NP gene through the L gene to confirm the viral genomic sequence and proper insertion of the RSV F protein or other antigen gene.

The pre-MVS prior to use in cGMP production of the MVS was tested at CRL for sterility (direct method), bacteriostasis and fungistasis, mycoplasma, mycobacteria, presence of porcine and bovine circoviruses, and in apparent viruses.

### Step 4: Manufacturing Process for MVS

The production of the MVS was performed in adherence to cGMP.

Vaccine bulk substance manufacture: The Vero cells grown in T225 flasks in serum free media (VP-SFM) were infected with pre-MVS (using 2 vials) at MOI of 0.001 to 0.002 at 37° C. for 7 days. The cell culture fluid of infected cells constitutes the crude vaccine bulk substance (3.0 liter). Samples of the MVS crude bulk virus fluid were taken for testing of: Sterility (direct method), bacteriostasis and fungistasis, in vitro mycoplasma testing (agar cultivable and non-cultivable), tissue culture safety testing, in vitro mycobacterium testing, inapparent viruses, PBERT, potency.

The vaccine bulk substance was clarified by centrifugation at 1,500 rpm for 10 min at 2-8° C. resulting in 1.2 L followed by filtration through a 0.45 μM PES filter unit from ThermoFisher resulting in 1.2 L. The clarified and filtered vaccine bulk substance is immediately formulated with 10×SPG to stabilize the virus prior to fill. There is no hold of the vaccine bulk substance before formulation and fill.

During the virus production process, control harvest fluid was prepared using uninfected Vero cells fromthe same production lot. The control fluids were stored at −60° C. or below. The production control fluid was harvested and tested for the Bacteriostasis and Fungistasis Bulk Product (Direct Method), Sterility, Detection and Quantitation of Residual Vero DNA and Determination of Endotoxin Levels (LAL).

Vaccine drug product manufacture (formulation and fill) MVS: The filtered viruses were stabilized by formulation with 10% 10×SPG buffer and dispensed using a calibrated repeater pipette in 1 mL volumes into 2 mL cryovials to make the MVS stock/Vaccine Product. The dispensed MVS/Vaccine product was flash frozen in dry ice/methanol bath and stored at −60° C. or below.

MVS characterization: In addition, genetic identity was confirmed by sequencing of viral genomic cDNA from the NP gene through the L gene region to confirm identity.

### Example 4: GLP Intranasal Safety Study of Candidate RSV Vaccine CPI-RSV-F and CVB-RSV-F in Cotton Rats

The objective of the single and repeat dose GLP tox study conducted in cotton rats was to determine the potential toxicity of single or repeat dose of CPI-RSV-F when administered intranasally to male and female cotton rats.

The study was conducted in 48 cotton rats (24 male and 24 females). PIV5 vectored RSV vaccine viruses (CPI-RSV-F and CVB-RSV-F) at 10⁷ PFU dose level were evaluated following a single dose (Day 1) or a repeat dose (Day 1 and Day 15) instilled by the intranasal route.

Test Article 1: CPI-RSV-F, 1×10^{7.0} PFU/100 μL dose (50 μl each nostril) (8.0 log₁₀ PFU/mL; lot number CPI-RSV-F-210519PQ10MVS) formulated in 1×SPG.

Test Article 2: Active comparator CVB-RSV-F; 1×10^{7.1} PFU/100 μL dose (50 μl each nostril) (8.1 log 10 PFU/mL; lot number 211007CHD) formulated in 1×SPG.

Control: SPG 1×100 μL dose (50 μl each nostril)

On Study Day 1, Animals were dosed via intranasal instillation using a calibrated pipette. A fresh pipette tip was used for each animal. Prior to administration of test or control articles, rats were anesthetized via injection of 0.1 mL of a ketamine (25 mg/mL)/xylazine (10 mg/mL) mixture. The dosing groups are shown in Table 8.

TABLE 8
Study groups of GLP Intranasal Safety Study of Candidate
RSV Vaccine CPI-RSV-F and CVB-RSV-F in Cotton Rats

| | | | | Number of Rats |
|---|---|---|---|---|
| Group | Dose level | Dosing Day | Necropsy | (M + F) |
| 1 | Control (1 × SPG) | Day 1 | Day 15 | 4 + 4 |
| 2 | CPI-RSV-F (1 × 10⁷ PFU) | Day 1 | Day 15 | 4 + 4 |
| 3 | CVB-RSV-F (1 × 10⁷ PFU) | Day 1 | Day 15 | 4 + 4 |
| 4 | Control (1 × SPG) | Day 1& 15 | Day 29 | 4 + 4 |
| 5 | CPI-RSV-F (1 × 10⁷ PFU) | Day 1 & 15 | Day 29 | 4 + 4 |
| 6 | CVB-RSV-F (1 × 10⁷ PFU) | Day 1 & 15 | Day 29 | 4 + 4 |

### Evaluation:

Experimental endpoints consisted of moribundity/mortality and cage-side clinical observations; physical examination observations; body weight and body weight change; body temperature; food consumption; clinical pathology (clinical chemistry, hematology, and coagulation) parameters; blood serum immunogenicity analysis, brain weight; gross necropsy observations; and histopathological evaluations.

### Pathology:

Tissues required for microscopic evaluation were trimmed, processed routinely, embedded in paraffin, and stained with hematoxylin and eosin by Charles River Laboratories, Inc (See Table 9). Light microscopic evaluation was conducted by the Contributing Scientist, a board-certified veterinary pathologist, on protocol-specified tissues from all animals.

TABLE 9
Tissue samples evaluated.

| Provantis | | | | Microscopic | |
|---|---|---|---|---|---|
| TissueTerm | Protocol Tissue Term | Collect | Weigh | Evaluation | Comment |
| — | Animal identification | X | — | — | Ear with metal tag |
| BODY CAVITY, | Nasal cavity including | X | — | X | Three sections |
| NASAL | olfactory bulbs | | | | wereprepared to |
| | | | | | assesslocal |
| | | | | | reactogenicity |
| — | Bone marrow | X | — | — | Fixed in methanol |
| | smear(femur) | | | | |
| BRAIN | Brain | X | X | X | |
| ESOPHAGUS | Esophagus | X | — | X | |
| GLAND, | Gland, salivary | X | — | X | |
| SALIVARY | (paired) | | | | |
| HEART | Heart | X | — | X | |
| LUNG | Lung with | X | — | X | |
| | largebronchi | | | | |
| LYMPHNODE, | Lymph | X | — | X | |
| MANDIBULAR | node, | | | | |
| | Mandibular | | | | |
| THYMUS | Thymus | X | — | X | |
| TONGUE | Tongue | X | — | X | |
| TRACHEA | Trachea | X | — | X | |
| — | Gross lesions (if any) | X | — | X | |

For all study animals, the tissues/organs listed above were examined, sampled, and fixed in 1000 neutral buffered formalin with the exceptions of the bone marrow smears, which were fixed in methanol. Prior to being sampled, the brain of each animal was weighed. Brain-to-body weight ratios were calculated using the fasted body weight (taken on the day of necropsy).

### Statistical Analysis:

Statistical Procedures: Descriptive statistics (mean and standard deviation) were calculated and analyzed for statistical significance for the following quantitative data using the ToxData® system: body weights and body weight changes, food consumption, body temperature, clinical pathology (clinical chemistry, hematology, and coagulation) parameters, and absolute brain weights and brain-to-body weight ratios.

For all analyses, if the data set was normally distributed and of equal variance, statistical comparisons were conducted using a one-way analysis of variance (ANOVA), with post hoc comparisons made (if necessary) using Dunnett's test. If normality and/or equal variance failed fora data set, statistical comparisons were conducted using nonparametric Kruskal-Wallis ANOVA, with post hoc comparisons made (if necessary) using Dunn's test. Incidence data (e.g., clinical observations and physical examination observations) were evaluated using Chi-square analysis and/or Fisher's Exact test. A minimum significance level of p<0.05 was used for the statistical comparisons in this study.

For all statistical comparisons, data from Groups 2 and 3 were compared against Group 1 (Control) data from animals of the same sex, and data from Groups 5 and 6 were compared against Group 4 (Control) data from animals of the same sex.

### Results

Safety assessment Following single or double intranasal administration of CPI-RSV-F at 10⁷ PFU dose level to male and female cotton rats, no unscheduled treatment related mortalities or treatment-related clinical signs of toxicity were seen. In addition, no treatment-related findings were seen in body weight, body weight change, body temperature, food consumption, absolute or relative brain weight, gross necropsy or histopathology for both vaccine candidates (CPI-RSV-F and CVB-RSV-F).

### Immunogenicity

The CPI-RSV-F vaccine was shown to be immunogenic in this study. A summary of the immunogenicity data is shown in Table 10.

TABLE 10
Summary of serum antibody responses to RSV and PIV5

| | | RSV | | |
|---|---|---|---|---|
| | | Neutralizing | Anti-RSV-F | Anti-PIV5 |
| Group | Test Articles and study day | Ab titer | ELISA titer | ELISA titer |
| 1 | 1 × SPG control (N = 8); Day | <10 | <40 | <40 |
| | 15 | | | |
| 2 | CPI-RSV-F (10⁷ PFU, N = 8); | 160 | 1239 | 57 |
| | 1 dose; Day 15 | | | |
| 3 | CVB-RSV-F; (10⁷ PFU, | 431 | 1264 | 113 |
| | N = 7); 1 dose; Day 15 | | | |
| 4 | 1 × SPG control (N = 8); Day | <10 | <40 | <40 |
| | 29 | | | |
| 5 | CPI-RSV-F; (10⁷ PFU, | 247 | 1421 | 175 |
| | N = 8); 2 dose; Day 29 | | | |
| 6 | CVB-RSV-F; (10⁷ PFU, | 1974 | 2146 | 453 |
| | N = 8); 2 dose; Day 29 | | | |

RSV neutralizing antibody titer was measured using RSV-rLuc report virus per CVL protocol-038. RSV-F specific ELSIA antibody titer was determined per CVL protocol-049. The PIV5 antibody level was measured by ELISA assay using PIV5 virus particles per CVL protocol-048. CVB-RSV-F is more immunogenic than CPI-RSV-F in eliciting RSV neutralizing antibodies.

Single intranasal administration of CPI-RSV-F to cotton rats on Day 1 and repeat intranasal administration of CPI-RSV-F to cotton rats on Day 1 and Day 15 resulted in no early deaths by Day 29.

No treatment-related clinical signs of toxicity were seen or no treatment-related findings in body weight, body weight change, body temperature, food consumption, absolute or relative brain weight, gross necropsy, or histopathology. These findings were not considered toxicologically significant due to the small magnitude of changes and the lack of any correlating histopathological findings.

The study was validated by confirmation of antibody responses to PIV5, the vaccine vector and the RSV F protein expressed by the CPI-RSV-F vaccine.

### Repeat-Dose Toxicity

Repeat dose toxicity of CPI-RSV-F vaccine was assessed in Group 4 and 5 of the GLP toxicology study above.

Repeat intranasal administration of CPI-RSV-F to cotton rats on Day 1 and 15 resulted in no gross or microscopic findings on Day 29 or other treatment related signs of toxicity.

Vaccine virus-induced antibody responses were detected in Groups 2, 3, 5 and 6 animals which received CPI-RSV-F or CVB-RSV-F, and absent in Groups 1 and 3 which received the control only (1×SPG). Animals which received two doses of CPI-RSV-F showed slightly higher RSV F and PIV5 antibody responses to those observed after a single dose.

Single or double administration of CPI-RSV-F and CVB-RSV-F at 7.0 log₁₀ PFU was found to be immunogenic in cotton rats, with no toxicologically relevant adverse effects including local reactogenicity in the nasal cavity.

### Immunogenicity Summary

The vaccine virus induced antibody responses were detected in the animals that received one and two doses of either CPI-RSV-F or CVB-RSV-F, but not detected in the control animals that received the control article (1×SPG buffer). Animals that received a second dose of CPI-RSV-F or CVB-RSV-F two weeks after the first dose had higher anti-PIV5 antibody levels compared to those that received a single dose. A second dose of CPI-RSV-F did not significantly increase anti-RSV-F or RSV neutralizing antibody titers. A second dose of CVB-RSV-F significantly increase RSV neutralizing antibody titers, but not anti-RSV-F antibody titers. CVB-RSV-F is more immunogenic than CPI-RSV-F, producing higher anti-PIV5 antibody titers and RSV neutralizing antibody titers. Overall, the serology data indicate both CPI-RSV-F and CVB-RSV-F are immunogenic in cotton rats (see Table 1 for serology data summary), which validated this GLP toxicity study in that CPI-RSV-F and CVB-RSV-F were shown to be active.

### Example 5: Generation of CVB Vectored SARS-CoV-2 Constructs

### Materials:

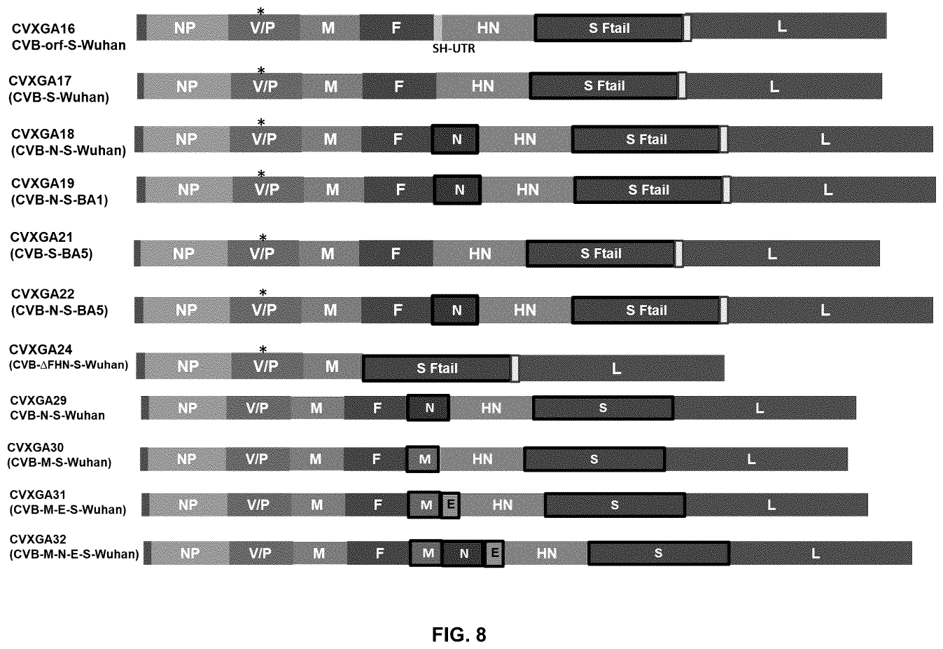

The CVB Vectored SARS-CoV-2 vaccine candidates generated are described herein and in FIG. 8.

The primers used in cDNA cloning of the CVB vaccine candidates are listed in Table 11.

TABLE 11
Primer sequences used in constructing CVB based COVID-19
N/S/M/E vaccine candidates

| | | | | SEQ |
|---|---|---|---|---|
| Vaccine | Construct | Primer | Sequence | ID NO: |
| CVXGA16 | CVL104 | oCVL433 | GCAAATTGCAGGTCGATGTTGTGCA | 32 |
| | | 0CVL294 | ATTTGAGCTTTCCGAGACGGTATCA | 33 |
| | | oCVL449 | GCTGATACCGTCTCGGAAAGCTCAA | 34 |
| | | | ATCCTGCTATAGGCTATCCACTGCAT | |
| | | | CATCTCTC | |
| | | oCVL451 | CCGCCGCTTGTTGCTAACCCGTCCGG | 35 |
| | | | GCC | |
| | | oCVL448 | GGCCCGGACGGGTTAGCAACAAGCG | 36 |
| | | | GCGGCCGCCATGTTCGTCTTCCTGGT | |
| | | | CCTGC | |
| | | oCVL293 | TAATGTTTGGTATGCATTCCATGAGT | 37 |
| | | | CGACTTCATTTATGATAAACAAAATT | |
| | | | CTC | |
| CVXGA17 | CVL87 | oCVL433 | GCAAATTGCAGGTCGATGTTGTGCA | 38 |
| | | oCVL445 | GCGGCCGCCATGTTCGTCTTCCTGGT | 39 |
| | | | CC | |
| | | oCVL446 | GGACCAGGAAGACGAACATGGCGGC | 40 |
| | | | CGCCGCTTGTTGCTAACC | |
| | | oCVL447 | ATAATGTTTGGTATGCATTCCATGAG | 41 |
| | | | TCGACTTCATTTATGATAAACAAAAT | |
| | | | TCTC | |
| CVXGA18 | CVL85 | oCVL433 | GCAAATTGCAGGTCGATGTTGTGCA | 42 |
| | | oCVL133 | GCGCGCCACCATGTCAGATAACGGA | 43 |
| | | | CCACAGAAC | |
| | | oCVL134 | TCAAGCCTGGGTGGAGTCGGCACTG | 44 |
| | | oCVL363 | GTCCGTTATCTGACATGGTGGCGCG | 45 |
| | | | CGATTTGAGCTTTCCGAGACGGTATC | |
| | | oCVL364 | CAGTGCCGACTCCACCCAGGCTTGAC | 46 |
| | | | GTACGACCTGCTATAGGCTATCCACTGC | |
| | | oCVL450 | AGAAGTGGGCCAAGTGTCAG | 47 |
| CVXGA19 | CVL83 | oCVL365 | GGCCCGGACGGGTTAGCAACAAGCGG | 48 |
| | | | CGGCCGCCACCATGTTCGTCTTCC | |
| | | oCVL293 | TAATGTTTGGTATGCATTCCATGAGTC | 49 |
| | | | GACTTCATTTATGATAAACAAAATTCTC | |
| CVXGA21 | CVL110 | oCVL616 | GGCCCGGACGGGTTAGCAACAAGCGC | 50 |
| | | | GCGGCCGCCACCATGTTCGTCTTCCTGG | |
| | | | TCCTGC | |
| | | oCVL444 | AATAATGTTTGGTATGCATTCCATGAG | 51 |
| | | | TCGACACGCGTTCATTTATGATAAACA | |
| | | | AAATTC | |
| CVXGA22 | CVL109 | oCVL617 | GGCCCGGACGGGTTAGCAACAAGCGG | 52 |
| | | | CGGCCGCCACCATGTTCGTCTTCCTGG | |
| | | | TCCTGC | |
| | | oCVL293 | TAATGTTTGGTATGCATTCCATGAGTC | 53 |
| | | | GACTTCATTTATGATAAACAAAATTCTC | |
| CVXGA24 | CVL112 | oCVL5 | ACAGTGGATTCCATAAGGTTCCTGC | 54 |
| | | oCVL622 | AGCTCATTCCAGCTCCGTCAGGTT | 55 |
| | | oCVL623 | AACCTGACGGAGCTGGAATGAGCTAGC | 56 |
| | | | GGCAGAAGCCTTCAGGTCTGACCC | |
| | | oCVL67 | CGATAGGCAGATCCACCAG | 57 |
| CVXGA29 | CVL119 | oCVL670 | GGCCCGGACGGGTTAGCAACAAGCGGC | 58 |
| | | | GGCCGCCGCCACCATGTTCGTCTTC | |
| | | oCVL671 | CAATAATGTTTGGTATGCATTCCATGAG | 59 |
| | | | TCGACCTTATTAGGTGTAATGCAGCT | |
| | | | TCACG | |
| CVXGA30 | CVL120 | oCVL672 | TGATACCGTCTCGGAAAGCTCAAATC | 60 |
| | | | GCGCGCCACCATGGCCGATAGCAACG | |
| | | | GCACC | |
| | | oCVL673 | ATGCAGTGGATAGCCTATAGCAGGTC | 61 |
| | | | GTACGTCATCACTGCACCAGCAGGGC | |
| | | | GATG | |
| CVXGA31 | CVL121 | oCVL674 | ACATCGCCCTGCTGGTGCAGTGATGA | 62 |
| | | | CGTACGGATCCCAATCTTAAATCGACAC | |
| | | oCVL675 | ACGCGTGGATTGTGTTATTGGCATCG | 63 |
| | | oCVL676 | CGATGCCAATAACACAATCCACGCGT | 64 |
| | | | GCCACCATGTATTCTTTCGTGTCTGAA | |
| | | oCVL677 | GCTAGCTTACACGAGCAGGTCGGGC | 65 |
| | | oCVL678 | GCCCGACCTGCTCGTGTAAGCTAGCAC | 66 |
| | | | CTGCTATAGGCTATCCACTGCAT | |
| | | oCVL450 | AGAAGTGGGCCAAGTGTCAG | 67 |
| CVXGA32 | CVL128 | oCVL723 | ATCGCCCTGCTGGTGCAGTGATGACGT | 68 |
| | | | ACGAGAAACACCCAATTACACTACACT | |
| | | | GGTATGACACTGTACTAACCCTGAGGG | |
| | | | TTTTAGAAA | |
| | | oCVL724 | CCTGAGGGTTTTAGAAAAAACGATTAA | 69 |
| | | | CGATAAATAAGCCCGAACACTACACACT | |
| | | | ACCTGAGGCAGCCACCATGTCAGATAAC | |
| | | | GGACCAC | |
| | | oCVL725 | GTGTGTCGATTTAAGATTGGGATCCGTA | 70 |
| | | | CGTCAAGCCTGGGTGGAG | |

SARS-CoV-2 candidate vaccines based on the CVB vector were evaluated in this study (Table 12).

TABLE 12
CVB SARS-CoV-2 vaccine candidate list virus

| Plasmid | | PIV5 | virus | SARS-CoV-2 strain, inserted gene and location, | Virus |
|---|---|---|---|---|---|
| name | vector | backbone | name | backbone modifications | obtained |
| pCVL104 | pUC | CVB | CVXGA16 | S from Wuhan strain inserted between HN and L in | ✓ |
| | | | | CVB backbone | |
| pCVL87 | pUC | CVB | CVXGA17 | S from Wuhan strain inserted between HN and L in | ✓ |
| | | | | CVB backbone | |
| pCVL85 | pUC | CVB | CVXGA18 | N from Wuhan strain inserted between F and HN, S | ✓ |
| | | | | from Wuhan strain inserted between HN and L in | |
| | | | | CVB backbone | |
| pCVL83 | pUC | CVB | CVXGA19 | N from Wuhan strain inserted between F and HN, S | ✓ |
| | | | | from Omicron BA.1 variant inserted between HN and | |
| | | | | L in CVB backbone | |
| pCVL110 | pUC | CVB | CVXGA21 | S from Omicron BA.5 variant inserted between HN | Virus |
| | | | | and L in CVB backbone | rescue |
| | | | | | ongoing |
| pCVL109 | pUC | CVB | CVXGA22 | N from Wuhan strain inserted between F and HN, S | Virus |
| | | | | from Omicron BA.5 variant inserted between HN and | rescue |
| | | | | L, backbone is CVB. | ongoing |
| pCVL112 | pUC | CVB | CVXGA24 | PIV5 F and HN genes are deleted. S from Wuhan | Virus |
| | | | | strain inserted between M and L in CVB backbone. | rescue |
| | | | | | ongoing |
| pCVL119 | pUC | CVB | CVXGA29 | N from Wuhan strain inserted between F and HN, wt S | ✓ |
| | | | | from Wuhan strain inserted between HN and L in | |
| | | | | CVB backbone | |
| pCVL120 | pUC | CVB | CVXGA30 | M from Wuhan strain inserted between F and HN, wt | ✓ |
| | | | | S from Wuhan strain inserted between HN and L in | |
| | | | | CVB backbone | |
| pCVL121 | pUC | CVB | CVXGA31 | M from Wuhan strain inserted after F of PIV5, E from | ✓ |
| | | | | Wuhan strain inserted between M of SARS-CoV-2 | |
| | | | | and HN, wt S from Wuhan strain inserted between HN | |
| | | | | and L in CVB backbone | |
| pCVL128 | pUC | CVB | CVXGA32 | M from Wuhan strain inserted after F of PIV5, N from | ✓ |
| | | | | Wuhan strain inserted between M and E of SARS- | |
| | | | | CoV-2, E from Wuhan strain inserted between N of | |
| | | | | SARS-CoV-2 and HN, wt S from Wuhan strain | |
| | | | | inserted between HN and L in CVB backbone | |

The vaccine virus sequences generated using the primers listed as described above.

CVL104 for CVXGA16: The S gene of SARS-CoV-2 Wuhan strain, S157F mutation in P/V gene, and SH open reading frame from (orf) deletion mutations were introduced into W3A backbone by Gibson assembly. The recombinant plasmid contains CVB backbone and the S gene of SARS-CoV-2 Wuhan strain between the HN and L genes of PIV5. The recombinant virus was obtained.

CVL87 for CVXGA17: The S gene of SARS-CoV-2 Wuhan strain, S157F mutation in P/V gene, and the entire SH gene transcript unit deletion mutations were introduced into W3A backbone by Gibson assembly. The recombinant plasmid contains CVB backbone and the S gene of SARS-CoV-2 Wuhan strain between the HN and L genes of PIV5. The recombinant virus was obtained.

CVL85 for CVXGA18: The N gene of SARS-CoV-2 Wuhan strain was amplified and introduced into CVL104 backbone by Gibson assembly. The recombinant plasmid contains CVB backbone, the N gene of SARS-CoV-2 Wuhan strain in the place of SH gene of PIV5, and the S gene of SARS-CoV-2 Wuhan strain between the HN and L genes of PIV5. CVXGA18 contains both the N and S genes of SARS-CoV-2 Wuhan strain in the CVB backbone. The recombinant virus was obtained.

CVL83 for CVXGA19: The S gene of SARS-CoV-2 Omicron BA.1 variant was amplified and inserted into CVL85 backbone to replace the S gene of SARS-CoV-2 Wuhan strain by Gibson assembly. The recombinant plasmid contains CVB backbone, N gene of SARS-CoV-2 Wuhan strain in the place of SH gene of PIV5, and S gene of SARS-CoV-2 Omicron BA.1 variant between the HN and L genes of PIV5. The recombinant virus was obtained.

CVL109 for CVXGA22: The S gene of SARS-CoV-2 Omicron BA.5 variant was amplified and inserted into CVL85 backbone to replace the S gene of Wuhan strain by Gibson assembly. The recombinant plasmid contains CVB backbone, N gene of SARS-CoV-2 Wuhan strain in the place of SH gene of PIV5, S gene of SARS-CoV-2 Omicron BA.5 variant between HN and L genes of PIV5. The recombinant plasmid was obtained and virus rescue is ongoing.

CVL110 for CVXGA21: The S gene of SARS-CoV-2 Omicron BA.5 variant was amplified and inserted into CVL87 backbone to replace the S gene of Wuhan strain by Gibson assembly. The recombinant plasmid contains CVB2 backbone and S gene of SARS-CoV-2 Omicron BA.5 variant between the HN and L genes of PIV5. The recombinant plasmid was obtained and virus rescue is ongoing.

CVL112 for CVXGA24: The PIV5 F and HN genes are deleted from CVL104 backbone by Gibson assembly. The recombinant plasmid contains CVB backbone without F and HN genes but the S gene of SARS-CoV-2 Wuhan strain between HN and L genes of PIV5. The recombinant plasmid was obtained and virus rescue is ongoing. The recombinant virus will only contain the glycoprotein from the SARS-COV-2 virus. This virus can serve either as the vaccine strain and/or the virus for microneutralization assay to measure S-specific antibody response.

CVL119 for CVXGA29: wt S gene of SARS-CoV-2 Wuhan strain was amplified and inserted into CVL85 backbone by Gibson assembly. The recombinant plasmid contains CVB backbone, the N gene of SARS-CoV-2 Wuhan strain in the place of SH gene of PIV5, and the wt S gene of SARS-CoV-2 Wuhan strain between the HN and L genes of PIV5. The recombinant virus was obtained.

CVL120 for CVXGA30: M gene of SARS-CoV-2 Wuhan strain was amplified and inserted into CVL119 backbone by Gibson assembly. The recombinant plasmid contains CVB backbone, the M gene of SARS-CoV-2 Wuhan strain in the place of SH gene of PIV5, and the wt S gene of SARS-CoV-2 Wuhan strain between the HN and L genes of PIV5. The recombinant virus was obtained.

CVL121 for CVXGA31: E gene of SARS-CoV-2 Wuhan strain was amplified and inserted into CVL120 backbone by Gibson assembly. The recombinant plasmid contains CVB backbone, the M gene of SARS-CoV-2 Wuhan strain in the place of SH gene of PIV5, E gene of SARS-CoV-2 Wuhan strain between M of SARS-CoV-2 and HN of PIV5, and the wt S gene of SARS-CoV-2 Wuhan strain between the HN and L genes of PIV5. The recombinant virus was obtained.

CVL128 for CVXGA32: N gene of of SARS-CoV-2 Wuhan strain was amplified and inserted into CVL121 backbone by Gibson assembly. The recombinant plasmid contains CVB backbone, the M gene of SARS-CoV-2 Wuhan strain in the place of SH gene of PIV5, N gene of SARS-CoV-2 Wuhan strain between M and E of SARS-CoV-2, E gene of SARS-CoV-2 Wuhan strain between N of SARS-CoV-2 and HN of PIV5, and the wt S gene of SARS-CoV-2 Wuhan strain between the HN and L genes of PIV5. The recombinant virus was obtained.

### Example 6: Immunogenicity of CVB Based COIVD-19 Vaccines in Murine and Hamster Models

To evaluate the immunogenicity of CVXGA16, PIV5 CVB with the SH open reading frame (ORF) deletion encoding the S protein from the Wuhan strain, CVXGA17, PIV5 CVB with the entire SH gene deletion encoding the S protein from the Wuhan strain, CVXGA18, PIV5 CVB with the SH ORF deletion encoding the N and S protein from the Wuhan strain compared to CVXGA1 in a Balb/c murine model (AE26) and Golden Syrian hamster model (AE25).

### Materials and Methods

For AE26, twenty-five female Balb/c mice were used in this study (N=5 each group). The mice were immunized intranasally with 50 μL of PBS or 10⁵ plaque forming units (PFU) of CVXGA1, CVXGA16, CVXGA17 and CVXGA18. At 32 days post-immunization (dpi), blood and spleens were collected. Anti-S or S RBD IgG antibodies were determined, as well as levels of IFNγ-secreting cells.

For AE25, twenty male Golden Syrian hamsters were used in this study (N=5 for group 1 and N=15 for Group 2 (COVID-19 mRNA). The hamsters were immunized intramuscularly with 50 μL of PBS (Group 1) or with 2 μg of COVID-19 mRNA vaccine per hamster (group 2). At 21 days post-immunization (dpi), the hamsters from Group 2 were boosted intramuscularly with 2 μg of COVID-19 mRNA. At day 42 post-immunization, hamsters from Group 1 were boosted intranasally with 50 μL of PBS, and hamsters from Group 2 were boosted a second time intramuscularly with 2 μg of COVID-19 mRNA (Group 2A), or intranasally with 2×10⁶ PFU of CVXGA1 (Group 2B), or CVXGA18 (Group 2C). At 63 days post-immunization, blood was collected. Anti-S IgG antibodies were determined.

### Results

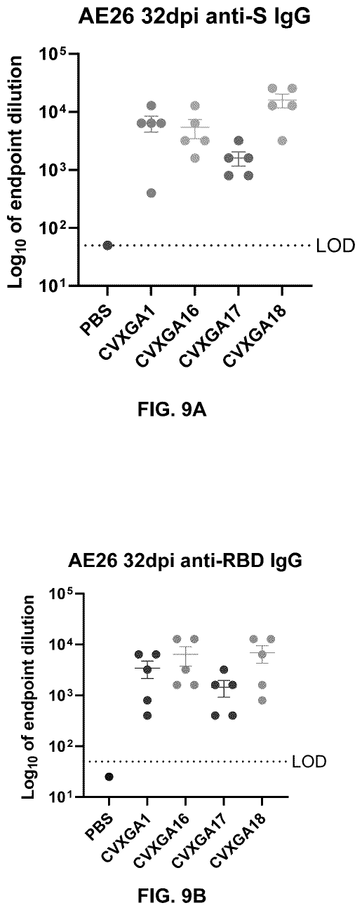

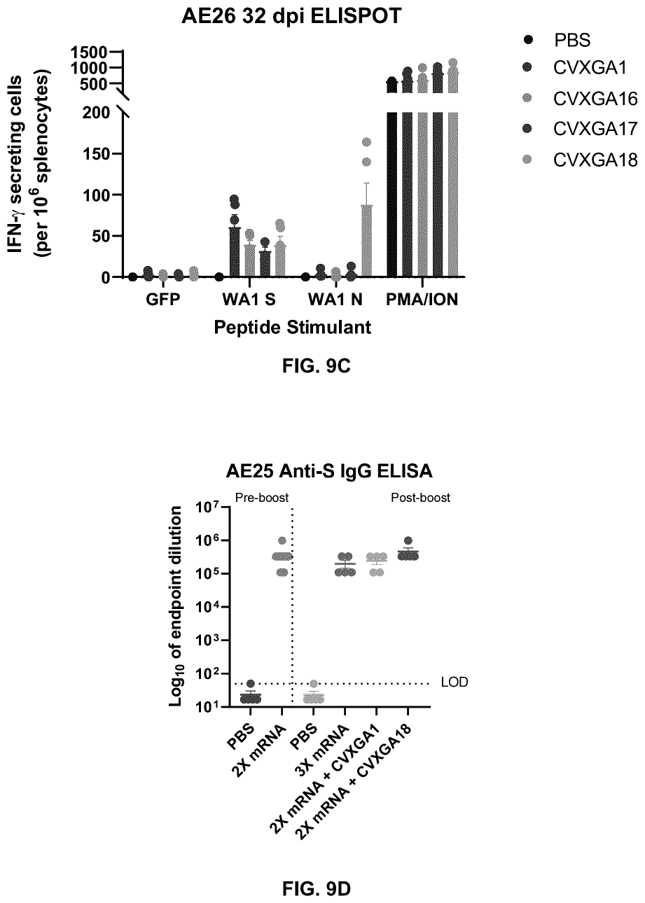

From AE26 study, CVXGA16, CVXGA17 and CVXGA18 vaccinated mice produced comparable IgG antibodies against S protein or S-RBD domain compared to CVXGA1 (FIG. 9A-9B). Animals from all vaccine groups elicited similar levels of S protein-specific IFNγ-secreting cells, with CVXGA1 group having slightly higher levels compared to other groups. Animals from CVXGA18-vaccinated group were the only ones to elicit N protein-specific IFNγ-secreting cells (FIG. 9C). From AE25 study, CVXGA1 or CVXGA18 boosted hamsters had comparable IgG antibodies against S protein compared to 3×COVID-19 mRNA group (FIG. 9D).

CVXGA16, CVXGA17 and CVXGA18 are immunogenic in Balb/c mice. CVXGA18 is also immunogenic in hamsters when given as a booster to hamsters that have received prime-boost immunization with COVID-19 mRNA vaccine.

### Example 7: CVB Based SARS-CoV-2 Vaccine Candidates

### Materials and Methods

We successfully rescued CVB based SARS-CoV-2 viruses expressing multiple antigens, CVXGA29, CVXGA30, CVXGA31, and CVXGA32 as indicated in Table 13. These rescued viruses were grown in T75 flasks of Vero cells with titer>7.2 Log 10 PFU/mL.

TABLE 13
PIV5 based SARS-CoV-2 vaccine virus candidates.

| plasmid | Virus | Vector and antigen | titer Log₁₀ PFU/mL |
|---|---|---|---|
| pCVL6 | CVXGA1 | CPI-S_{F tail} | 7.4 |
| pCVL87 | CVXGA17 | CVB-S_{F tail} | 7.7 |
| pCVL85 | CVXGA18 | CVB-N-S_{F tail} | 7.5 |
| pCVL119 | CVXGA29 | CVB-N-S_{WT} | 7.9 |
| pCVL120 | CVXGA30 | CVB-M-S_{WT} | 7.5 |
| pCVL121 | CVXGA31 | CVB-M-E-S_{WT} | 7.4 |
| pCVL122 | CVXGA32 | CVB-M-N-E-S_{WT} | 7.2 |

Viral replication in Vero cells: Vero cells in 6-well plates were infected with CVXGA17, CVXGA29, CVXGA30, CVXGA31, and CVXGA32 at a multiplicity of infection (MOI) of 0.1. The cells were then washed with PBS and maintained in DMEM-2% FBS. Media were collected at 1-6 days post infection (dpi). The titers of viruses were determined by plaque assay on Vero cells.

Mice study: To determine whether these viruses were immunogenic in vivo, five- to 7-week-old female Balb/c mice (Envigo) were used in the immunogenicity study. Following quarantine, five female mice were randomly grouped into 7 study groups (Table 14). Each mouse was anesthetized by intraperitoneal injection of 250 or 300 μl of Avertin (2,2,2-tribromoethanol in tert-amyl alcohol) and was inoculated intranasally with 100 μl (50 μl per nostril) of PBS, 1.7×10⁵ PFU of CVXGA1, CVXGA18, CVXGA29, CVXGA30, CVXGA31, or CVXGA32, and housed in HEPA-filtered isolators under ABSL2. Twenty-one days after immunization, the animals were euthanized, and blood was collected via cardiothoracic bleeds for serum isolation. All experiments were performed in the animal facility at the University of Georgia in accordance with protocols approved by the IACUC at the University of Georgia.

TABLE 14
Mouse immunogenicity study

| Group | D 0 | Dosage/volume/route | Procedures |
|---|---|---|---|
| 1 | PBS | 100 ul/in | Collect |
| 2 | CVXGA1 | 1.7 × 10⁵/100 ul/in | serum on |
| 3 | CVXGA18 | | D 21 |
| 4 | CVXGA29 | | |
| 5 | CVXGA30 | | |
| 6 | CVXGA31 | | |
| 7 | CVXGA32 | | |

ELISA: To quantify the anti-SARS-CoV-2-S or N antibody response, mouse serum was analyzed via enzyme-linked immunosorbent assay (ELISA). Immulon 2HB 96-well microtiter plates were coated with 25 ng of purified SARS-CoV-2-S trimmer protein or N protein for 24 hours. Mouse serum samples were inactivated at 56° C. and prediluted in 1:100 dilution then serially diluted in 2-fold dilutions and incubated on the plates for 1 hour. Following washes, the plates were incubated with horseradish peroxidase (HRP)-labeled goat anti-mouse IgG secondary antibody at 1:1250 dilution for 1 hour. The plates were developed with KPL SureBlue Reserve TMB microwell peroxidase substrate, and OD450 (optical density at 450 nm) values were read with a BioTek Epoch microplate spectrophotometer (BioTek, Winooski, VT). Antibody titers were calculated as the highest serum dilution at which the OD450 value was greater than 3 SD above the mean OD450 value of the negative controls.

### Results

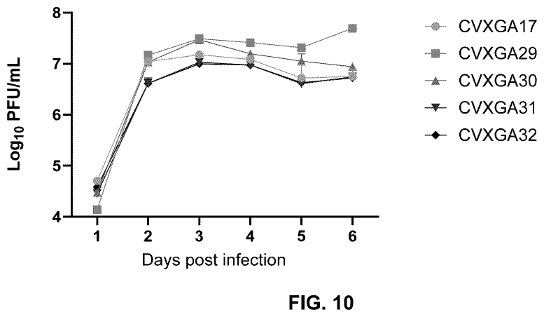

Growth curve: CVXGA29, CVXGA30, CVXGA31, and CVXGA32 expressing wt SARS-CoV-2 S grew to similar titers compared to CVXGA17 expressing SARS-CoV-2 S with PIV5 F tail (FIG. 10).

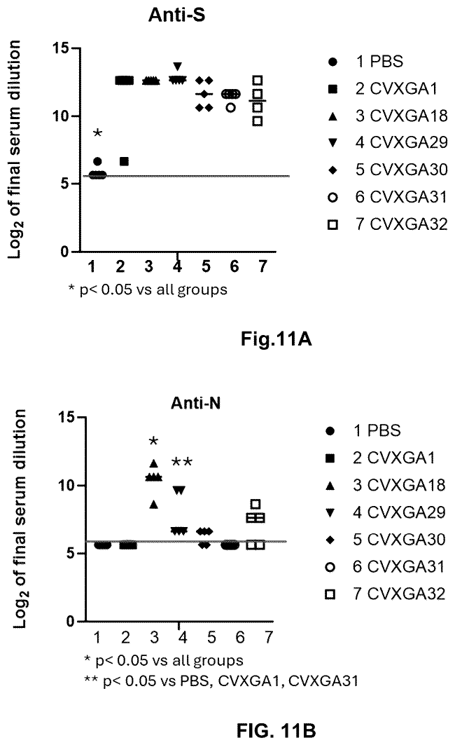

Immunogenicity: For anti-SARS-CoV-2 S immunogenicity (FIG. 11A), CVXGA1, CVXGA18, CVXGA29, CVXGA30, CVXGA31, or CVXGA32 elicited similar levels of antibody titers. There was no significant difference between vaccine groups.

For anti-SARS-CoV-2 N immunogenicity (FIG. 11B), CVXGA1, CVXGA30, and CVXGA31 without N gene insertion did not elicit N-specific antibody as expected. CVXGA18 expressing N and S with PIV5 F tail, CVXGA29, and CVXGA32 expressing N and wt S elicited N-specific antibody titers. CVXGA18 induced significantly high levels of antibodies compared to all other groups, CVXGA29 induced significantly high levels of antibodies compared to PBS, CVXGA1 and CVXGA31. CVXGA32 elicited lower levels of anti-N antibodies, there was no difference compared to other groups except CVXGA18. There was no significant difference between CVXGA29 and CVXGA32 which contains N and wt S.

From the study, we concluded that PIV5 is a good vector to express up to 4 antigens with total length of ˜6.4 kb. All recombinant viruses grew to titer >7 Log₁₀ PFU/mL in Vero cells. Anti-S immunogenicity from these viruses was similar. Anti-N immunogenicity from CVXGA29 and CVXGA32 was similar, but lower than that of CVXGA18 expressing N and S with PIV5 F tail. It is possible that S with PIV5 F tail may enhance immunogenicity of other genes.

### Example 8: CVB-RSV-F Pre-MVS RSV F Percent Expression

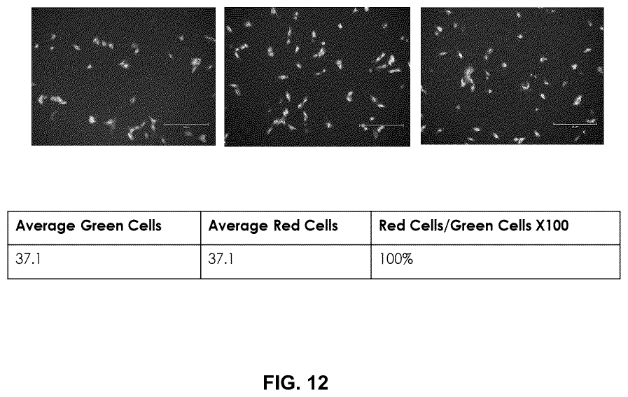

FIG. 12 show the determination of percent F expression in CVB-RSV-F infected cells. Vero-SF cells were infected with CVB-RSV-F pre-MVSS virus at dilutions −3 to −5. After 1-hour incubation at 37° C., media was changed, and cells were incubated for 18 hours at 37° C. Immunostaining was performed using mouse anti-CVB-HN and human anti-F (Palivizumab) antibodies followed by anti-mouse Alexa 488 and anti-human Cy3 secondary antibodies, respectively. These are representative images of wells infected with CVB-RSV-F pre-MVSS showing both green and red cells. The table shows the percent expression of RSV F protein in CVB-infected cells. Images were taken at 10×. Every infected cells (100%) expressed RSV F protein.

### Example 9: CVB-RSV-F Pre-MVS Growth Kinetics

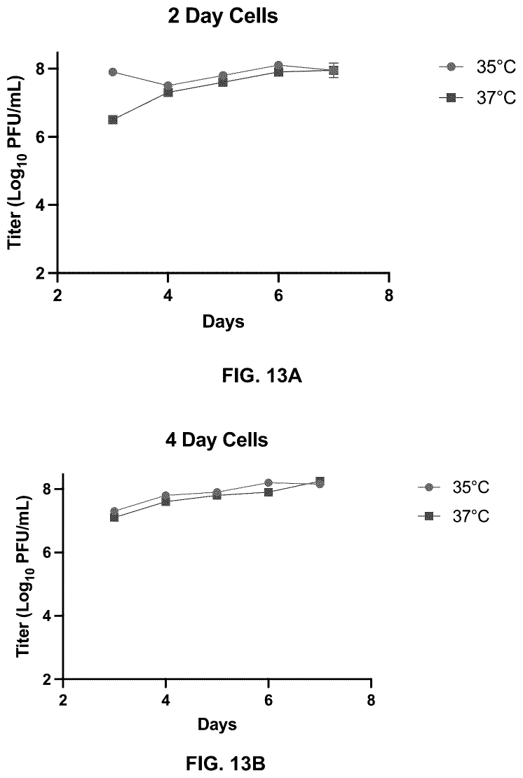

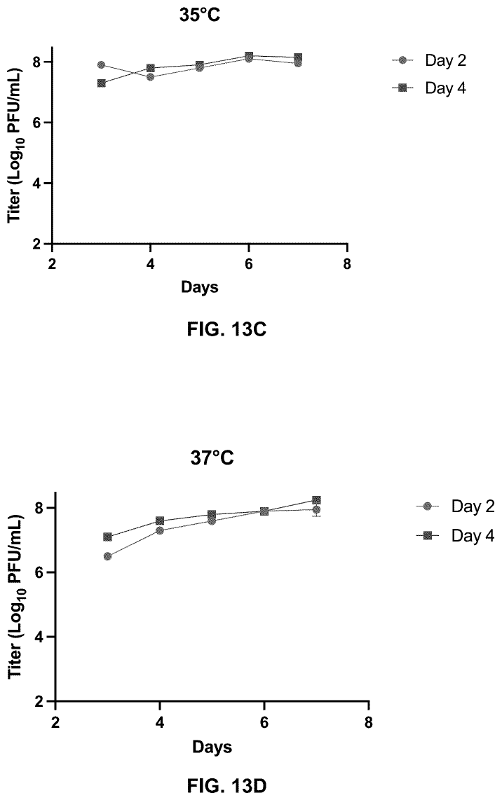

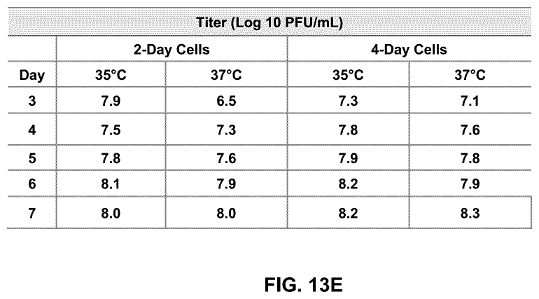

FIGS. 13A-13E show the replication of BLB-205 at 35° C. vs 37° C. and day 2 vs day 4 serum free Vero cells. The data shows that collection can be done at day 6 or 7 and at both at 35° C. and 37° C. Day 2 and Day 4 Vero cells produced similar amount of viruses.

The disclosure provided herein include the generation of a more potent and high yield PIV5 CVB backbone which contains the P/V gene S157F mutation or alternatively S156N mutation, the PIV5 SH deletion as either open reading frame or the entire S gene transcription unit in the PIV5 W3A strain. Multiple SARS-CoV-2 vaccine candidates in the CVB backbone have been produced showing that the antigenic genes can be inserted between the SH and HN gene junctions and/or the HN-L gene junctions without significantly affecting virus antigen expression. Both the N and S genes (˜5 kb) have been successfully inserted into CVB backbone, reaching titer of ˜10⁸ PFU/ml.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A composition comprising a modified PIV5 CVB viral expression vector having at least 98% sequence identity to SEQ ID NO: 1, wherein the CVB viral expression vector comprises mutations of a PIV5 viral genome at amino acid residue S157 or S156 of the P/V gene resulting in the removal of a phosphorylation site and higher transcription activities thereby improving virus titer in cell culture, and wherein the CVB viral expression vector is highly immunogenic and can be used as an effective vaccine platform.

2. The composition of claim 1, wherein the mutation at amino acid residue S157 or S156 comprises the substitution of serine (S) with an amino acid residue selected from a group consisting of alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), selenocysteine (U), valine (V), tryptophan (W), and tyrosine (Y).

3. The composition of claim 1, wherein the mutation at amino acid residue S157 or S156 comprises the substitution of the serine (S) amino acid residue S157 to phenylalanine (F) or the substitution of the serine (S) amino acid residue S156 to asparagine (N).

4. The composition of claim 1, wherein the CVB viral expression vector expresses a heterologous polypeptide comprising a viral antigen selected from a group consisting of SARS-CoV-2, RSV or viral or bacterial antigens.

5. The composition of claim 1, wherein the PIV5 genome has a heterologous nucleic acid sequence with at least 98% sequence identity to SEQ ID NOs: 2, 3, 4, 5, 6, 7, 8, 9, 10, 11, 12, or 13 and wherein the viral expression vector expresses a heterologous polypeptide comprising a coronavirus spike (S) and/or nucleocapsid (N) proteins or the RSV-F protein.

6. The composition of claim 5, wherein the coronavirus spike (S) and/or nucleocapsid (N) proteins are S or N proteins of a severe acute respiratory syndrome coronavirus 2 (SARS-CoV-2), a SARS-CoV-2 Wuhan strain, or a variant of SARS-CoV-2, where the variant of SARS-CoV-2 is a SARS-CoV-2 beta variant, a SARS-CoV-2 gamma variant, a SARS-CoV-2 delta variant, a SARS-CoV-2 omicron variant, a SARS-CoV-2 Omicron BA.1, a SARS-CoV-2 Omicron BA.5, BQ1 or XBB1 or any future emerging variants.

7. The composition of claim 5, wherein the coronavirus S gene comprises the coronavirus S gene of SARS-CoV-2 and wherein a cytoplasmic tail of the coronavirus S gene has been replaced with a cytoplasmic tail of the fusion (F) gene of the CVB or a CPI vector.

8. The composition of claim 5, wherein the PIV5 genome with S157F or S156N in the P/V gene of the PIV5 viral genome (CVB) further comprises an open reading frame deletion mutations of a SH gene, and wherein the N gene of the SARS-CoV-2 Wuhan strain is inserted to replace the SH gene of PIV5 and the S gene of SARS-CoV-2 Wuhan strain or other variant strains is inserted between a PIV5 hemagglutinin (HN) gene and a polymerase (L) gene of CVB.

9. The composition of claim 5, wherein an entire SH gene transcript unit of a PIV5 viral genome is deleted and the S gene of the SARS-CoV-2 Wuhan strain or other variant strains is placed between a HN gene and a L gene of CVB.

10. The composition of claim 5, wherein the S gene of the SARS-CoV-2 variant is inserted to replace the S gene of the SARS-CoV-2 Wuhan strain and the N gene of the SARS-CoV-2 Wuhan strain is inserted in the place of the SH gene of PIV5.

11. The composition of claim 5, wherein the S gene of the SARS-CoV-2 Omicron BA.5 variant is inserted between the HN and L genes of PIV5 to replace the S gene of Wuhan strain.

12. The composition of claim 5, wherein the S gene of the SARS-CoV-2 Omicron BA.5 variant is inserted to replace the S gene of the SARS-CoV-2 Wuhan strain and the N gene of SARS-CoV-2 Wuhan strain is inserted to replace the SH gene of CVB.

13. The composition of claim 5, wherein the PIV5 F and HN genes are deleted and wherein the S gene of SARS-CoV-2 Wuhan strain is between HN and L genes of CVB.

14. The composition of claim 5, wherein the N gene of the SARS-CoV-2 Wuhan strain is inserted between F and HN, and the S gene of the SARS-CoV-2 Wuhan strain is inserted between the HN and L genes of CVB.

15. The composition of claim 5, wherein the M gene from the SARS-CoV-2 Wuhan strain is inserted between F and HN, and the S gene of the SARS-CoV-2 Wuhan strain is inserted between HN and L of CVB.

16. The composition of claim 5, wherein the M gene from the SARS-CoV-2 Wuhan strain is inserted after F of PIV5, the E gene from the SARS-CoV-2 Wuhan strain inserted between the M gene of SARS-CoV-2 and HN, and the S gene of SARS-CoV-2 Wuhan strain is inserted between HN and L of CVB.

17. The composition of claim 5, wherein the M gene from the SARS-CoV-2 Wuhan strain is inserted after F of PIV5, the N gene from the SARS-CoV-2 Wuhan strain is inserted between the M gene and the E gene of SARS-CoV-2, the E gene from the SARS-CoV-2 Wuhan strain inserted between the N gene of SARS-CoV-2 and HN, and the S gene of SARS-CoV-2 Wuhan strain is inserted between HN and L of CVB.

18. The composition of claim 5, wherein the RSV-F protein in inserted in place of PIV5 SH gene (ASH) or inserted between PIV5 SH and NH (SH-NH) or between HN and L (HN-L).

19. A method of inducing an immune response in a subject having or at risk of having SARS-COV-2, RSV or other viral or bacterial infections, the method comprising administering the composition of claim 1 to the subject as a primary vaccine or a booster vaccine, wherein the immune response comprises a humoral immune response and/or a cellular immune response.

20. The method of claim 19, wherein the composition is administered intranasally, intramuscularly, topically, or orally.
